(19) United States
(12) Patent Application Publication
(10) Pub. No.: US 2010/0022559 A1
(43) Pub. Date: Jan. 28, 2010

(54) BISOXIMES AS FUNGICIDES

(75) Inventors: Silvia Cerezo-Galvez, Langenfeld (DE); Mazen Es-Sayed, Langenfeld (DE); Herbert Gayer, Monheim (DE); Oliver Guth, Leverkusen (DE); Stefan Herrmann, Langenfeld (DE); Kerstin Ilg, Koln (DE); James Darren Mansfield, Kurten (DE); Amos Mattes, Langenfeld (DE); Ahmed Wahed Moradi, Monheim (DE); Hans-Georg Schwarz, Langenfeld (DE); Thomas Seitz, Langenfeld (DE); Astrid Ullmann-Koppold, Leverkusen (DE); Klaus-Günther Tietjen, Langenfeld (DE); Ulrike Wachendorff-Neumann, Neuwied (DE); Peter Dahmen, Neuss (DE); Arnd Voerste, Koln (DE); Eva-Maria Franken, Lyon (FR); Olga Malsam, Rosrath (DE); Peter Lösel, Leverkusen (DE); Anne Suty-Heinze, Langenfeld (DE)

Correspondence Address: Baker Donelson Bearman, Caldwell & Berkowitz, PC, 555 Eleventh Street, NW, Sixth Floor, Washington, DC, 20004, US

(73) Assignee: BAYER CROPSCIENCE AG, Monheim (DE)

(21) Appl. No.: 12/520,211

(22) PCT Filed: Dec. 11, 2007
(86) PCT No.: PCT/EP07/10798

§ 371 Date: Sep. 25, 2009

(30) Foreign Application Priority Data
Dec. 19, 2006 (DE) 102006059970.5
May 16, 2007 (DE) 102007023102.6

(51) Int. Cl. A01N 43/54; A01N 43/40; C07D 403/12; C07D 413/12

(52) U.S. Cl. 514/256; 514/340; 544/333; 546/272.1

(57) ABSTRACT

The invention relates to the novel use of bisoximes, some of which are known, for controlling unwanted microorganisms, and to processes for their preparation, and also to novel bisoximes, to processes for their preparation and to their use for controlling unwanted microorganisms and insects in crop protection and in the protection of materials.

The invention relates to the novel use of bisoximes, some of which are known, for controlling unwanted microorganisms, and to processes for their preparation, and also to novel bisoximes, to processes for their preparation and to their use for controlling unwanted microorganisms and insects in crop protection and in the protection of materials.

It is already known that certain bisoximes can be used in crop protection. Thus, DE-A 42 04 205 describes the use of cyclohexenone oxime ethers as herbicides. DE-A 28 20 361 describes alkanyl-azolyl oxime carbamates and DE-A 42 13 149 describes (hetero)aryl alkyl ketone oxime O-ethers, in each case as insecticidally or acaricidally or nematocidally active compounds. The use of pyrimidine derivatives which may be substituted by two oximes as MLR (mixed lymphocyte reaction) inhibitors is described in WO 03/091223. In the context of screening methods for discovering new lead structures, WO 99/49314 lists bisoximes as examples, but a fungicidal use of such compounds is not described.

The synthesis of various bisoxime derivatives is described in the literature. Thus, for example, alkyl heteroaryl ketoximes are reacted with 1,2-dichloroethane in superbasic medium to give 1,2-bis(alkylideneaminooxy)ethanes (Khimiya Geterotsiklicheskikh Soedinenii 1989, 7, 901; Zhurnal Organicheskoi Khimii 1988, 24, 2538). The preparation of methylene dioxime derivatives via phase-transfer catalysis is demonstrated in J. Org. Chem. 1979, 44, 3970. Bisaldoximes as chemotherapeutics are described in Arzneim.-Forsch. 1977, 27, 2251. J. Heterocyclic Chem. 1979, 16, 1459 also mentions bisaldoxime structures. A fungicidal action is likewise not described.

Since the ecological and economical demands made on modern active compounds, for example fungicides, are increasing constantly, for example with respect to activity spectrum, toxicity, selectivity, application rate, formation of residues and favourable manufacture, and there can furthermore be problems, for example, with resistance, there is a constant need to develop novel fungicides which, at least in some areas, have advantages over those of the prior art. Surprisingly, it has now been found that bisoximes of the formula (I) (see below) are suitable as fungicides and, at least in some aspects, have improved properties compared to known fungicidally active compounds.

It has now been found that bisoximes of the general formula (I)

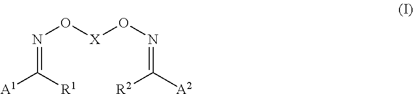

in which
R¹ and R² independently of one another represent hydrogen, halogen, C₁-C₈-alkyl, C₃-C₈-cycloalkyl, C₁-C₈-haloalkyl, OH, C₁-C₈-alkoxy, (C₁-C₆-alkoxy)carbonyl, SH, C₁-C₈-alkylthio, C₁-C₈-alkylsulphinyl, C₁-C₈-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₈-alkyl)amino, (C₁-C₈-alkyl)amino, C₂-C₈-alkenyl, C₂-C₈-alkynyl, in each case optionally substituted phenyl, benzyl, heterocyclyl or hetaryl,
A¹ and A² independently of one another represent one of the cycles below

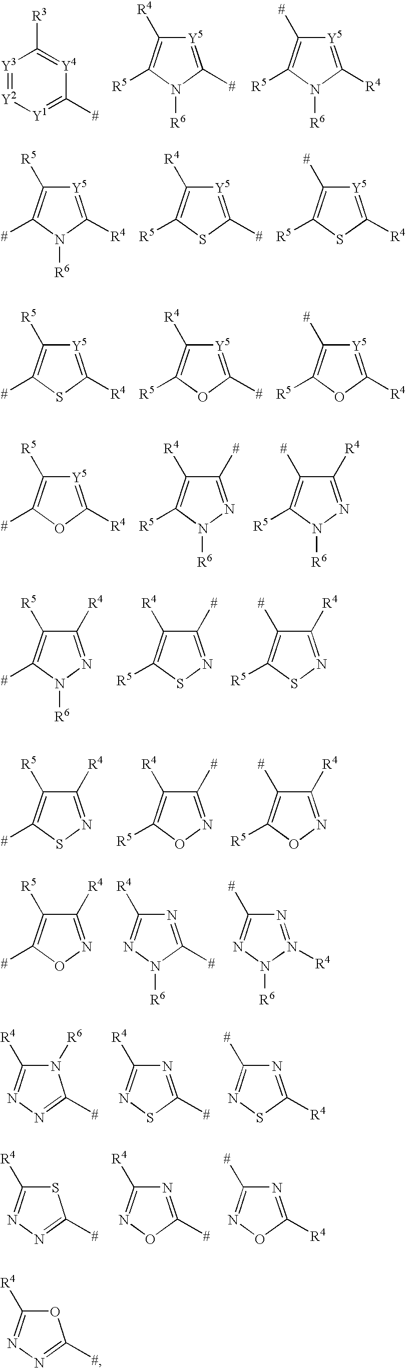

where A¹ and A² do not simultaneously represent optionally substituted phenyl,
Y¹ represents N or CR⁶,
Y² represents N or CR⁷,
Y³ represents N or CR⁸,
Y⁴ represents N or CR⁹,
Y⁵ represents N or CR¹⁰,
R³, R⁴, R⁵, R⁶, R⁷, R⁸, R⁹ and R¹⁰ independently of one another represent hydrogen, halogen, cyano, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹OR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, phenyl-(di-C₁-C₄-alkyl)-silyl, substituted or unsubstituted phenyl or hetaryl, substituted or unsubstituted benzyl or —CH₂-hetaryl,
   moreover represent substituted C₂-C₈-alkenyl or C₂-C₈-alkynyl,
   moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together with the carbon atoms to which they are attached form an unsubstituted or substituted unsaturated or fully or partially saturated heterocyclic or carbocyclic 5- to 7-membered ring,
R¹¹ represents hydrogen, unsubstituted or substituted C₁-C₈-alkyl, C₂-C₈-alkenyl, C₂-C₈-alkynyl, C₁-C₈-haloalkyl, C₃-C₈-cycloalkyl, tri-C₁-C₄-alkyl-silyl, in each case substituted or unsubstituted aryl, —CH₂-aryl, hetaryl or —CH₂-hetaryl,
   moreover represents C₃-C₈-cycloalkyl-C₁-C₄-alkyl,
   where, if two radicals R¹¹ are attached to a carbon or nitrogen atom, these radicals R¹¹ can be identical or different,
   moreover, if two radicals R¹¹ are attached to a nitrogen atom, these two radicals R¹¹ together with the nitrogen atom to which they are attached form a 3- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
   moreover the two radicals R¹¹ in the grouping NR¹¹COR¹¹ together with this grouping to which they are attached form a 4- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
R¹² represents hydrogen, unsubstituted or substituted C₁-C₈-alkyl, C₁-C₈-haloalkyl, —(CH₂)ₙOR¹¹, tri-C₁-C₄-alkyl-silyl,
m represents the number 1, 2, 3, 4, 5, 6, 7 or 8,
n represents the number 1, 2, 3 or 4,
X represents one of the groupings X-1 to X-10 below

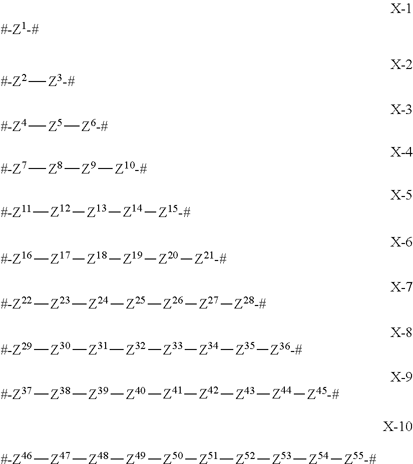

in which
Z¹ represents CR¹³R¹⁴ or SiR¹⁵R¹⁶, where in each case two radicals R¹³, R¹⁴ or R¹⁵, R¹⁶ together with the carbon or silicon atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z² and Z³ independently of one another represent CR¹³R¹⁴ or SiR¹⁵R¹⁶, but two silicon atoms are not adjacent, where in each case two radicals R¹³, R¹⁴ or R¹⁵, R¹⁶ or R¹³, R¹⁵ together with the carbon or silicon atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z⁴ and Z⁶ independently of one another represent CR¹³R¹⁴ or SiR¹⁵R¹⁶ and Z⁵ represents CR¹³R¹⁴, SiR¹⁵R¹⁶, O or NR¹⁷, but two silicon atoms are not adjacent, or in each case two adjacent radicals Z⁴, Z⁵ or Z⁵, Z⁶ represent a group selected from the group consisting of —CR¹³═CR¹⁴—, —CR¹³═N—, —N═CR¹³— or —C≡C—,
   where X-3 may contain at most one of those groups,
   where in each case two radicals R¹³, R¹⁴ or R¹⁵, R¹⁶ or R¹³, R¹⁵ or R¹³R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z⁷ and Z¹⁰ independently of one another represent CR¹³R¹⁴ or SiR¹⁵R¹⁶ and Z⁸ and Z⁹ independently of one another represent CR¹³R¹⁴, SiR¹⁵R¹⁶, O or NR¹⁷, but two silicon or two oxygen atoms are not adjacent, or in each case two adjacent radicals Z⁷, Z⁸ or Z⁸, Z⁹ or Z⁹, Z¹⁰ represent a group selected from the group consisting of —CR¹³═CR¹⁴—, —CR¹³═N—, —N═CR¹³— or —C≡C—, where X-4 may contain at most one of those groups,
   where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z¹¹ and Z¹⁵ independently of one another represent CR¹³R¹⁴ or SiR¹⁵R¹⁶ and Z¹², Z¹³ and Z¹⁴ independently of one another represent CR¹³R¹⁴, SiR¹⁵R¹⁶, O or NR¹⁷, but two silicon or two oxygen atoms are not adjacent, or in each case two adjacent radicals Z¹¹, Z¹² or Z¹², Z¹³ or Z¹³, Z¹⁴ or Z¹⁴, Z¹⁵ represent a group selected from the group consisting of —CR¹³═CR¹⁴—, —CR¹³═N—, —N═CR¹³— or —C≡C—, where X-5 may contain not more than 2 of those groups, which for their part must not be cumulated,
   where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z¹⁶ and Z²¹ independently of one another represent CR¹³R¹⁴ or SiR¹⁵R¹⁶ and Z¹⁷, Z¹⁸, Z¹⁹ and Z²⁰ independently of one another represent CR¹³R¹⁴, SiR¹⁵R¹⁶, O or NR¹⁷, but two silicon or two oxygen atoms are not adjacent, or in each case two adjacent radicals Z¹⁶, Z¹⁷ or Z¹⁷, Z¹⁸ or Z¹⁸, Z¹⁹ or Z¹⁹, Z²⁰ or Z²⁰, Z²¹ represent a group selected from the group consisting of —CR¹³═CR¹⁴—, —CR¹³═N—, —N═CR¹³— or —C≡C—, where X-6 may not contain more than 2 of those groups, which for their part must not be cumulated, where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z²² and Z²⁸ independently of one another represent CR¹³R¹⁴ or SiR¹⁵R¹⁶ and Z²³, Z²⁴, Z²⁵, Z²⁶ and Z²⁷ independently of one another represent CR¹³R¹⁴, SiR¹⁵R¹⁶, O or NR¹⁷, but two silicon or two oxygen atoms are not adjacent, or in each case two adjacent radicals Z²², Z²³ or Z²³, Z²⁴ or Z²⁴, Z²⁵ or Z²⁵, Z²⁶ or Z²⁶, Z²⁷ or Z²⁷, Z²⁸ represent a group selected from the group consisting of —CR¹³═CR¹⁴—, —CR¹³═N—, —N═CR¹³— or —C≡C—, where X-7 may not contain more than 2 of those groups, which for their part must not be cumulated,
   where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z²⁹ and Z³⁶ independently of one another represent CR¹³R¹⁴ or SiR¹⁵R¹⁶ and Z³⁰, Z³¹, Z³², Z³³, Z³⁴ and Z³⁵ independently of one another represent CR¹³R¹⁴, SiR¹⁵R¹⁶, O or NR¹⁷, but two silicon or two oxygen atoms are not adjacent, or in each case two adjacent radicals Z²⁹, Z³⁰ or Z³⁰, Z³¹ or Z³¹, Z³² or Z³², Z³³ or Z³³, Z³⁴ or Z³⁴, Z³⁵ or Z³⁵, Z³⁶ represent a group selected from the group consisting of —CR¹³═CR¹⁴—, —CR¹³═N—, —N═CR¹³— or —C≡C—, where X-8 may not contain more than 2 of those groups, which for their part must not be cumulated,
   where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z³⁷ and Z⁴⁵ independently of one another represent CR¹³R¹⁴ or SiR¹⁵R¹⁶ and Z³⁸, Z³⁹, Z⁴⁰, Z⁴¹, Z⁴², Z⁴³ and Z⁴⁴ independently of one another represent CR¹³R¹⁴, SiR¹⁵R¹⁶, O or NR¹⁷, but two silicon or two oxygen atoms are not adjacent, or in each case two adjacent radicals Z³⁷, Z³⁸ or Z³⁸, Z³⁹ or Z³⁹, Z⁴⁰ or Z⁴⁰, Z⁴¹ or Z⁴¹, Z⁴² or Z⁴², Z⁴³ or Z⁴³, Z⁴⁴ or Z⁴⁴, Z⁴⁵ represent a group selected from the group consisting of —CR¹³═CR¹⁴—, —CR¹³═N—, —N═CR¹³— or —C≡C—, where X-9 may not contain more than 2 of those groups, which for their part must not be cumulated,
   where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z⁴⁶ and Z⁵⁵ independently of one another represent CR¹³R¹⁴ or SiR¹⁵R¹⁶ and Z⁴⁷, Z⁴⁸, Z⁴⁹, Z⁵⁰, Z⁵¹, Z⁵², Z⁵³ and Z⁵⁴ independently of one another represent CR¹³R¹⁴, SiR¹⁵R¹⁶, O or NR¹⁷, but two silicon or two oxygen atoms are not adjacent, or in each case two adjacent radicals Z⁴⁶, Z⁴⁷ or Z⁴⁷, Z⁴⁸ or Z⁴⁸, Z⁴⁹ or Z⁴⁹, Z⁵⁰ or Z⁵⁰, Z⁵¹ or Z⁵¹, Z⁵² or Z⁵², Z⁵³ or Z⁵³, Z⁵⁴ or Z⁵⁴, Z⁵⁵ represent a group selected from the group consisting of —CR¹³═CR¹⁴—, —CR¹³═N—, —N═CR¹³— or μC≡C—, where X-10 may not contain more than 2 of those groups, which for their part must not be cumulated,
   where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
      and in which the groupings X-2 to X-10 contain at most one ring which, as ring members, contains either only one of the radicals Z² to Z⁵⁵ (spiro compounds) or two radicals Z² to Z⁵⁵ or three radicals Z² to Z⁵⁵ or four radicals Z² to Z⁵⁵,
R¹³, R¹⁴, R¹⁵, R¹⁶ and R¹⁷ independently of one another represent hydrogen, halogen, cyano, hydroxyl, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹⁵SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², unsubstituted or substituted C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, substituted or unsubstituted phenyl or hetaryl, substituted or unsubstituted benzyl or —CH₂-hetaryl,
R¹³ and R¹⁴ moreover together represent ═CR¹⁸R¹⁹, ═O, ═S or ═NOR²⁰,
R¹⁸, R¹⁹ and R²⁰ independently of one another represent hydrogen, unsubstituted or substituted C₁-C₈-alkyl, C₁-C₈-haloalkyl, C₂-C₈-alkenyl, C₂-C₈-alkynyl, C₃-C₈-cycloalkyl, substituted or unsubstituted phenyl or hetaryl,
R¹⁸ and R¹⁹ moreover together with the carbon atom to which they are attached form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
   are suitable for controlling unwanted microorganisms in crop protection and the protection of materials.

Furthermore, it has been found that bisoximes of the formula (I) are obtained when

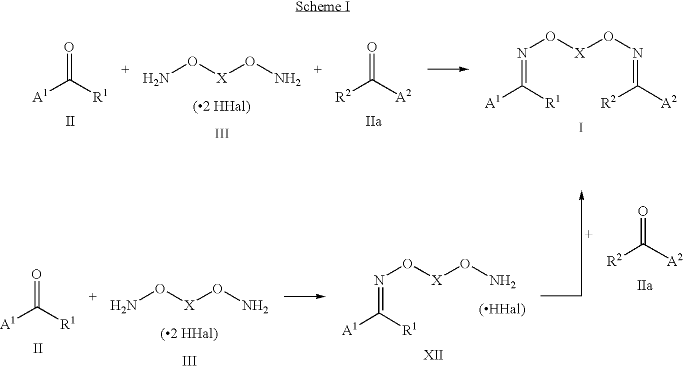

(A-1) acyl(het)aryls of the formula (II)

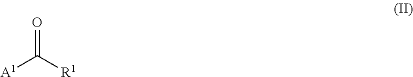

in which A¹ and R¹ have the meanings given above
are reacted with acyl(het)aryls of the formula (IIa)

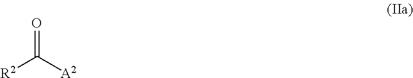

in which R² and A² have the meanings given above
and with bishydroxylamines (or bishydrohalides thereof) of the formula (III)

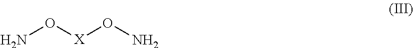

in which X has the meanings given above,
if appropriate in the presence of a catalyst and if appropriate in the presence of a diluent (cf. Scheme 1),
   or
   (A-2) acyl(het)aryls of the formula (II)

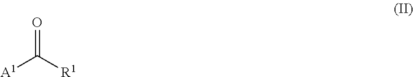

in which A¹ and R¹ have the meanings given above,
are reacted initially with bishydroxylamines (or bishydrohalides thereof) of the formula (III)

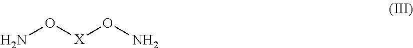

in which X has the meanings given above,
to give hydroxylamines (or hydrohalides thereof) of the formula (XII)

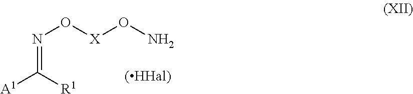

in which A¹, R¹ and X have the meanings given above
and then with acyl(het)aryls of the formula (IIa)

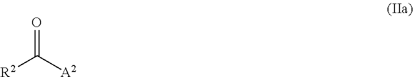

in which R² and A² have the meanings given above,
if appropriate in the presence of a catalyst and if appropriate in the presence of a diluent (cf. Scheme 1),
   or

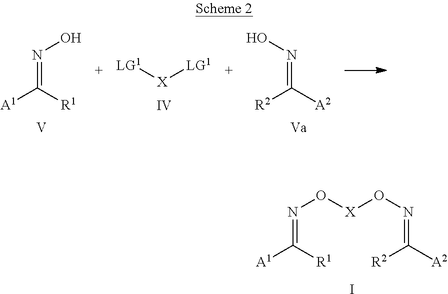

(B) free oximes of the formula (V)

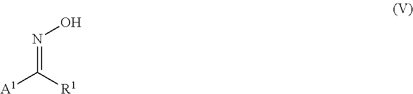

in which A¹ and R¹ have the meanings given above
are reacted with free oximes of the formula (Va)

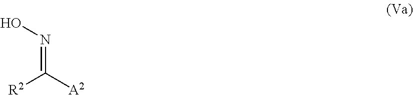

in which R² and A² have the meanings given above
and with bifunctional compounds of the formula (IV)

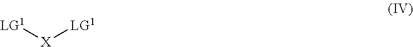

in which
LG¹ represents halogen or sulphonate, where the two LG¹ can be identical or different, and
X has the meanings given above,
if appropriate in the presence of a catalyst, if appropriate in the presence of an acid binder and if appropriate in the presence of a diluent, (cf. Scheme 2)
   or

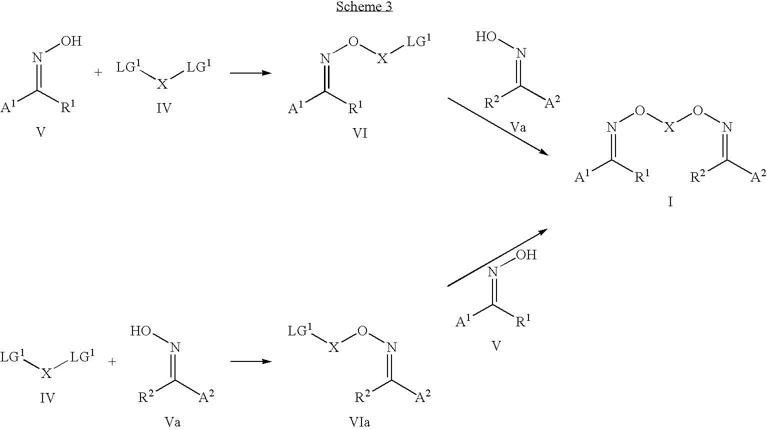

(C) free oximes of the formula (V)

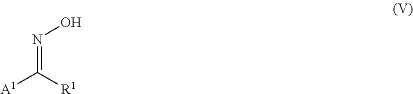

in which A¹ and R¹ have the meanings given above
or free oximes of the formula (Va)

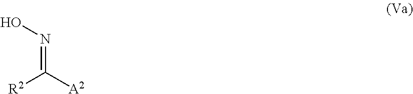

in which R² and A² have the meanings given above
are reacted initially with bifunctional compounds of the formula (IV)

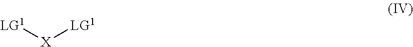

in which
LG¹ represents halogen or sulphonate, where the two LG¹ can be identical or different, and
X has the meanings given above,
if appropriate in the presence of a catalyst, if appropriate in the presence of an acid binder and if appropriate in the presence of a diluent (cf. Monatshefte für Chemie 1959, 90, 799; J. Org. Chem. 1977, 42, 3952),
giving the intermediates of the formula (VI)

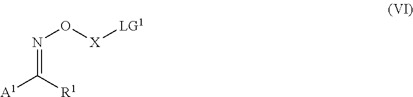

in which A¹, R¹, X and LG¹ have the meanings given above
or the intermediates of the formula (VIa)

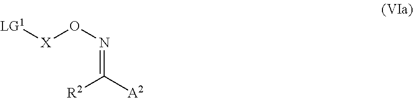

in which R², A², X and LG¹ have the meanings given above,
which are then reacted with a free oxime of the formula (Va) or (V),
if appropriate in the presence of a catalyst, if appropriate in the presence of an acid binder and if
appropriate in the presence of a diluent, (cf. Scheme 3).

Moreover, it has been found that bisoximes of the formula (Ia)

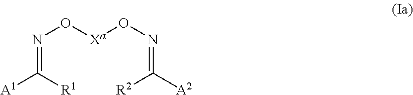

in which
A¹, R¹, R² and A² have the meanings given above,
X^{a} represents one of the groupings X-3 to X-10 given above, with the proviso that exactly one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴ represents

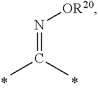

in which R²⁰ has the meanings given above, are obtained when

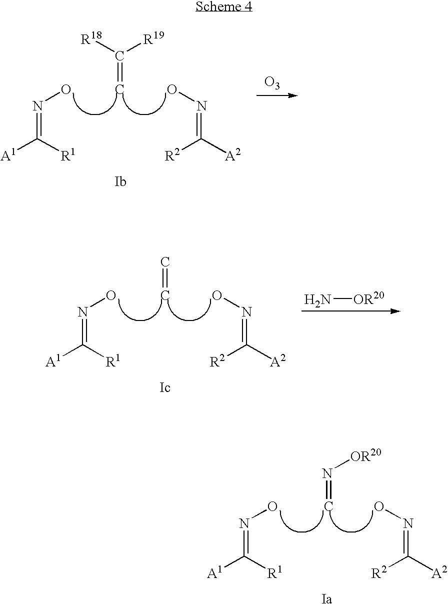

(D) bisoximes of the formula (Ib)

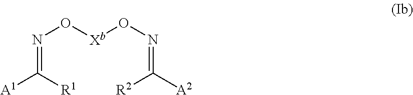

in which
A¹, R¹, R² and A² have the meanings given above,
X^{b} represents one of the groupings X-3 to X-10 given above, with the proviso that exactly one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴
   represents

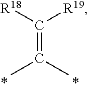

in which R¹⁸ and R¹⁹ have the meanings given above,
are reacted initially with ozone, if appropriate in the presence of a catalyst and if appropriate in the presence of a diluent, and then reacting the bisoximes, obtained as intermediates, of the formula (Ic)

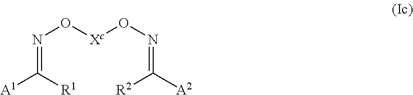

in which
A¹, R¹, R² and A² have the meanings given above,
X^{c} represents one of the groupings X-3 to X-10 given above, with the proviso that exactly one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴
   represents

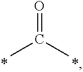

with a free oxime (or its hydrohalide) of the formula (VII)

H₂N—OR²⁰ (VII)
in which R²⁰ has the meanings given above,
if appropriate in the presence of a catalyst, if appropriate in the presence of an acid binder and if appropriate in the presence of a diluent, (cf. Scheme 4).

Moreover, it has been found that bisoximes of the formula (Id)

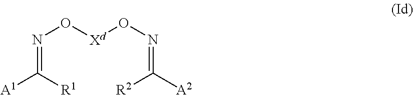

in which
A¹, R¹, R² and A² have the meanings given above,
X^{d} represents one of the groupings X-3 to X-10 given above, with the proviso that at least one of the radicals Z⁵, Z⁸, Z⁹, Z¹² to Z¹⁴, Z¹⁷ to Z²⁰, Z²³ to Z²⁷, Z³⁰ to Z³⁵ Z³⁸ to Z⁴⁴, Z⁴⁷ to Z⁵⁴ represents

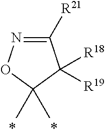

or represents

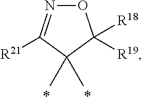

in which
R¹⁸ and R¹⁹ have the meanings given above and
R²¹ represents hydrogen, C₁-C₈-alkyl, C₂-C₈-alkenyl, C₂-C₈-alkynyl, in each case optionally substituted phenyl, benzyl, heterocyclyl or hetaryl,
are obtained when

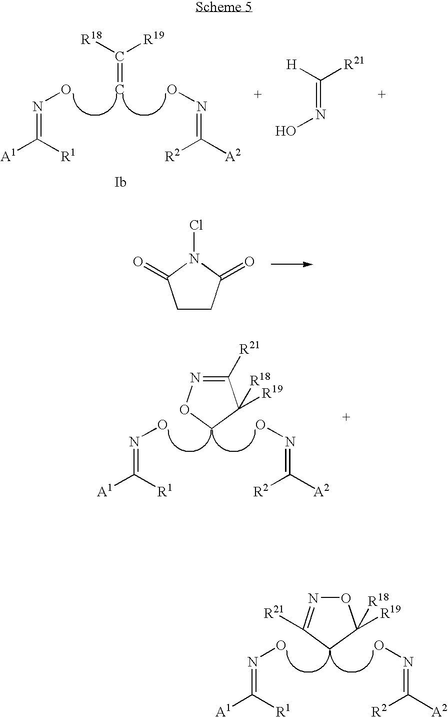

(E) bisoximes of the formula (Ib)

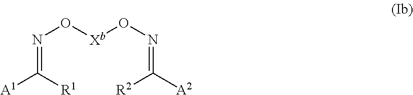

in which A¹, R¹, R², A² and X^{b} have the meanings given above
are reacted with an aldoxime of the formula (VIII)

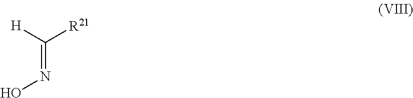

in which R²¹ has the meanings given above,
in the presence of N-chlorosuccinimide, if appropriate in the presence of a catalyst and if
appropriate in the presence of a diluent, (cf. Scheme 5).

Moreover, it has been found that bisoximes of the formula (Ie)

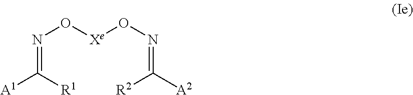

in which
A¹, R¹, R² and A² have the meanings given above,
X^{e} represents one of the groupings X-3 to X-10 given above, with the proviso that at least one of the radicals Z⁵, Z⁸, Z⁹, Z¹² to Z¹⁴, Z¹⁷ to Z²⁰, Z²³ to Z²⁷, Z³⁰ to Z³⁵, Z³¹ to Z⁴⁴, Z⁴⁷ to Z⁵⁴ represents

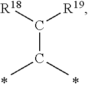

in which R¹⁸ and R¹⁹ have the meanings given above,
are obtained when

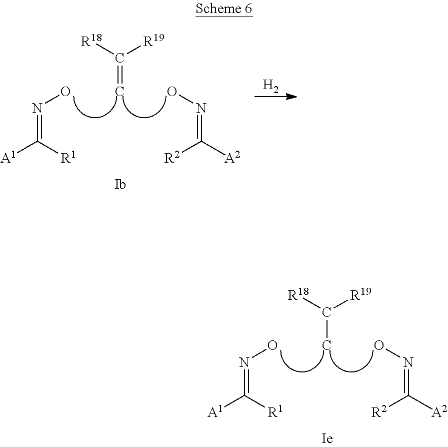

(F) bisoximes of the formula (Ib)

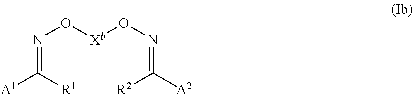

in which A¹, R¹, R², A² and X^{b} have the meanings given above,
are reacted with hydrogen or a hydrogen source, if appropriate in the presence of a catalyst and if appropriate in the presence of a diluent (cf. Scheme 6).

Bisoximes which can be used according to the invention can, if appropriate, be present as mixtures of various possible isomeric forms, in particular of stereoisomers, such as, for example, E and Z, threo and erythro, and also optical isomers, and, if appropriate, also of tautomers. What is claimed are both the E and the Z isomers and the threo and erythro and also the optical isomers, any mixtures of these isomers, and also the possible tautomeric forms.

The compounds of the formula (I) can be present, in particular, in the form of (E/E), (E/Z), (Z/E) and (Z/Z) isomers or mixtures thereof, for example according to the following formulae:

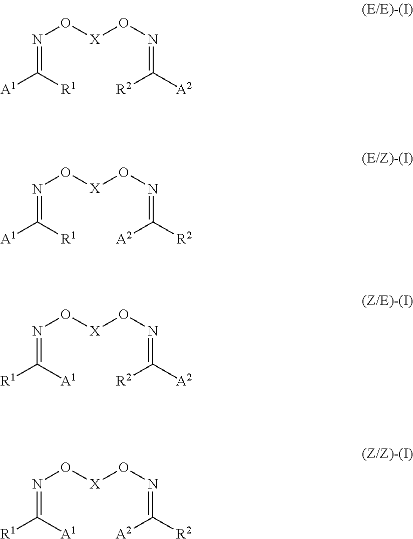

For the sake of simplicity, in each case only one form is shown, preferably the (E/E) form.

This also applies to the compounds of the formulae (Ia), (Ib), (Ic), (Id), (Ie) and (I-1).

Depending on the nature of the substituents defined above, the bisoximes of the formula (I) have acidic or basic properties and can form salts, if appropriate also inner salts or adducts, with inorganic or organic acids or with inorganic or organic bases or with metal ions. Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in the various valencies they can assume.

If the bisoximes of the formula (I) carry hydroxyl groups, carboxyl groups or other groups which induce acidic properties, these compounds can be converted with bases into salts. Suitable bases are, for example, the hydroxides, carbonates and bicarbonates of the alkali metals and the alkaline earth metals, in particular those of sodium, potassium, magnesium and calcium, furthermore ammonia, primary, secondary and tertiary amines having C₁-C₄-alkyl radicals, mono-, di- and trialkanolamines of C₁-C₄-alkanols, choline and also chlorocholine.

If the bisoximes of the formula (I) carry amino groups, alkylamino groups or other groups which induce basic properties, these compounds can be converted with acids into salts. Examples of inorganic acids are hydrohalic acids, such as hydrofluoric acid, hydrochloric acid, hydrobromic acid and hydroiodic acid, sulphuric acid, phosphoric acid and nitric acid, and acidic salts, such as NaHSO₄ and KHSO₄—Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or -disulphonic acids (aromatic radicals, such as phenyl and naphthyl which carry one or two sulphonic acid groups), alkylphosphonic acids (phosphonic acids having straight-chain or branched alkyl radicals of 1 to 20 carbon atoms), arylphosphonic acids or -diphosphonic acids (aromatic radicals, such as phenyl and naphthyl, which carry one or two phosphonic acid radicals, where the alkyl radicals and aryl radicals may carry further substituents, for example p-toluenesulphonic acid, salicylic acid, p-aminosalicylic acid, 2-phenoxybenzoic acid, 2-acetoxybenzoic acid, etc.

The salts which can be obtained in this manner also have fungicidal properties.

The formula (I) provides a general definition of the bisoximes which can be used according to the invention. Preferred radical definitions of the formulae given above and below are indicated below. These definitions apply both to the end products of the formula (I) and likewise to all intermediates.
R¹ and R² independently of one another preferably represent hydrogen, chlorine, bromine, iodine, C₁-C₆-alkyl, C₃-C₆-cycloalkyl, C₁-C₆-haloalkyl having 1 to 13 fluorine, chlorine and/or bromine atoms, OH, C₁-C₆-alkoxy, (C₁-C₄-alkoxy)carbonyl, SH, C₁-C₆-alkylthio, C₁-C₆-alkylsulphinyl, C₁-C₆-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₆-alkyl)amine, (C₁-C₆-alkyl)amine, C₂-C₆-alkenyl, C₂-C₆-alkynyl, represent phenyl, benzyl, heterocyclyl or hetaryl, each of which is optionally substituted by halogen, cyano, C₁-C₄-haloalkyl having 1 to 9 fluorine, chlorine and/or bromine atoms, C₁-C₄-alkyl, C₂-C₄-alkenyl, C₂-C₄-alkynyl, C₁-C₄-alkoxy.
R¹ and R² independently of one another particularly preferably represent hydrogen, chlorine, bromine, C₁-C₄-alkyl, cyclopropyl, cyclobutyl, cyclopentyl, cyclohexyl, C₁-C₄-haloalkyl having 1 to 9 fluorine, chlorine and/or bromine atoms, OH, C₁-C₄-alkoxy, (C₁-C₄-alkoxy)carbonyl, SH, C₁-C₄-alkylthio, C₁-C₄-alkylsulphinyl, C₁-C₄-alkylsulphonyl, C₁-C₂-alkoxy-C₁-C₂-alkyl, (C₁-C₂-alkyl)thio(C₁-C₂-alkyl), (C₁-C₂-alkyl)sulphinyl(C₁-C₂-alkyl), (C₁-C₂-alkyl)sulphonyl(C₁-C₂-alkyl), amino, di(C₁-C₄-alkyl)amine, (C₁-C₄-alkyl)amine, C₂-C₄-alkenyl, C₂-C₄-alkynyl, represent phenyl, benzyl, thienyl, piperazinyl or morpholinyl, each of which is optionally substituted by fluorine, chlorine, bromine, cyano, trifluoromethyl, difluoromethyl, trichloromethyl, dichloromethyl, C₁-C₄-alkyl, C₁-C₄-alkoxy.
R¹ and R² independently of one another very particularly preferably represent hydrogen, chlorine, methyl, ethyl, n-propyl, i-propyl, n-, i-, s- or t-butyl, cyclopropyl, cyclopentyl, cyclohexyl, trifluoromethyl, di-fluoromethyl, trichloromethyl, 2,2,2-trifluoroethyl, pentafluoroethyl, heptafluoro-n-propyl, heptafluoro-i-propyl, OH, methoxy, ethoxy, n-propoxy, i-propoxy, n-, i-, s- or t-butoxy, methoxycarbonyl, ethoxycarbonyl, SH, methylthio, ethylthio, n-propylthio, i-propylthio, n-, i-, s- or t-butylthio, methylsulphinyl, ethylsulphinyl, n-propylsulphinyl, i-propylsulphinyl, n-, i-, s- or t-butylsulphinyl, methylsulphonyl, ethylsulphonyl, n-propylsulphonyl, i-propylsulphonyl, n-, i-, s- or t-butylsulphonyl, methoxymethyl, methoxyethyl, ethoxyethyl, ethoxymethyl, methylthiomethyl, methylthioethyl, ethylthioethyl, ethylthiomethyl, methylsulphonylmethyl, methylsulphonylethyl, ethylsulphonylethyl, ethylsulphonylmethyl, amino, dimethylamine, diethylamine, diisopropylamine, di-n-propylamine, methylamine, ethylamine, isopropylamine, n-propylamine, vinyl, allyl, butenyl, ethynyl, propargyl, represent phenyl, benzyl, thienyl, piperazinyl or morpholinyl, each of which is optionally substituted by fluorine, chlorine, bromine, cyano, trifluoromethyl, methyl, ethyl, n-propyl, i-propyl, n-, i-, s-, t-butyl, methoxy, ethoxy, n-propoxy, i-propoxy, n-, i-, s-, t-butoxy.
R¹ and R² independently of one another especially preferably represent chlorine, methyl, ethyl, n-propyl, i-propyl, i-butyl, cyclopropyl, trifluoromethyl, difluoromethyl, trichloromethyl, methoxycarbonyl, ethoxycarbonyl, methoxymethyl, methylthiomethyl, methylsulphonylmethyl, amino, 3-buten-1-yl, phenyl, benzyl, thienyl, 4-methylpiperazinyl or morpholinyl.
R¹ and R² most preferably each represent methyl, ethyl, n-propyl or amino, in particular methyl or ethyl.
A¹ and A² independently of one another preferably represent one of the cycles A-1 to A-43 below:

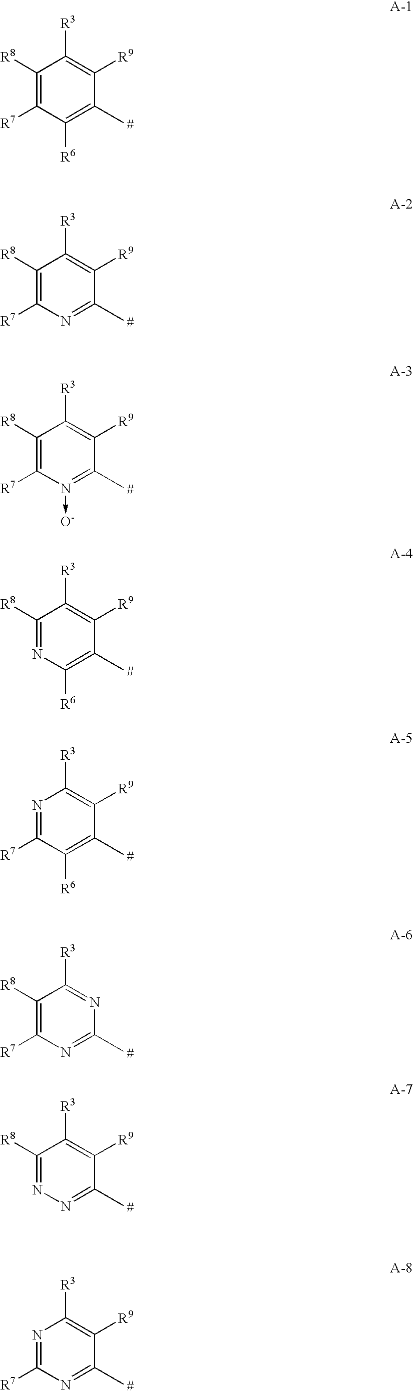

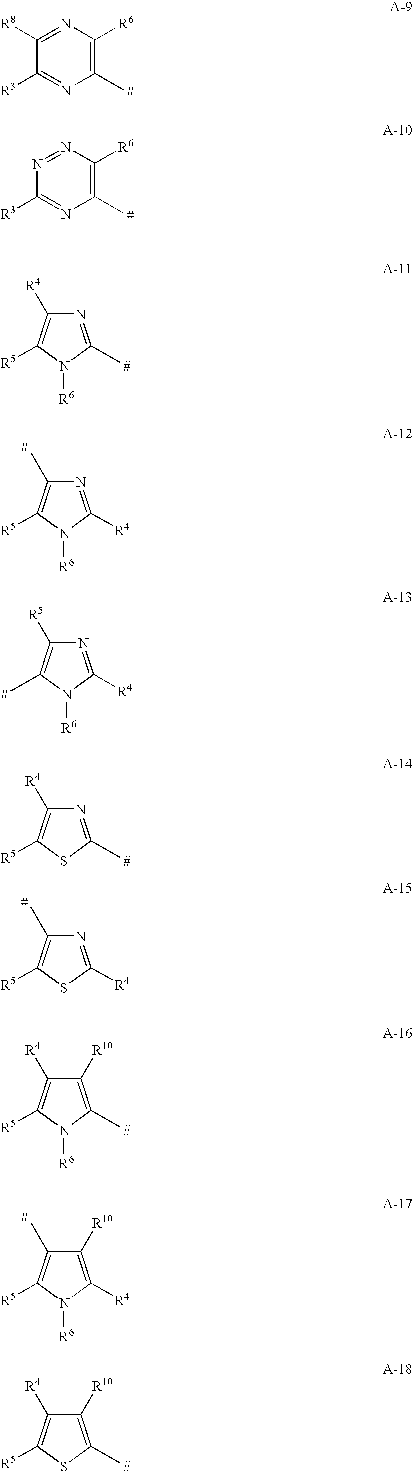

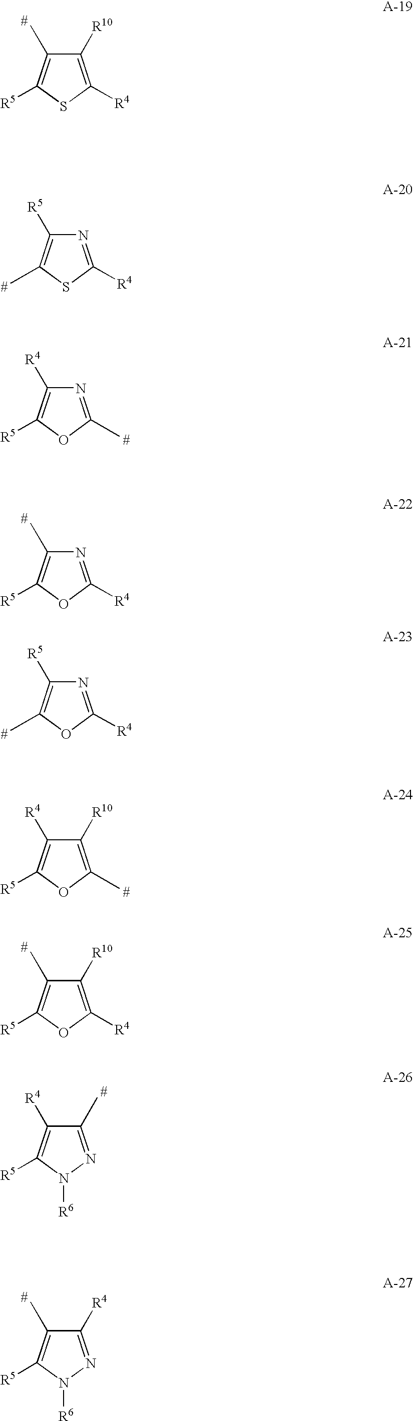

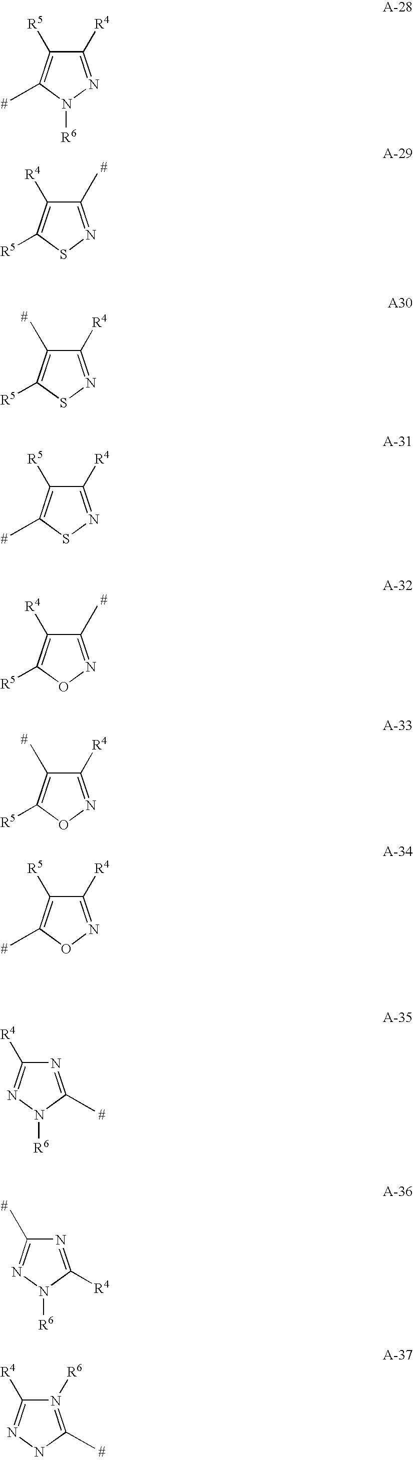

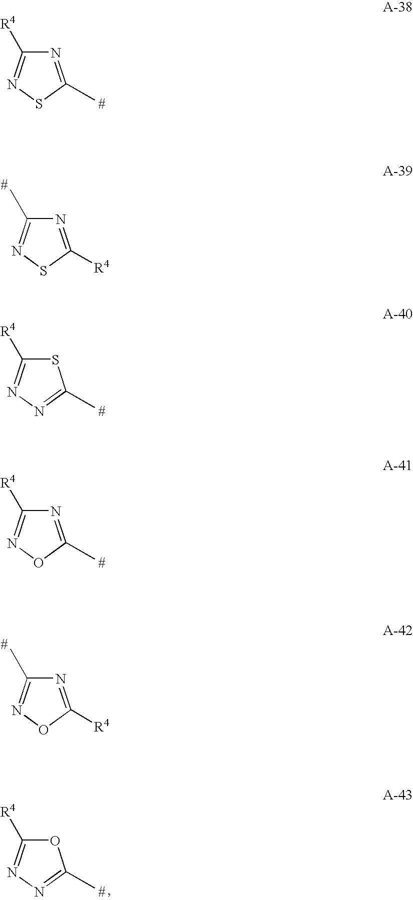

where A¹ and A² do not simultaneously represent the cycle A-1.
A¹ and A² independently of one another particularly preferably represent one of the cycles A-1, A-2, A-4, A-5, A-6, A-7, A-8, A-9, A-10, A-11, A-12, A-13, A-14, A-15, A-16, A-17, A-18, A-19, A-20, A-21, A-22, A-23, A-24, A-25, A-26, A-27, A-28, A-29, A-30, A-31, A-32, A-33 or A-34, where A¹ and A² do not simultaneously represent the cycle A-1.
A¹ and A² independently of one another very particularly preferably represent one of the cycles A-1, A-2, A-4, A-5, A-6, A-7, A-8, A-9, A-10, A-11, A-14, A-15, A-16, A-18, A-20, A-21, A-24, A-26, A-30 or A-33, where A¹ and A² do not simultaneously represent the cycle A-1.
A¹ and A² independently of one another especially preferably represent one of the cycles A-1, A-2, A-4, A-5, A-6, A-7, A-8, A-9 or A-10, where A¹ and A² do not simultaneously represent the cycle A-1.
A¹ and A² most preferably each represent A-2.
R³, R⁴, R⁵, R⁶, R⁷, R⁸, R⁹ and R¹⁰ independently of one another preferably represent hydrogen, fluorine, chlorine, bromine, iodine, cyano, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², C₁-C₆-alkyl, C₂-C₆-alkenyl, C₂-C₆-alkynyl, C₁-C₆-haloalkyl having in each case 1 to 13 fluorine, chlorine and/or bromine atoms, C₃-C₆-cycloalkyl, C₃-C₆-cycloalkenyl, tri-C₁-C₄-alkyl-silyl, phenyl-(di-C₁-C₄-alkyl)-silyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl or pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms.
R³, R⁴, R⁵, R⁶, R⁷, R⁸, R⁹ and R¹⁰ moreover independently of one another preferably represent C₁-C₄-alkoxy-C-₂-C₆-alkenyl, C₁-C₄-alkoxy-C₂-C₆-alkynyl, (C₁-C₄-alkyl)carbonyloxy-C₂-C₆-alkenyl or (C₁-C₄-alkyl)carbonyloxy-C₂-C₆-alkynyl.
Moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together preferably form an unsubstituted or substituted unsaturated or fully or partially saturated heterocyclic or carbocyclic 5- to 7-membered ring.
R¹¹ preferably represents hydrogen, C₁-C₆-alkyl, vinyl, allyl, ethynyl, propargyl, C₁-C₆-haloalkyl having in each case 1 to 13 fluorine, chlorine and/or bromine atoms, C₃-C₆-cycloalkyl, tri-C₁-C₄-alkyl-silyl, hydroxy-C₁-C₄-alkyl, C₁-C₄-alkoxy-C₁-C₄-alkyl, (C₁-C₄-alkyl)thio(C₁-C₄-alkyl), phenyl, benzyl, pyridinyl, pyridinylmethyl, represents phenyl or benzyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms, where, if two radicals R¹¹ are attached to a carbon or nitrogen atom, these radicals R¹¹ may be identical or different.
Moreover, if two radicals R¹¹ are attached to a nitrogen atom, these two radicals R¹¹ together with the nitrogen atom to which they are attached preferably form a 3- to 6-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent.
Moreover the two radicals R¹¹ in the grouping NR¹¹COR¹¹ together with this grouping to which they are attached preferably form a 4- to 6-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent.
R¹² preferably represents hydrogen, C₁-C₆-alkyl, C₁-C₆-haloalkyl having in each case 1 to 13 fluorine, chlorine and/or bromine atoms, —(CH₂)ₙOR¹¹, tri-C₁-C₄-alkyl-silyl.
m preferably represents the number 1, 2, 3, 4, 5 or 6.
n preferably represents the number 1, 2, 3 or 4.
R³, R⁴, R⁵, R⁶, R⁷, R⁸, R⁹ and R¹⁰ independently of one another particularly preferably represent hydrogen, fluorine, chlorine, bromine, cyano, hydroxyl, methoxy, ethoxy, n-propoxy, i-propoxy, n-, i-, s-, t-butoxy, —O(CH₂)₂OH, —O(CH₂)₂OCH₃, —O(CH₂)₃OH, —O(CH₂)₃OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-Pr, SO-iPr, SO-Bu, SO-secBu, SO-isoBu, SO-tBu, SO₂-Me, SO₂-Et, SO₂—Pr, SO₂-iPr, SO₂-Bu, SO₂-secBu, SO₂-isoBu, SO₂-tBu, SONHMe, SONMe₂, SONHEt, SONEt₂, SONHPr, SONPr₂, SONHBu, SONBu₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, CH₂OH, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂CO₂Me, (CH₂)₂CO₂Me, (CH₂)₃CO₂Me, CH₂CO₂Et, (CH₂)₂CO₂Et, (CH₂)₃CO₂Et, CH₂CO₂Pr, (CH₂)₂CO₂Pr, (CH₂)₃CO₂Pr, CH₂CO₂iPr, (CH₂)₂CO₂iPr, (CH₂)₃CO₂iPr, CH₂CO₂tBu, (CH₂)₂CO₂tBu, (CH₂)₃CO₂tBu, CH₂CO₂(CH₂)₂OH CH₂CO₂(CH₂)₂OCH₃, CH₂CO₂(CH₂)₃OH, CH₂CO₂(CH₂)₃OCH₃, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, cyclohexyl, phenyl, benzyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one.
R³, R⁴, R⁵, R⁶, R⁷, R⁹ and R¹⁰ moreover independently of one another particularly preferably represent iodine, vinyl, allyl, prop-1-en-1-yl, ethynyl, propargyl, vinyloxy, allyloxy, propargyloxy, methoxyprop-1-en-1-yl, methoxyprop-1-yn-1-yl, acetyloxyprop-1-en-1-yl, acetyloxyprop-1-yn-1-yl and pyrrolidin-1-yl.
Moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together particularly preferably form an unsubstituted or substituted unsaturated or fully or partially saturated heterocyclic or carbocyclic 5- to 7-membered ring.
R³, R⁴, R⁶, R⁷, R⁸, R⁹ and R¹⁰ independently of one another very particularly preferably represent hydrogen, fluorine, chlorine, bromine, cyano, hydroxyl, methoxy, ethoxy, n-propoxy, i-propoxy, n-, i-, s-, t-butoxy, —O(CH₂)₂OH, —O(CH₂)₂OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-tBu, SO₂-Me, SO₂-Et, SO₂—Pr, SO₂-iPr, SONHMe, SONMe₂, SONHEt, SONEt₂, SONHPr, SONPr₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCO(CH₂)₂OH, NHCO(CH₂)₂OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCOMe, OCOEt, OCOPr, OCOiPr, OCOBu, OCOsecBu, OCOisoBu, OCOtBu, CONHEt, CONEt₂, CONHMe, CONMe₂, CONHPr, CONPr₂, CONHBu, CONHsecBu, CONHisoBu, CONHtBu, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CH₂OH, (CH₂)₂OH, CH₂OMe, (CH₂)₂OMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, CH₂NMe₂, (CH₂)₂NHMe, (CH₂)₂NMe₂, CH₂CO₂Me, (CH₂)₂CO₂Me, CH₂CO₂Et, (CH₂)₂CO₂Et, CH₂CO₂Pr, (CH₂)₂CO₂Pr, CH₂CO₂iPr, (CH₂)₂CO₂iPr, CH₂CO₂tBu, (CH₂)₂CO₂tBu, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethyl-butyl, 1,1,2-trimethylpropyl, 1,2,2-trimethylpropyl, 1-ethyl-1-methylpropyl and 1-ethyl-2-methyl-propyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, cyclohexyl, phenyl, benzyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one.
R³, R⁴, R⁵, R⁶, R⁷, R⁸, R⁹ and R¹⁰ moreover independently of one another very particularly preferably represent iodine, vinyl, allyl, prop-1-en-1-yl, ethynyl, propargyl, vinyloxy, allyloxy, propargyloxy, methoxyprop-1-yn-1-yl, acetyloxyprop-1-yn-1-yl and pyrrolidin-1-yl,
Moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together very particularly preferably form an unsubstituted or substituted unsaturated or fully or partially saturated heterocyclic or carbocyclic 5- or 6-membered ring,

Thus, A¹ and A² independently of one another may also represent the following cycles which are in each case optionally substituted by methyl: tetrahydroquinazolinyl (preferably 5,6,7,8-tetrahydroquinazolin-2-yl), tetrahydroquinolinyl (preferably 5,6,7,8-tetrahydroquinolin-2-yl), quinolinyl (preferably quinolin-2-yl), 6,7-dihydro-5H-cyclopenta[b]pyridin-2-yl, 2,3-dihydro-1-benzofuran-5-yl, naphthyl (preferably 2-naphthyl), benzimidazolyl (preferably 1H-benzimidazol-2-yl), 6,7,8,9-tetrahydro-5H-cyclohepta[b]pyridin-2-yl.
X preferably represents one of the groupings X-1, X-2, X-3, X-4, X-5, X-6, X-7 or X-10.
X particularly preferably represents one of the groupings X-1, X-3, X-5, X-6 or X-10.
X very particularly preferably represents one of the groupings X-3, X-5, X-6 or X-10.
X especially preferably represents one of the groupings X-3, X-5 or X-6.
X most preferably represents the grouping X-3.
Z¹ preferably represents CR¹³R¹⁴ or SiR¹⁵R¹⁶, or represents in each case unsubstituted or substituted oxetane-3,3-diyl, cyclopropane-1,1-diyl, cyclobutane-1,1-diyl, cyclopentane-1,1-diyl, cyclohexane-1,1-diyl.
Z¹ particularly preferably represents CR¹³R¹⁴ or SiR¹⁵R¹⁶, or represents oxetane-3,3-diyl, oxirane-2,2-diyl, cyclopropane-1,1-diyl, cyclobutane-1,1-diyl, cyclopentane-1,1-diyl, cyclohexane-1,1-diyl.
Z¹ very particularly preferably represents —CH₂—, —CH(Me)-, —CH(OH)—, —C(Me₂)- or —Si(Me₂)-.
Z¹ especially preferably represents —CH₂— or —CH(Me)-.
Z² and Z³ independently of one another preferably represent CR¹³R¹⁴ or SiR¹⁵R¹⁶, but where two silicon atoms are not adjacent, or represent in each case unsubstituted or substituted oxetane-3,3-diyl, cyclopropane-1,1-diyl, cyclobutane-1,1-diyl, cyclopentane-1,1-diyl, cyclohexane-1,1-diyl, where X-2 contains at most one spiro group.
Z² and Z³ independently of one another particularly preferably represent CR¹³R¹⁴ or SiR¹⁵R¹⁶, or represent oxetane-3,3-diyl, cyclopropane-1,1-diyl, cyclobutane-1,1-diyl, cyclopentane-1,1-diyl, cyclohexane-1,1-diyl, where X-2 contains at most one spiro group.
X-2 very particularly preferably represents —(CH₂)₂—, —CH₂C(O)—, —CH(Me)CH₂—, —CH(Me)CH(Me)—, —C(Me₂)CH₂— or —Si(Me₂)CH₂—
X-2 especially preferably represents —(CH₂)₂— or —CH₂C(O)—.
Z⁴, Z⁵ and Z⁶ independently of one another preferably represent CR¹³R¹⁴ or SiR¹⁵R¹⁶, but where two silicon atoms are not adjacent, or Z⁴ and Z⁶ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z⁵ independently thereof preferably represents in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl or Z⁵ moreover preferably represents 1,3-oxazolidine-4,4-diyl, where X-3 contains at most one spiro group, or two adjacent groups Z⁴, Z⁵ or Z⁵, Z⁶ preferably represent —CR¹³═CR¹⁴—, —CR¹³═N—, —N═CR¹³— or —C≡C— or pyridinediyl, or two adjacent groups Z⁴, Z⁵ or Z⁵, Z⁶ moreover preferably represent cyclopentane or cyclohexane, or X-3 preferably represents optionally halogen- or C₁-C₃-alkyl-substituted pyrimidinediyl, or X-3 moreover preferably represents cyclopentane-1,3-diyl or cyclohexane-1,3-diyl, where X-3 may contain at most one such group or such cycle. If Z⁴ and Z⁶ independently of one another preferably represent C═O or C═S, Z⁵ preferably represents CR¹³R¹⁴ or NR¹⁷.
Z⁴, Z⁵ and Z⁶ independently of one another particularly preferably represent CR¹³R¹⁴ or SiR¹⁵R¹⁶, but where two silicon atoms are not adjacent, or Z⁴ and Z⁶ independently of one another particularly preferably represent oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z⁵ independently thereof particularly preferably represents oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represents 4,5-dihydroisoxazole-5,5-diyl, which is optionally mono- or polysubstituted by identical or different substituents from the group consisting of C₁-C₄-alkyl, C₂-C₆-alkenyl, phenyl and halophenyl, where X-3 contains at most one spiro group, or two adjacent groups Z⁴, Z⁵ or Z⁵, Z⁶ particularly preferably represent —CR¹³═CR¹⁴—, —CR¹³═N—, —N═CR¹³— or —C≡C— or pyridine-2,3-diyl, or X-3 represents pyrimidine-4,6-diyl which is optionally substituted in the 5-position by fluorine, chlorine or methyl, where X-3 may contain at most one such group or such cycle. If Z⁴ and Z⁶ independently of one another particularly preferably represent C═O or C═S, Z⁵ particularly preferably represents CR¹³R¹⁴ or NR¹⁷.
X-3 very particularly preferably represents —CH₂—C(R¹³R¹⁴)—CH₂—, —C(R¹³R¹⁴)—CH₂—C(R¹³R¹⁴)—, —C(R¹³R¹⁴)—C(R¹³R¹⁴)—CH₂—, —C(R¹³R¹⁴)—C(R¹³R¹⁴)—C(R¹³R¹⁴)—, —C(R¹³R¹⁴)—C(═CH₂)—C(R¹³R¹⁴)—, —C(R¹³R¹⁴)—C(═O)—C(R¹³R¹⁴)—, —[C(R¹³R¹⁴)]₂—C(═O)—, —C(═O)—C(R¹³R¹⁴)—C(═O)—, —C(═O)—NR¹⁷—C(═O)—, —CH₂—Si(R¹⁵R¹⁶)—CH₂— or —CH₂—C(═NOR²⁰)—CH₂—
X-3 especially preferably represents —CH₂—CHR²²—CH₂—, —CH₂—C(R²²R²³)—CH₂—, —CH(R²²)—CH₂—CH(R²²)—, —CH(R²²)—CH(R²²)—CH(R²²)—, —CH₂—C(═CH₂)—CH₂—, —CH₂—C(═O)—CH₂—, —(CH₂)₂—C(═O)—, —C(═O)—C(R²²)₂—C(═O)—, —C(═O)—NR²²—C(═O)—, —CH₂—Si(CH₃)₂—CH₂—, —CH₂—C(═NOH)—CH₂—, —CH₂—C(═NOCH₃)—CH₂—, or represents one of the groupings below

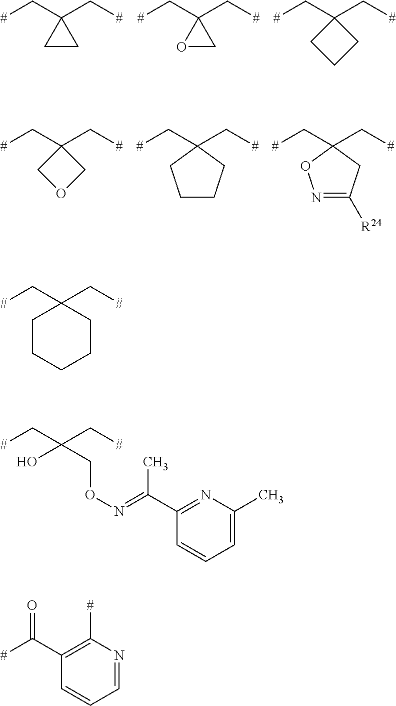

in which R²² represents hydrogen, fluorine, chlorine, hydroxyl, methyl, ethyl, n-propyl, i-propyl, n-butyl, i-butyl, s-butyl, t-butyl, methoxy, ethoxy, phenyl or benzyl, R²³ represents fluorine, chlorine, methyl or ethyl and R²⁴ represents methyl, ethyl, n-propyl, i-propyl, i-butyl, t-butyl, 2-methyl-propen-1-yl or represents phenyl which is optionally substituted by 1 to 3 chlorine atoms,
or moreover represents one of the groupings below

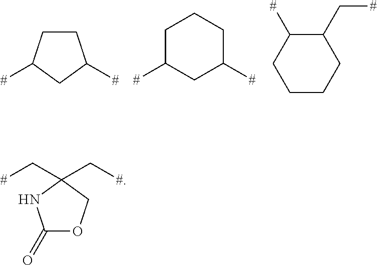

Z⁷, Z⁸, Z⁹ and Z¹⁰ independently of one another preferably represent CR¹³R¹⁴ or SiR¹⁵R¹⁶, but where two silicon or two oxygen atoms are not adjacent, or Z⁷ and Z¹⁰ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z⁸ and Z⁹ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-4 contains at most one spiro group, or two adjacent groups Z⁷, Z⁸ or Z⁸, Z⁹ or Z⁹, Z¹⁰ preferably represent —CR¹³═CR¹⁴—CR¹³═N—, —N═CR¹³— or —C≡C— or represent in each case unsubstituted or substituted 1,3-dioxolane-4,5-diyl, 1,4-dioxane-2,3-diyl, oxirane-2,3-diyl, 1,2-phenylene, 2,3-pyridinediyl or 3,4-pyridinediyl, where X-4 may contain at most one such group or such cycle. If Z⁷ and Z¹⁰ independently of one another preferably represent C═O or C═S, Z⁸ and Z⁹ preferably represent CR¹³R¹⁴ or NR¹⁷.
Z⁷, Z⁸, Z⁹ and Z¹⁰ independently of one another particularly preferably represent CR¹³R¹⁴ or SiR⁵R⁶, but where two silicon or two oxygen atoms are not adjacent, or Z⁷ and Z¹⁰ independently of one another particularly preferably represent oxetane-3,3-diyl, cyclopropane-1,1-diyl, cyclobutane-1,1-diyl, cyclopentane-1,1-diyl, cyclohexane-1,1-diyl, and Z⁸ and Z⁹ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl, which is optionally mono- or polysubstituted by C₁-C₄-alkyl, C₂-C₆-alkenyl, phenyl or halophenyl, where X-4 contains at most one spiro group, or two adjacent groups Z⁸, Z⁹ particularly preferably represent —CR¹³═CR¹⁴—, —CR¹³═N—, —N═CR¹³— or —C≡C— or represent in each case unsubstituted or substituted 1,3-dioxolane-4,5-diyl, 1,4-dioxane-2,3-diyl, oxirane-2,3-diyl or 1,2-phenylene, where X-4 may contain at most one such group or such cycle. If Z⁷ and Z¹⁰ independently of one another particularly preferably represent C═O or C═S, Z⁸ and Z⁹ particularly preferably represent CR¹³R¹⁴ or NR¹⁷.
X-4 very particularly preferably represents —(CH₂)₂—C(R¹³R¹⁴)—CH₂—, —C(R¹³R¹⁴)—(CH₂)₂—C(R¹³R¹⁴)—, —(CH₂)₃—C(═O)—, —C(═O)—(CR¹³R¹⁴)₂—C(═O)—, —(CR¹³R¹⁴)₂—C(═O)—(CR¹³R¹⁴)—, —(CR¹³R¹⁴)—CH═CH—(CR¹³R¹⁴)—, —(CH₂)₂—Si(R¹⁵R¹⁶)—CH₂— or one of the groupings below

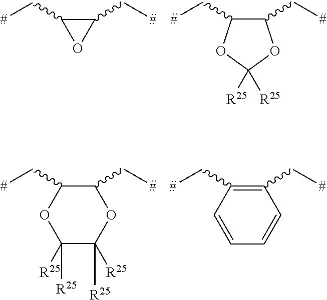

in which R²⁵ represents hydrogen or methyl.
X-4 especially preferably represents —(CH₂)₄—, —(CH₂)₃—C(═O)—, —C(═O)—(CH₂)₂—C(═O)—, —CH₂—CH═CH—CH₂—, or one of the groupings below

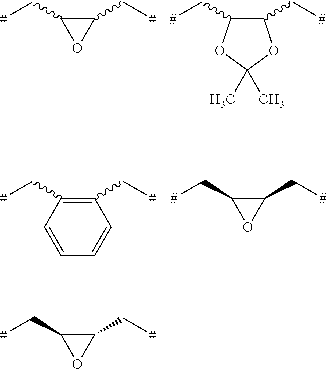

Z¹¹, Z¹², Z¹⁴ and Z¹⁵ independently of one another preferably represent CR¹³R¹⁴ or SiR¹⁵R¹⁶ and Z¹³ independently thereof preferably represents CR¹³R¹⁴, SiR¹⁵R¹⁶, O or NR¹⁷, but where two silicon or two oxygen atoms are not adjacent, or Z¹¹ and Z¹⁵ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z¹², Z¹³ and Z¹⁴ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-5 contains at most one spiro group, or in each case two adjacent groups Z¹¹, Z¹² or Z¹², Z¹³ or Z¹³, Z¹⁴ or Z¹⁴, Z¹⁵ preferably represent —CR¹³═CR¹⁴—, —CR¹³═N—, —N═CR¹³— or —C≡C— or represent in each case unsubstituted or substituted 1,3-dioxolane-4,5-diyl, 1,4-dioxane-2,3-diyl, oxirane-2,3-diyl, or Z¹², Z¹³ and Z¹⁴ together represent in each case substituted or unsubstituted 1,3-phenylene, pyridine-2,6-diyl, pyridine-2,4-diyl or pyridine-3,5-diyl, where X-5 may contain at most one such group or one such cycle. If Z¹¹ and Z¹⁵ independently of one another preferably represent C═O or C═S, Z¹² and Z¹⁴ preferably represent CR¹³R¹⁴ or NR¹⁷.
Z¹¹, Z¹², Z¹⁴ and Z¹⁵ independently of one another particularly preferably represent CR¹³R¹⁴ or SiR¹⁵R¹⁶ and Z¹³ independently thereof particularly preferably represents CR¹³R¹⁴, SiR¹⁵R¹⁶, O or NR¹⁷, but where two silicon or two oxygen atoms are not adjacent, or Z¹¹ and Z¹⁵ independently of one another particularly preferably represent oxetane-3,3-diyl, cyclopropane-1,1-diyl, cyclobutane-1,1-diyl, cyclopentane-1,1-diyl, cyclohexane-1,1-diyl, and Z¹², Z¹³ and Z¹⁴ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by C₁-C₄-alkyl, C₂-C₆-alkenyl, phenyl or halophenyl, where X-5 contains at most one spiro group, or in each case two adjacent groups Z¹¹, Z¹² or Z¹², Z¹³ or Z¹³, Z¹⁴ or Z¹⁴, Z¹⁵ particularly preferably represent —CR¹³═CR¹⁴—, —CR¹³═N—, —N═CR¹³— or —C≡C— or represent in each case unsubstituted or substituted 1,3-dioxolane-4,5-diyl, 1,4-dioxane-2,3-diyl or oxirane-2,3-diyl, or Z¹², Z¹³ and Z¹⁴ together represent 1,3-phenylene or pyridine-2,6-diyl, each of which is mono- or polysubstituted by identical or different substituents from the group consisting of fluorine, chlorine, methyl and trifluoromethyl, where X-5 may contain at most one such group or such cycle. If Z¹¹ and Z¹⁵ independently of one another particularly preferably represent C═O or C═S, Z¹² and Z¹⁴ particularly preferably represent CR¹³R¹⁴ or NR¹⁷.
X-5 very particularly preferably represents —(CH₂)₂—C(R¹³R¹⁴)—(CH₂)₂—, —(CH₂)₂—O—(CH₂)₂—, —C(R¹³R¹⁴)—(CH₂)₃—C(R¹³R¹⁴)—, —(CH₂)₄—C(═O)—, —(CH₂)—C(═O)—(CH₂)₃, —(CH₂)₂—C(═O)—(CH₂)₂—, —C(═O)—(CH₂)₃—C(═O)— or —(CH₂)₂—Si(R¹⁵R¹⁶)—(CH₂)₂— or one of the groupings below

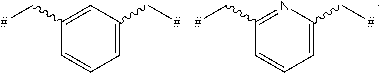

X-5 especially preferably represents —(CH₂)₅—, —(CH₂)₂—O—(CH₂)₂— or —(CH₂)₂—CH(CH₃)—(CH₂)₂—.
Z¹⁶, Z¹⁷, Z²⁰ and Z²¹ independently of one another preferably represent CR¹³R¹⁴ or SiR¹⁵R¹⁶ and Z¹⁸ and Z¹⁹ independently of one another preferably represent CR¹³R¹⁴, SiR¹⁵R¹⁶, O or NR¹⁷, but where two silicon or two oxygen atoms are not adjacent, or Z¹⁶ and Z²¹ independently of one another preferably represent in each case unsubstituted or substituted, oxetane-3,3-diyl, cyclopropane-1,1-diyl, cyclobutane-1,1-diyl, cyclopentane-1,1-diyl, cyclohexane-1,1-diyl, and Z¹⁷, Z¹⁸, Z¹⁹ and Z²⁰ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-6 contains at most one spiro group, or in each case two adjacent radicals Z¹⁶, Z¹⁷ or Z¹⁷, Z¹⁸ or Z¹⁸, Z¹⁹ or Z¹⁹, Z²⁰ or Z²⁰, Z²¹ preferably represent —CR¹³═CR¹⁴—, —CR¹³═N—, —N═CR¹³— or —C≡C—, where X-6 may not contain more than 2 of such groups, which for their part must not be cumulated, or represent in each case unsubstituted or substituted 1,3-dioxolane-4,5-diyl, 1,4-dioxane-2,3-diyl or oxirane-2,3-diyl, or Z¹⁶, Z¹⁷ and Z¹⁸ or Z¹⁷, Z¹⁸ and Z¹⁹ together represent in each case substituted or unsubstituted 1,4-phenylene or pyridine-2,5-diyl, where X-6 may contain at most one such cycle. If Z¹⁶ and Z²¹ independently of one another preferably represent C═O or C═S, Z¹⁷ and Z²⁰ preferably represent CR¹³R¹⁴ or NR¹⁷.
Z¹⁶, Z¹⁷, Z²⁰ and Z²¹ independently of one another particularly preferably represent CR¹³R¹⁴ or SiR¹⁵R¹⁶ and Z¹⁸ and Z¹⁹ independently of one another particularly preferably represent CR¹³R¹⁴, SiR¹⁵R¹⁶, O or NR¹⁷, but where two silicon or two oxygen atoms are not adjacent, or Z¹⁶ and Z²¹ independently of one another particularly preferably represent oxetane-3,3-diyl, cyclopropane-1,1-diyl, cyclobutane-1,1-diyl, cyclopentane-1,1-diyl, cyclohexane-1,1-diyl, and Z¹⁷, Z¹⁸, Z¹⁹ and Z²⁰ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by C₁-C₄-alkyl, C₂-C₆-alkenyl, phenyl or halophenyl, where X-6 contains at most one spiro group, or in each case two adjacent radicals Z¹⁷, Z¹⁸ or Z¹⁸, Z¹⁹ or Z¹⁹, Z²⁰ particularly preferably represent —CR¹³═CR¹⁴—, —CR¹³═N—, —N═CR¹³— or —C≡C—, where X-6 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, or Z¹⁶, Z¹⁷ and Z¹⁸ or Z¹⁷, Z¹⁸ and Z¹⁹ together represent 1,4-phenylene which is mono- or polysubstituted by identical or different substituents from the group consisting of fluorine, chlorine, methyl and trifluoromethyl, where X-6 may contain at most one such cycle. If Z¹⁶ and Z²¹ independently of one another particularly preferably represent C═O or C═S, Z¹⁷ and Z²⁰ particularly preferably represent CR¹³R¹⁴ or NR¹⁷.
X-6 very particularly preferably represents —(CH₂)₃—C(R¹³R¹⁴)—(CH₂)₂—, —C(R¹³R¹⁴)—(CH₂)₄—C(R¹³R¹⁴)—, —(CH₂)₅—C(═O)—, —C(═O)—(CH₂)₄—C(═O)—, —(CH₂)—C(═O)—(CH₂)₄—, —(CH₂)₂—C(═O)—(CH₂)₃— or —(CH₂)₃—Si(R¹⁵R¹⁶)—(CH₂)₂— or represents 1,4-phenylene.
X-6 especially preferably represents —(CH₂)₆—.
Z²², Z²³, Z²⁷ and Z²⁸ independently of one another preferably represent CR¹³R¹⁴ or SiR¹⁵R¹⁶ and Z²⁴, Z²⁵, and Z²⁶ independently of one another preferably represent CR¹³R¹⁴, SiR¹⁵R¹⁶, O or NR¹⁷, but where two silicon or two oxygen atoms are not adjacent, or Z²² and Z²⁸ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z²³, Z²⁴, Z²⁵, Z²⁶ and Z²⁷ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-7 contains at most one spiro group, or in each case two adjacent radicals Z²², Z²³ or Z²³, Z²⁴ or Z²⁴, Z²⁵ or Z²⁵, Z²⁶ or Z²⁶, Z²⁷ or Z²⁷, Z²⁸ preferably represent —CR¹³═CR¹⁴—, —CR¹³═N—, —N═CR¹³— or —C≡C—, where X-7 may not contain more than 2 of such groups, which for their part must not be cumulated, or represent in each case unsubstituted or substituted 1,3-dioxolane-4,5-diyl, 1,4-dioxane-2,3-diyl or oxirane-2,3-diyl, where X-7 may contain at most one such cycle. If Z²² and Z²⁸ independently of one another preferably represent C═O or C═S, Z²³ and Z²⁷ preferably represent CR¹³R¹⁴ or NR¹⁷
Z²², Z²³, Z²⁷ and Z²⁸ independently of one another particularly preferably represent CR¹³R¹⁴ or SiR¹⁵R¹⁶ and Z²⁴, Z²⁵ and Z²⁶ independently of one another particularly preferably represent CR¹³R¹⁴, SiR¹³R¹⁴, O or NR¹⁷, but where two silicon or two oxygen atoms are not adjacent, or Z²² and Z²⁸ independently of one another particularly preferably represent oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z²³, Z²⁴, Z²⁵, Z²⁶, and Z²⁷ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by C₁-C₄-alkyl, C₂-C₆-alkenyl, phenyl or halophenyl, where X-7 contains at most one spiro group, or in each case two adjacent radicals Z²³, Z²⁴ or Z²⁴, Z²⁵ or Z²⁵, Z²⁶ or Z²⁶, Z²⁷ particularly preferably represent —CR¹³═CR¹⁴—, —CR¹³═N—, —N═CR¹³— or —C≡C—, where X-7 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-7 may contain at most one such cycle. If Z²² and Z²⁸ independently of one another particularly preferably represent C═O or C═S, Z²³ and Z²⁷ particularly preferably represent CR¹³R¹⁴ or NR¹⁷.
X-7 very particularly preferably represents —(CH₂)₃—C(R¹³R¹⁴)—(CH₂)₃—, —(CH₂)₃—O—(CH₂)₃—, —(CH₂)₂—O—CH₂—O—(CH₂)₂—, —C(R¹³R¹⁴)—(CH₂)₅—C(R¹³R¹⁴)—, —(CH₂)₆—C(═O)—, —C(═O)—(CH₂)₅—C(═O)— or —(CH₂)₃—Si(R¹⁵R¹⁶)—(CH₂)₃—.
X-7 especially preferably represents —(CH₂)₇— or —(CH₂)₂—O—CH₂—O—(CH₂)₂—.
Z²⁹, Z³⁰, Z³⁵ and Z³⁶ independently of one another preferably represent CR¹³R¹⁴ or SiR¹⁵R¹⁶, Z³⁰ and Z³⁵ moreover preferably represent NR¹⁷, and Z³¹, Z³², Z³³, and Z³⁴ independently of one another preferably represent CR¹³R¹⁴, SiR¹⁵R¹⁶, O or NR¹⁷, but where two silicon or two oxygen atoms are not adjacent, or Z²⁹ and Z³⁶ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, and Z³⁰, Z³¹, Z³², Z³³, Z³⁴ and Z³⁵ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-8 contains at most one spiro group, or in each case two adjacent radicals Z²⁹, Z³⁰ or Z³⁰, Z³¹ or Z³¹, Z³² or Z³², Z³³ or Z³³, Z³⁴ or Z³⁴, Z³⁵ or Z³⁵, Z³⁶ preferably represent —CR¹³═CR¹⁴—, —CR¹³═N—, —N═CR¹³— or —C≡C—, where X-8 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-8 may contain at most one such cycle. If Z²⁹ and Z³⁶ independently of one another preferably represent C═O or C═S, Z³⁰ and Z³⁵ preferably represent CR¹³R¹⁴ or NR¹⁷.
Z²⁹, Z³⁰, Z³⁵ and Z³⁶ independently of one another particularly preferably represent CR¹³R¹⁴ or SiR¹⁵R¹⁶, Z³⁰ and Z³⁵ moreover particularly preferably represent NR¹⁷, and Z³¹, Z³², Z³³, and Z³⁴ independently of one another particularly preferably represent CR¹³R¹⁴, SiR¹⁵R¹⁶, O or NR¹⁷, but where two silicon or two oxygen atoms are not adjacent, or Z²⁹ and Z³⁶ independently of one another particularly preferably represent oxetane-3,3-diyl, cyclopropane-1,1-diyl, cyclobutane-1,1-diyl, cyclopentane-1,1-diyl, cyclohexane-1,1-diyl, and Z³⁰, Z³¹, Z³², Z³³, Z³⁴ or Z³⁵ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by identical or different substituents from the group consisting of C₁-C₄-alkyl, C₂-C₆-alkenyl, phenyl or halophenyl, where X-8 contains at most one spiro group, or in each case two adjacent radicals Z³⁰, Z³¹ or Z³¹, Z³² or Z³², Z³³ or Z³³, Z³⁴ or Z³⁴, Z³⁵ particularly preferably represent —CR¹³═CR¹⁴—, —CR¹³═N—, —N═CR¹⁶— or —C≡C—, where X-8 may contain not more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-8 may contain at most one such cycle. If Z²⁹ and Z³⁶ independently of one another particularly preferably represent C═O or C═S, Z³⁰ and Z³⁵ particularly preferably represent CR¹³R¹⁴ or NR¹⁷.
X-8 very particularly preferably represents —(CH₂)₄—C(R¹³R¹⁴)—(CH₂)₃—, —(CH₂)₄—O—(CH₂)₃—, —(CH₂)₂—O—(CH₂)₂—O—(CH₂)₂—, —C(R¹³R¹⁴)—(CH₂)₆—C(R¹³R¹⁴)—, —(CH₂)₇—C(═O)—, —C(═O)—(CH₂)₆—C(═O)— or —(CH₂)₄—Si(R¹⁵R¹⁶)—(CH₂)₃—.
X-8 moreover very particularly preferably represents —C(═O)—NH—(CH₂)₄—NH—C(═O)—.
X-8 especially preferably represents —(CH₂)₈—.
Z³⁷, Z³⁸, Z⁴⁴ and Z⁴⁵ independently of one another preferably represent CR¹³R¹⁴ or SiR¹⁵R¹⁶, Z³⁸ and Z⁴⁴ moreover preferably represent NR¹⁷, and Z³⁹, Z⁴⁰, Z⁴¹, Z⁴², and Z⁴³ independently of one another preferably represent CR¹³R¹⁴, SiR¹⁵R¹⁶, O or NR¹⁷, but where two silicon or two oxygen atoms are not adjacent, or Z³⁷ and Z⁴⁵ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, or Z³⁸, Z³⁹, Z⁴⁰, Z⁴¹, Z⁴², Z⁴², Z⁴³ or Z⁴⁴ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-9 contains at most one spiro group, or in each case two adjacent radicals Z³⁷, Z³⁸ or Z³⁸, Z³⁹ or Z³⁹, Z⁴⁰ or Z⁴⁰, Z⁴¹ or Z⁴¹, Z⁴² or Z⁴², Z⁴³ or Z⁴³. Z⁴⁴ or Z⁴⁴, or Z⁴⁵ preferably represent —CR¹³═CR¹⁴—, —CR¹³═N—, —N═CR¹³— or —C≡C—, where X-9 may contain not more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-9 may contain at most one such cycle. If Z³⁷ and Z⁴⁵ independently of one another preferably represent C═O or C═S, Z³⁸ and Z⁴⁴ preferably represent CR¹³R¹⁴ or NR¹⁷.
Z³⁷, Z³⁸, Z⁴⁴ and Z⁴⁵ independently of one another particularly preferably represent CR¹³R¹⁴ or SiR¹⁵R¹⁶, Z³⁸ and Z⁴⁴ moreover particularly preferably represent NR¹⁷, and Z³⁹, Z⁴⁰, Z⁴¹, Z⁴², and Z⁴³ independently of one another particularly preferably represent CR¹³R¹⁴, SiR¹⁵R¹⁶, O or NR¹⁷, but where two silicon or two oxygen atoms are not adjacent, or Z³⁷ and Z⁴⁵ independently of one another particularly preferably represent oxetane-3,3-diyl, cyclopropane-1,1-diyl, cyclobutane-1,1-diyl, cyclopentane-1,1-diyl, cyclohexane-1,1-diyl, or Z³⁸, Z³⁹, Z⁴⁰, Z⁴¹, Z⁴², Z⁴³ and Z⁴⁴ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by C₁-C₄-alkyl, C₂-C₆-alkenyl, phenyl or halophenyl, where X-9 contains at most one spiro group, or in each case two adjacent radicals Z³⁸, Z³⁹ or Z³⁹, Z⁴⁰ or Z⁴⁰, Z⁴¹ or Z⁴¹, Z⁴² or Z⁴², Z⁴³ or Z⁴³, Z⁴⁴ particularly preferably represent —CR¹³═CR¹⁴—, —CR¹³═N—, —N═CR¹³— or —C≡C—, where X-9 may contain not more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-9 may contain at most one such cycle. If Z³⁷ and Z⁴⁵ independently of one another particularly preferably represent C═O or C═S, Z³⁸ and Z⁴⁴ particularly preferably represent CR¹³R¹⁴ or NR¹⁷.
X-9 very particularly preferably represents —(CH₂)₄—C(R¹³R¹⁴)—(CH₂)₄—, —(CH₂)₄—O—(CH₂)₄—, —C(R¹³R¹⁴)—(CH₂)₇—C(R¹³R¹⁴)—, —(CH₂)₈—C(═O)—, —C(═O)—(CH₂)₇—C(═O)— or —(CH₂)₄—Si(R¹⁵R¹⁶)—(CH₂)₄—
X-9 moreover very particularly preferably represents —C(═O)—NH—(CH₂)₃—CH(CH₃)—CH₂—NH—C(═O)—.
X-9 especially preferably represents —(CH₂)₉—.
Z⁴⁶, Z⁴⁷, Z⁵⁴ and Z⁵⁵ independently of one another preferably represent CR¹³R¹⁴ or SiR¹⁵R¹⁶ and Z⁴⁸, Z⁴⁹, Z⁵⁰, Z⁵¹, Z⁵² and Z⁵³ independently of one another preferably represent CR¹³R¹⁴, SiR¹⁵R¹⁶, O or NR¹⁷, but where two silicon or two oxygen atoms are not adjacent, or Z⁴⁶ and Z⁵⁵ independently of one another preferably represent in each case unsubstituted or substituted oxetane-3,3-diyl, cyclopropane-1,1-diyl, cyclobutane-1,1-diyl, cyclopentane-1,1-diyl, cyclohexane-1,1-diyl, or Z⁴⁷, Z⁴⁸, Z⁴⁹, Z⁵⁰, Z⁵¹, Z⁵², Z⁵³, or Z⁵⁴ independently of one another preferably represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-10 contains at most one spiro group, or in each case two adjacent radicals Z⁴⁶, Z⁴⁷ or Z⁴⁷, Z⁴⁸ or Z⁴⁸, Z⁴⁹ or Z⁴⁹, Z⁵⁰ or Z⁵⁰, Z⁵¹ or Z⁵¹, Z⁵² or Z⁵², Z⁵³ or Z⁵³, Z⁵⁴ or Z⁵⁴, Z⁵⁵ preferably represent —CR¹³═CR¹⁴—, —CR¹³═N—, —N═CR¹³— or —C≡C—, where X-10 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-10 may contain at most one such cycle. If Z⁴⁶ and Z⁵⁵ independently of one another preferably represent C═O or C═S, Z⁴⁷ and Z⁵⁴ preferably represent CR¹³R¹⁴ or NR¹⁷.
Z⁴⁶, Z⁴⁷, Z⁵⁴ and Z⁵⁵ independently of one another particularly preferably represent CR¹³R¹⁴ or SiR¹⁵R¹⁶ and Z⁴⁸, Z⁴⁹, Z⁵⁰, Z⁵¹, Z⁵² and Z⁵³ independently of one another particularly preferably represent CR¹³R¹⁴, SiR¹⁵R¹⁶, O or NR¹⁷, but where two silicon or two oxygen atoms are not adjacent, or Z⁴⁶ and Z⁵⁵ independently of one another particularly preferably represent oxetane-3,3-diyl, cyclopropane-1,1-diyl, cyclobutane-1,1-diyl, cyclopentane-1,1-diyl, cyclohexane-1,1-diyl, or Z⁴⁷, Z⁴⁸, Z⁴⁹, Z⁵⁰, Z⁵¹, Z⁵², Z⁵³ and Z⁵⁴ independently of one another particularly preferably represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by identical or different substituents from the group consisting of C₁-C₄-alkyl, C₂-C₆-alkenyl, phenyl and halophenyl, where X-10 contains at most one spiro group, or in each case two adjacent radicals Z⁴⁷, Z⁴⁸ or Z⁴⁸, Z⁴⁹ or Z⁴⁹, Z⁵⁰ or Z⁵⁰, Z⁵¹ or Z⁵¹, Z⁵² or Z⁵², Z⁵³ or Z⁵³, Z⁵⁴ particularly preferably represent —CR¹³═CR¹⁴—, —CR¹³═N—, —N═CR¹³— or —C≡C—, where X-10 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-10 may contain at most one such cycle. If Z⁴⁶ and Z⁵⁵ independently of one another particularly preferably represent C═O or C═S, Z⁴⁷ and Z⁵⁴ particularly preferably represent CR¹³R¹⁴ or NR¹⁷.
X-10 very particularly preferably represents —(CH₂)₅—C(R¹³R¹⁴)—(CH₂)₄—, —(CH₂)₅—O—(CH₂)₄—, —(CH₂)₃—O—(CH₂)₂—O—(CH₂)₃—, —C(R¹³R¹⁴)—(CH₂)₈—C(R¹³R¹⁴)—, —(CH₂)₉—C(═O)—, —C(═O)—(CH₂)₈—C(═O)— or —(CH₂)₅—Si(R¹⁵R¹⁶)—(CH₂)₄—.
X-10 especially preferably represents —(CH₂)₁₀—.
R¹³, R¹⁴, R¹⁵, R¹⁶ and R¹⁷ independently of one another preferably represent hydrogen, fluorine, chlorine, bromine, iodine, cyano, hydroxyl, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹¹, C₁-C₆-alkyl, C₂-C₆-alkenyl, C₂-C₆-alkynyl, C₁-C₆-haloalkyl having in each case 1 to 13 fluorine, chlorine and/or bromine atoms, C₃-C₆-cycloalkyl, C₃-C₆-cycloalkenyl, tri-C₁-C₄-alkyl-silyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms.
R¹³ and R¹⁴ moreover together preferably represent ═CR¹⁸R¹⁹, ═O, ═S, ═NOR²⁰.
R¹⁸, R¹⁹ and R²⁰ independently of one another preferably represent hydrogen, C₁-C₆-alkyl, C₁-C₆-haloalkyl having 1 to 13 fluorine, chlorine and/or bromine atoms, C₂-C₆-alkenyl, C₂-C₆-alkynyl, C₃-C₆-cycloalkyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms.
R¹⁸ and R¹⁹ moreover together with the carbon atom to which they are attached preferably form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent.
R¹³, R¹⁴, R¹⁵, R¹⁶ and R¹⁷ independently of one another particularly preferably represent hydrogen, fluorine, chlorine, bromine, hydroxyl, methoxy, ethoxy, n-propoxy, i-propoxy, n-, i-, s-, t-butoxy, —O(CH₂)₂OH, —O(CH₂)₂OCH₃, —O(CH₂)₃OH, —O(CH₂)₃OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-Pr, SO-iPr, SO-Bu, SO-secBu, SO-isoBu, SO-tBu, SO₂-Me, SO₂-Et, SO₂—Pr, SO₂-iPr, SO₂-Bu, SO₂-secBu, SO₂-isoBu, SO₂-tBu, SONHMe, SONMe₂, SONHEt, SONEt₂, SONHPr, SONPr₂, SONHBu, SONBu₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, CH₂OH, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂CO₂Me, (CH₂)₂CO₂Me, (CH₂)₃CO₂Me, CH₂CO₂Et, (CH₂)₂CO₂Et, (CH₂)₃CO₂Et, CH₂CO₂Pr, (CH₂)₂CO₂Pr, (CH₂)₃CO₂Pr, CH₂CO₂iPr, (CH₂)₂CO₂iPr, (CH₂)₃CO₂iPr, CH₂CO₂tBu, (CH₂)₂CO₂tBu, (CH₂)₃CO₂tBu, CH₂CO₂(CH₂)₂OH CH₂CO₂(CH₂)₂OCH₃, CH₂CO₂(CH₂)₃OH, CH₂CO₂(CH₂)₃OCH₃, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, cyclopropyl, cyclobutyl, cyclopentyl, cyclohexyl, 1-cyclohexenyl, 2-cyclohexenyl, 3-cyclohexenyl, phenyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one.
R¹³ and R¹⁴ moreover independently of one another particularly preferably represent vinyloxymethyl, allyloxymethyl, ethynyloxymethyl and propargyloxymethyl.
R¹³ and R¹⁴ moreover together particularly preferably represent ═CR¹⁸R¹⁹, ═O, ═S, ═NOR²⁰.
R¹⁸, R¹⁹ and R²⁰ independently of one another particularly preferably represent hydrogen, methyl, ethyl, n-propyl, i-propyl, n-, i-, s-, t-butyl, trifluoromethyl, cyclopropyl, cyclobutyl, cyclopentyl, cyclohexyl, phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of phenyl, benzyl, pyridinyl, pyridinylmethyl, thienyl, thenyl, furyl, furfuryl, pyrrolyl, pyrrolylmethyl, fluorine, chlorine, bromine, methyl, ethyl, n-propyl, i-propyl, methoxy, trifluoromethyl.
R¹³, R¹⁴, R¹⁵, R¹⁶ and R¹⁷ independently of one another very particularly preferably represent hydrogen, fluorine, chlorine, bromine, hydroxyl, methoxy, ethoxy, n-propoxy, i-propoxy, n-, i-, s-, t-butoxy, —O(CH₂)₂OH, —O(CH₂)₂OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-tBu, SO₂-Me, SO₂-Et, SO₂—Pr, SO₂-iPr, SONHMe, SONMe₂, SONHEt, SONEt₂, SONHPr, SONPr₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCO(CH₂)₂OH, NHCO(CH₂)₂OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCOMe, OCOEt, OCOPr, OCOiPr, OCOBu, OCOsecBu, OCOisoBu, OCOtBu, CONHEt, CONEt₂, CONHMe, CONMe₂, CONHPr, CONPr₂, CONHBu, CONHsecBu, CONHisoBu, CONHtBu, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CH₂OH, (CH₂)₂OH, CH₂OMe, (CH₂)₂OMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, CH₂NMe₂, (CH₂)₂NHMe, (CH₂)₂NMe₂, CH₂CO₂Me, (CH₂)₂CO₂Me, CH₂CO₂Et, (CH₂)₂CO₂Et, CH₂CO₂Pr, (CH₂)₂CO₂Pr, CH₂CO₂iPr, (CH₂)₂CO₂iPr, CH₂CO₂tBu, (CH₂)₂CO₂tBu, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, cyclohexyl, 1-cyclohexenyl, 2-cyclohexenyl, 3-cyclohexenyl, phenyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one.
R¹³ and R¹⁴ moreover independently of one another very particularly preferably represent allyloxymethyl and propargyloxymethyl.
R¹³ and R¹⁴ moreover together very particularly preferably represent ═CR¹⁸R¹⁹, ═O, ═S, ═NOR²⁰.
R¹⁸, R¹⁹ and R²⁰ independently of one another very particularly preferably represent hydrogen, methyl, ethyl, n-propyl, i-propyl, n-, i-, s-, t-butyl, trifluoromethyl, phenyl, cyclopropyl.
R²¹ preferably represents hydrogen, C₁-C₆-alkyl, C₂-C₆-alkenyl, C₂-C₆-alkynyl, represents phenyl, benzyl, heterocyclyl or hetaryl, each of which is optionally substituted by halogen, cyano, C₁-C₄-alkyl, C₁-C₄-alkoxy.
R²¹ particularly preferably represents hydrogen, methyl, ethyl, n-propyl, i-propyl, n-, i-, s-, t-butyl, vinyl, allyl, 1-butenyl, 2-butenyl, 2-methylpropen-1-yl, represents phenyl or benzyl, each of which is optionally mono- or polysubstituted by identical or different substituents from the group consisting of fluorine, chlorine, bromine, cyano, methyl, ethyl, n-propyl, i-propyl, methoxy.

However, the general or preferred radical definitions or illustrations given above can also be combined with one another as desired, i.e. including combinations between respective ranges and preferred ranges. They apply both to the end products and, correspondingly, to precursors and intermediates. Moreover, individual definitions may not apply.

Some of the bisoximes of the formula (I) which can be used according to the invention are already known. Novel and thus also part of the subject-matter of the present invention are bisoximes of the formula (I-1)

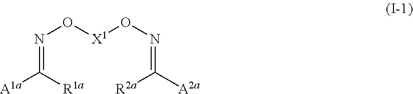

in which
R^{1a} and R^{2a} independently of one another represent halogen, C₁-C₈-alkyl, C₃-C₈-cycloalkyl, C₁-C₈-haloalkyl, OH, C₁-C₈-alkoxy, (C₁-C₆-alkoxy)carbonyl, SH, C₁-C₈-alkylthio, C₁-C₈-alkylsulphinyl, C₁-C₈-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₈-alkyl)amine, (C₁-C₈-alkyl)amine, C₂-C₈-alkenyl, C₂-C₈-alkynyl, in each case optionally substituted phenyl, benzyl, heterocyclyl or hetaryl,
A^{1a} and A^{2a} independently of one another represent one of the cycles below

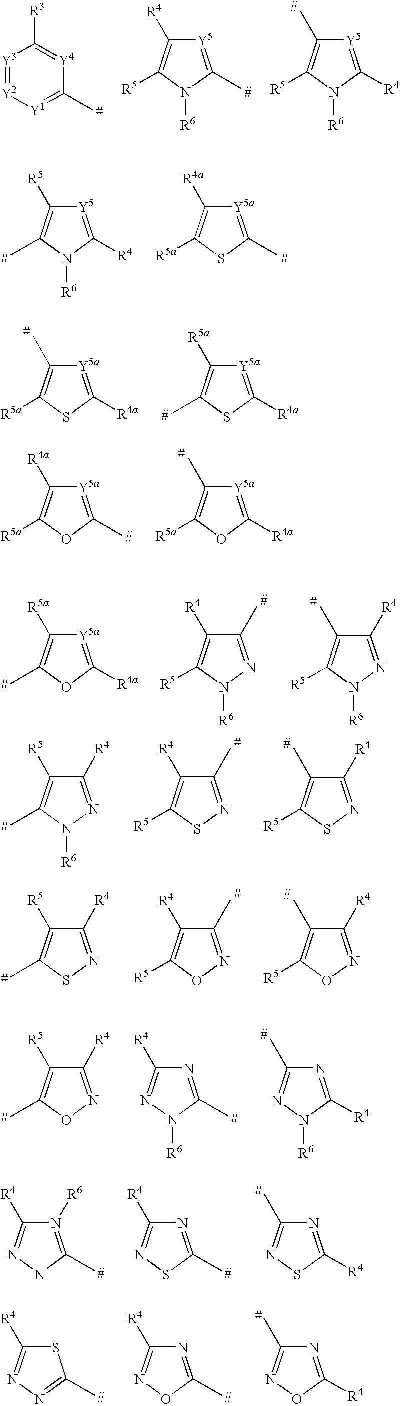

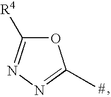

where A^{1a} and A^{2a} do not simultaneously represent optionally substituted phenyl,
Y¹, Y², Y³, Y⁴ and Y⁵ have the meanings given above,
Y^{5a} represents N or CR^{10a}, where at least one substituent R^{4a} or R^{5a} or R^{10a} does not represent hydrogen if Y^{5a} represents CR^{10a},
R³, R⁴, R⁵, R⁶, R⁷, R⁸, R⁹ and R¹⁰ have the meanings given above,
R^{4a}, R^{5a} and R^{10a} have the meanings of R⁴, R⁵ and R¹⁰, respectively, given above, where at least one substituent R^{4a} or R^{5a} or R^{10a} does not represent hydrogen,
X¹ has the meanings of X given above, with the proviso that X¹ does not represent pyrimidine-2,4-diyl or pyrimidine-4,6-diyl.

The novel compounds of the formula (I-1) can be used for controlling unwanted microorganisms and insects in crop protection and in the protection of materials.

The preferred, particularly preferred, very particularly preferred and especially preferred meanings of the individual radical definitions given above for the compounds of the formula (I) apply correspondingly to the compounds of the formula (I-1).
R^{1a} and R^{2a} independently of one another preferably represent chlorine, bromine, iodine, C₁-C₆-alkyl, C₃-C₆-cycloalkyl, C₁-C₆-haloalkyl having 1 to 13 fluorine, chlorine and/or bromine atoms, OH, C₁-C₆-alkoxy, (C₁-C₄-alkoxy)carbonyl, SH, C₁-C₆-alkylthio, C₁-C₆-alkylsulphinyl, C₁-C₆-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₆-alkyl)amine, (C₁-C₆-alkyl)amine, C₂-C₆-alkenyl, C₂-C₆-alkynyl, represent phenyl, benzyl, heterocyclyl or hetaryl, each of which is optionally substituted by halogen, cyano, C₁-C₄-haloalkyl having 1 to 9 fluorine, chlorine and/or bromine atoms, C₁-C₄-alkyl, C₂-C₄-alkenyl, C₂-C₄-alkynyl, C₁-C₄-alkoxy.
R^{1a} and R^{2a} independently of one another particularly preferably represent chlorine, bromine, C₁-C₄-alkyl, cyclopropyl, cyclobutyl, cyclopentyl, cyclohexyl, C₁-C₄-haloalkyl having 1 to 9 fluorine, chlorine and/or bromine atoms, OH, C₁-C₄-alkoxy, (C₁-C₄-alkoxy)carbonyl, SH, C₁-C₄-alkylthio, C₁-C₄-alkylsulphinyl, C₁-C₄-alkylsulphonyl, C₁-C₂-alkoxy-C₁-C₂-alkyl, (C₁-C₂-alkyl)thio(C₁-C₂-alkyl), (C₁-C₂-alkyl)sulphinyl(C₁-C₂-alkyl), (C₁-C₂-alkyl)sulphonyl(C₁-C₂-alkyl), amino, di(C₁-C₄-alkyl)amine, (C₁-C₄-alkyl)amine, C₂-C₄-alkenyl, C₂-C₄-alkynyl, represent phenyl, benzyl, thienyl, piperazinyl or morpholinyl, each of which is optionally substituted by fluorine, chlorine, bromine, cyano, trifluoromethyl, difluoromethyl, trichloromethyl, dichloromethyl, C₁-C₄-alkyl, C₁-C₄-alkoxy.
R^{1a} and R^{2a} independently of one another very particularly preferably represent chlorine, methyl, ethyl, n-propyl, i-propyl, n-, i-, s- or t-butyl, cyclopropyl, cyclopentyl, cyclohexyl, trifluoromethyl, difluoro-methyl, trichloromethyl, pentafluoroethyl, heptafluoro-n-propyl, heptafluoro-i-propyl, OH, methoxy, ethoxy, n-propoxy, i-propoxy, n-, i-, s- or t-butoxy, methoxycarbonyl, ethoxycarbonyl, SH, methylthio, ethylthio, n-propylthio, i-propylthio, n-, i-, s- or t-butylthio, methylsulphinyl, ethylsulphinyl, n-propylsulphinyl, i-propylsulphinyl, n-, i-, s- or t-butylsulphinyl, methylsulphonyl, ethylsulphonyl, n-propylsulphonyl, i-propylsulphonyl, n-, i-, s- or t-butylsulphonyl, methoxymethyl, methoxyethyl, ethoxyethyl, ethoxymethyl, methylthiomethyl, methylthioethyl, ethylthioethyl, ethylthiomethyl, methylsulphonylmethyl, methylsulphonylethyl, ethylsulphonylethyl, ethylsulphonylmethyl, amino, di(C₁-C₄-alkyl)amine, (C₁-C₄-alkyl)amine, C₂-C₄-alkenyl, C₂-C₄-alkynyl, represent phenyl, benzyl, thienyl, piperazinyl or morpholinyl each of which is optionally substituted by fluorine, chlorine, bromine, halogen, cyano, trifluoromethyl, methyl, ethyl, n-propyl, i-propyl, n-, i-, s-, t-butyl, methoxy, ethoxy, n-propoxy, i-propoxy, n-, i-, s-, t-butoxy.
R^{1a} and R^{2a} independently of one another represent chlorine, methyl, ethyl, n-propyl, i-propyl, i-butyl, cyclopropyl, trifluoromethyl, difluoromethyl, trichloromethyl, methoxycarbonyl, ethoxycarbonyl, methoxymethyl, methylthiomethyl, methylsulphonylmethyl, amino, 3-buten-1-yl, phenyl, benzyl, thienyl, 4-methylpiperazinyl or morpholinyl.
R^{1a} and R^{2a} most preferably each represent methyl, ethyl, n-propyl or amino, in particular methyl or ethyl.

A^{1a} and A^{2a} independently of one another preferably represent one of the cycles A-1 to A-43 below:

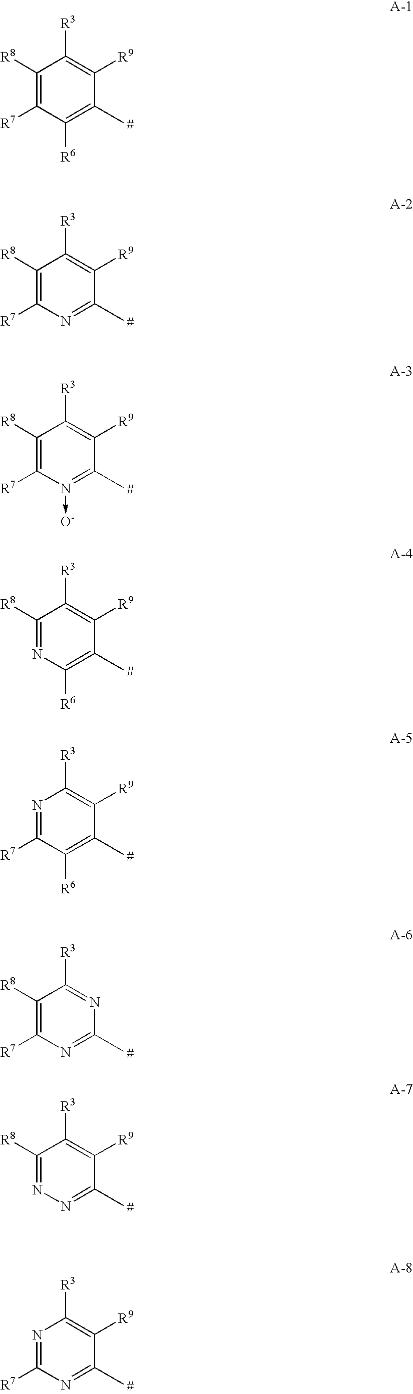

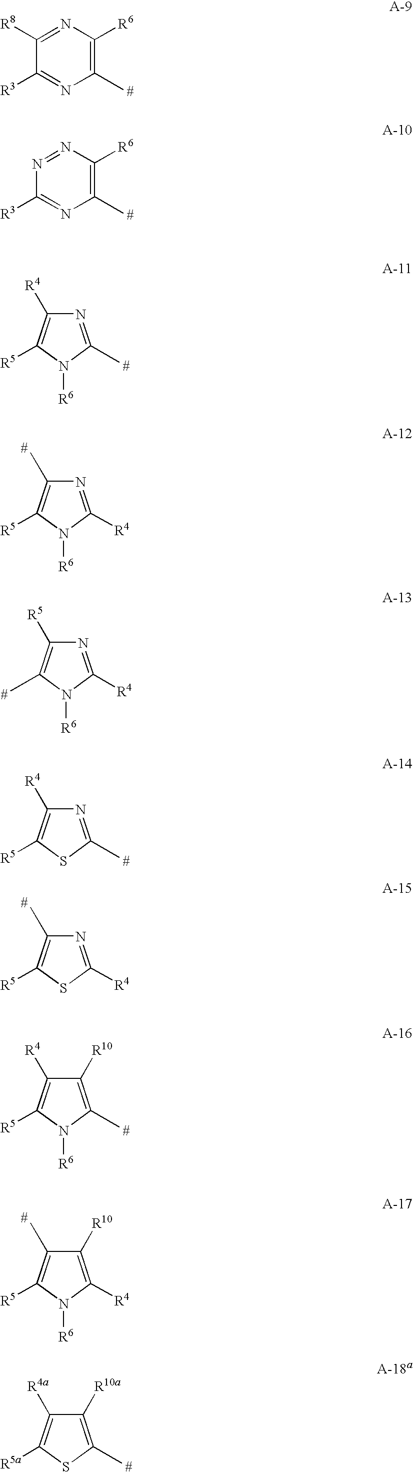

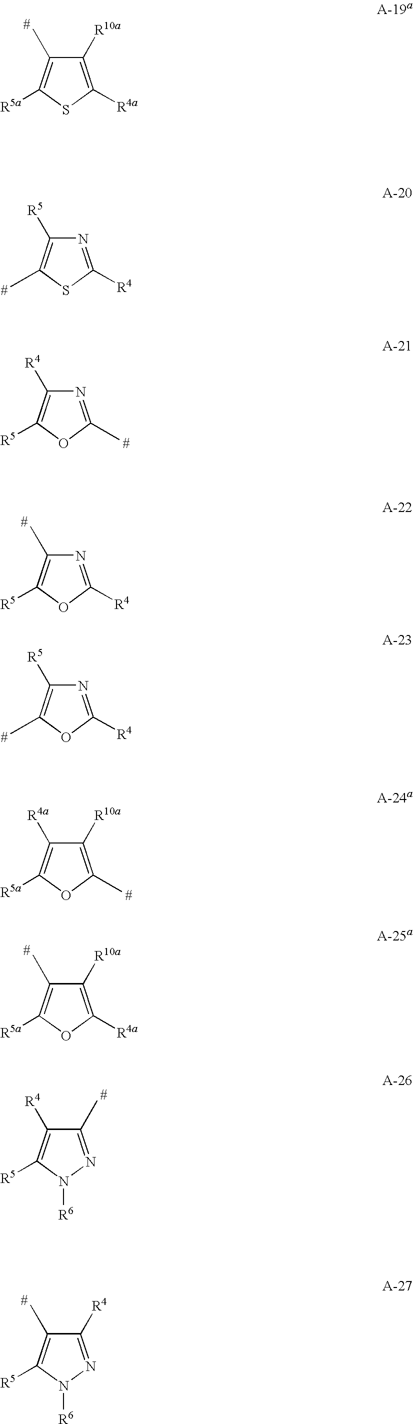

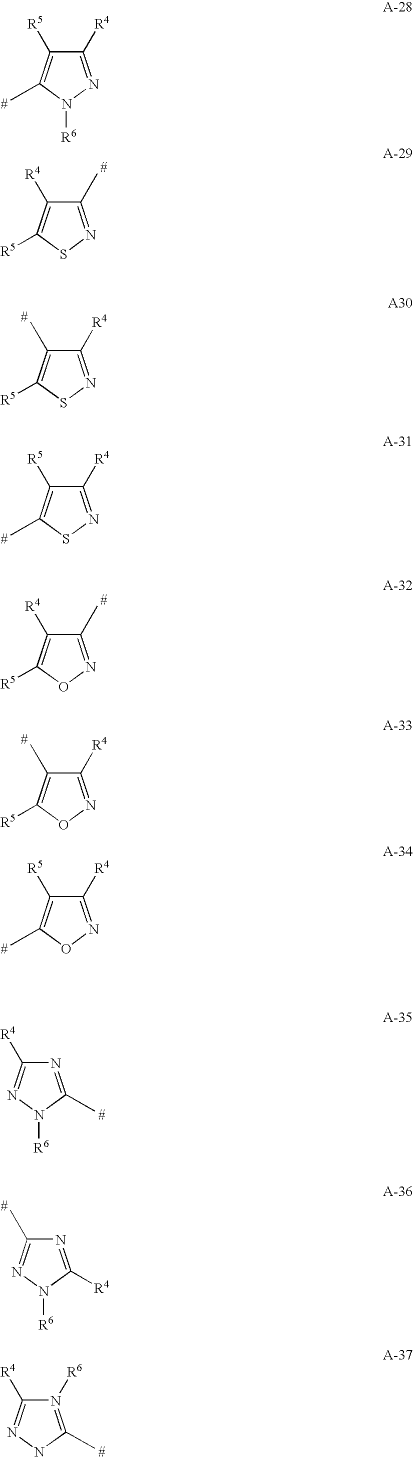

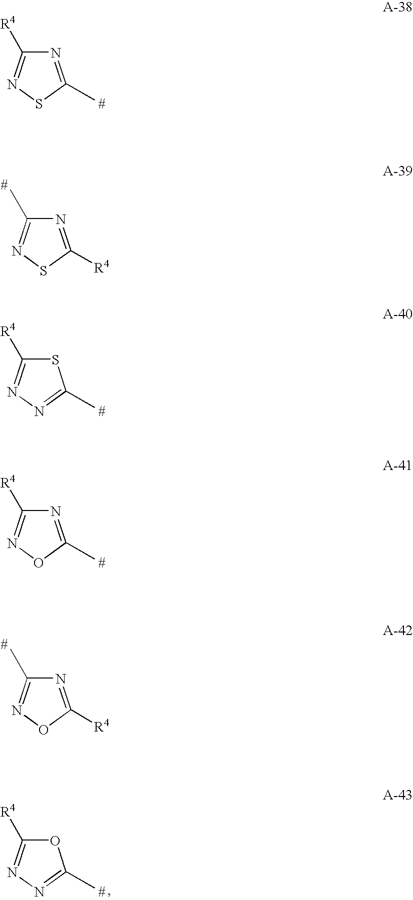

where A^{1a} and A^{2a} do not simultaneously represent the cycle A-1 and where in A-18^{a}, A-19^{a}, A-24^{a} and A-25^{a} at least one substituent R^{4a} or R^{5a} or R^{10a} does not represent hydrogen.
A^{1a} and A^{2a} independently of one another particularly preferably represent one of the cycles A-1, A-2, A-4, A-5, A-6, A-7, A-8, A-9, A-10, A-11, A-12, A-13, A-14, A-15, A-16, A-17, A-18^{a}, A-19^{a}, A-20, A-21, A-22, A-23, A-24^{a}, A-25^{a}, A-26, A-27, A-28, A-29, A-30, A-31, A-32, A-33 or A-34, where A^{1a} and A^{2a} do not simultaneously represent the cycle A-1 and where in A-18^{a}, A-19^{a}, A-24^{a} and A-25^{a} at least one substituent R^{4a} or R^{5a} or R^{10a} does not represent hydrogen.
A^{1a} and A^{2a} independently of one another very particularly preferably represent one of the cycles A-1, A-2, A-4, A-5, A-6, A-7, A-8, A-9, A-10, A-11, A-14, A-15, A-16, A-18^{a}, A-20, A-21, A-24^{a}, A-26, A-30 or A-33, where A^{1a} and A^{2a} do not simultaneously represent the cycle A-1 and where in A-18^{a} and A-24^{a} at least one substituent R^{4a} or R^{5a} or R^{10a} does not represent hydrogen.
A^{1a} and A^{2a} independently of one another especially preferably represent one of the cycles A-1, A-2, A-4, A-5, A-6, A-7, A-8, A-9 or A-10, where A^{1a} and A^{2a} do not simultaneously represent the cycle A-1.
A^{1a} and A^{2a} most preferably each represent A-2.

Preference is given to using those compounds of the formula (I) in which all radicals each have the meanings mentioned above as being preferred.

Particular preference is given to using those compounds of the formula (I) in which all radicals each have the meanings mentioned above as being particularly preferred.

Very particular preference is given to using those compounds of the formula (I) in which all radicals each have the meanings mentioned above as being very particularly preferred.

Special preference is given to using those compounds of the formula (I) in which all radicals each have the meanings mentioned above as being especially preferred.

Preference is given to those compounds of the formula (I-1) in which all radicals each have the meanings mentioned above as being preferred.

Particular preference is given to those compounds of the formula (I-1) in which all radicals each have the meanings mentioned above as being particularly preferred.

Very particular preference is given to those compounds of the formula (I-1) in which all radicals each have the meanings mentioned above as being very particularly preferred.

Special preference is given to those compounds of the formula (I-1) in which all radicals each have the meanings mentioned above as being especially preferred.

Moreover, preference is given to using compounds of the formula (I) in which R¹ and R² are each methyl.

Moreover, preference is given to compounds of the formula (I-1) in which R^{1a} and R^{2a} are each methyl.

Moreover, preference is given to using compounds of the formula (I), in which A¹ and A² are each cycle A-2.

Moreover, preference is given to compounds of the formula (I-1) in which A^{1a} and A^{2a} are each cycle A-2.

Moreover, preference is given to using compounds of the formula (I) in which R¹ and R² are each methyl and A¹ and A² are each cycle A-2.

Moreover, preference is given to compounds of the formula (I-1), in which R^{1a} and R^{2a} are each methyl and A^{1a} and A^{2a} are each cycle A-2.

In the definitions of the symbols given in the formulae above, collective terms were used which are generally representative for the following substituents:

halogen: fluorine, chlorine, bromine and iodine;
alkyl: saturated straight-chain or branched hydrocarbon radicals having 1 to 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; heptyl, octyl.
haloalkyl: straight-chain or branched alkyl groups having 1 to 8 carbon atoms (as mentioned above), where in these groups some or all of the hydrogen atoms may be replaced by halogen atoms as mentioned above, for example C₁-C₃-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.
alkenyl: unsaturated straight-chain or branched hydrocarbon radicals having 2 to 8 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.
alkynyl: straight-chain or branched hydrocarbon groups having 2 to 8 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.
cycloalkyl: monocyclic saturated hydrocarbon groups having 3 to 8 carbon ring members, such as cyclopropyl, cyclobutyl, cyclopentyl, cyclohexyl, cycloheptyl and cyclooctyl.
cycloalkenyl: monocyclic nonaromatic hydrocarbon groups having 3 to 8 carbon ring members and at least one double bond, such as cyclopenten-1-yl, cyclohexen-1-yl, cyclohepta-1,3-dien-1-yl.
alkoxycarbonyl: an alkoxy group having 1 to 6 carbon atoms (as mentioned above) which is attached to the skeleton via a carbonyl group (—CO—).
oxyalkyleneoxy: divalent straight-chain chains of 1 to 3 CH₂ groups where both valencies are attached to the skeleton via an oxygen atom, for example OCH₂O, OCH₂CH₂O and OCH₂CH₂CH₂O.
heterocyclyl/hetaryl: an unsubstituted or substituted unsaturated or fully or partially saturated heterocyclic 5- to 7-membered ring or an unsaturated or fully or partially saturated heterocyclic 3- to 8-membered ring which contains up to 4 nitrogen atoms or alternatively 1 nitrogen atom and up to 2 further heteroatoms selected from the group consisting of N, O and S: for example oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,3-dihydrothien-2-yl, 2,3-dihydrothien-3-yl, 2,4-dihydrothien-2-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetra-hydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl, 1-pyrrolyl, 1-pyrazolyl, 1,2,4-triazol-1-yl, 1-imidazolyl, 1,2,3-triazol-1-yl, 1,3,4-triazol-1-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Using 1-(6-methylpyridin-2-yl)ethanone and 1,3-bis(aminooxy)-2-methylpropane as starting materials, the course of the process (A-1) according to the invention can be illustrated by the formula scheme below.

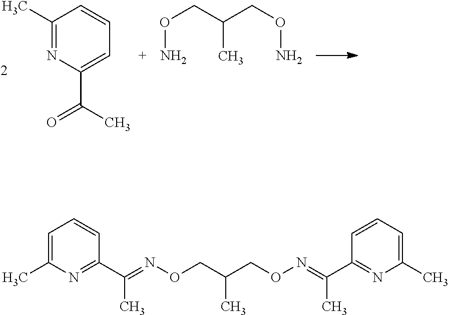

Using 1-(5,6-dimethylpyridin-2-yl)ethanone and 1,1-bis[(aminooxy)methyl]cyclopropane as starting materials for preparing (1E)-1-(5,6-dimethylpyridin-2-yl)ethanone O-({1-[(aminooxy)methyl]-cyclopropyl}methyl)oxime which is then reacted with 1-(4,6-dimethylpyridin-2-yl)ethanone, the course of the process (A-2) according to the invention can be illustrated by the formula scheme below.

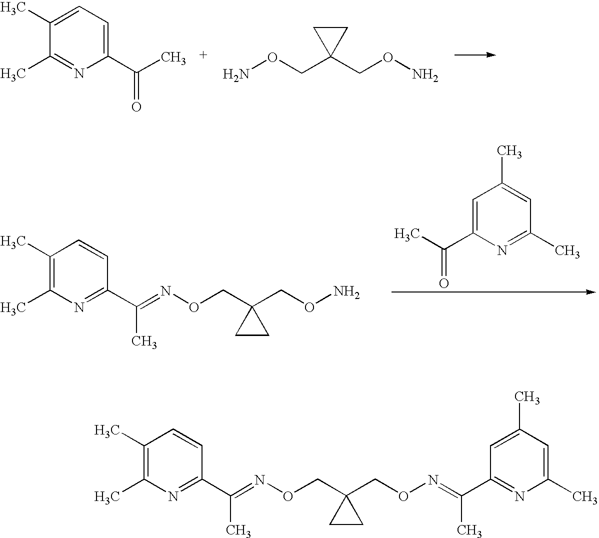

Using (1E)-1-(6-methylpyridin-2-yl)ethanone oxime and bis(chloromethyl)(dimethyl)silane as starting materials, the course of the process (B) according to the invention can be illustrated by the formula scheme below.

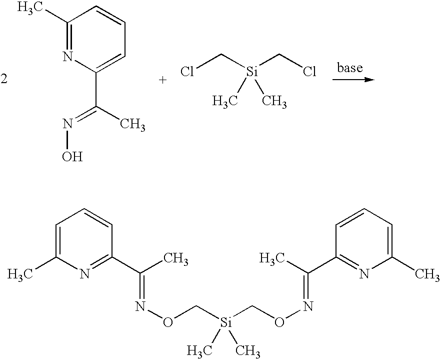

Using (1E)-1-(6-methylpyridin-2-yl)ethanone oxime and 1-bromo-3-chloro-2,2-dimethylpropane as starting materials to give (1E)-1-(6-methylpyridin-2-yl)ethanone O-(3-chloro-2,2-dimethylpropyl)oxime which is then reacted with (1E/Z)-1-(4,6-dimethylpyridin-2-yl)ethanone oxime, the course of the process (C) according to the invention can be illustrated by the formula scheme below.

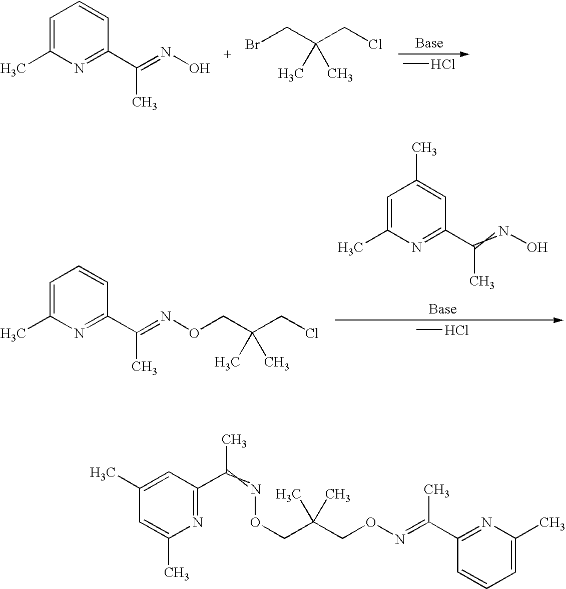

Using (1E)-1-(6-methylpyridin-2-yl)ethanone 0-{2-[({[(1E)-1-(6-methylpyridin-2-yl)ethylidene]-amino}oxy)methyl]prop-2-en-1-yl}oxime, ozone and hydroxylammonium chloride as starting materials the course of the process (D) according to the invention can be illustrated by the formula scheme below.

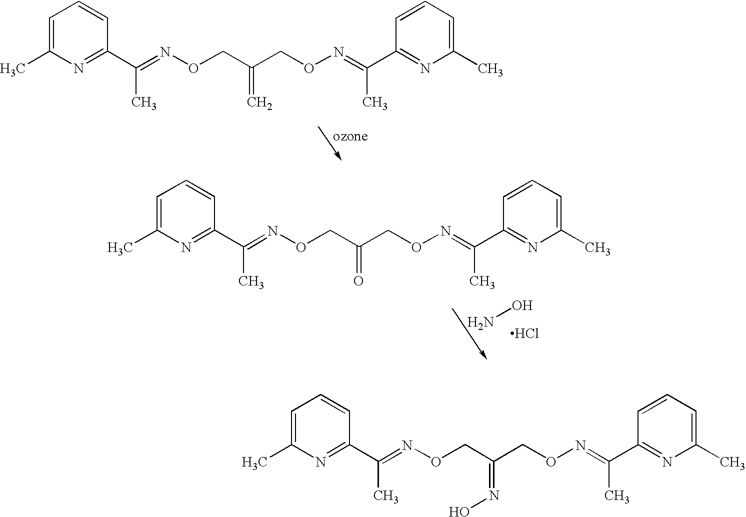

Using (1E)-1-(6-methylpyridin-2-yl)ethanone 0-{2-[({[(1E)-1-(6-methylpyridin-2-yl)ethylidene]-amino}oxy)methyl]prop-2-en-1-yl}oxime and (1E)-propanal oxime as starting materials, the course of the process (E) according to the invention can be illustrated by the formula scheme below.

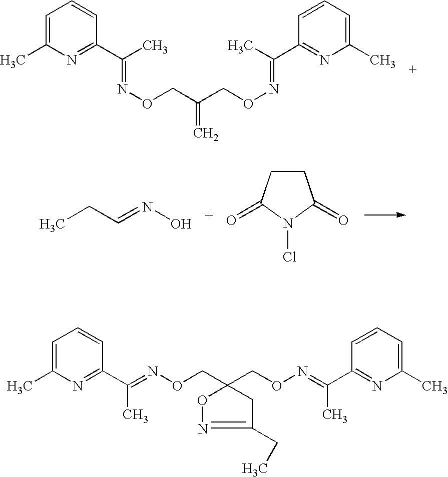

Using N′-({2-[({[(1Z)-amino-(4,6-dimethylpyrimidin-2-yl)methylene]amino}oxy)methyl]prop-2-en-1-yl}oxy)-4,6-dimethylpyrimidine-2-carboximidamide and hydrogen as starting materials, the course of the process (F) according to the invention can be illustrated by the formula scheme below.

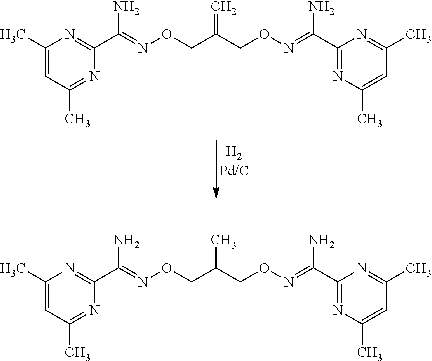

### Illustration of the Processes and Intermediates

The formulae (II) and (IIa) provide general definitions of the acyl(het)aryls required as starting materials for carrying out the processes (A-1) and (A-2) according to the invention. In these formulae, A¹, R¹, R² and A² preferably, particularly preferably, very particularly preferably and especially preferably have those meanings which have already been mentioned in connection with the description of the compounds of the formula (I) which can be used according to the invention as being preferred, particularly preferred etc., for these radicals.

The acyl(het)aryls of the formulae (II) and (IIa) are either commercially available or described in the literature or can be obtained by procedures analogously to the literature. Thus, for example, acyl(het)aryls of the formulae (II) and (IIa) can be synthesized according to the Grignard method by reacting appropriately substituted aromatic or heteroaromatic nitrites with the corresponding Grignard reagents R¹MgHal or R²MgHal, where R¹ and R² have the meanings given above and Hal represents halogen (preferably chlorine or bromine) (cf. J. Am. Chem. Soc. 1956, 78, 2141). Alternatively, the acyl(het)aryls of the formulae (II) and (IIa) can be prepared by Friedel-Crafts acylation of the (hetero)aromatics (for example J. Am. Chem. Soc. 1947, 69, 3018) or from carboxylic acid derivatives (esters, amides, acids) (for example iron-catalysed addition of methyl Grignard compound to carbonyl chlorides: J. Org. Chem. 2004, 69, 3943; addition of methyl Grignard compound to Weinreb amides: Synlett 1999, 1091; addition of malonic esters to carbonyl chlorides followed by double-decarboxylation: Tetrahedron 1992, 48, 9233).

Novel and thus also part of the subject-matter of the present application are the acyl(het)aryls of the formulae (II-1), (II-2) and (II-3)

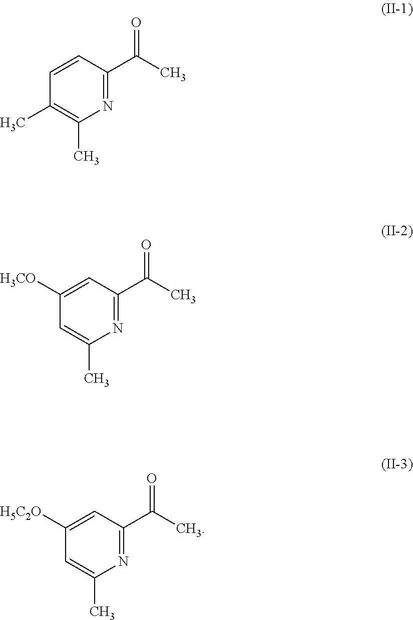

The formula (III) provides a general definition of the bishydroxylamines furthermore required as starting materials for carrying out the processes (A-1) and (A-2) according to the invention. In this formula, X preferably, particularly preferably, very particularly preferably and especially preferably has those meanings which have already been mentioned in connection with the description of the compounds of the formula (I) which can be used according to the invention as being preferred, particularly preferred, etc., for this radical. It is also possible to use the bishydrohalides of the bishydroxylamines of the formula (III) as starting materials. Here, the bishydrochlorides or bishydrobromides are particularly suitable.

Some of the bishydroxylamines of the formula (III) are known. Novel and thus also part of this invention are bishydroxylamines of the formula (IIIa)

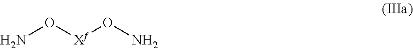

in which
X^{f} has the meanings of X,
except for compounds of the formula (IIIa), in which
X^{f} represents a saturated or unsaturated aliphatic straight-chain or branched hydrocarbon radical of 2 to 12 carbon atoms, which may be interrupted by 1 to 3 non-adjacent oxygen atoms [in particular —CH₂—, —(CH₂)₂—, —(CH₂)₃—, —(CH₂)₄—, —(CH₂)₅—, —(CH₂)₆—, —(CH₂)₈—, —(CH₂)₁₀—, —(CH₂)₂—O—(CH₂)₂—, —(CH₂)₂—O—(CH₂)₂—O—(CH₂)₂—], or represents —SiMe₂-, —SitBu₂-, —Si(Me)tBu-, —SiH₂—, —C(═O)—C(═O)—, (1-phenyl)ethane-1,2-diyl, —CH(CO₂Et)—CH₂—, —CH₂—CH(OH)—CH₂—, —CH₂—CHCl—CH₂, —CH₂—N(NO₂)—CH₂, —CH(CO₂Et)-CH₂—CH(CO₂Et)-, —C(═O)—CH(OH)—CH₂—C(═O)—, —C(═O)—(CH₂)₂—C(═O)—, —CH(CO₂H)—(CH₂)₂—CH(CO₂H)—, —(CH₂)₂—CHBr—(CH₂)₂—, —CH₂—N(NO₂)—CH₂—N(NO₂)—CH₂—, —(CH₂)₂—N[(CH₂)₄NH₂]-(CH₂)₂—, —CH(CO₂H)—(CH₂)₃—CH(CO₂H)—, —CH(CO₂H)—(CH₂)₄—CH(CO₂H)—, —CH₂—C(═O)—NH—NH—C(═O)—CH₂—, —CH₂—CH(CO₂H)—NH—C(═O)—C(NO₂)═CH—CH₂—, —CH₂—CH(OH)—CH═N—CH₂—CH(OH)—CH₂, —CH(CO₂H)—(CH₂)₆—CH(CO₂H)—, —(CH₂)₂—NH—(CH₂)₄—NH—(CH₂)₂—, —C(═O)—NH—(CH₂)₆—NH—C(O)—, —CH₂—C(═O)—NH—(CH₂)₂—NH—C(═O)—CH₂—, —CH₂—C(═O)—NH—NH—C(═O)—CH₂—O—NH—C(═O)—CH₂—,
or represents one of the groupings below

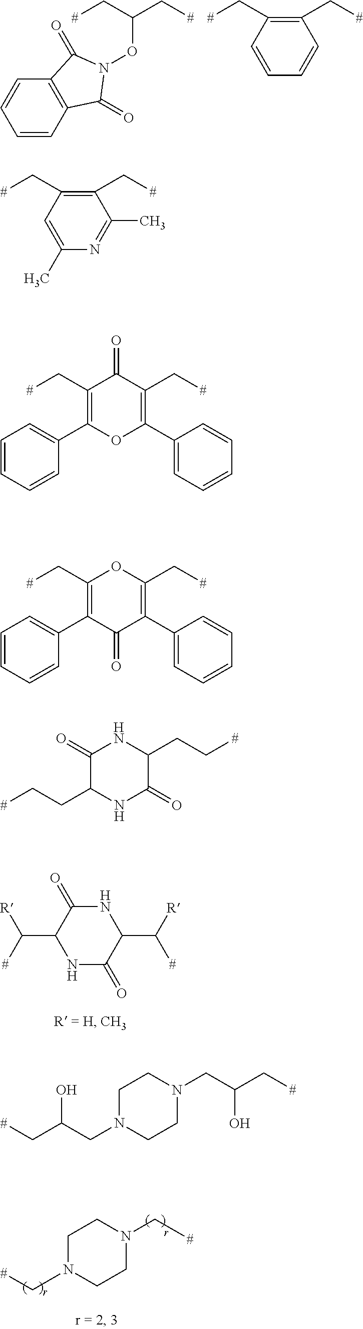

Bishydroxylamines of the formulae (III) and (IIIa) can be prepared by

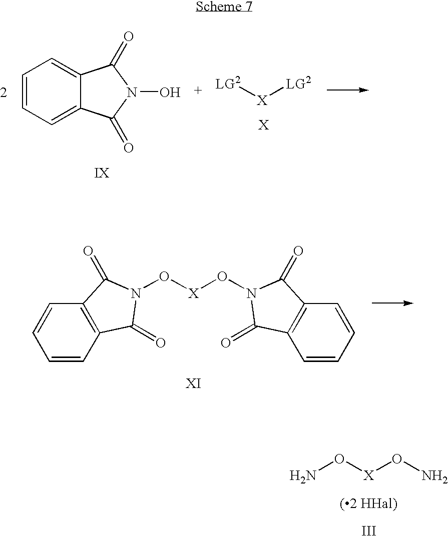

(G) reacting N-hydroxyphthalimide of the formula (IX)

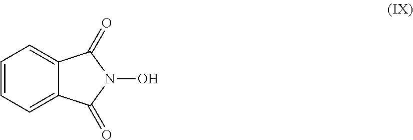

with compounds of the formula (X)

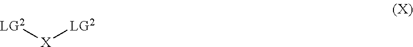

in which
LG² represents halogen, sulphonate or hydroxyl,
X has the meanings given above,
to give bishydroxyphthalimide derivatives of the formula (XI)

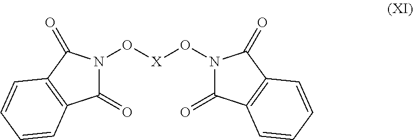

in which X has the meanings given above
(for example analogously to Chemistry A European Journal 2005, 11, 6974, Central European Journal of Chemistry 2003, 1, 57) and then releasing therefrom the bishydroxylamines of the formula (III) and (IIIa) (cf. Scheme 7).

When the bishydroxyphthalimide derivatives of the formula (XI) are reacted with hydrazine, the free bishydroxylamines of the formula (III) are obtained (for example analogously to J. Med. Chem. 1998, 41, 3220). Other releasing methods afford the corresponding salts (for example analogously to Asian Journal of Chemistry 2000, 12, 14; Central European Journal of Chemistry 2003, 1, 57).

The formula (XII) provides a general definition of the hydroxylamines (or their hydrohalides) which are formed as intermediates when carrying out the process (A-2) according to the invention. In this formula, A¹, R¹ and X preferably, particularly preferably, very particularly preferably and especially preferably have those meanings which have already been mentioned in connection with the description of the compounds of the formula (I) which can be used according to the invention as being preferred, particularly preferred, etc., for these radicals.

If appropriate, the hydroxylamines of the formula (XII) according to the invention can be present as mixtures of various possible isomeric forms, in particular of stereoisomers, such as, for example, E and Z, threo and erythro, and also optical isomers, and, if appropriate, also of tautomers. What is claimed are both the E and the Z isomers, and the threo and erythro, and also the optical isomers, any mixtures of these isomers, and also the possible tautomeric forms.

The compounds of the formula (XII) can be present in particular in the form of E or Z isomers or mixtures thereof, for example according to the formulae below:

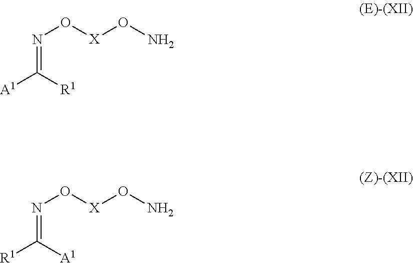

For the sake of simplicity, only one form is shown in each case, preferably the E form.

Novel and thus also part of the subject-matter of the present invention are the hydroxylamines (or their hydrohalides) of the formula (XIIa)

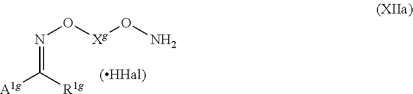

in which
R^{1g}, A^{1g} and X^{g} each have the meanings given above for R¹, A¹ and X, where
a) A^{1g} does not represent phenyl, 4-fluorophenyl, 4-chlorophenyl or 2,4-dichlorophenyl if R^{1g} represents hydrogen and X^{g} represents —(CH₂)₄—, or
b) A^{1g} does not represent phenyl, 4-fluorophenyl, 4-chlorophenyl, 2,4-dichlorophenyl, 3,4-dichlorophenyl, 2-hydroxyphenyl, 2-hydroxy-5-bromophenyl, 3-{[(2S)-2-hydroxy-propyl]oxy}phenyl, 2-hydroxy-3,5-bis-tert-butylphenyl, 2-hydroxy-3-methylthiophenyl, 2-hydroxy-3-methoxyphenyl or 4-nitrophenyl, if R^{1g} represents hydrogen and X^{g} represents —(CH₂)₂—.

It is also novel that the hydroxylamines (or their hydrohalides) of the formula (XII) have fungicidal properties (see Preparation Examples and Use Examples).

Preferred, particularly preferred, very particularly preferred and especially preferred are the compounds of the formula (XIIa), in which the radicals R^{1g}, A^{1g} and X^{g} each have the preferred, particularly preferred, very particularly preferred and especially preferred meanings of the radicals R¹, A¹ and X which have already been mentioned in connection with the description of the compounds of the formula (I) which can be used according to the invention as being preferred, particularly preferred, etc., for these radicals, with the proviso that in each case
a) A^{1g} does not represent phenyl, 4-fluorophenyl, 4-chlorophenyl or 2,4-dichlorophenyl, if R^{1g} represents hydrogen and X^{g} represents —(CH₂)₄—, or
b) A^{1g} does not represent phenyl, 4-fluorophenyl, 4-chlorophenyl, 2,4-dichlorophenyl, 3,4-dichlorophenyl, 2-hydroxyphenyl, 2-hydroxy-5-bromophenyl, 3-{[(2S)-2-hydroxy-propyl]oxy}phenyl, 2-hydroxy-3,5-bis-tert-butylphenyl, 2-hydroxy-3-methylthiophenyl, 2-hydroxy-3-methoxyphenyl or 4-nitrophenyl, if R^{1g} represents hydrogen and X^{g} represents —(CH₂)₂—

The formula (IV) provides a general definition of the bifunctional compounds required as starting materials for carrying out the processes (B) and (C) according to the invention. In this formula, X preferably, particularly preferably, very particularly preferably and especially preferably has those meanings which have already been mentioned in connection with the description of the compounds of the formula (I) which can be used according to the invention as being preferred, particularly preferred, etc., for this radical. LG¹ preferably represents chlorine, bromine, iodine, mesylate or tosylate, particularly preferably chlorine or bromine.

Bifunctional compounds of the formula (IV) are either commercially available or described in the literature or obtainable by procedures analogously to the literature. Thus, for example, bischlorides can be obtained by reacting diols with thionyl chloride/pyridine (for example analogously to Journal of Organic Chemistry 1995, 60, 4666), whereas bisbromides are obtainable by reacting diols with glacial acetic acid/hydrobromic acid (for example analogously to Journal of Organic Chemistry 1995, 60, 4946). Bissulphonates of the formula (IV) are likewise either commercially available or described in the literature or obtainable by procedures analogously to the literature (for example synthesis of bismesylates analogously to Chem. Pharm. Bull. 2006, 54, 141; bistrifluoromethanesulphonates analogously to Journal of Organic Chemistry 2006, 71, 5423; bistosylates analogously to Carbohydrate Research 2005, 340, 2494).

The formulae (V) and (Va) provide a general definition of the free oximes furthermore required as starting materials for carrying out the processes (B) and (C) according to the invention. In these formulae, A¹, R¹, R² and A² preferably, particularly preferably, very particularly preferably and especially preferably have those meanings which have already been mentioned in connection with the description of the compounds of the formula (I) which can be used according to the invention as being preferred, particularly preferred, etc., for these radicals.

The free oximes of the formulae (V) and (Va) can be obtained by condensing the acyl(het)aryls of the formulae (II) and (IIa) with hydroxylamine (for example analogously to Journal of Organic Chemistry 2003, 68, 3546) or they can be obtained by reaction of aromatic or heteroaromatic nitriles with hydroxylamine (for example synthesis of amidoximes analogously to EP-A 0 490 188).

The formulae (VI) and (VIa) provide a general definition of the compounds which occur as intermediates when carrying out the process (C) according to the invention. In these formulae, A¹, R¹, R², A² and X preferably, particularly preferably, very particularly preferably and especially preferably have those meanings which have already been mentioned in connection with the description of the compounds of the formula (I) which can be used according to the invention as being preferred, particularly preferred, etc., for these radicals. LG¹ preferably represents chlorine, bromine, iodine, mesylate or tosylate, particularly preferably chlorine or bromine.

Intermediates of the formulae (VI) and (VIa) are novel and also form part of the subject-matter of the present invention.

If appropriate, the intermediates of the formulae (VI) and (VIa) according to the invention can be present as mixtures of various possible isomeric forms, in particular of stereoisomers, such as, for example, E and Z, threo and erythro, and also optical isomers, and, if appropriate, also of tautomers. What is claimed are both the E and the Z isomers, and also the threo and erythro, and also the optical isomers, any mixtures of these isomers, and also the possible tautomeric forms.

The intermediates of the formulae (VI) and (VIa) can be present in particular in the form of E or Z isomers or mixtures thereof, for example according to the formulae below:

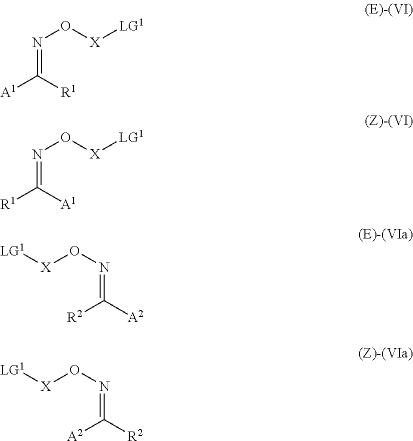

For the sake of simplicity, only one form is shown in each case.

Process (B) is the one-pot variant (tandem reaction) of process (C).

The formulae (Ib) and (Ic) provide general definitions of the bisoximes which are required as starting materials or occur as intermediates when carrying out the process (D) according to the invention. In these formulae, A¹, R¹, R² and A² preferably, particularly preferably, very particularly preferably and especially preferably have those meanings which have already been mentioned in connection with the description of the compounds of the formula (I) which can be used according to the invention as being preferred, particularly preferred, etc., for these radicals. In this formula, X^{b} preferably, particularly preferably, very particularly preferably and especially preferably has those meanings which have already been mentioned in connection with the description of the compounds of the formula (I) which can be used according to the invention as being preferred, particularly preferred, etc., for the radical X, with the proviso that X^{b} contains a group CR¹³R¹⁴ which is not adjacent to the oxime groups in formula (I) and where for their part R¹³ and R¹⁴ together represent ═CR¹⁸R¹⁹ and R¹⁸ and R¹⁹ have the general, preferred, particularly preferred, very particularly preferred and especially preferred meanings given above. The definition of X^{b} given above is equivalent herewith. X^{c} has preferably, particularly preferably, very particularly preferably and especially preferably those meanings which have already been mentioned in connection with the description of the compounds of the formula (I) which can be used according to the invention as being preferred, particularly preferred, etc., for the radical X, with the proviso that X^{c} contains a group CR¹³R¹⁴ which is not adjacent to the oxime groups in formula (I) and where for their part R¹³ and R¹⁴ together represent ═O. The definition of X^{c} given above is equivalent herewith.

Bisoximes of the formulae (Ib) and (Ic) are likewise compounds according to the invention and are a subgroup of the bisoximes of the formula (I).

The formula (VII) provides a general definition of the free oximes furthermore required as starting materials for carrying out the process (D) according to the invention. In this formula, R²⁰ preferably, particularly preferably, very particularly preferably and especially preferably has those meanings which have already been mentioned in connection with the description of the compounds of the formula (I) which can be used according to the invention as being preferred, particularly preferred, etc., for this radical.

Free oximes of the formula (VII) are known or can be obtained by known processes.

The bisoximes of the formula (Ib) required as starting materials for carrying out the processes (E) and (F) according to the invention have already been described in connection with process (D).

The formula (VIII) provides a general definition of the aldoximes furthermore required as starting materials for carrying out the process (E) according to the invention. In this formula, R²¹ preferably, particularly preferably, very particularly preferably and especially preferably has those meanings which have already been mentioned in connection with the description of the compounds of the formula (I) which can be used according to the invention as being preferred, particularly preferred etc., for this radical.

The N-hydroxyphthalimide of the formula (IX) used for carrying out the process (G) according to the invention is a known chemical synthesis.

The formula (X) provides a general definition of the compounds furthermore required as starting materials for carrying out the process (G) according to the invention. In this formula, X preferably, particularly preferably, very particularly preferably and especially preferably has those meanings which have already been mentioned in connection with the description of the compounds of the formula (I) which can be used according to the invention as being preferred, particularly preferred, etc., for this radical. LG² preferably represents chlorine, bromine, iodine, mesylate, tosylate or hydroxyl, particularly preferably chlorine, bromine or hydroxyl.

Compounds of the formula (X) are known or can be obtained analogously to the preparation of compounds of the formula (IV) by known processes.

The formula (XI) provides a general definition of the bishydroxyphthalimide derivatives which furthermore occur as intermediates when carrying out the process (G) according to the invention. In this formula, X preferably, particularly preferably, very particularly preferably and especially preferably has those meanings which have already been mentioned in connection with the description of the compounds of the formula (I) which can be used according to the invention as being preferred, particularly preferred, etc., for this radical.

Some of the bishydroxyphthalimide derivatives of the formula (XI) are known. Novel and thus also part of this invention are bishydroxyphthalimide derivatives of the formula (XIa)

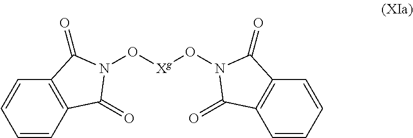

in which
X^{g} has the meanings of X,
except for compounds of the formula (XIa), in which
X^{g} represents —C(═O)—, —C(═NH)—, —(CH₂)₂—, —(CH₂)₃—, —(CH₂)₄—, —(CH₂)₅—, —(CH₂)₆—, —C(═O)—C(═O)—, (1-phenyl)ethane-1,2-diyl, —CH₂—CCl₂—, —CH₂—CH(CH₂—ON═CH₂)— —CH₂—CH(CH₂OH)—, —CH₂—CH(OH)—CH₂—, —CH(CH₃)—(CH₂)₂—, —CH(CO₂Et)-CH₂—CH(CO₂Et)-, —CH(CO₂Et)-(CH₂)₂—CH(CO₂Et)-, —CH(CO₂Et)-(CH₂)₃—CH(CO₂Et)-, —CH(CO₂Et)-(CH₂)₄—CH(CO₂Et)-, —(CH₂)₂—O—(CH₂)₂—O—(CH₂)₂—, —(CH₂)₂—O—(CH₂)₂—, —CH(CO₂Et)-(CH₂)₆—CH(CO₂Et)-, —CH₂—C(═O)—NH—(CH₂)₂—NH—C(═O)—CH₂—,
or represents one of the groupings below

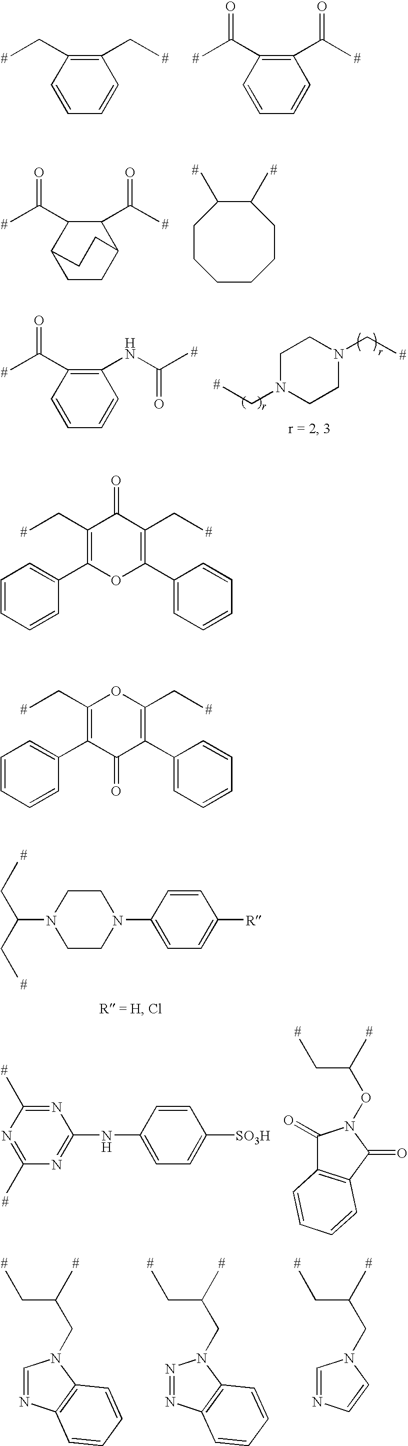

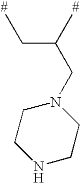

Bishydroxyphthalimide derivatives of the formula (XIa) can be prepared by process (G).

The processes (A-1), (A-2), (D) and (F) according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methyl-pyrrolidone, and also dimethyl sulphoxide, tetramethylene sulphone and hexamethylphosphoric triamide. However, it is also possible to use alcohols, such as, for example, methanol, ethanol, propanol, i-propanol, butanol, i-butanol, 2-methoxyethanol. Preference is given to using ethanol and methanol and mixtures thereof with dichloromethane.

The processes (B), (C), (E) and (G) according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl and dibutyl ether, glycol dimethyl ether and diglycol dimethyl ether, tetrahydrofuran and dioxane, ketones. such as acetone, methyl ethyl ketone, methyl isopropyl ketone or methyl isobutyl ketone, esters, such as methyl acetate or ethyl acetate, nitriles such as, for example, acetonitrile or propionitrile, amides, such as, for example, dimethylformamide, dimethylacetamide and N-methyl-pyrrolidone, and also dimethyl sulphoxide, tetramethylene sulphone and hexamethylphosphoric triamide. Preference is given to using acetonitrile, diethyl ether or tetrahydrofuran.

When carrying out the processes (A-1), (A-2), (B), (C), (D), (F) and (G) according to the invention, the reaction temperatures can be varied within a relatively wide range. In general, the processes are carried out at temperatures between 0° C. and 250° C., preferably at temperatures between 0° C. and 100° C.

When carrying out the process (E) according to the invention, the reaction temperatures can be varied within a relatively wide range. In general, the process is carried out at temperatures between −78° C. and 25° C., preferably at temperatures between −40° C. and 0° C.

Suitable catalysts for carrying out the processes (A-1), (A-2) and (D) according to the invention are, if appropriate, the customary inorganic or organic acids. These preferably include para-toluenesulphonic acid, methanesulphonic acid, hydrochloric acid or sulphuric acid.

Suitable reaction auxiliaries for carrying out the processes (B) and (C) according to the invention are, if appropriate, the customary inorganic or organic bases or acid binders. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, calcium carbonate or caesium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, -i-, -s- or -t-butoxide or potassium methoxide, ethoxide, n- or i-propoxide, n-, -i-, -s- or -t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diaza-bicyclo[2.2.2]octane (DABCO), 1,5-diazabicyclo[4.3.0]non-5-ene (DBN), 1,8-diazabicyclo[5.4.0]undec-7-ene (DBU), or phosphazene bases, such as, for example, P[N═P(NMe₂)₃]=NtBu. In process (C), preference is given to using P[N═P(NMe₂)₃]=NtBu.

The process (F) according to the invention is, if appropriate, carried out in the presence of a catalyst. Suitable catalysts are all catalysts which are customarily used for hydrogenations. Examples which may be mentioned are: Raney nickel, palladium or platinum, if appropriate on a support, such as, for example, activated carbon.

If LG² in formula (X) represents halogen or sulphonate, suitable reaction auxiliaries for carrying out the first step of the process (G) according to the invention are the customary inorganic or organic bases or acid binders. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, -i-, -s- or -t-butoxide or potassium methoxide, ethoxide, n- or i-propoxide, n-, -i-, -s- or -t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN), 1,8-diazabicyclo[5.4.0]undec-7-ene (DBU). Preference is given to using 1,8-diazabicyclo[5.4.0]undec-7-ene (DBU).

If LG² in formula (X) represents hydroxyl, suitable reaction auxiliaries for carrying out the process (G) according to the invention are combinations of trialkyl- or triarylphosphines and azodicarboxylates. These preferably include triphenylphosphine and diethyl azodicarboxylate (DEAD) or diisopropyl azodicarboxylate (DIAD).

Suitable for carrying out the second step of the process (G) according to the invention are either hydrazine hydrate or a mixture of organic and inorganic acids. These preferably include glacial acetic acid, hydrochloric acid, hydrobromic acid. Preference is given to using a mixture of glacial acetic acid and hydrobromic acid.

The processes (A-1), (A-2), (B), (C), (D), (E), (F) and (G) according to the invention are generally carried out under atmospheric pressure. However, it is also possible to operate under elevated or reduced pressure—in general between 0.1 bar and 10 bar.

When carrying out the process (A-1) according to the invention, in general 1 mol of acyl(het)aryl of the formula (IIa) and from 0.9 to 1.1 mol, preferably 1 mol, of bishydroxylamine of the formula (III) and also 0.1 mol of a catalyst are employed per mole of acyl(het)aryl of the formula (II). If a bishydrohalide of the bishydroxylamine of the formula (III) is employed, from 0.1 to 0.5 mol of catalyst is used. However, it is also possible to employ the reaction components in other ratios. Work-up is carried out by customary methods. In general, water is added to the reaction mixture and the organic phase is removed and, after drying, concentrated under reduced pressure. If appropriate, the residue that remains can be freed from any impurities that may still be present using customary methods, such as chromatography or recrystallization.

When carrying out the process (A-2) according to the invention, in the first step from 0.9 to 1.1 mol, preferably 1 mol, of bishydroxylamine of the formula (III) and also 0.1 mol of a catalyst are employed per mole of acyl(het)aryl of the formula (II). If a bishydrohalide of the bishydroxylamine of the formula (III) is employed, from 0.1 to 0.5 mol of catalyst is used. However, it is also possible to employ the reaction components in other ratios. Work-up is carried out by customary methods. In general, water is added to the reaction mixture and the organic phase is removed and, after drying, concentrated under reduced pressure. If appropriate, the residue that remains can be freed from any impurities that may still be present using customary methods, such as chromatography or recrystallization. In the second step of the process (A-2) according to the invention, from 0.9 to 1.1 mol, preferably 1 mol, of acyl(het)aryl of the formula (IIa) and also 0.1 mol of a catalyst are employed per mole of the hydroxylamine of the formula (XII). However, it is also possible to employ the reaction components in other ratios. Work-up is carried out by customary methods. In general, water is added to the reaction mixture and the organic phase is removed and, after drying, concentrated under reduced pressure. If appropriate, the residue that remains can be freed from any impurities that may still be present using customary methods, such as chromatography or recrystallization.

When carrying out the process (B) according to the invention, in general 1 mol of free oxime of the formula (Va) and 1 mol of bishalo compound (IV) and also from 1 to 10 mol of acid binder are employed per mole of the free oxime of the formula (V). However, it is also possible to employ the reaction components in other ratios. Work-up is carried out by customary methods. In general, water is added to the reaction mixture and the organic phase is removed and, after drying, concentrated under reduced pressure. If appropriate, the residue that remains can be freed from any impurities that may still be present using customary methods, such as chromatography or recrystallization.

When carrying out the process (C) according to the invention, 1 mol of bishalide of the formula (IV) and also from 1 to 10 mol of acid binder are employed per mole of the free oxime of the formula (V) or (Va). In the second step, 1 mol of the free oxime of the formula (V) or (Va) and also from 1 to 10 mol of acid binder are employed per mole of the intermediate of the formula (VI) or (VIa). However, it is also possible to employ the reaction components in other ratios. Work-up is carried out by customary methods. In general, water is added to the reaction mixture and the organic phase is removed and, after drying, concentrated under reduced pressure. If appropriate, the residue that remains can be freed from any impurities that may still be present using customary methods, such as chromatography or recrystallization.

When carrying out the process (D) according to the invention, in the first step, per mole of bisoxime of the formula (Ib), such an amount of ozone is used that, according to thin-layer chromatography, the conversion into a bisoxime of the formula (Ic) has ended. In the second step, 1 mol of hydroxylamine of the formula (VII) is employed per mole of bisoxime of the formula (Ic). However, it is also possible to employ the reaction components in other ratios. Work-up is carried out by customary methods. In general, water is added to the reaction mixture and the precipitate is removed and dried. If appropriate, the residue that remains can be freed from any impurities that may still be present using customary methods, such as chromatography or recrystallization.

When carrying out the process (E) according to the invention, 1.3 mol of aldoxime of the formula (III) and 1.3 mol of N-chlorosuccinimide are employed per mole of the bisoxime of the formula (Ib). However, it is also possible to employ the reaction components in other ratios. Work-up is carried out by customary methods. In general, water is added to the reaction mixture and the organic phase is removed and, after drying, concentrated under reduced pressure. If appropriate, the residue that remains can be freed from any impurities that may still be present using customary methods, such as chromatography or recrystallization.

When carrying out the process (F) according to the invention, 0.1 mol of catalyst and excess hydrogen are employed per mole of the bisoxime of the formula (Ib). However, it is also possible to employ the reaction components in other ratios. Work-up is carried out by customary methods. In general, the reaction mixture is filtered off and the organic phase is concentrated under reduced pressure. If appropriate, the residue that remains can be freed from any impurities that may still be present using customary methods, such as chromatography or recrystallization.

When carrying out the process (G) according to the invention, in the first step 0.5 mol of bisfunctional compound X and from 1 mol to 1.5 mol of phosphine and also from 1 mol to 1.5 mol of dialkyl azodicarboxylate are employed per mole of N-hydroxyphthalimide of the formula (IX). Preferably, 1.1 mol of phosphine and 1.1 mol of dialkyl azodicarboxylate are used. In the second step, 2 mol of hydrazine hydrate or an excess of a 2:3 mixture of glacial acetic acid/hydrobromic acid are used per mole of the intermediate (XI). Work-up is carried out by customary methods. In general, in the first step, water is added to the reaction mixture and the precipitate is separated off and dried. In the second step, the reaction mixture is generally filtered and the filtrate is concentrated under reduced pressure. If appropriate, the residue that remains can be freed from any impurities that may still be present using customary methods, such as chromatography or recrystallization.

The compounds according to the invention exhibit a potent microbicidal activity and can be employed in crop protection and in the protection of materials for controlling undesirable microorganisms such as fungi and bacteria.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Examples which may be mentioned, but not by limitation, of some pathogens of fungal and bacterial diseases which come under the abovementioned general terms are:

diseases caused by powdery mildew pathogens, such as, for example Blumeria species such as, for example, Blumeria graminis; Podosphaera species such as, for example, Podosphaera leucotricha; Sphaerotheca species such as, for example, Sphaerotheca fuliginea; Uncinula species such as, for example, Uncinula necator;
diseases caused by rust pathogens such as, for example, Gymnosporangium species such as, for example, Gymnosporangium sabinae; Hemileia species such as, for example, Hemileia vastatrix; Phakopsora species such as, for example, Phakopsora pachyrhizi and Phakopsora meibomiae; Puccinia species such as, for example, Puccinia recondita or Puccinia graminis; Uromyces species such as, for example, Uromyces appendiculatus;
diseases caused by pathogens from the Oomycetes group such as, for example, Bremia species such as, for example, Bremia lactucae; Peronospora species such as, for example, Peronospora pisi or P. brassicae; Phytophthora species such as, for example, Phytophthora infestans; Plasmopara species such as, for example, Plasmopara viticola; Pseudoperonospora species such as, for example, Pseudoperonospora humuli or Pseudoperonospora cubensis; Pythium species such as, for example, Pythium ultimum;
leaf spot diseases and leaf wilts caused by, for example, Alternaria species such as, for example, Alternaria solani; Cercospora species such as, for example, Cercospora beticola; Cladosporium species such as, for example, Cladosporium cucumerinum; Cochliobolus species such as, for example, Cochliobolus sativus (conidial form: Drechslera, Syn: Helminthosporium); Colletotrichum species such as, for example, Colletotrichum lindemuthanium; Cycloconium species such as, for example, Cycloconium oleaginum; Diaporthe species such as, for example, Diaporthe citri; Elsinoe species such as, for example, Elsinoe fawcettii; Gloeosporium species such as, for example, Gloeosporium laeticolor; Glomerella species such as, for example, Glomerella cingulata; Guignardia species such as, for example, Guignardia bidwelli; Leptosphaeria species such as, for example, Leptosphaeria maculans; Magnaporthe species such as, for example, Magnaporthe grisea; Mycosphaerella species such as, for example, Mycosphaerella graminicola and Mycosphaerella fijiensis; Phaeosphaeria species such as, for example, Phaeosphaeria nodorum; Pyrenophora species such as, for example, Pyrenophora teres; Ramularia species such as, for example, Ramularia collocygni; Rhynchosporium species such as, for example, Rhynchosporium secalis; Septoria species such as, for example, Septoria apii; Typhula species such as, for example, Typhula incamata; Venturia species such as, for example, Venturia inaequalis;
root and stem diseases caused by, for example, Corticium species such as, for example, Corticium graminearum; Fusarium species such as, for example, Fusarium oxysporum; Gaeumannomyces species such as, for example, Gaeumannomyces graminis; Rhizoctonia species such as, for example, Rhizoctonia solani; Tapesia species such as, for example, Tapesia acuformis or Tapesia yallundae; Thielaviopsis species such as, for example, Thielaviopsis basicola;
ear and panicle diseases (including maize cobs), caused by, for example, Alternaria species such as, for example, Alternaria spp.; Aspergillus species such as, for example, Aspergillus flavus; Cladosporium species such as, for example, Cladosporium cladosporioides; Claviceps species such as, for example, Claviceps purpurea; Fusarium species such as, for example, Fusarium culmorum; Gibberella species such as, for example, Gibberella zeae; Monographella species such as, for example, Monographella nivalis;
diseases caused by smuts such as, for example, Sphacelotheca species such as, for example, Sphacelotheca reiliana; Tilletia species such as, for example, Tilletia caries; Urocystis species such as, for example, Urocystis occulta; Ustilago species such as, for example, Ustilago nuda;
fruit rots caused by, for example, Aspergillus species such as, for example, Aspergillus flavus; Botrytis species such as, for example, Botrytis cinerea; Penicillium species such as, for example, Penicillium expansum and Penicillium purpurogenum; Sclerotinia species such as, for example, Sclerotinia sclerotiorum; Verticilium species such as, for example, Verticilium alboatrum;
seed- and soil-borne rot and wilts, and seedling diseases, caused by, for example, Fusarium species such as, for example, Fusarium culmorum; Phytophthora species, such as, for example, Phytophthora cactorum; Pythium species such as, for example, Pythium ultimum; Rhizoctonia species such as, for example, Rhizoctonia solani; Sclerotium species such as, for example, Sclerotium rolfsii;
cancers, galls and witches' broom disease, caused by, for example, Nectria species such as, for example, Nectria galligena;
wilts caused by, for example, Monilinia species such as, for example, Monilinia laxa;
deformations of leaves, flowers and fruits, caused by, for example, Taphrina species such as, for example, Taphrina deformans;
degenerative diseases of woody species, caused by, for example, Esca species such as, for example, Phaeomoniella chlamydospora and Phaeoacremonium aleophilum and Fomitiporia mediterranea;
diseases of flowers and seeds, caused by, for example, Botrytis species such as, for example, Botrytis cinerea; diseases of the plant tubers, caused by, for example, Rhizoctonia species such as, for example, Rhizoctonia solani; Helminthosporium species such as, for example, Helminthosporium solani;
diseases caused by bacterial pathogens, such as, for example, Xanthomonas species such as, for example, Xanthomonas campestris pv. oryzae; Pseudomonas species such as, for example, Pseudomonas syringae pv. lachrymans; Erwinia species such as, for example, Erwinia amylovora;

The following diseases of soybeans can preferably be controlled:

Fungal diseases on leaves, stems, pots and seeds caused by, for example,
alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola),
fungal diseases on roots and the stem base caused by, for example,
black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also have a potent strengthening effect in plants. They are therefore suitable for mobilizing the plants' defenses against attack by undesired microorganisms.

Plant-strengthening (resistance-inducing) substances are understood as meaning, in the present context, those substances which are capable of stimulating the defense system of plants in such a way that, when subsequently inoculated with undesired microorganisms, the treated plants display a substantial degree of resistance to these microorganisms.

In the present case, undesired microorganisms are understood as meaning phytopathogenic fungi, bacteria and viruses. Thus, the substances according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Puccinia species and Pyrenophora species and diseases in viticulture, fruit production and vegetable production such as, for example, against Botrytis, Venturia or Alternaria species.

The active compounds according to the invention are also suitable for increasing the yield. Moreover, they display a low degree of toxicity and are well tolerated by plants.

If appropriate, the active compounds according to the invention can also be used in certain concentrations and application rates as herbicides, for influencing plant growth and for controlling animal pests. If appropriate, they can also be employed as intermediates and precursors for the synthesis of further active compounds.

All plants and plant parts can be treated in accordance with the invention. Plants are understood as meaning, in the present context, all plants and plant populations, such as desired and undesired wild plants or crop plants (including naturally occurring crop plants). Crop plants may be plants which can be obtained by conventional breeding and optimization methods or else by biotechnological and genetic engineering methods or by combinations of these methods, including the transgenic plants and including the plant varieties capable or not capable of being protected by Plant Breeders' rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention with the active compounds, of the plants and plant parts, is carried out directly or by acting on their environment, habitat, or store by the customary treatment methods, for example by immersion, spraying, vaporizing, fogging, broadcasting, painting on and, in the case of propagation material, in particular in the case of seeds, furthermore by coating with one or more coats.

In the protection of materials, the compounds according to the invention can be employed for protecting industrial materials against attack and destruction by undesired microorganisms.

In the present context, industrial materials are understood as meaning non live materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example, cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood.

Microorganisms which are capable of bringing about a degradation or modification of the industrial materials and which may be mentioned are, for example, bacteria, fungi, yeasts, algae and slime organisms. The active compounds according to the invention are preferably active against fungi, in particular moulds, wood-discolouring and wood-destroying fungi (Basidiomycetes) and against slime organisms and algae.

Examples which may be mentioned are microorganisms of the following genera:

Alternaria, such as Alternaria tenuis, Aspergillus, such as Aspergillus niger, Chaetomium, such as Chaetomium globosum, Coniophora, such as Coniophora puetana, Lentinus, such as Lentinus tigrinus, Penicillium, such as Penicillium glaucum, Polyporus, such as Polyporus versicolor, Aureobasidium, such as Aureobasidium pullulans, Sclerophoma, such as Sclerophoma pityophila, Trichoderma, such as Trichoderma viride, Escherichia, such as Escherichia coli, Pseudomonas, such as Pseudomonas aeruginosa, Staphylococcus, such as Staphylococcus aureus.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and mollusks, which are encountered in agriculture, in horticulture, in animal husbandry, in forests, in gardens and leisure facilities, in the protection of stored products and of materials, and in the hygiene sector. They may be preferably employed as crop protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnostema consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofinannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

Depending on their particular physical and/or chemical properties, the active compounds can be converted into the customary formulations, such as solutions, emulsions, suspensions, powders, foams, pastes, granules, aerosols, microencapsulations in polymeric substances and in coating materials for seed, and also ULV cold and warm fogging formulations.

The active compounds can be used as such, in the form of their formulations or in the use forms prepared therefrom, such as ready-to-use solutions, emulsions, water- or oil-based suspensions, powders, wettable powders, pastes. soluble powders, dusts, soluble granules, granules for broadcasting, suspoemulsion concentrates, natural substances impregnated with active compound, synthetic substances impregnated with active compound, fertilizers and also microencapsulations in polymeric substances. Application is carried out in a customary manner, for example by watering, spraying, atomizing, broadcasting, dusting, foaming, spreading, etc. It is furthermore possible to apply the active compounds by the ultra-low volume method or to inject the preparation of active compound or the active compound itself into the soil. It is also possible to treat the seed of the plants.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Liquefied gaseous extenders or carriers refer to liquids which are gaseous at standard temperature and standard pressure, for example aerosol propellants, such as halogenated hydrocarbons, and also butane, propane, nitrogen and carbon dioxide.

Suitable solid carriers are: for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

Further additives may be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.1 and 95% and particularly preferably between 0.5 and 90%.

The active compounds according to the invention, as such or in their formulations, can also be used as a mixture with known fungicides, bactericides, acaricides, nematicides, or insecticides, for example, to improve the activity spectrum or prevent the development of resistance.

Examples of co-components in mixtures are the following compounds:

### Fungicides:

1) Nucleic acid synthesis inhibitors: for example benalaxyl, benalaxyl-M, bupirimate, clozylacon, dimethirimol, ethirimol, furalaxyl, hymexazol, mefenoxam, metalaxyl, metalaxyl-M, ofurace, oxadixyl, oxolinic acid;
2) mitosis and cell division inhibitors: for example benomyl, carbendazim, diethofencarb, ethaboxam, fuberidazole, pencycuron, thiabendazole, thiophanate-methyl, zoxamide;
3) respiration inhibitors (inhibitors of the respiratory chain):
3.1) inhibitors which act on complex I of the respiratory chain: for example diflumetorim;
3.2) inhibitors which act on complex II of the respiratory chain: for example boscalid, carboxin, fenfuram, flutolanil, furametpyr, furmecyclox, mepronil, oxycarboxin, penthiopyrad, thifluzamide;
3.3) inhibitors which act on complex III of the respiratory chain: for example amisulbrom, azoxystrobin, cyazofamid, dimoxystrobin, enestrobin, famoxadone, fenamidone, fluoxastrobin, kresoxim-methyl, meto-minostrobin, orysastrobin, picoxystrobin, pyraclostrobin, trifloxystrobin;
4) decouplers: for example dinocap, fluazinam, meptyldinocap;
5) ATP production inhibitors: for example fentin acetate, fentin chloride, fentin hydroxide, silthiofam;
6) amino acid and protein biosynthesis inhibitors: for example andoprim, blasticidin-S, cyprodinil, kasugamycin, kasugamycin hydrochloride hydrate, mepanipyrim, pyrimethanil;
7) signal transduction inhibitors: for example fenpiclonil, fludioxonil, quinoxyfen;
8) lipid and membrane synthesis inhibitors: for example biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl, vinclozolin;
9) ergosterol biosynthesis inhibitors: for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;
10) cell wall synthesis inhibitors: for example benthiavalicarb, dimethomorph, flumorph, iprovalicarb, mandipropamid polyoxins, polyoxorim, validamycin A;
11) melanin biosynthesis inhibitors: for example carpropamid, diclocymet, fenoxanil, phthalide, pyroquilon, tricyclazole;
12) resistance inductors: for example acibenzolar-S-methyl, probenazole, tiadinil;
13) compounds with multi-site activity: for example Bordeaux mixture, captafol, captan, chlorothalonil, copper naphthenate, copper oxide, copper oxychloride, copper preparations such as, for example, copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations such as, for example, calcium polysulphide, thiram, tolylfluanid, zineb, ziram;
14) a compound selected from the following enumeration: N-methyl-(2E)-2-(2-{[6-(3-chloro-2-methylphenoxy)-5-fluoropyrimidin-4-yl]oxy}phenyl)-2-(methoxyimino)acetamide, N-methyl-(2E)-2-{2-[({[(1E)-1-(3-{[(E)-1-fluoro-2-phenylvinyl]oxy}phenyl)ethylidene]amino}oxy)methyl]phenyl}-2-(methoxyimino)acetamide, 1-(4-chlorophenyl)-2-(1H-1,2,4-triazol-1-yl)cycloheptanol, 1-[(4-methoxyphenoxy)methyl]-2,2-dimethylpropyl-1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulphonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts thereof, 3-(difluoromethyl)-1-methyl-N-[2-(1,1,2,2-tetrafluoroethoxy)phenyl]-1H-pyrazole-4-carboxamide, 3-(difluoromethyl)-N-[(9R)-9-isopropyl-1,2,3,4-tetrahydro-1,4-methanonaphthalen-5-yl]-1-methyl-1H-pyrazole-4-carboxamide, 3-(difluoromethyl)-N-[(9S)-9-isopropyl-1,2,3,4-tetrahydro-1,4-methanonaphthalen-5-yl]-1-methyl-1H-pyrazole-4-carboxamide, 3-(difluoromethyl)-N-[4′-(3,3-dimethylbut-1-yn-1-yl)-biphenyl-2-yl]-1-methyl-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-chloro-7-(4-methylpiperidin-1-yl)-6-(2,4,6-trifluorophenyl)[1,2,4]triazolo[1,5-a]pyrimidine, 8-hydroxyquinoline sulphate, benthiazole, bethoxazin, capsimycin, carvone, quinomethionate, cufraneb, cyflufenamid, cymoxanil, dazomet, debacarb, dichlorophen, diclomezine, dicloran, difenzoquat, difenzoquat methylsulphate, diphenylamine, ecomate, ferimzone, flumetover, fluopicolide, fluoroimide, flusulfamide, fosetyl-aluminium, fosetyl-calcium, fosetyl-sodium, hexachlorobenzene, irumamycin, isotianil, methasulfocarb methyl (2E)-2-{2-[({cyclopropyl[(4-methoxyphenyl)imino]methyl}thio)methyl]phenyl}-3-methoxyacrylate, methyl 1-(2,2-dimethyl-2,3-dihydro-1H-inden-1-yl)-1H-imidazole-5-carboxylate, methyl isothiocyanate, metrafenone, mildiomycin, N-[2-(1,3-dimethylbutyl)phenyl]-5-fluoro-1,3-dimethyl-1H-pyrazole-4-carboxamide, N-(3′,4′-dichloro-5-fluorobiphenyl-2-yl)-3-(difluoromethyl)-1-methyl-1H-pyrazole-4-carboxamide(bixafen), N-(3-ethyl-3,5,5-trimethylcyclohexyl)-3-(formylamino)-2-hydroxybenzamide, N-(4-chloro-2-nitrophenyl)-N-ethyl-4-methylbenzenesulphonamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N-(methylsulphonyl)valinamide, N-{(Z)-[(cyclopropylmethoxy)imino][6-(difluoromethoxy)-2,3-difluorophenyl]methyl}-2-phenylacetamide, N-{2-[1,1′-bi(cyclopropyl)-2-yl]phenyl}-3-(difluoromethyl)-1-methyl-1H-pyrazole-4-carboxamide, N-{2-[3-chloro-5-(trifluoromethyl)pyridin-2-yl]ethyl}-2-(trifluoromethyl)benzamide(fluopyram), natamycin, N-ethyl-N-methyl-N′-{2-methyl-5-(trifluoromethyl)-4-[3-(trimethyl-silyl)propoxy]phenyl}imidoformamide, N-ethyl-N-methyl-N′-{2-methyl-5-(difluoromethyl)-4-[3-(trimethylsilyl)propxy]phenyl}imidoformamide, nickel dimethyl dithiocarbamate, nitrothal-isopropyl, O-{1-[(4-methoxyphenoxy)methyl]-2,2-dimethylpropyl} 1H-imidazole-1-carbothioate, octhilinone, oxamocarb, oxyfenthiin, pentachlorophenol and salts, phosphoric acid and its salts, piperalin, propamocarb fosetylate, propanosine-sodium, proquinazid, pyribencarb, pyrrolnitrine, quintozene, S-allyl-5-amino-isopropyl-4-(2-methylphenyl)-3-oxo-2,3-dihydro-1H-pyrazole-1-carbothioate, tecloftalam, tecnazene, triazoxide, trichlamide, valiphenal, zarilamid.

### Bactericides:

Bronopol, dichlorophen, nitrapyrin, nickel dimethyl dithiocarbamate, kasugamycin, octhilinone, furan-carboxylic acid, oxytetracyclin, probenazole, streptomycin, tecloftalam, copper sulphate and other copper preparations.

### Insecticides/Acaricides/Nematicides:

### 1. Acetylcholine Esterase (AChE) Inhibitors

1.1 Carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbo-sulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 Organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2. Sodium Channel Modulators/Voltage-Dependent Sodium Channel Blockers

2.1 Pyrethroids (for example acrinathrin, allethrin (d-cis-trans, d-trans), beta-cyfluthrin, bifenthrin, bio-allethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum))
2.2 Oxadiazines (for example indoxacarb)

### 3. Acetylcholine Receptor Agonists/Antagonists

3.1 Chloronicotinyls/neonicotinoids (for example acetamiprid, clothianidin, dinotefuran, imidacloprid, nitenpyram, nithiazine, thiacloprid, thiamethoxam)
3.2 Nicotine, bensultap, cartap

### 4. Acetylcholine Receptor Modulators

4.1 Spinosyns (for example spinosad)

### 5. GABA-Controlled Chloride Channel Antagonists

5.1 Cyclodiene organochlorines (for example camphechlor, chlordane, endosulfan, gamma-HCH, HCH, heptachlor, lindane, methoxychlor)
5.2 Fiproles (for example acetoprole, ethiprole, fipronil, vaniliprole)

### 6. Chloride Channel Activators

6.1 Mectins (for example abamectin, avermectin, emamectin, emamectin benzoate, ivermectin, milbemectin, milbemycin)

### 7. Juvenile Hormone Mimetics

(for example diofenolan, epofenonane, fenoxycarb, hydroprene, kinoprene, methoprene, pyriproxifen, triprene)

### 8. Ecdysone Agonists/Disruptors

8.1 Diacylhydrazines (for example chromafenozide, halofenozide, methoxyfenozide, tebufenozide)

### 9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)

### 9.2 Buprofezin

### 9.3 Cyromazine

### 10. Inhibitors of Oxidative Phosphorylation, ATP Disruptors

### 10.1 Diafenthiuron

10.2 Organotins (for example azocyclotin, cyhexatin, fenbutatin oxide)

### 11. Uncouplers of Oxidative Phosphorylation by Interrupting the H-Proton Gradient

11.1 Pyrroles (for example chlorfenapyr)
11.2 Dinitrophenols (for example binapacyrl, dinobuton, dinocap, DNOC)

### 12. Site-I Electron Transport Inhibitors

12.1 METIs (for example fenazaquin, fenpyroximate, pyrimidifen, pyridaben, tebufenpyrad, tolfenpyrad)

### 12.2 Hydramethylnone

### 12.3 Dicofol

### 13. Site-II Electron Transport Inhibitors

### 13.1 Rotenone

### 14. Site-III Electron Transport Inhibitors

14.1 Acequinocyl, fluacrypyrim

### 15. Microbial Disruptors of the Insect Gut Membrane

Bacillus thuringiensis strains

### 16. Fat Synthesis Inhibitors

16.1 Tetronic acids (for example spirodiclofen, spiromesifen)
16.2 Tetramic acids [for example 3-(2,5-dimethylphenyl)-8-methoxy-2-oxo-1-azaspiro[4.5]dec-3-en-4-yl ethyl carbonate (also known as: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg.-No.: 203313-25-1)]

### 17. Carboxamides

(for example flonicamid)

### 18. Octopaminergic Agonists

(for example amitraz)

### 19. Magnesium-Stimulated ATPase Inhibitors

(for example propargite)

### 20. Ryanodin Receptor Agonists

20.1 benzoic acid dicarboxamides (for example N²-[1,1-dimethyl-2-(methylsulphonyl)ethyl]-3-iodo-N¹-[2-methyl-4-[1,2,2,2-tetrafluoro-1-(trifluoromethyl)ethyl]phenyl]-1,2-benzenedicarboxamide (CAS Reg.-No.: 272451-65-7), flubendiamide)
20.2 Anthranilamide (for example DPX E2Y45-3-bromo-N-{4-chloro-2-methyl-6-[(methylamino)carbonyl]-phenyl}-1-(3-chloropyridin-2-yl)-1H-pyrazol-5-carboxamide)

### 21. Nereistoxin Analogues

(for example thiocyclam hydrogen oxalate, thiosultap sodium)

### 22. Biologicals, Hormones or Pheromones

(for example azadirachtin, Bacillus spec., Beauveria spec., codlemone, Metarrhizium spec., Paecilomyces spec., thuringiensin, Verticillium spec.)
23. Active Compounds with Unknown or Unspecific Mechanisms of Action
23.1 Fumigants (for example aluminium phosphide, methyl bromide, sulphuryl fluoride)
23.2 Selective antifeedants (for example cryolite, flonicamid, pymetrozine)
23.3 Mite growth inhibitors (for example clofentezine, etoxazole, hexythiazox)
23.4 Amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methyl-phenyl propylcarbamate (tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo isomer (CAS Reg. No. 185984-60-5) (cf. WO 96/37494, WO 98/25923), and preparations which contain insecticidally active plant extracts, nematodes, fungi or viruses.

A mixture with other known active compounds such as herbicides, or with fertilizers and growth regulators, safeners or semiochemicals is also possible.

In addition, the compounds of the formula (I) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

The application rate of the active compounds according to the invention is
when treating leaves: from 0.1 to 10 000 g/ha, preferably from 10 to 1000 g/ha, particularly preferably from 50 to 300 g/ha (when the application is carried out by watering or dripping, it is even possible to reduce the application rate, especially when inert substrates such as rock wool or perlite are used);
when treating seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
when treating the soil: from 0.1 to 10 000 g/ha, preferably from 1 to 5000 g/ha.

These application rates are mentioned only in an exemplary manner and not limiting in the sense of the invention.

It is thus possible to employ the active compounds or compositions according to the invention to protect plants for a certain period of time after treatment against attack by the pathogens mentioned. In general, the period for which protection is provided extends over 1 to 28 days, preferably 1 to 14 days, after the treatment of the plants with the active compounds, or over up to 200 days after a seed treatment.

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec. inter alia.

The control of phytopathogenic fungi by treating the seeds of plants has been known for a long time and is subject-matter of continuous improvements. However, the treatment of seed frequently entails a series of problems which cannot always be solved in a satisfactory manner. Thus, it is desirable to develop methods for protecting the seed and the germinating plant which dispense with the additional application of crop protection agents after sowing or after the emergence of the plants or where additional applications are at least reduced. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore in particular also relates to a method for the protection of seed and germinating plants from attack by phytopathogenic fungi, by treating the seed with a composition according to the invention.

The invention likewise relates to the use of the compositions according to the invention for the treatment of seed for protecting the seed and the germinating plant from phytopathogenic fungi.

Furthermore, the invention relates to seed which has been treated with a composition according to the invention so as to afford protection from phytopathogenic fungi.

One of the advantages of the present invention is that the particular systemic properties of the compositions according to the invention mean that treatment of the seed with these compositions not only protects the seed itself, but also the resulting plants after emergence, from phytopathogenic fungi. In this manner, the immediate treatment of the crop at the time of sowing or shortly thereafter can be dispensed with.

Furthermore, it must be considered as advantageous that the mixtures according to the invention can also be employed in particular in transgenic seed.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawns and ornamental plants. The treatment of seed of cereals (such as wheat, barley, rye and oats), maize and rice is of particular importance.

In the context of the present invention, the composition according to the invention is applied to the seed either alone or in a suitable formulation. Preferably, the seed is treated in a state which is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, for example, it is possible to use seed which has been harvested, cleaned and dried to a moisture content of below 15% by weight. Alternatively, it is also possible to use seed which, after drying, has, for example, been treated with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the composition to the seed in the form of a suitable formulation. Suitable formulations and methods for the treatment of seed are known to the skilled worker and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations which can be used according to the invention can be converted into customary seed dressing formulations, such as solutions, emulsions, suspensions, powders, foams, slurries or other coating materials for seed, and also ULV formulations.

These formulations are prepared in a known manner by mixing the active compounds or active compound combinations with customary additives, such as, for example, customary extenders and also solvents or diluents, colorants, wetting agents, dispersants, emulsifiers, defoamers, preservatives, secondary thickeners, adhesives, gibberellins and water as well.

Suitable colorants that may be present in the seed dressing formulations which can be used according to the invention include all colorants customary for such purposes. Use may be made both of pigments, of sparing solubility in water, and of dyes, which are soluble in water. Examples that may be mentioned include the colorants known under the designations rhodamine B, C.I. Pigment Red 112, and C.I. Solvent Red 1.

Suitable wetting agents that may be present in the seed dressing formulations which can be used according to the invention include all substances which promote wetting and are customary in the formulation of active agrochemical substances. With preference it is possible to use alkylnaphthalene-sulphonates, such as diisopropyl- or diisobutylnaphthalene-sulphonates.

Suitable dispersants and/or emulsifiers that may be present in the seed dressing formulations which can be used according to the invention include all nonionic, anionic, and cationic dispersants which are customary in the formulation of active agrochemical substances. With preference, it is possible to use nonionic or anionic dispersants or mixtures of nonionic or anionic dispersants. Particularly suitable nonionic dispersants are ethylene oxide-propylene oxide block polymers, alkylphenol polyglycol ethers, and tristyrylphenol polyglycol ethers, and their phosphated or sulphated derivatives. Particularly suitable anionic dispersants are lignosulphonates, polyacrylic salts, and arylsulphonate-formaldehyde condensates.

Suitable defoamers that may be present in the seed dressing formulations which can be used according to the invention include all foam-inhibiting substances which are customary in the formulation of active agrochemical substances. With preference it is possible to use silicone defoamers and magnesium stearate.

Suitable preservatives that may be present in the seed dressing formulations which can be used according to the invention include all substances which can be used for such purposes in agrochemical compositions. By way of example, mention may be made of dichlorophen and benzyl alcohol hemiformal.

Suitable secondary thickeners that may be present in the seed dressing formulations which can be used according to the invention include all substances which can be used for such purposes in agrochemical compositions. Preferred suitability is possessed by cellulose derivatives, acrylic acid derivatives, xanthan, modified clays, and highly disperse silica.

Suitable adhesives that may be present in the seed dressing formulations which can be used according to the invention include all customary binders which can be used in seed dressing. With preference, mention may be made of polyvinylpyrrolidone, polyvinyl acetate, polyvinyl alcohol and tylose.

Suitable gibberellins that may be present in the seed dressing formulations which can be used according to the invention are preferably the gibberellins A1, A3 (=gibberellinic acid), A4 and A7; particularly preferably, gibberellinic acid is used. The gibberellins are known (cf. R. Wegler “Chemie der Pflanzenschutz-und Schädlingsbekämpfungsmittel”, Vol. 2, Springer Verlag, 1970, pp. 401-412).

The seed dressing formulations which can be used according to the invention may be used directly or after dilution with water beforehand to treat seed of any of a very wide variety of types. For instance, the concentrates or the preparations obtainable therefrom by dilution with water may be used to dress the seed of cereals, such as wheat, barley, rye, oats, and triticale, and also the seed of maize, rice, oilseed rape, peas, field beans, cotton, sunflowers, and beets, or else vegetable seed of any of a very wide variety of kinds. The seed dressing formulations which can be used according to the invention or their dilute preparations may also be used to dress seed of transgenic plants. In this context, synergistic effects may also arise in interaction with the substances formed by expression.

Suitable mixing equipment for treating seed with the seed dressing formulations which can be used according to the invention or the preparations prepared from them by adding water includes all mixing equipment which can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

The application rate of the seed dressing formulations which can be used according to the invention may be varied within a relatively wide range. It depends on the respective content of the active compounds in the formulations and on the seed. In general, the application rates of active compound combination are between 0.001 and 50 g per kilogram of seed, preferably between 0.01 and 15 g per kilogram of seed.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues. The active compound content of the use forms prepared from the commercially available formulations can vary within wide limits. The active compound concentration of the use forms can be from 0.00000001 to 95% by weight of active compound, preferably between 0.00001 and 1% by weight. The compounds are employed in a customary manner appropriate for the use forms.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, further developed root system, higher resistance of the plant species or plant cultivars, increased growth of the shoots, higher plant vitality, increased tolerance to high or low temperatures, increased tolerance to drought or to water or soil salt content, increased flowering performance, easier harvesting, accelerated maturation, higher harvest yields, larger fruits, larger plant sizes, greener colour of the leaves, earlier flowering, higher quality and/or a higher nutritional value of the harvested products, higher sugar concentration in the fruits, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particular advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defense of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defense of plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reductions in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100- to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, in particular hulls, screens, nets, buildings, moorings and signalling systems, against fouling.

Furthermore, the compounds according to the invention, alone or in combinations with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages.

These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

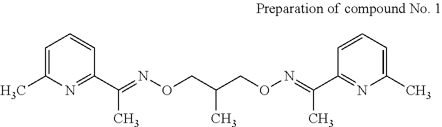

A solution of 6.00 g (44.4 mmol) of 2-acetyl-6-methylpyridine (II-3), 7.00 g (23.3 mmol) of [(2-methylpropane-1,3-diyl)bis(oxy)]diammonium dibromide (III-1) and 300 mg of p-toluenesulphonic acid in 240 ml of ethanol was heated under reflux for 4 h. After cooling, the reaction mixture was poured into ice-water and neutralized by addition of triethylamine. After extraction with dichloromethane, the combined organic phases were dried over Na₂SO₄ and freed from the solvent under reduced pressure.

Purification by column chromatography on silica gel using petroleum ether/methyl t-butyl ether gave 5.50 g (15.5 mmol, 70% of theory) of 1-(6-methylpyridin-2-yl)ethanone O,O′-(2-methylpropane-1,3-diyl)oxime of logP (HCO₂H)=2.31.

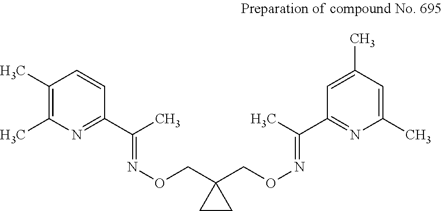

At room temperature, a solution of 2.05 g (2.0 mmol) of 2-acetyl-5,6-dimethylpyridine (II-1) in 50 ml of ethanol and 30 mg of p-toluenesulphonic acid were added to a solution of 0.53 g (2.0 mmol) of (1E)-1-(5,6-dimethylpyridin-2-yl)ethanone O-({1-[(aminooxy)methyl]cyclopropyl}methyl)oxime (XII-8) in 10 ml of ethanol, and the mixture was heated under reflux for 4 h. After cooling, the reaction mixture was poured into ice-water and neutralized by addition of triethylamine. After extraction with dichloromethane, the combined organic phases were dried over Na₂SO₄ and freed from the solvent under reduced pressure.

Purification by column chromatography on silica gel using cyclohexane/ethyl acetate gave 0.73 g (1.6 mmol, 79% of theory) of (1E)-1-(4,6-dimethylpyridin-2-yl)ethanone O-({1-[({[(1E)-1-(5,6-dimethylpyridin-2-yl)ethylidene]amino}oxy)methyl]cyclopropyl}methyl)oxime of logP (HCO₂H)=2.61.

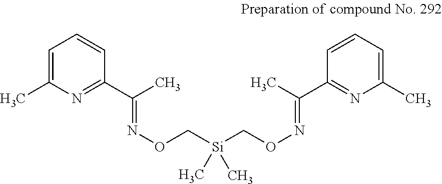

0.2 g (1.33 mmol) of 1-(6-methylpyridin-2-yl)ethanone oxime (V-1) was initially charged in 10 ml of acetonitrile, and 0.87 g (2.66 mmol) of caesium carbonate and 0.22 g (1.33 mmol) of potassium iodide were added. After 30 min of stirring at room temperature, 0.1 g of bis(chloromethyl)dimethylsilane (0.67 mmol) was added. The reaction mixture was stirred at 80° C. for 6 h and, after cooling to room temperature, the solids were filtered off 50 ml of water were added to the filtrate, which was extracted twice with dichloromethane. The combined organic phases were dried and concentrated on a rotary evaporator. The residue was chromatographed on a silica gel cartridge (cyclohexane/ethyl acetate=6:1).

This gave 80 mg (0.21 mmol, 19.8% of theory) of (1E,1E′)-1-(6-methylpyridin-2-yl)ethanone O,O′-[(dimethylsilanediyl)bis(methylene)]oxime as an oil of logP (HCO₂H)=4.5.

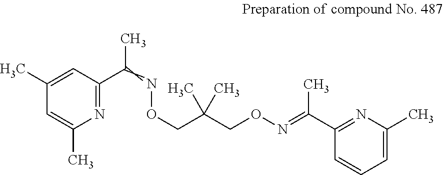

A solution of 32 mg (0.196 mmol) of (1E/Z)-1-(4,6-dimethylpyridin-2-yl)ethanone oxime (V-3) and 0.24 ml (0.236 mmol) of a 1M solution of P4 phosphazene in hexane in tetrahydrofuran was stirred at room temperature for 10 min, and 50 mg (0.196 mmol) of (1E)-1-(6-methylpyridin-2-yl)ethanone O-(3-chloro-2,2-dimethylpropyl)oxime were then added dropwise. The reaction mixture was heated at 75° C. for 10 h and stirred at room temperature for another 48 h. 2 ml of water/5 ml of ethyl acetate were added, and the mixture was stirred for 10 min and filtered through a small cartridge charged with Extrelut (2.3 g) and silica gel which was eluted using 10 ml of ethyl acetate. The crude product was, after concentration under reduced pressure, purified by column chromatography (gradient cyclohexane/ethyl acetate).

This gave 56 mg (0.15 mmol, 66% of theory) of (1E/Z)-1-(4,6-dimethylpyridin-2-yl)ethanone O-[2,2-dimethyl-3-({[(1E)-1-(6-methylpyridin-2-yl)ethylidene]amino}oxy)propyl]oxime of logP (HCO₂H)=3.33.

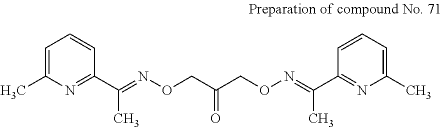

6.0 g (17 mmol) of (1E)-1-(6-methylpyridin-2-yl)ethanone 0-{2-[({[(1E)-1-(6-methylpyridin-2-yl)-ethylidene]amino}oxy)methyl]prop-2-en-1-yl}oxime were initially charged in dichloromethane/methanol 1:1 and cooled to −40° C. At this temperature, ozone was introduced for about 15 min, and the reaction was monitored by thin-layer chromatography. After complete conversion of the starting material, excess ozone was destroyed at −20° C. using sodium bisulphite solution (38% strength) until the peroxide test was negative. The reaction mixture obtained was concentrated, triturated with ethyl acetate and filtered off with suction. The residue obtained was washed with plenty of ethyl acetate, and in this manner the desired product was isolated as a white solid and subsequently purified chromatographically using cyclohexane/ethyl acetate 3:1.

This gave 5.2 g (1.5 mmol, 86% of theory) of (2E,9E)-2,10-bis(6-methylpyridin-2-yl)-4,8-dioxa-3,9-diazaundeca-2,9-dien-6-one as a solid of logP (HCO₂H)=2.20.

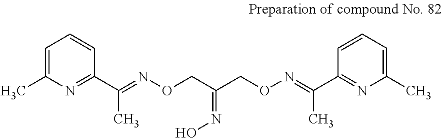

130 mg (0.37 mmol) of (2E,9E)-2,10-bis(6-methylpyridin-2-yl)-4,8-dioxa-3,9-diazaundeca-2,9-dien-6-one were initially charged in 5 ml of ethanol, 38 mg (0.55 mmol) of hydroxylammonium chloride and 55 mg (0.55 mmol) of triethylamine were added and the mixture was stirred at 60° C. for 2.5 h. The reaction mixture was concentrated using a rotary evaporator, and the residue obtained was extracted repeatedly with ethyl acetate/semiconcentrated sodium chloride solution. The combined organic phases were dried over sodium sulphate and concentrated.

This gave 130 mg (0.35 mmol, 92% of theory) of (2E,9E)-2,10-bis(6-methylpyridin-2-yl)-4,8-dioxa-3,9-diazaundeca-2,9-dien-6-one oxime as a colourless oil of logP (HCO₂H)=1.91.

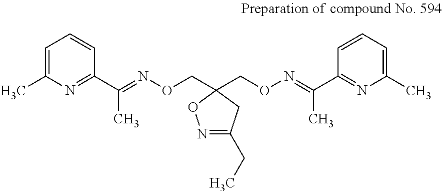

250 mg (0.7 mmol) of (1E)-1-(6-methylpyridin-2-yl)ethanone O-{2-[({[(1E)-1-(6-methylpyridin-2-yl)-ethylidene]amino}oxy)methyl]prop-2-en-1-yl}oxime and 67.5 mg (0.92 mmol) of propanal oxime were initially charged in 10 ml of dichloromethane, and 123 mg of N-chlorosuccinimide were added. 72 mg (0.7 mmol) of triethylamine in 1 ml of dichloromethane were added dropwise. The reaction mixture was stirred at room temperature for 12 hours, and 10 ml of water were then added. The organic phase was removed using a phase-separation cartridge, concentrated and chromatographed on a silica gel cartridge (cyclohexane/ethyl acetate, vol. 2:1).

This gave 79 mg (0.19 mmol, 26% of theory) of (1E,1′E)-1-(6-methylpyridin-2-yl)ethanone O,O′-[(3-ethyl-4,5-dihydroisoxazole-5,5-diyl)bis(methylene)]oxime as a colourless oil of logP (HCO₂H)=2.68.

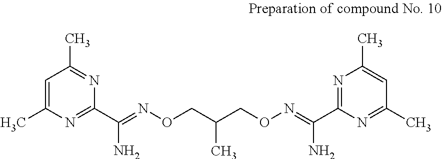

200 mg (0.52 mmol) of N′-({2-[({[(Z)-amino-(4,6-dimethylpyrimidin-2-yl)methylene]amino}oxy)methyl]-prop-2-en-1-yl}oxy)-4,6-dimethylpyrimidine-2-carboximidamide were dissolved in 5 ml of ethanol, 55 mg of palladium/carbon (10%) were added and the mixture was then stirred under a hydrogen atmosphere at a pressure of 10 bar for 16 h. The reaction mixture was then filtered through a sodium sulphate cartridge, which was washed with methanol, and the solvent was removed.

This gave 175 mg (0.45 mmol, 87% of theory) of N′,N″-[(2-methylpropane-1,3-diyl)bis(oxy)]bis(4,6-dimethylpyrimidine-2-carboximidamide) as an oil of logP (HCO₂H)=1.81.

The compounds of the formula (I) listed in Table 1 below can be obtained analogously to the preceding examples and in accordance with the general descriptions of the processes according to the invention.

TABLE 1

| | |
|---|---|
| | (I) |
| 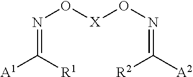 | |

| No. | A¹ | R¹ | X | R² | A² | logP |
|---|---|---|---|---|---|---|
| 1 | 6-methylpyridin-2-yl | Me | 2-methylpropane-1,3-diyl | Me | 6-methylpyridin-2-yl | 2.31^{[a]} |
| | | | | | | 5.44^{[c]} |
| 2 | 2-pyridyl | Me | 2-hydroxy-2-methylpropane-1,3-diyl | Me | 2-pyridyl | 1.73^{[a]} |
| 3 | 2-pyridyl | Me | 2-hydroxypropane-1,3-diyl | Me | 2-pyridyl | 1.50^{[a]} |
| 4 | 4,6-dimethylpyrimidin-2-yl | NH₂ | —(CH₂)₄— | NH₂ | 4,6-dimethylpyrimidin-2-yl | 1.73^{[b]} |
| 5 | 4.6-dimethylpyrimidin-2-yl | NH₂ | —CH₂— | NH₂ | 4,6-dimethylpyrimidin-2-yl | 1.49^{[b]} |
| 6 | 6-methylpyridin-2-yl | Me | —(CH₂)₄— | Me | 6-methylpyridin-2-yl | 3,16^{[b]} |
| 7 | 6-methylpyridin-2-yl | Me | —CH₂— | Me | 6-methylpyridin-2-yl | 2.39^{[b]} |
| 8 | 4,6-dimethylpyrimidin-2-yl | NH₂ | 2-methylenepropane-1,3-diyl | NH₂ | 4,6-dimethylpyrimidin-2-yl | 1.80^{[b]} |
| 9 | 6-methylpyridin-2-yl | Me | 2-methylenepropane-1,3-diyl | Me | 6-methylpyridin-2-yl | 3.33^{[b]} |
| 10 | 4,6-dimethylpyrimidin-2-yl | NH₂ | 2-methylpropane-1,3-diyl | NH₂ | 4,6-dimethylpyrimidin-2-yl | 1.81^{[b]} |
| 11 | 6-methylpyridin-2-yl | Me | pentane-1,5-diyl | Me | 6-methylpyridin-2-yl | 3.85^{[b]} |
| 12 | 6-methylpyridin-2-yl | Me | 2-hydroxy-2-methylpropane-1,3-diyl | Me | 6-methylpyridin-2-yl | 1.66^{[a]} |
| 13 | 6-methylpyridin-2-yl | Me | —CH₂—CH₂— | Me | 6-methylpyridin-2-yl | 2.22^{[a]} |
| | | | | | | (isomer mixture) |
| 14 | 6-methylpyridin-2-yl | Me | —CH₂—CH₂—CH₂— | Me | 6-methylpyridin-2-yl | 2.66^{[a]} |
| 15 | 6-methylpyridin-2-yl | Me | cyclopropane-1,1-diylbis(methylene) | Me | 6-methylpyridin-2-yl | 3.44^{[b]} |
| 16 | 6-methylpyridin-2-yl | Me | —CH₂—CH₂—CH₂— | Me | 4-methyl-1,3-thiazol-2-yl | 3.35^{[a]} |
| | | | | | | 4.47^{[c]} |
| | | | | | | (isomer mixture) |
| 17 | 2-pyridyl | Me | 2-methylenepropane-1,3-diyl | Me | 6-methylpyridin-2-yl | 3.10^{[b]} (isomer A) |
| 18 | 6-methylpyridin-2-yl | Me | 2-methylenepropane-1,3-diyl | Me | 4,5-dimethyl-1,3-thiazol-2-yl | 4.41^{[b]} |
| 19 | 6-methylpyridin-2-yl | Me | oxetane-3,3-diylbis(methylene) | Me | 6-methylpyridin-2-yl | 2.45^{[b]} |
| 20 | 5-chloro-2-thienyl | Me | 2-methylenepropane-1,3-diyl | Me | 5-chloro-2-thienyl | 5.88^{[b]} |
| 21 | 1-methyl-1H-pyrrol-2-yl | Me | 2-methylenepropane-1,3-diyl | Me | 1-methyl-1H-pyrrol-2-yl | 5.03^{[b]} |
| 22 | 6-methylpyridin-3-yl | Me | 2-methylenepropane-1,3-diyl | Me | 6-methylpyridin-3-yl | 1.37^{[b]} |
| 23 | 6-methylpyridin-2-yl | Me | 2-methylenepropane-1,3-diyl | NH₂ | 6-methylpyridin-2-yl | 2.75^{[b]} |
| 24 | 3,5-Cl₂-Ph | Me | 2-methylenepropane-1,3-diyl | Me | 6-methylpyridin-2-yl | 6.30^{[b]} |
| 25 | 3,5-dimethylisoxazol-4-yl | Me | 2-methylenepropane-1,3-diyl | Me | 3,5-dimethylisoxazol-4-yl | 3,57^{[b]} |
| 26 | 2-pyridiyl | Me | oxetane-2,2-diylbis(methylene) | Me | 6-methylpyridin-2-yl | 2.29^{[a]} |
| | | | | | | 2.43^{[b]} |
| 27 | 2-methyl-1,3-thiazol-4-yl | NH₂ | 2-methylenepropane-1,3-diyl | NH₂ | 2-methyl-1,3-thiazol-4-yl | 3.19^{[b]} |
| 28 | pyridin-4-yl | Me | —CH₂—CH₂— | Me | pyridin-4-yl | 0.66^{[b]} |
| 29 | pyridin-4-yl | Me | —CH₂—CH₂—CH₂— | Me | pyridin-4-yl | 1.23^{b]} |
| 30 | 2-pyridyl | Me | —CH₂—CH₂— | Me | 2-pyridyl | 2.21^{[b]} |
| 31 | 2-pyridyl | Me | —CH₂—CH₂—CH₂— | Me | 2-pyridyl | 2.67^{[b]} |
| 32 | 3-pyridyl | Me | —CH₂—CH₂— | Me | 3-pyridyl | 1.32^{[b]} |
| 33 | 3-pyridyl | Me | —CH₂—CH₂—CH₂— | Me | 3-pyridyl | 1.51^{[b]} |
| 34 | 4-methylpyridin-2-yl | Me | —CH₂—CH₂— | Me | 4-methylpyridin-2-yl | 1.71^{[b]} |
| 35 | 4-methylpyridin-2-yl | Me | —CH₂—CH₂—CH₂— | Me | 4-methylpyridin-2-yl | 1.94^{[b]} |
| 36 | 4-methyl-5,6,7,8-tetra- | NH₂ | —(CH₂)₄— | NH₂ | 4-methyl-5,6,7,8-tetra- | 2.58^{[b]} |
| | hydroquinazolin-2-yl | | | | hydroquinazolin-2-yl | |
| 37 | 4,6-bis(methoxymethyl)-5- | NH₂ | —(CH₂)₄— | NH₂ | 4,6-bis(methoxymethyl)-5- | 1.75^{[b]} |
| | methylpyrimidin-2-yl | | | | methylpyrimidin-2-yl | |
| 38 | 3,5-difluoropyridin-2-yl | NH₂ | —(CH₂)₄— | NH₂ | 3,5-difluoropyridin-2-yl | 2.45^{[b]} |
| 39 | 4-methyl-5,6,7,8-tetra- | NH₂ | —CH₂— | NH₂ | 4-methyl-5,6,7,8-tetra- | 2.29^{[b]} |
| | hydroquinazolin-2-yl | | | | hydroquinazolin-2-yl | |
| 40 | 4,6-bis(methoxymethyl)-5- | NH₂ | —CH₂— | NH₂ | 4,6-bis(methoxymethyl)-5- | 1.35^{[b]} |
| | methylpyrimidin-2-yl | | | | methylpyrimidin-2-yl | |
| 41 | 5-chloro-4,6-dimethylpyrimidin- | NH₂ | —CH₂— | NH₂ | 5-chloro-4,6-dimethylpyrimidin-2-yl | 2.36^{[b]} |
| | 2-yl | | | | | |
| 42 | 4,6-dicyclopropylpyrimidin-2-yl | NH₂ | —CH₂— | NH₂ | 4,6-dicyclopropylpyrimidin-2-yl | 2.92^{[b]} |
| 43 | 4-methyl-5,6,7,8-tetra- | NH₂ | 2-methylenepropane-1,3-diyl | NH₂ | 4-methyl-5,6,7,8-tetrahydro- | 2.67^{[b]} |
| | hydroquinazolin-2-yl | | | | quinazolin-2-yl | |
| 44 | 4,6-bis(methoxymethyl)-5- | NH₂ | 2-methylenepropane-1,3-diyl | NH₂ | 4,6-bis(methoxymethyl)-5- | 1.83^{[b]} |
| | methylpyrimidin-2-yl | | | | methylpyrimidin-2-yl | |
| 45 | 3,5-difluoropyridin-2-yl | NH₂ | 2-methylenepropane-1,3-diyl | NH₂ | 3,5-difluoropyridin-2-yl | 2.42^{[b]} |
| 46 | 5-chloro-4,6-dimethylpyrimidin- | NH₂ | 2-methylenepropane-1,3-diyl | NH₂ | 5-chloro-4,6-dimethylpyrimidin-2-yl | 2.84^{[b]} |
| | 2-yl | | | | | |
| 47 | 4,6-dicylcopropylpyrimidin-2-yl | NH₂ | 2-methylenepropane-1,3-diyl | NH₂ | 4,6-dicyclopropylpyrimidin-2-yl | 3.33^{[b]} |
| 48 | 2-phenyl-1,3-thiazol-5-yl | Me | —CH₂—CH₂— | Me | 2-phenyl-1,3-thiazol-5-yl | 6.51^{[b]} |
| 49 | 2-phenyl-1,3-thiazol-5-yl | Me | —CH₂—CH₂—CH₂— | Me | 2-phenyl-1,3-thiazol-5-yl | 7.07^{[b]} |
| 50 | 4,5-dimethyl-1,3-thiazol-2-yl | Me | —CH₂—CH₂— | Me | 4,5-dimethyl-1,3-thiazol-2-yl | 4.35^{[b]} |
| 51 | 4,5-dimethyl-1,3-thiazol-2-yl | Me | —CH₂—CH₂—CH₂— | Me | 4,5-dimethyl-1,3-thiazol-2-yl | 5.03^{[b]} |
| 52 | 4-methyl-1,3-thiazol-2-yl | Me | —CH₂—CH₂— | Me | 4-methyl-1,3-thiazol-2-yl | 3.63^{[b]} |
| 53 | 2,4-dimethyl-1,3-thiazol-5-yl | Me | —CH₂—CH₂— | Me | 2,4-dimethyl-1,3-thiazol-5-yl | 2.70^{[b]} |
| 54 | 2,4-dimethyl-1,3-thiazol-5-yl | Me | —CH₂—CH₂—CH₂— | Me | 2,4-dimethyl-1,3-thiazol-5-yl | 3.10^{[b]} |
| 55 | 3,5-dichloropyridin-2-yl | Me | —CH₂—CH₂— | Me | 3,5-dichloropyridin-2-yl | 5.39^{[b]} |
| 56 | 3,5-dichloropyridin-2-yl | Me | —CH₂—CH₂—CH₂— | Me | 3,5-dichloropyridin-2-yl | 5.78^{[b]} |
| 57 | 6-chloro-3-pyridiyl | Me | 2-methylenepropane-1,3-diyl | Me | 6-chloro-3-pyridyl | 4.38^{[b]} |
| 58 | 4,6-dimethylpyridin-2-yl | NH₂ | —CH₂— | NH₂ | 4,6-dimethylpyridin-2-yl | 1.87^{[b]} |
| 59 | 2-pyridyl | Me | —CH₂— | Me | 2-pyridyl | 2.23^{[b]} |
| 60 | 2-pyridyl | Me | 2-methylenepropane-1,3-diyl | Me | 2-pyridyl | 3.00^{[b]} |
| 61 | 5-(trifluoromethyl)pyridin-2-yl | Me | 2-methylenepropane-1,3-diyl | Me | 5-(trifluoromethyl)pyridin-2-yl | 6.18^{[b]} |
| 62 | 6-propylpyridin-2-yl | Me | 2-methylenepropane-1,3-diyl | Me | 6-propylpyridin-2-yl | 6.89^{[b]} |
| 63 | quinolin-2-yl | Me | 2-methylenepropane-1,3-diyl | Me | quinolin-2-yl | 6.51^{[b]} |
| 64 | 4-methoxy-6-methylpyrimidin-2- | Me | 2-methylenepropane-1,3-diyl | Me | 4-methoxy-6-methylpyrimidin-2-yl | 3.17^{[b]} |
| | yl | | | | | |
| 65 | 3-methylpyridin-2-yl | Me | 2-methylenepropane-1,3-diyl | Me | 3-methylpyridin-2-yl | 2.90^{[b]} |
| 66 | 5-(trifluoromethyl)pyridin-2-yl | Me | 2-methylenepropane-1,3-diyl | Me | 6-methylpyridin-2-yl | 5.15^{[b]} |
| 67 | 5,6-dimethylpyridin-2-yl | Me | 2-methylenepropane-1,3-diyl | Me | 5,6-dimethylpyridin-2-yl | 2.82^{[b]} |
| 68 | 4,5-dimethyl-1,3-thiazol-2-yl | Me | 2-methylenepropane-1,3-diyl | Me | 4,5-dimethyl-1,3-thiazol-2-yl | 5.25^{[b]} |
| | | | | | | (isomer B) |
| 69 | 4,5-dimethyl-1,3-thiazol-2-yl | Me | 2-methylenepropane-1,3-diyl | Me | 4,5-dimethyl-1,3-thiazol-2-yl | 4.99^{[b]} (isomer A) |
| 70 | 6-methylpyridin-2-yl | Me | 2-methylpropane-1,3-diyl | Me | 6-methylpyridin-2-yl | CuCl₂ salt, m.p. |
| | | | | | | 186-188° C. |
| | | | | | | (decomp.) |
| 71 | 6-methylpyridin-2-yl | Me | 2-oxopropane-1,3-diyl | Me | 6-methylpyridin-2-yl | 2.20^{[b]} |
| 72 | 5,6-dimethylpyridin-2-yl | Me | —CH₂—CH₂—CH₂— | Me | 5,6-dimethylpyridin-2-yl | 2.23^{[a]} |
| 73 | 6-methylpyridin-2-yl | Me | hexane-1,6-diyl | Me | 6-methylpyridin-2-yl | 4.47^{[b]} |
| 74 | 6-methylpyridin-2-yl | Me | decane-1,10-diyl | Me | 6-methylpyridin-2-yl | 7.36^{[b]} |
| 75 | 6-methylpyridin-2-yl | Me | 3-methylpentane-1,5-diyl | Me | 6-methylpyridin-2-yl | 4.38^{[b]} |
| 76 | 6-methylpyridin-2-yl | CO₂Et | —CH₂—CH₂—CH₂— | CO₂Et | 6-methylpyridin-2-yl | 4.31^{[a]} |
| 77 | 2-thienyl | Me | 2-methylenepropane-1,3-diyl | Me | 2-thienyl | 4.84^{[b]} (isomer B) |
| 78 | 2-thienyl | Me | 2-methylenepropane-1,3-diyl | Me | 1,3-thiazol-2-yl | 4.31^{[b]} |
| 79 | 2-thienyl | Me | 2-methylenepropane-1,3-diyl | Me | 2-thienyl | 4,56^{[b]} (isomer A) |
| 80 | 6-bromopyridin-2-yl | Me | —CH₂—CH₂—CH₂— | Me | 6-bromopyridin-2-yl | 5.99^{[a]} |
| 81 | 5-methylpyridin-2-yl | Me | —CH₂—CH₂—CH₂— | Me | 5-methylpyridin-2-yl | 2.90^{[a]} |
| 82 | 6-methylpyridin-2-yl | Me | 2-(hydroxyimino)propane-1,3-diyl | Me | 6-methylpyridin-2-yl | 1.91^{[b]} |
| 83 | 2-methyl-1,3-thiazol-4-yl | NH₂ | 2-methylpropane-1,3-diyl | NH₂ | 2-methyl-1,3-thiazol-4-yl | 3.54^{[b]} |
| 84 | 3-methylpyridin-2-yl | Me | —CH₂—CH₂—CH₂— | Me | 3-methylpyridin-2-yl | 2.51^{[a]} (isomer B) |
| 85 | 3-methylpyridin-2-yl | Me | —CH₂—CH₂—CH₂— | Me | 3-methylpyridin-2-yl | 2.15^{[a]} (isomer A) |
| 86 | 6-methylpyridin-2-yl | Me | 2,2-difluoropropane-1,3-diyl | Me | 6-methylpyridin-2-yl | 4.00^{[a]} |
| 87 | 4-methyl-1,3-thiazol-2-yl | Me | —CH₂—CH₂—CH₂— | Me | 4-methyl-1,3-thiazol-2-yl | 4.22^{[a]} |
| 88 | 6-methylpyridin-2-yl | iPr | —CH₂—CH₂—CH₂— | iPr | 6-methylpyridin-2-yl | 5.52^{[a]} (isomer B) |
| 89 | 6-methylpyridin-2-yl | iPr | —CH₂—CH₂—CH₂— | iPr | 6-methylpyridin-2-yl | 4.45^{[a]} (isomer A) |
| 90 | 6-methylpyridin-2-yl | Et | —CH₂—CH₂—CH₂— | Et | 6-methylpyridin-2-yl | 4.44^{[a]} |
| 91 | 6-methylpyridin-2-yl | nPr | —CH₂—CH₂—CH₂— | nPr | 6-methylpyridin-2-yl | 5.83^{[a]} |
| 92 | 3-methylpyridin-2-yl | iPr | —CH₂—CH₂—CH₂— | iPr | 3-methylpyridin-2-yl | 4.13^{[a]} |
| | | | | | | (isomer mixture) |
| 93 | 3-methylpyridin-2-yl | iBu | —CH₂—CH₂—CH₂— | iBu | 3-methylpyridin-2-yl | 5.96^{[a]} |
| 94 | 3-methylpyridin-2-yl | Et | —CH₂—CH₂—CH₂— | Et | 3-methylpyridin-2-yl | 3.65^{[a]} (isomer B) |
| 95 | 3-methylpyridin-2-yl | Et | —CH₂—CH₂—CH₂— | Et | 3-methylpyridin-2-yl | 3.11^{[a]} (isomer A) |
| 96 | 3-methylpyridin-2-yl | Bzl | —CH₂—CH₂—CH₂— | Bzl | 3-methylpyridin-2-yl | 5.54^{[a]} (isomer B) |
| 97 | 3-methylpyridin-2-yl | Bzl | —CH₂—CH₂—CH₂— | Bzl | 3-methylpyridin-2-yl | 4.89^{[a]} (isomer A) |
| 98 | 3-methylpyridin-2-yl | nPr | —CH₂—CH₂—CH₂— | nPr | 3-methylpyridin-2-yl | 4.83^{[a]} (isomer B) |
| 99 | 3-methylpyridin-2-yl | nPr | —CH₂—CH₂—CH₂— | nPr | 3-methylpyridin-2-yl | 4.22^{[a]} (isomer A) |
| 100 | 3-methylpyridin-2-yl | Et | 2-methylpropane-1,3-diyl | Et | 3-methylpyridin-2-yl | 3.58^{[a]} (isomer A) |
| 101 | 3-methylpyridin-2-yl | Et | 2-methylpropane-1,3-diyl | Et | 3-methylpyridin-2-yl | 4.23^{[a]} (isomer B) |
| 102 | 3-methylpyridin-2-yl | iBu | 2-methylpropane-1,3-diyl | iBu | 3-methylpyridin-2-yl | 6.50^{[a]} (isomer B) |
| 103 | 3-methylpyridin-2-yl | iPr | 2-methylpropane-1,3-diyl | iPr | 3-methylpyridin-2-yl | 4.28^{[a]} |
| | | | | | | (isomer mixture) |
| 104 | 4-tert-butylpyridin-2-yl | Me | 2-methylpropane-1,3-diyl | Me | 4-tert-butylpyridin-2-yl | 5.88^{[a]} |
| 105 | 4-ethylpyridin-2-yl | Me | 2-methylpropane-1,3-diyl | Me | 4-ethylpyridin-2-yl | 3.62^{[a]} |
| 106 | 6-chloro-3-pyridyl | Me | 2-methylpropane-1,3-diyl | Me | 6-chloro-3-pyridyl | 4.79^{[a]} |
| 107 | 2-chloro-3-pyridyl | Me | 2-methylpropane-1,3-diyl | Me | 2-chloro-3-pyridyl | 3.87^{[a]} |
| | | | | | | (isomer mixture) |
| 108 | 3-methylpyridin-2-yl | Bzl | 2-methylpropane-1,3-diyl | Bzl | 3-methylpyridin-2-yl | 5.32^{[a]} (isomer A) |
| 109 | 3-methylpyridin-2-yl | Bzl | 2-methylpropan3-1,3-diyl | Bzl | 3-methylpyridin-2-yl | 6.00^{[a]} (isomer B) |
| 110 | 3-methylpyridin-2-yl | iBu | 2-methylpropane-1,3-diyl | iBu | 3-methylpyridin-2-yl | 5.78^{[a]} (isomer A) |
| 111 | 3-methylpyridin-2-yl | nPr | 2-methylpropane-1,3-diyl | nPr | 3-methylpyridin-2-yl | 4.73^{[a]} |
| | | | | | | (isomer mixture) |
| 112 | 6-methylpyridin-2-yl | nPr | 2-methylpropane-1,3-diyl | nPr | 6-methylpyridin-2-yl | 6.61^{[a]} |
| 113 | 6-methylpyridin-2-yl | Me | 2-(methoxyimino)propane-1,3-diyl | Me | 6-methylpyridin-2-yl | 3.26^{[b]} |
| 114 | 6-methylpyridin-2-yl | iBu | —CH₂—CH₂—CH₂— | iBu | 6-methylpyridin-2-yl | 5.92^{[a]} (isomer A) |
| 115 | 6-methylpyridin-2-yl | iBu | —CH₂—CH₂—CH₂— | iBu | 6-methylpyridin-2-yl | 7.08^{[a]} (isomer B) |
| 116 | 6-methylpyridin-2-yl | Et | 2-methylpropane-1,3-diyl | Et | 6-methylpyridin-2-yl | 5.40^{[a]} |
| 117 | 6-acetylpyridin-2-yl | Me | 2-methylenepropane-1,3-diyl | Me | 6-methylpyridin-2-yl | 4.43^{[b]} |
| 118 | 5,6-dimethylpyridin-2-yl | Me | 2-methylpropane-1,3-diyl | Me | 5,6-dimethylpyridin-2-yl | 2.99^{[a]} |
| 119 | 4-phenylpyridin-2-yl | Me | 2-methylpropane-1,3-diyl | Me | 4-phenylpyridin-2-yl | 6.33^{[a]} |
| 120 | 6-acetylpyridin-2-yl | Me | 2-methylenepropane-1,3-diyl | Me | 6-acetylpyridin-2-yl | 5.15^{[b]} |
| 121 | 2,4-dimethyl-1,3-thiaozl-5-yl | Me | 2-methylpropane-1,3-diyl | Me | 2,4-dimethyl-1,3-thiazol-5-yl | 4.02^{[a]} |
| | | | | | | (isomer mixture) |
| 122 | 4-methyl-1,3-thiazol-2-yl | Me | 2-methylpropane-1,3-diyl | Me | 4-methyl-1,3-thiazol-2-yl | 4.98^{[a]} |
| | | | | | | (isomer mixture) |
| 123 | 4,6-dimethylpyridin-2-yl | NH₂ | —(CH₂)₄— | NH₂ | 4,6-dimethylpyridin-2-yl | 2.67^{[b]} |
| 124 | 2-pyridyl | Me | —(CH₂)₄— | Me | 2-pyridyl | 3.00^{[b]} |
| 125 | 4,5-dimethyl-1,3-thiazol-2-yl | Me | —(CH₂)₄— | Me | 4,5-dimethyl-1,3-thiaozl-2-yl | 5.59^{[b]} |
| 126 | 4,5-dimethyl-1,3-thiazol-2-yl | Me | —CH₂— | Me | 4,5-dimethyl-1,3-thiazol-2-yl | 4.46^{[b]} |
| 127 | 4,6-dimethylpyridin-2-yl | NH₂ | 2-methylenepropane-1,3-diyl | NH₂ | 4,6-dimethylpyridin-2-yl | 2.7^{[b]} |
| 128 | 3-methylpyridin-2-yl | Me | 2-methylpropane-1,3-diyl | Me | 3-methylpyridin-2-yl | 3.12^{[a]} |
| 129 | 3-methylpyridin-2-yl | But-3-en-1- | —CH₂—CH₂—CH₂— | But-3-en-1-yl | 3-methylpyridin-2-yl | 4.59^{[a]} |
| | | yl | | | | |
| 130 | 3-methylpyridin-2-yl | But-3-en-1- | 2-methylpropane-1,3-diyl | But-3-en-1-yl | 3-methylpyridin-2-yl | 5.06^{[a]} |
| | | yl | | | | |
| 131 | 6-benzylpyridin-2-yl | Me | —CH₂—CH₂—CH₂— | Me | 6-benzylpyridin-2-yl | 6.95^{[a]} |
| 132 | 4-phenylpyridin-2-yl | Me | —CH₂—CH₂—CH₂— | Me | 4-phenylpyridin-2-yl | 5.74^{[a]} |
| 133 | 5-methylpyridin-2-yl | Me | 2-methylpropane-1,3-diyl | Me | 5-methylpyridin-2-yl | 3.67^{a]} |
| 134 | 6-isobutylpyridin-2-yl | Me | —CH₂—CH₂—CH₂— | Me | 6-isobutylpyridin-2-yl | 7.36^{[a]} |
| 135 | 6-methylpyridin-2-yl | Me | 2-methylenepropane-1,3-diyl | Me | 3-chloro-5-(trifluoromethyl)- | 4.95^{[b]} (isomer B) |
| | | | | | pyridin-2-yl | |
| 136 | 4-ethylpyridin-2-yl | Me | —CH₂—CH₂—CH₂— | Me | 4-ethylpyridin2--yl | 2.98^{[a]} |
| 137 | 6-methylpyridin-2-yl | CH₂SO₂Me | 2-methylpropane-1,3-diyl | CH₂SO₂Me | 6-methylpyridin-2-yl | 3.08^{[a]} |
| 138 | 6-methylpyridin-2-yl | iPr | 2-methylpropane-1,3-diyl | iPr | 6-methylpyridin-2-yl | 6.22^{a]} |
| 139 | 6-methylpyridin-2-yl | Me | 2-methylenepropane-1,3-diyl | Me | 3-chloropyridin-2-yl | 3.39^{[b]} (isomer B) |
| 140 | 6-methylpyridin-2-yl | Me | 2-methylenepropane-1,3-diyl | Me | 3-chloropyridin-2-yl | 4.40^{[b]} (isomer A) |
| 141 | 6-methylpyridin-2-yl | Me | 2,2-dimethylpropane-1,3-diyl | Me | 6-methylpyridin-2-yl | 4.40^{[b]} (isomer A) |
| 142 | 6-methylpyridin-2-yl | Me | 2-methylenepropane-1,3-diyl | Et | 3-methylpyridin-2-yl | 3.84^{[b]} (isomer )B |
| 143 | 6-methylpyridin-2-yl | Me | 2-methylenepropane-1,3-diyl | Et | 3-methylpyridin-2-yl | 3.23^{[b]} (isomer A) |
| 144 | 3-chloropyridin-2-yl | Me | 2-methylenepropane-1,3-diyl | Me | 3-chloropyridin-2-yl | 3.64^{[b]} |
| 145 | 6-methoxypyridin-2-yl | Me | 2-methylenepropane-1,3-diyl | Me | 6-methoxypyridin-2-yl | 5.89^{[b]} |
| 146 | 4,6-dimethylpyrimidin-2-yl | Me | 2-methylenepropane-1,3-diyl | Me | 4,6-dimethylpyrimidin-2-yl | 2.91^{[b]} |
| 147 | 2-chloro-3-pyridyl | Me | 2-methylenepropane-1,3-diyl | Me | 6-methylpyridin-2-yl | 3.34^{[b]} |
| 148 | 2-chloro-3-pyridyl | Me | 2-methylenepropane-1,3-diyl | Me | 2-chloro-3-pyridyl | 3.53^{[b]} |
| 149 | 3-chloro-5-(trifluoromethyl)- | Me | 2-methylenepropane-1,3-diyl | Me | 3-chloro-5-(triifluoromethyl)- | 5.82^{[b]} |
| | pyridin-2-yl | | | | pyridin-2-yl | |
| 150 | 6-bromopyridin-2-yl | Me | 2-methylenepropane-1,3-diyl | Me | 6-bromopyridin-2-yl | 5.96^{[b]} |
| 151 | 6-bromopyridin-3-yl | Me | 2-methylenepropane-1,3-diyl | Me | 6-bromopyridin-3-yl | 4.56^{[b]} |
| 152 | 5-methylpyridin-2-yl | Me | 2-methylenepropane-1,3-diyl | Me | 5-methylpyridin-2-yl | 3.36^{[b]} |
| 153 | 3,5-dichloropyridin-2-yl | Me | 2-methylenepropane-1,3-diyl | Me | 3,5-dichloropyridin-2-yl | 5.75^{[b]} |
| 154 | 6-methylpyridin-2-yl | Me | 2-methylenepropane-1,3-diyl | iBu | 3-methylpyridin-2-yl | 5.08^{[b]} |
| | | | | | | (isomer mixture) |
| 155 | 6-methylpyridin-2-yl | Me | 2-methylenepropane-1,3-diyl | iBu | 3-methylpyridin-2-yl | 4.40^{[b]} (isomer A) |
| 156 | 6-methylpyridin-2-yl | Me | —CH₂—CH₂—CH₂— | Et | 3-methylpyridin-2-yl | 3.28^{[b]} |
| 157 | 6-methylpyridin-2-yl | Me | 2-methylenepropane-1,3-diyl | Me | 3-chloro-5-(trifluoromethyl)- | 4.42^{[b]} (isomer A) |
| | | | | | pyridin-2-yl | |
| 158 | 6-methylpyridin-2-yl | Me | 1-oxoethane-1,2-diyl | Me | 6-methylpyridin-2-yl | 2.50^{[a]} |
| | | | | | | 2.57^{b]} |
| 159 | 6-methylpyridin-2-yl | Me | 1,4-dioxobutane-1,4-diyl | Me | 6-methylpyridin-2-yl | 2.40^{[a]} |
| 160 | 4-methylquinolin-2-yl | Me | 2-methylenepropane-1,3-diyl | Me | 4-methylquinolin-2-yl | 7.00^{b]} |
| 161 | 2-methylpyrimidin-4-yl | Me | —CH₂—CH₂—CH₂— | Me | 2-methylpyrimidin-4-yl | 3.06^{[a]} |
| 162 | 6-methylpyridin-2-yl | cPr | 2-methylpropane-1,3-diyl | cPr | 6-methylpyridin-2-yl | 3.28^{[a]} (isomer A) |
| 163 | 5-methylpyridin-3-yl | Me | —CH₂—CH₂—CH₂— | Me | 5-methylpyridin-3-yl | 1.54^{a]} |
| 164 | 6-methylpyridin-2-yl | cPr | 2-methylpropane-1,3-diyl | cPr | 6-methylpyridin-2-yl | 3.51^{]a]} (isomer B) |
| 165 | 2-methylpyridin-4-yl | Me | —CH₂—CH₂—CH₂— | Me | 2-methylpyridin-4-yl | |
| 166 | 5-methylpyridin-3-yl | Me | 2-methylpropane-1,3-diyl | Me | 5-methylpyridin-3-yl | 1.78^{a]} |
| 167 | 6-methylpyridin-2-yl | cPr | —CH₂—CH₂—CH₂— | cPr | 6-methylpyridin-2-yl | 2.85^{[a]} |
| | | | | | | (isomer mixture) |
| 168 | 2-methylpyrimidin-4-yl | Me | 2-methylpropane-1,3-diyl | Me | 2-methylpyrimidin-4-yl | 3.57^{a]} |
| 169 | 6-(trifluoromethyl)pyridin-2-yl | Me | 2-methylpropane-1,3-diyl | Me | 6-(trifluoromethyl)pyridin-2-yl | 6.41^{[a]} |
| 170 | 6-methylpyridin-2-yl | CH₂SO₂Me | —CH₂—CH₂—CH₂— | CH₂SO₂Me | 6-methylpyridin-2-yl | 2.75^{[a]} |
| 171 | 6-methylpyridin-2-yl | Me | 1-oxobutane-1,4-diyl | Me | 6-methylpyridin-2-yl | 2.44^{[a]} |
| 172 | 6-isobutylpyridin-2-yl | Me | 2-methylpropane-1,3-diyl | Me | 6-isobutylpyridin-2-yl | 7.36^{[a]} |
| 173 | 6-benzylpyridin-2-yl | Me | 2-methylpropane-1,3-diyl | Me | 6-benzylpyridin-2-yl | 7.36^{[a]} |
| 174 | 6-propylpyridin-2-yl | Me | —CH₂—CH₂—CH₂— | Me | 6-propylpyridin-2-yl | 6.78^{[a]} |
| 175 | 6-propylpyridin-2-yl | Me | 2-methylpropane-1,3-diyl | Me | 6-propylpyridin-2-yl | 7.36^{[a]} |
| 176 | 6-(trifluoromethyl)pyridin-2-yl | Me | 2-methylenepropane-1,3-diyl | Me | 6-(trifluoromethyl)pyridin-2-yl | 6.02^{[b]} |
| 177 | 6-methylpyridin-2-yl | Me | 2-methylenepropane-1,3-diyl | Me | 4-methoxy-6-methylpyrimidin-2-yl | 3.08^{[b]} (isomer A) |
| 178 | 4-(methoxycarbonyl)-6- | Me | 2-methylenepropane-1,3-diyl | Me | 4-(methoxycarbonyl)-6- | 5.46^{[b]} |
| | methylpyridin-2-yl | | | | methylpyridin-2-yl | |
| 179 | 6-ethylpyridin-2-yl | Me | 2-methylpropane-1,3-diyl | Me | 6-ethylpyridin-2-yl | 6.21^{[a]} |
| 180 | 6-ethylpyridin-2-yl | Me | —CH₂—CH₂—CH₂— | Me | 6-ethylpyridin-2-yl | 5.43^{[a]} |
| 181 | 6-methylpyridin-2-yl | Me | oxydiethane-1,5-diyl | Me | 6-methylpyridin-2-yl | 1.84^{[b]} |
| 182 | 6-methylpyridin-2-yl | Me | 2-methylenepropane-1,3-diyl | NH₂ | 4-methyl-5,6,7,8-tetra- | 2.55^{[b]} |
| | | | | | hydroquinazolin-2-yl | |
| 183 | 6-methylpyridin-2-yl | Me | 2-methylenepropane-1,3-diyl | NH₂ | 4,6-bis(methoxymethyl)-5- | 1.92^{[b]} |
| | | | | | methylpyrimidin-2-yl | |
| 184 | 6-methylpyridin-2-yl | Me | 2-methylenepropane-1,3-diyl | NH₂ | 5-chloro-4,6-dimethylpyrimidin-2-yl | 2.61^{[b]} |
| 185 | 6-methylpyridin-2-yl | Me | 2-methylenepropane-1,3-diyl | NH₂ | 3,5-Me-phenyl | 3.17^{[b]} |
| 186 | 6-methylpyridin-2-yl | Me | 2-methylenepropane-1,3-diyl | NH₂ | 4-methoxy-6-methylpyrimidin-2-yl | 2.11^{[b]} |
| 187 | 6-methylpyridin-2-yl | Me | 2-methylenepropane-1,3-diyl | NH₂ | 4-methylpyrimidin-2-yl | 1.73^{[b]} |
| 188 | 6-methylpyridin-2-yl | Me | 2-methylenepropane-1,3-diyl | Me | 4,6-dimethylpyrimidin-2-yl | 2.69^{[b]} |
| 189 | 6-methylpyridin-2-yl | Me | 2-methylenepropane-1,3-diyl | NH₂ | 4,6-dimethylpyridin-2-yl | 2.92^{[b]} |
| 190 | 6-methylpyridin-2-yl | Me | 2-methyleneproapne-1,3-diyl | NH₂ | 3,5-Cl₂-phenyl | 4.32^{[b]} |
| 191 | 6-methylpyridin-2-yl | Me | 2-methylenepropane-1,3-diyl | NH₂ | 4,5,6-trimethylpyrimidin-2-yl | 2.13^{[b]} |
| 192 | 6-methylpyridin-2-yl | Me | 2-methylenepropane-1,3-diyl | NH₂ | 4,6-dimethylpyrimidin-2-yl | 1.97^{[b]} |
| 193 | 6-methylpyridin-2-yl | Me | 2-methylenepropane-1,3-diyl | NH₂ | 4-methyl-1,3-thiazol-2-yl | 2.70^{b]} |
| 194 | 6-methylpyridin-2-yl | Me | 2-methylenepropane-1,3-diyl | Me | 3,5-dichloropyridin-2-yl | 4.72^{b]} |
| 195 | 6-methylpyridin-2-yl | Me | 2-methylenepropane-1,3-diyl | Me | 5-chloro-2-thienyl | 4.82^{[b]} (isomer A) |
| 196 | 6-methylpyridin-2-yl | Me | 2-methylenepropane-1,3-diyl | Me | 2-thienyl | 4.23^{[b]} |
| 197 | 6-methylpyridin-2-yl | Me | 2-methylenepropane-1,3-diyl | Me | 1-methyl-1H-pyrrol-2-yl | 4.37^{[b]} |
| 198 | 6-methylpyridin-2-yl | Me | 2-methylenepropane-1,3-diyl | Me | 3,5-dimethylisoxazol-4-yl | 3.50^{[b]} |
| 199 | 6-methylpyridin-2-yl | Me | 2-methylenepropane-1,3-diyl | NH₂ | 1,5-dimethyl-1H-pyrrol-2-yl | 2.23^{[b]} |
| 200 | 6-methylpyridin-3-yl | Me | 2-methylenepropane-1,3-diyl | Me | 6-methylpyridin-2-yl | 1.92^{[b]} (isomer A) |
| 201 | 6-chloro-3-pyridyl | Me | 2-methylenepropane-1,3-diyl | Me | 6-methylpyridin-2-yl | 3.94^{[b]} |
| 202 | quinolin-2-yl | Me | 2-methylenepropane-1,3-diyl | Me | 6-methylpyridin-2-yl | 5.25^{[b]} |
| 203 | 6-methylpyridin-2-yl | Me | 2-methylenepropane-1,3-diyl | Me | 3-methylpyridin-2-yl | 3.03^{[b]} (isomer A) |
| 204 | 5,6-dimethylpyridin-2-yl | Me | 2-methylenepropane-1,3-diyl | Me | 6-methylpyridin-2-yl | 3.10^{[b]} |
| 205 | 3-Me-Ph | Me | 2-methylenepropane-1,3-diyl | Me | 6-methylpyridin-2-yl | 5.03^{[b]} |
| 206 | 6-methylpyridin-2-yl | Me | 2-methylenepropane-1,3-diyl | Me | 6-bromopyridin-2-yl | 4.98^{[b]} |
| 207 | 6-methoxypyridin-2-yl | Me | 2-methylenepropane-1,3-diyl | Me | 6-methylpyridin-2-yl | 4.82^{[b]} |
| 208 | 6-bromopyridin-3-yl | Me | 2-methylenepropane-1,3-diyl | Me | 6-methylpyridin-2-yl | 4.08^{[b]} |
| 209 | 5-methylpyridin-2-yl | Me | 2-methylenepropane-1,3-diyl | Me | 6-methylpyridin-2-yl | 3.33^{b]} |
| 210 | 6-methylpyridin-2-yl | Me | 2-methylenepropane-1,3-diyl | NH₂ | 6-methyl-4-(trifluoromethyl)- | 4.37^{[b]} |
| | | | | | pyridin-2-yl | |
| 211 | 6-methylpyridin-2-yl | Me | 2-methylenepropane-1,3-diyl | Me | 6-methyl-4-(trifluoromethyl)- | 5.70^{[b]} |
| | | | | | pyridin-2-yl | |
| 212 | 6-methylpyridin-2-yl | Me | 2-fluoropropane-1,3-diyl | Me | 6-methylpyridin-2-yl | 2.94^{[b]} |
| 213 | 1,3-benzothiazol-2-yl | Me | 2-methylpropane-1,3-diyl | Me | 1,3-benzothiazol-2-yl | 6.71^{[b]} |
| 214 | 6-methylpyridin-2-yl | CF₃ | 2-methylpropane-1,3-diyl | CF₃ | 6-methylpyridin-2-yl | 4.83^{[b]} |
| 215 | 6-methylpyridin-2-yl | Me | (2,2-dimethyl-1,3-dioxolan-4,5-diyl)bis(methylene) | Me | 6-methylpyridin-2-yl | 3.09^{[b]} |
| 216 | 6-methylpyridin-2-yl | Me | methylenebis(oxyethane-2,1-diyl) | Me | 6-methylpyridin-2-yl | 2.02^{[b]} |
| 217 | 6-methylpyridin-2-yl | Me | heptane-1,7-diyl | Me | 6-methylpyridin-2-yl | 5.37^{[b]} |
| 218 | 6-methylpyridin-2-yl | Me | 2-methylenepropane-1,3-diyl | Me | 1,3-benzothiazol-2-yl | 4.79^{[b]} |
| 219 | 4,6-dimethylpyridin-2-yl | Me | 2-methylenepropane-1,3-diyl | Me | 4,6-dimethylpyridin-2-yl | 1.98^{[b]} |
| 220 | 6-methylpyridin-2-yl | Me | oxirane-2,2-diylbis(methylene) | Me | 6-methylpyridin-2-yl | 2.24^{[a]} |
| 221 | 6-methylpyridin-2-yl | Me | 2-hydroxy-2-[({[(1E)-1-(6-methypyridin-2-yl)ethylidene]amino}oxy)meth- | Me | 6-methylpyridin-2-yl | 2.65^{[a)} |
| | | | yl]propane-1,3-diyl | | | |
| 222 | 6-methylpyridin-2-yl | Et | —CH₂—CH₂— | Et | 6-methylpyridin-2-yl | 3.77^{[a]} |
| | | | | | | (isomer mixture) |
| 223 | 6-methylpyridin-2-yl | Et | —(CH₂)₄— | Et | 6-methylpyridin-2-yl | 4.86^{[a]} |
| | | | | | | (isomer mixture) |
| 224 | 4,6-dimethylpyridin-2-yl | Me | —CH₂—CH₂—CH₂— | Me | 4,6-dimethylpyridin-2-yl | 1.65^{[a]} |
| 225 | 4,6-dimethylpyridin-2-yl | Me | 2-methylpropane-1,3-diyl | Me | 4,6-dimethylpyridin-2-yl | 2.11^{[a]} |
| 226 | 6-methylpyridin-2-yl | nPr | —CH₂—CH₂— | nPr | 6-methylpyridin-2-yl | 5.18^{[a]} |
| 227 | 6-methylpyridin-2-yl | nPr | —(CH₂)₄— | nPr | 6-methylpyridin-2-yl | 6.26^{[a]} |
| 228 | 6-methylpyridin-3-yl | Me | —CH₂—CH₂—CH₂— | Me | 6-methylpyridin-3-yl | 1.27^{[a]} |
| 229 | 6-isopropylpyridin-2-yl | Me | 2-methylpropane-1,3-diyl | Me | 6-Isopropylpyridin-2-yl | 7.36^{[a]} |
| 230 | 6-isopropylpyridin-2-yl | Me | —CH₂—CH₂—CH₂— | Me | 6-Isopropylpyridin-2-yl | 7.36^{[a]} |
| 231 | 2-chloro-1,3-thiazol-4-yl | Me | 2-methylpropane-1,3-diyl | Me | 2-chloro-1,3-thiazol-4-yl | 5.16^{[a]} |
| | | | | | | (isomer mixture) |
| 232 | 2-chloro-1,3-thiazol-4-yl | Me | —CH₂—CH₂—CH₂— | Me | 2-chloro-1,3-thiazol-4-yl | 4.69^{[a]} |
| | | | | | | (isomer mixture) |
| 233 | 6-methylpyridin-3-yl | Me | 2-methylpropane-1,3-diyl | Me | 6-methylpyridin-3-yl | 1.50^{[a]} |
| | | | | | | (isomer mixture) |
| 234 | 2-methylpyridin-3-yl | Me | 2-methylpropane-1,3-diyl | Me | 2-methylpyridin-3-yl | 1.22^{[a]} |
| | | | | | | (isomer mixture) |
| 235 | 5,6-dimethylpyridin-2-yl | Et | 2-methylpropane-1,3-diyl | Et | 5,6-dimethylpyridin-2-yl | 4.88^{[a]} |
| 236 | 5,6-dimethypyridin-2-yl | Et | —CH₂—CH₂—CH₂— | Et | 5,6-dimethylpyridin-2-yl | 3.80^{[a]} |
| | | | | | | (isomer mixture) |
| 237 | 5,6-dimethylpyridin-2-yl | iPr | —CH₂—CH₂—CH₂— | iPr | 5,6-dimethypyridin-2-yl | 4.00^{[a]} |
| | | | | | | (isomer mixture) |
| 238 | 5,6-dimethypyridin-2-yl | iPr | —CH₂—CH₂—CH₂— | iPr | 5,6-dimethypyridin-2-yl | 4.94^{[a]} |
| | | | | | | (isomer mixture) |
| 239 | 6-methylpyridin-2-yl | Et | 2-methylenepropane-1,3-diyl | Et | 6-methylpyridin-2-yl | 5.16^{[a]} |
| 240 | 6-methylpyridin-2-yl | nPr | 2-methylenepropane-1,3-diyl | nPr | 6-methylpyridin-2-yl | 6.27^{[a]} |
| | | | | | | (isomer mixture) |
| 241 | 2-methylpyridin-3-yl | Me | —CH₂—CH₂—CH₂— | Me | 2-methylpyridin-3-yl | |
| 242 | 6-methylpyridin-2-yl | Me | 2-methylenepropane-1,3-diyl | NH₂ | 3,5-difluoropyridin-2-yl | 2.48^{[b)} |
| 243 | 6-methylpyridin-2-yl | Me | 2-methylenepropane-1,3-diyl | NH₂ | 4,6-dicylclopropylpyrimidin-2-yl | 3.09^{[b]} |
| 244 | 6-methylpyridin-2-yl | Me | 2-methylenepropane-1,3-diyl | NH₂ | 5-sec-butyl-4-cyclopropyl-6-methyl- | 3.99^{[b]} |
| | | | | | pyrimidin-2-yl | |
| 245 | 6-methylpyridin-2-yl | Me | 2-methylenepropane-1,3-diyl | NH₂ | 3-chloro-5-(trifluoromethyl)- | 3.50^{[b]} |
| | | | | | pyridin-2-yl | |
| 246 | 6-methylpyridin-2-yl | Me | 2-methylenepropane-1,3-diyl | NH₂ | 3,5-dichloropyridin-2-yl | 3.00^{[b]} |
| 247 | 6-methoxypyridin-2-yl | Me | 2-oxopropane-1,3-diyl | Me | 6-methoxypyridin-2-yl | 4.61^{[b]} |
| 248 | 6-methylpyridin-2-yl | Me | pentane-2,4-diyl | Me | 6-methylpyridin-2-yl | 4.10^{[b]} |
| 249 | 6-methylpyridin-2-yl | Me | 2-methylenepropane-1,3-diyl | Me | 4-ethoxypyrimidin-2-yl | 3.17^{[b]} |
| 250 | 6-methylpyridin-2-yl | Me | 2,2-dimethylpropane-1,3-diyl | Me | 6-methylpyridin-2-yl | 4.74^{[b]} |
| | | | | | | (isomer mixture) |
| 251 | 3-methoxy-6-methylpyridin-2-yl | Me | 2-methylpropane-1,3-diyl | Me | 3-methoxy-6-methylpyridin-2-yl | 2.04^{[b]} |
| 252 | 4-ethoxypyrimidin-2-yl | Me | 2-methylenepropane-1,3-diyl | Me | 4-ethoxypyrimidin-2-yl | 3.42^{[b]} |
| 253 | 6-ethylpyridin-2-yl | Et | 2-methylpropane-1,3-diyl | Et | 6-ethylpyridin-2-yl | (isomer A) |
| 254 | 4,5,6-trimethylpyrimidin-2-yl | Et | 2-methylpropane-1,3-diyl | Et | 4,5,6-trimethylpyrimidin-2-yl | 4.38^{[b]} |
| 255 | 3,5,6-trimethylpyridin-2-yl | Me | 2-methylpropane-1,3-diyl | Me | 3,5,6-trimethylpyridin-2-yl | 2.19^{[a]} |
| 256 | 3,5,6-trimethylpyridin-2-yl | Me | —CH₂—CH₂—CH₂— | Me | 3,5,6-trimethylpyridin-2-yl | 1.84^{[a]} |
| 257 | 6-(ethoxycarbonyl)pyridin-2-yl | Me | —CH₂—CH₂—CH₂— | Me | 6-(ethoxycarbonyl)pyridin-2-yl | 4.06^{[a]} |
| 258 | 6-(ethoxycarbonyl)pyridin-2-yl | Me | 2-methylpropane-1,3-diyl | Me | 6-(ethoxycarbonyl)pyridin-2-yl | 5.03^{[a]} |
| 259 | 1,5-dimethyl-1H-pyrazol-3-yl | Me | —CH₂—CH₂—CH₂— | Me | 1,5-dimethyl-1H-pyrazol-3-yl | 2.70^{[a]} |
| 260 | 5,6-dimethylpyridin-2-yl | nPr | —CH₂—CH₂—CH₂— | nPr | 5,6-dimethylpyridin-2-yl | 5.22^{[a]} |
| | | | | | | (isomer mixture) |
| 261 | 1,5-dimethyl-1H-pyrazol-3-yl | Me | 2-methylpropane-1,3-diyl | Me | 1,5-dimethyl-1H-pyrazol-3-yl | 3.09^{[a]} |
| | | | | | | (isomer mixture) |
| 262 | 5,6-dimethylpyridin-2-yl | nPr | 2-methylpropane-1,3-diyl | nPr | 5,6-dimethylpyridin-2-yl | 6.26^{[a]} |
| 263 | 6-ethylpyridin-2-yl | Et | 2-methylpropane-1,3-diyl | Et | 6-ethylpyridin-2-yl | 6.33^{[b]} |
| | | | | | | (isomer mixture) |
| 264 | 6-methylpyridin-2-yl | Me | 2-phenylpropane-1,3-diyl | Me | 6-methylpyridin-2-yl | 4.63^{[b]} |
| 265 | 6-ethylpyridin-2-yl | Et | 2-methylenepropane-1,3-diyl | Et | 6-ethylpyridin-2-yl | 7.08^{[b]} |
| 266 | 4,5,6-trimethylpyrimidin-2-yl | Me | 2-methylpropane-1,3-diyl | Me | 4,5,6-trimethylpyrimidn-2-yl | 3.41^{[b]} |
| 267 | 3,6-dimethylpyridin-2-yl | Me | 2-methylpropane-1,3-diyl | Me | 3,6-dimethylpyridin-2-yl | 2.5^{[b]} (isomer B) |
| 268 | 3,6-dimethylpyridin-2-yl | Me | 2-methylpropane-1,3-diyl | Me | 3,6-dimethylpyridin-2-yl | 2.08^{[b]} (isomer A) |
| 269 | 6-methylpyridin-2-yl | Me | 2-tert.butyl-propane-1,3-diyl | Me | 6-methylpyridin-2-yl | 5.51^{[b]} |
| 270 | 6-methylpyridin-2-yl | Me | 2-n-propyl-propane-1,3-diyl | Me | 6-methylpyridin-2-yl | 5.14^{[b]} |
| 271 | 4,6-dimethoxypyrimidin-2-yl | Me | 2-methylpropane-1,3-diyl | Me | 4-hydroxy-6-methoxypyrimidin-2-yl | 3.05^{[b]} |
| 272 | 4,6-dimethoyxpyrimidin-2-yl | Me | 2-methylpropane-1,3-diyl | Me | 4,6-dimethoxypyrimidin-2-yl | 4.49^{[b]} |
| 273 | 4-methyl-6-(trifluoromethyl)- | Me | 2-methylpropane-1,3-diyl | Me | 4-methyl-6-(trifluoro- | 4.86^{[b]} |
| | pyrimidin-2-yl | | | | methyl)pyrimidin-2-yl | |
| 274 | 6-methylpyridin-2-yl | iPr | 2-methylenepropane-1,3-diyl | iPr | 6-methylpyridin-2-yl | 4.87^{[a]} (isomer B) |
| 275 | 6-methylpyridin-2-yl | iPr | 2-methylenepropane-1,3-diyl | iPr | 6-methylpyridin-2-yl | 4.90^{[a]} (isomer A) |
| 276 | 4,6-dimethylpyridin-2-yl | Me | 2,2-dimethylpropane-1,3-diyl | Me | 4,6-dimethylpyridin-2-yl | 2.54^{[a]} |
| 277 | 6-methylpyridin-2-yl | Et | 2,2-dimethylpropane-1,3-diyl | Et | 6-methylpyridin-2-yl | 6.11^{[a]} |
| 278 | 5,6-dimethylpyridin-2-yl | Me | 2,2-dimethylpropane-1,3-diyl | Me | 5,6-dimethylpyridin-2-yl | 3.81^{[a]} |
| 279 | 6-methylpyridin-2-yl | nPr | 2,2-dimethylpropane-1,3-diyl | nPr | 6-methylpyridin-2-yl | 7.25^{[a]} |
| 280 | 5,6-dimethylpyridin-2-yl | Et | 2,2-dimethylpropane-1,3-diyl | Et | 5,6-dimethylpyridin-2-yl | 5.58^{[a]} |
| 281 | 6-methylpyridin-2-yl | cPr | 2,2-dimethylpropane-1,3-diyl | cPr | 6-methylpyridin-2-yl | 4.04^{[a]} |
| | | | | | | (isomer mixture) |
| 282 | 6-methylpyridin-2-yl | cPr | 2,2-dimethylpropane-1,3-diyl | cPr | 6-methylpyridin-2-yl | 3.75^{[a]} (isomer A) |
| 283 | 3-methoxy-6-methylpyridin-2-yl | Me | 2-methyleneproane-1,3-diyl | Me | 3-methoxy-6-methylpyridin-2-yl | 2.05^{[b]} |
| 284 | 5,6-dimethylpyridazin-3-yl | Me | 2-methylenepropane-1,3-diyl | Me | 5,6-dimethylpyridazin-3-yl | 2.33^{[b]} |
| 285 | 6-methylpyridin-2-yl | Me | (2S,3S)-oxirane-2,3-diylbis(methylene) | Me | 6-methylpyridin-2-yl | 2.17^{[b]} |
| 286 | 6-methylpyridin-2-yl | Me | Rac-oxirane-2,3-diylbis(methylene) | Me | 6-methylpyridin-2-yl | 2.08^{[b]} |
| 287 | 6-methylpyridin-2-yl | Me | (2R,3S)-oxirane-2,3-diylbis(methylene) | Me | 6-methylpyridin-2-yl | 2.42^{[b]} |
| 288 | 6-methylpyridin-2-yl | iPr | —CH₂—CH₂— | iPr | 6-methylpyridin-2-yl | 4.03^{[a]} |
| | | | | | | (isomer mixture) |
| 289 | 6-methylpyridin-2-yl | Me | cyclobutan-1,1-diylbis(methylene) | Me | 6-methylpyridin-2-yl | 4.77^{[b]} |
| 290 | 6-(methoxymethyl)pyridin-2-yl | Me | 2-methylpropane-1,3-diyl | Me | 6-(methoxymethyl)pyridin-2-yl | 4.92^{[b]} |
| 291 | 6-(methoxymethyl)pyridin-2-yl | Me | 2-methylenepropane-1,3-diyl | Me | 6-(methoxymethyl)pyridin-2-yl | 4.44^{[b]} |
| 292 | 6-methylpyridin-2-yl | Me | (dimethylsilanediyl)bis(methylene) | Me | 6-methylpyridin-2-yl | 4.50^{[a]} |
| 293 | 6-methylpyridin-2-yl | Me | 2-hydroxypropane-1,3-diyl | Me | 6-methylpyridin-2-yl | 1.46^{[b]} |
| 294 | 6-methylpyridin-2-yl | Me | 2-methoxypropane-1,3-diyl | Me | 6-methylpyridin-2-yl | 2.22^{[b]} |
| 295 | 6-methylpyridin-2-yl | Me | butane-1,3-didyl | Me | 6-methylpyridin-2-yl | 3.34^{[b]} |
| 296 | 6-methylpyridin-2-yl | Me | 2-methylpropane-1,3-diyl | Me | 6-methylpyridin-2-yl | CoCl₂ salt |
| 297 | 6-methylpyridin-2-yl | Me | 2-methylpropane-1,3-diyl | Me | 6-methylpyridin-2-yl | ZnCl₂ salt |
| 298 | 6-methylpyridin-2-yl | Me | 2-methylpropane-1,3-diyl | Me | 6-methylpyridin-2-yl | FeCl₂ salt |
| 299 | 5,6-dimethylpyridin-2-yl | iPr | 2-methylpropane-1,3-diyl | iPr | 5,6-dimethylpyridin-2-yl | 4.82^{[a]} |
| | | | | | | (isomer mixture) |
| 300 | 6,7-dihydro-5H-cyclo- | Me | 2-methylpropane-1,3-diyl | Me | 6,7-dihydro-5H-cyclo- | 4.14^{[a]} |
| | penta[b]pyridin-2-yl | | | | penta[b]pyridin-2-yl | |
| 301 | 6,7-dihydro-5H-cyclo- | Me | 2,2-dimethylpropane-1,3-diyl | Me | 6,7-dihydro-5H-cyclo- | 4.78^{[a]} |
| | penta[b]pyridin-2-yl | | | | penta[b]pyridin-2-yl | |
| 302 | 6,7-dihydro-5H-cyclo- | Me | —CH₂—CH₂—CH₂— | Me | 6,7-dihydro-5H-cyclo- | 3.28^{[a]} |
| | penta[b]pyridin-2-yl | | | | penta[b]pyridin-2-yl | |
| 303 | 5,6,7,8-tetrahydroquinolin-2-yl | Me | 2,2-dimethylpropane-1,3-diyl | Me | 5,6,7,8-tetrahydroquinolin-2-yl | 5.49^{[a]} |
| 304 | 5,6,7,8-tetrahydroquinolin-2-yl | Me | 2-methylpropane-1,3-diyl | Me | 5,6,7,8-tetrahydroquinolin-2-yl | 4.69^{[a]} |
| 305 | 5,6,7,8-tetrahydroquinolin-2-yl | Me | —CH₂—CH₂—CH₂— | Me | 5,6,7,8-tetrahydroquinolin-2-yl | 3.96^{[a]} |
| 306 | 6-(difluoromethyl)pyridin-2-yl | Me | 2-methylpropane-1,3-diyl | Me | 6-(difluoromethyl)pyridin-2-yl | 5.49^{[a]} |
| 307 | 3,6-dimethylpyridin-2-yl | Me | 2-methylenepropane-1,3-diyl | Me | 3,6-dimethylpyridin-2-yl | 2.37^{[b]} (isomer A) |
| 308 | 3,6-dimethylpyridin-2-yl | Me | 2-methylenepropane-1,3-diyl | Me | 3,6-dimethylpyridin-2-yl | 2.94^{[c]} |
| | | | | | | 1.84^{[b]} |
| | | | | | | (isomer B) |
| 309 | 5,6-dimethylpyridazin-3-yl | Me | 2-methylpropane-1,3-diyl | Me | 5,6-dimethylpyridazin-3-yl | 2.58^{[b]} |
| 310 | 6-ethyl-5-methylpyridin-2-yl | Me | 2-methylpropane-1,3-diyl | Me | 6-ethyl-5-methylpyridin-2-yl | 6.48^{[b]} |
| 311 | 6-methylpyridin-2-yl | cPr | 2-methylenepropane-1,3-diyl | cPr | 6-methylpyridin-2-yl | 3.29^{[a]} (isomer B) |
| 312 | 6-methylpyridin-2-yl | cPr | 2-methylenepropane-1,3-diyl | cPr | 6-methylpyridin-2-yl | 3.10^{[a]} |
| | | | | | | (isomer mixture) |
| 313 | 6-methylpyridin-2-yl | Me | 2-methylpropane-1,3-diyl | Me | 6-methylpyridin-2-yl | CuCl salt |
| 314 | 6-methyl-1-oxidopyridin-2-yl | Me | 2-methylpropane-1,3-diyl | Me | 6-methyl-1-oxidopyridin-2-yl | 1.69^{[b]} |
| 315 | 5,6-dimethylpyridin-2-yl | Me | cyclopropane-1,1-diylbis(methylene) | Me | 5,6-dimethylpyridin-2-yl | 6.08^{[c]} |
| | | | | | | 2.84^{[b]} |
| 316 | 5,6-dimethylpyridin-2-yl | Me | 2-n-propyl-propane-1,3-diyl | Me | 5,6-dimethylpyridin-2-yl | 7.23^{[c]} |
| | | | | | | 4.48^{[b]} |
| 317 | 5,6-dimethylpyridin-2-yl | Me | 2-tert.butyl-propane-1,3-diyl | Me | 5,6-dimethylpyridin-2-yl | 7.36^{[c]} |
| | | | | | | 4.91^{[b]} |
| 318 | 5,6-dimethylpyridin-2-yl | Me | 2-phenylpropane-1,3-diyl | Me | 5,6-dimethylpyridin-2-yl | 6.45^{[c]} |
| | | | | | | 4.02^{[b]} |
| 319 | 5,6-dimethylpyridin-2-yl | Me | cyclobutane-1,1-diylbis(methylene) | Me | 5,6-dimethylpyridin-2-yl | 7.05^{[c]} |
| | | | | | | 4.07^{b]} |
| 320 | 5,6-dimethylpyridin-2-yl | Me | cyclohexane-1,1-diylbis(methylene) | Me | 5,6-dimethylpyridin-2-yl | 7.36^{[c]} |
| | | | | | | 5.34^{[b]} |
| 321 | 5,6-dimethylpyridin-2-yl | Me | 2-methoxypropane-1,3-diyl | Me | 5,6-dimethylpyridin-2-yl | 5.06^{[c]} |
| | | | | | | 1.95^{[b]} |
| 322 | 5,6-dimethylpyridin-2-yl | Me | pentane-2,4-diyl | Me | 5,6-dimethylpyridin-2-yl | 6.79^{[c]} |
| | | | | | | 3.4^{[b]} |
| 323 | 5,6-dimethylpyridin-2-yl | Me | 2-hydroxypropane-1,3-diyl | Me | 5,6-dimethylpyridin-2-yl | 3.81^{[c]} |
| | | | | | | 1.36^{[b]} |
| 324 | 6-benzylpyridin-2-yl | Me | —(CH₂)₄— | Me | 6-methylpyridin-2-yl | 5.84^{[b]} |
| 325 | 6-methylpyridin-2-yl | Me | —(CH₂)₄— | Ph | 3-methylpyridin-2-yl | 3.89^{[b]} |
| 326 | 6-isobutylpyridin-2-yl | Me | —(CH₂)₄— | Me | 6-methylpyridin-2-yl | 6.16^{[b]} |
| 327 | 6-methylpyridin-2-yl | Me | —(CH₂)₄— | nPr | 3-methylpyridin-2-yl | 3.61^{[b]} (isomer A) |
| 328 | 6-methylpyridin2--yl | Me | —(CH₂)₄— | Et | 3-methylpyridin-2-yl | 3.65^{[b]} (isomer B) |
| 329 | 6-ethylpyridin-2-yl | Me | —(CH₂)₄— | Me | 6-methylpyridin2--yl | 4.72^{[b]} |
| 330 | 6-methylpyridin-2-yl | nPr | —(CH₂)₄— | Me | 6-methylpyridin-2-yl | 4.82^{[b]} |
| 331 | 6-methylpyridin-2-yl | Me | —(CH₂)₄— | iBu | 6-methylpyridin-2-yl | 5.46^{[b]} (isomer A) |
| 332 | 6-methylpyridin-2-yl | Me | —(CH₂)₄— | Ph | 6-methylpyridin-2-yl | 3.51^{[b]} |
| 333 | 6-methylpyridin-2-yl | Et | —(CH₂)₄— | Me | 6-methylpyridin-2-yl | 4.10^{[b]} |
| 334 | 6-methylpyridin-2-yl | Me | —(CH₂)₄— | iPr | 6-methylpyridin-2-yl | 4.72^{[b]} (isomer B) |
| 335 | 6-methylpyridin-2-yl | Me | —(CH₂)₄— | cPr | 6-methylpyridin-2-yl | 3.28^{[b]} |
| 336 | 6-methylpyridin-2-yl | Me | —(CH₂)₄— | Me | 2-methylpyrimidin-4-yl | 3.28^{[b]} |
| 337 | 5-methylpyridin-3-yl | Me | —(CH₂)₄— | Me | 6-methylpyridin-2-yl | 2.20^{[b]} |
| 338 | 2-methylpyridin-4-yl | Me | —(CH₂)₄— | Me | 6-methylpyridin-2-yl | 1.71^{[b]} |
| 339 | 6-methylpyridin-2-yl | Me | hexane-1,6-diyl | Bzl | 3-methylpyridin-2-yl | 5.90^{[b]} (isomer B) |
| 340 | 6-benzylpyridin-2-yl | Me | hexane-1,6-diyl | Me | 6-methylpyridin-2-yl | 6.86^{[b]} |
| 341 | 6-methylpyridin-2-yl | Me | hexane-1,6-diyl | Ph | 3-methylpyridin-2-yl | 4.98^{b]} |
| 342 | 6-isobutylpyridin-2-yl | Me | hexane-1,6-diyl | Me | 6-methylpyridin-2-yl | 7.36^{[b]} |
| 343 | 6-methylpyridin-2-yl | Me | hexane-1,6-diyl | Et | 3-methylpyridin-2-yl | 4.87^{[b]} (isomer B) |
| 344 | 6-methylpyridin-2-yl | Me | hexane-1,6-diyl | Me | 3-methyl-1,2,4-triazin-5-yl | 4.15^{[b]} |
| 345 | 6-methylpyridin-2-yl | nPr | hexane-1,6-diyl | Me | 6-methylpyridin-2-yl | 6.16^{[b]} |
| 346 | 6-methylpyridin-2-yl | Et | hexane-1,6-diyl | Me | 6-methylpyridin-2-yl | 5.59^{[b]} |
| 347 | 6-methylpyridin-2-yl | Me | hexane-1,6-diyl | cPr | 6-methylpyridin-2-yl | 4.56^{[b]} |
| 348 | 6-methylpyridin-2-yl | Me | hexane-1,6-diyl | Me | 2-methylpyrimidin-4-yl | 4.51^{[b]} |
| 349 | 6-methylpyridin-2-yl | Me | 2-methylenepropane-1,3-diyl | Bzl | 3-methylpyridin2--yl | 4.82^{[b]} |
| 350 | 6-benzylpyridin-2-yl | Me | 2-methylenepropane-1,3-diyl | Me | 6-methylpyridin-2-yl | 5.71^{[b]} |
| 351 | 6-methylpyridin-2-yl | Me | 2-methylenepropane-1,3-diyl | Ph | 3-methylpyridin-2-yl | 3.89^{[b]} |
| 352 | 6-isobutylpyridin-2-yl | Me | 2-methylenepropane-1,3-diyl | Me | 6-methylpyridin-2-yl | 6.03^{[b]} |
| 353 | 6-methylpyridin-2-yl | Me | 2-methylenepropane-1,3-diyl | But-3-en-1-yl | 3-methlpyridin-2-yl | 4.56^{[b]} |
| 354 | 6-ethylpyridin-2-yl | Me | 2-methylenepropane-1,3-diyl | Me | 6-methylpyridin-2-yl | 4.67^{[b]} |
| 355 | 6-methylpyridin-2-yl | nPr | 2-methylenepropane-1,3-diyl | Me | 6-methylpyridin-2-yl | 4.87^{[b]} |
| 356 | 6-methylpyridin-2-yl | Me | 2-methylenepropane-1,3-diyl | iBu | 6-methylpyridin-2-yl | 5.52^{[b]} |
| 357 | 6-methylpyridin-2-yl | Me | 2-methylenepropane-1,3-diyl | Ph | 6-methylpyridin-2-yl | 3.65^{[b]} |
| 358 | 6-methylpyridin-2-yl | Et | 2-methylenepropane-1,3-diyl | Me | 6-methylpyridin-2-yl | 4.30^{[b]} |
| 359 | 6-methylpyridin-2-yl | Me | 2-methylenepropane-1,3-diyl | iPr | 6-methylpyridin-2-yl | 4.72^{[b]} (isomer B) |
| 360 | 2-methylpyrimidin-4-yl | Me | 2-methylenepropane-1,3-diyl | Me | 6-methylpyridin-2-yl | 3.24^{[b]} |
| 361 | 5-methylpyridin-3-yl | Me | 2-methylenepropane-1,3-diyl | Me | 6-methylpyridin-2-yl | 2.20^{[b]} |
| 362 | 2-methylpyridin-4-yl | Me | 2-methylenepropane-1,3-diyl | Me | 6-methylpyridin-2-yl | 1.71^{[b]} (isomer B) |
| 363 | 6-methylpyridin-2-yl | Me | 2,2-dimethylpropane-1,3-diyl | Bzl | 3-methylpyridin-2-yl | 5.71^{[b]} |
| 364 | 6-benzylpyridin-2-yl | Me | 2,2-dimethylpropane-1,3-diyl | Me | 6-methylpyridin-2-yl | 6.47^{[a]} |
| 365 | 6-methylpyridin-2-yl | Me | 2,2-dimethylpropane-1,3-diyl | Ph | 3-methylpyridin-2-yl | 4.51^{[b]} |
| 366 | 6-methylpyridin-2-yl | Me | 2,2-dimethylpropane-1,3-diyl | iBu | 3-methylpyridin-2-yl | 5.97^{[b]} |
| 367 | 6-isobutylpyridin-2-yl | Me | 2,2-dimethylpropane-1,3-diyl | Me | 6-methylpyridin-2-yl | 7.07^{[b]} |
| 368 | 6-methylpyridin-2-yl | Me | 2,2-dimethylpropane-1,3-diyl | iPr | 3-methylpyridin-2-yl | 4.56^{[b]} |
| 369 | 6-methypyridin-2-yl | Me | 2,2-dimethylpropane-1,3-diyl | Et | 3-methylpyridin-2-yl | 4.51^{[b]} |
| 370 | 6-ethylpyridin-2-yl | Me | 2,2-dimethylpropane-1,3-diyl | Me | 6-methylpyridin-2-yl | 5.78^{[b]} |
| 371 | 6-methylpyridin-2-yl | Me | 2,2-dimethylpropane-1,3-diyl | Ph | 6-methylpyridin-2-yl | 4.51^{[b]} |
| 372 | 6-methylpyridin-2-yl | Et | 2,2-dimethylpropane-1,3-diyl | Me | 6-methylpyridin-2-yl | 5.33^{[b]} |
| 373 | 4-methylpyridin-2-yl | Me | —(CH₂)₄— | Me | 6-methylpyridin-2-yl | 2.66^{[b]} |
| 374 | 6-methylpyridin-2-yl | Me | —(CH₂)₄— | Me | 2,4-dimethyl-1,3-thiazol-5-yl | 3.56^{[b]} (isomer B) |
| 375 | 3-CF₃-phenyl | Me | —(CH₂)₄— | Me | 6-methylpyridin-2-yl | 5.54^{[b]} |
| 376 | 2-pyridyl | Me | —(CH₂)₄— | Me | 6-methylpyridin-2-yl | 3.11^{[b]} |
| 377 | 6-methylpyridin-2-yl | Me | —(CH₂)₄— | Me | 4,5-dimethyl-1,3-thiazol-2-yl | 4.62^{[b]} |
| 378 | 3,4-methylenedioxy-Ph | Me | —(CH₂)₄— | Me | 6-methylpyridin-2-yl | 4.35^{[b]} |
| 379 | 2-Cl-phenyl | Me | —(CH₂)₄— | Me | 6-methylpyridin-2-yl | 4.85^{[b]} |
| 380 | 3,4-OMe-phenyl | Me | —(CH₂)₄— | Me | 6-methylpyridin-2-yl | 3.85^{[b]} |
| 381 | 6-methylpyridin-2-yl | Me | —(CH₂)₄— | Me | benzofuran-2-yl | 4.74^{[b]} |
| 382 | 6-methylpyridin-3-yl | Me | —(CH₂)₄— | Me | 6-methylpyridin-2-yl | 1.90^{[b]} |
| 383 | 1-naphthyl | Me | —(CH₂)₄— | Me | 6-methylpyridin-2-yl | 5.19^{[b]} |
| 384 | 3-pyridyl | Me | —(CH₂)₄— | Me | 6-methylpyridin-2-yl | 2.18^{[b]} |
| 385 | phenyl | Me | —(CH₂)₄— | Me | 6-methylpyridin-2-yl | 4.68^{[b]} |
| 386 | 4-methylpyridin-2-yl | Me | hexane-1,6-diyl | Me | 6-methylpyridin-2-yl | 3.94^{[b]} |
| 387 | 3-CF₃-phenyl | Me | hexane-1,6-diyl | Me | 6-methylpyridin-2-yl | 6.51^{[b]} |
| 388 | 2-pyridyl | Me | hexane-1,6-diyl | Me | 6-methylpyridin-2-yl | 4.41^{[b]} |
| 389 | 6-methylpyridin-2-yl | Me | hexane-1,6-diyl | Me | 4,5-dimethyl-1,3-thiazol-2-yl | 5.84^{[b]} (isomer A) |
| 390 | 3,4-methylenedioxy-Ph | Me | hexane-1,6-diyl | Me | 6-methypyridin-2-yl | 5.42^{[b]} |
| 391 | 2-Cl-phenyl | Me | hexane-1,6-diyl | Me | 6-methylpyridin-2-yl | 5.84^{[b]} |
| 392 | 3,4-OMe-phenyl | Me | hexane-1,6-diyl | Me | 6-methylpyridin-2-yl | 4.91^{[b]} |
| 393 | 6-methylpyridin-2-yl | Me | hexane-1,6-diyl | Me | benzofuran-2-yl | 5.78^{[b]} |
| 394 | 6-methylpyridin-3-yl | Me | hexane-1,6-diyl | Me | 6-methylpyridin-2-yl | 2.62^{[b]} |
| 395 | 1-naphthyl | Me | hexane-1,6-diyl | Me | 6-methylpyridin-2-yl | 6.16^{[b]} |
| 396 | 3-pyridyl | Me | hexane-1,6-diyl | Me | 6-methylpyridin-2-yl | 3.15^{b]} |
| 397 | phenyl | Me | hexane-1,6-diyl | Me | 6-methlpyridin-2-yl | 5.78^{[b]} |
| 398 | 4-methylpyridin-2-yl | Me | 2-methylenepropane-1,3-diyl | Me | 6-methylpyridin-2-yl | 2.76^{[b]} |
| 399 | 3-CF₃-phenyl | Me | 2-methylenepropane-1,3-diyl | Me | 6-methylpyridin-2-yl | 5.36^{[b]} |
| 400 | 2-pyridyl | Me | 2-methylenepropane-1,3-diyl | Me | 6-methylpyridin-2-yl | 3.15^{[b]} (isomer B) |
| 401 | 3,4-methylenedioxy-phenyl | Me | 2-methylenepropane-1,3-diyl | Me | 6-methylpyridin-2-yl | 4.20^{[b]} |
| 402 | 2-Cl-phenyl | Me | 2-methylenepropane-1,3-diyl | Me | 6-methylpyridin-2-yl | 4.74^{[b]} |
| 403 | 3,4-OMe-phenyl | Me | 2-methylenepropane-1,3-diyl | Me | 6-methylpyridin-2-yl | 3.75^{[b]} |
| 404 | 6-methylpyridin-2-yl | Me | 2-methylenepropane-1,3-diyl | Me | benzofuran-2-yl | 4.57^{[b]} |
| 405 | 6-methylpyridin-3-yl | Me | 3-methylenepropane-1,3-diyl | Me | 6-methylpyridin-2-yl | 1.90^{[b]} (isomer B) |
| 406 | 1-naphthyl | Me | 2-methylenepropane-1,3-diyl | Me | 6-methylpyridin-2-yl | 5.02^{[b]} |
| 407 | 3-pyridyl | Me | 2-methylenepropane-1,3-diyl | Me | 6-methylpyridin-2-yl | 2.15^{[b]} |
| 408 | phenyl | Me | 2-methylenepropane-1,3-diyl | Me | 6-methylpyridin-2-yl | 4.51^{[b]} |
| 409 | 4-methypyridin-2-yl | Me | 2,2-dimethylpropane-1,3-diyl | Me | 6-methylpyridin-2-yl | 3.65^{[b]} |
| 410 | 3-CF₃-phenyl | Me | 2,2-dimethylpropane-1,3-diyl | Me | 6-methylpyridin-2-yl | 6.16^{[b]} |
| 411 | 6-methylpyridin-2-yl | Me | 2,2-dimethylpropane-1,3-diyl | Me | 4,5-dimethyl-1,3-thiazol-2-yl | 5.36^{[b]} |
| 412 | 3,4-methylenedioxy-phenyl | Me | 2,2-dimethylpropane-1,3-diyl | Me | 6-methylpyridin-2-yl | 5.08^{[b]} |
| 413 | 2-Cl-phenyl | Me | 2,2-dimethylpropane-1,3-diyl | Me | 6-methylpyridin-2-yl | 5.59^{[b]} |
| 414 | 3,4-OMe-phenyl | Me | 2,2-dimethylpropane-1,3-diyl | Me | 6-methylpyridin-2-yl | 4.57^{[b]} |
| 415 | 3,4-OMe-phenyl | Me | 2,2-dimethylpropane-1,3-diyl | Me | benzofuran-2-yl | 5.42^{[b]} |
| 416 | 6-methylpyridin-3-yl | Me | 2,2-dimethylpropane-1,3-diyl | Me | 6-methypyridin-2-yl | 2.41^{[b]} |
| 417 | 1-naphthyl | Me | 2,2-dimethylpropane-1,3-diyl | Me | 6-methylpyridin-2-yl | 5.90^{[b]} |
| 418 | 3-pyridyl | Me | 2,2-dimethylpropane-1,3-diyl | Me | 6-methylpyridin-2-yl | 2.80^{[b]} |
| 419 | phenyl | Me | 2,2-dimethylpropane-1,3-diyl | Me | 6-methylpyridin-2-yl | 5.42^{[b]} |
| 420 | 6-benzylpyridin-2-yl | Me | 2-methylpropane-1,3-diyl | Me | 6-methylpyridin-2-yl | 6.09^{[b]} |
| 421 | 6-methylpyridin-2-yl | Me | 2-methylpropane-1,3-diyl | Ph | 3-methylpyridin-2-yl | 4.15^{[b]} |
| 422 | 6-methylpyridin-2-yl | Me | 2-methylpropane-1,3-diyl | iBu | 3-methylpyridin-2-yl | 4.51^{[b]} |
| 423 | 6-isobutylpyridin-2-yl | Me | 2-methylpropane-1,3-diyl | Me | 6-methylpyridin-2-yl | 6.51^{[b]} |
| 424 | 6-methylpyridin-2-yl | Me | 2-methylpropane-1,3-diyl | but-3-en-1-yl | 3-methylpyridin-2-yl | 4.10^{[b]} |
| 425 | 6-methylpyridin-2-yl | Me | 2-methylpropane-1,3-diyl | Et | 3-methylpyridin-2-yl | 3.99^{[b]} |
| 426 | 6-methylpyridin-2-yl | Me | 2-methylpropane-1,3-diyl | Me | 3-methyl-1,2,4-triazin-5-yl | 3.24^{[b]} |
| 427 | 6-methylpyridin-2-yl | nPr | 2-methylpropane-1,3-diyl | Me | 6-methylpyridin-2-yl | 5.31^{[b]} |
| 428 | 6-methypyridin-2-yl | Et | 2-methylpropane-1,3-diyl | Me | 6-methylpyridin-2-yl | 4.51^{[b]} |
| 429 | 6-methylpyridin-2-yl | Me | 2-methylpropane-1,3-diyl | cPr | 6-methylpyridin-2-yl | 3.61^{[b]} |
| 430 | 2-methylpyrimidin-4-yl | Me | 2-methylpropane-1,3-diyl | Me | 6-methylpyridin-2-yl | 3.56^{[b]} |
| 431 | 5-methylpyridin-3-yl | Me | 2-methylpropane-1,3-diyl | Me | 6-methylpyridin-2-yl | 2.37^{[b]} |
| 432 | 2-methylpyridin-4-yl | Me | 2-methylpropane-1,3-diyl | Me | 6-methylpyridin-2-yl | 1.80^{[b]} |
| 433 | 3-CF₃-phenyl | Me | 2-methylpropane-1,3-diyl | Me | 6-methylpyridin-2-yl | 5.71^{[b]} |
| 434 | 6-methylpyridin-2-yl | Me | 2-methylpropane-1,3-diyl | Me | 4,5-dimethyl-1,3-thiazol-2-yl | 4.85^{[b]} |
| 435 | 3,4-methylenedioxy-phenyl | Me | 2-methylpropane-1,3-diyl | Me | 6-methypyridin-2-yl | 4.57^{[b]} |
| 436 | 2-Cl-phenyl | Me | 2-methylpropane-1,3-diyl | Me | 6-methylpyridin-2-yl | 5.08^{[b]} |
| 437 | 1-naphthyl | Me | 2-methylpropane-1,3-diyl | Me | 6-methylpyridin2--yl | 5.36^{[b]} |
| 438 | 3-pyridyl | Me | 2-methylpropane-1,3-diyl | Me | 6-methylpyridin-2-yl | 2.34^{[b]} |
| 439 | phenyl | Me | 2-methylpropane-1,3-diyl | Me | 6-methylpyridin-2-yl | 4.91^{[b]} |
| 440 | pyridin-4-yl | Me | —(CH₂)₄— | Me | 6-methylpyridin-2-yl | 1.88^{[b]} |
| 441 | 2-methyl-2,3-dihydro-1- | Me | —(CH₂)₄— | Me | 6-methylpyridin-2-yl | 4.91^{[b]} |
| | benzofuran-5-yl | | | | | |
| 442 | 3-Cl-phenyl | Me | —(CH₂)₄— | Me | 6-methylpyridin-2-yl | 5.54^{[b]} |
| 443 | 3-OMe-phenyl | Me | —(CH₂)₄— | Me | 6-methylpyridin-2-yl | 4.57^{[b]} |
| 444 | 4-Cl-phenyl | Me | —(CH₂)₄— | Me | 6-methylpyridin-2-yl | 5.54^{[b]} |
| 445 | 2-naphthyl | Me | —(CH₂)₄— | Me | 6-methylpyridin-2-yl | 5.72^{[a]} |
| 446 | 6-methylpyridin-2-yl | Me | —(CH₂)₄— | Me | 5-chloro-2-thienyl | 4.97^{[b]} |
| 447 | 6-methylpyridin-2-yl | Me | —(CH₂)₄— | Me | 4,6-dimethylpyridin-2-yl | 2.41^{[b]} |
| 448 | 2-methylpyridin-3-yl | Me | —(CH₂)₄— | Me | 6-methylpyridin-2-yl | 1.69^{[b]} |
| 449 | 6-methylpyridin-2-yl | Me | —(CH₂)₄— | Me | 4-methoxy-6-methylpyridin-2-yl | 3.11^{[b]} |
| 450 | 6-propylpyridin-2-yl | Me | —(CH₂)₄— | Me | 6-methylpyridin-2-yl | 5.48^{[b]} |
| 451 | 6-methylpyridin-2-yl | Me | —(CH₂)₄— | Me | 3-methylpyridin-2-yl | 3.02^{[b]} |
| 452 | 6-isopropylpyridin-2-yl | Me | —(CH₂)₄— | Me | 6-methylpyridin-2-yl | 5.97^{[b]} |
| 453 | 6-methylpyridin-2-yl | Me | —(CH₂)₄— | Me | 6-methylpyrazin-2-yl | 1.88^{[b]} |
| 454 | pyridin-4-yl | Me | hexane-1,6-diyl | Me | 6-methylpyridin-2-yl | 2.62^{[b]} |
| 455 | 2-methyl-2,3-dihydro-1- | Me | hexane-1,6-diyl | Me | 6-methylpyridin-2-yl | 5.97^{[b]} |
| | benzofuran-5-yl | | | | | |
| 456 | 3-Cl-phenyl | Me | hexane-1,6-diyl | Me | 6-methylpyridin-2-yl | 6.58^{[b]} |
| 457 | 3-OMe-phenyl | Me | hexane-1,6-diyl | Me | 6-methylpyridin-2-yl | 5.59^{[b]} |
| 458 | 4-Cl-phenyl | Me | hexane-1,6-diyl | Me | 6-methylpyridin-2-yl | 6.58^{[b]} |
| 459 | 6-methylpyridin-2-yl | Me | hexane-1,6-diyl | Me | 5-chloro-2-thienyl | 6.16^{[b]} |
| 460 | 6-methylpyridin-2-yl | Me | hexane-1,6-diyl | Me | 4,6-dimethylpyridin-2-yl | 3.51^{[b]} |
| 461 | 2-methylpyridin-3-yl | Me | hexane-1,6-diyl | Me | 6-methylpyridin-2-yl | 2.27^{[b]} |
| 462 | 6-methylpyridin-2-yl | Me | hexane-1,6-diyl | Me | 4-methoxy-6-methylpyridine-2-yl | 4.15^{[b]} |
| 463 | 6-propylpyridin-2-yl | Me | hexane-1,6-diyl | Me | 6-methylpyridin-2-yl | 6.79^{[b]} |
| 464 | 6-methylpyridin-2-yl | Me | hexane-1,6-diyl | Me | 3-methylpyridin-2-yl | 4.25^{[b]} |
| 465 | 6-isopropylpyridin-2-yl | Me | hexane-1,6-diyl | Me | 6-methylpyridin-2-yl | 7.12^{[b]} |
| 466 | 6-methylpyridin-2-yl | Me | hexane-1,6-diyl | Me | 6-methylpyrazin-2-yl | 4.8^{[b]} |
| 467 | pyridin-4-yl | Me | 2-methylenepropane-1,3-diyl | Me | 6-methylpyridin-2-yl | 1.88^{[b]} |
| 468 | 2-methyl-2,3-dihydro-1- | Me | 2-methylenepropane-1,3-diyl | Me | 6-methylpyridin-2-yl | 4.85^{[b]} |
| | benzofuran-5-yl | | | | | |
| 469 | 3-Cl-phenyl | Me | 2-methylenepropane-1,3-diyl | Me | 6-methylpyridin-2-yl | 5.31^{[b]} |
| 470 | 3-OMe-phenyl | Me | 2-methylenepropane-1,3-diyl | Me | 6-methylpyridin-2-yl | 4.46^{[b]} |
| 471 | 4-Cl-phenyl | Me | 2-methylene-propane-1,3-diyl | Me | 6-methylpyridin-2-yl | 5.48^{[b]} |
| 472 | 2-naphthyl | Me | 2-methylenepropane-1,3-diyl | Me | 6-methylpyridin-2-yl | 5.48^{[b]} |
| 473 | 6-methylpyridin-2-yl | Me | 2-methylenepropane-1,3-diyl | Me | 5-chloro-2-thienyl | 4.91^{[b]} (isomer B) |
| 474 | 6-methylpyridi-2-yl | Me | 2-methylenepropane-1,3-diyl | Me | 4,6-dimethylpyridin-2-yl | 2.55^{[b]} |
| 475 | 2-methylpyridin-2-yl | Me | 2-methylenepropane-1,3-diyl | Me | 6-methylpyridin-2-yl | 1.69^{[b]} |
| 476 | 6-methylpyridin-2-yl | Me | 2-methylenepropane-1,3-diyl | Me | 4-methoxy-6-methylpyridin-2-yl | 3.33^{[b]} (isomer B) |
| 477 | 6-propylpyridin-2-yl | Me | 2-methylenepropane-1,3-diyl | Me | 6-methylpyridin-2-yl | 5.42^{[b]} |
| 478 | 6-methylpyridin-2-yl | Me | 2-methylenepropane-1,3-diyl | Me | 3-methylpyridin-2-yl | 3.11^{[b]} (isomer B) |
| 479 | 6-isopropylpyridin-2-yl | Me | 2-methylenepropane-1,3-diyl | Me | 6-methylpyridin-2-yl | 5.78^{[b]} |
| 480 | 6-methylpyridin-2-yl | Me | 2-methylenepropane-1,3-diyl | Me | 6-methylpyrazin-2-yl | 3.46^{[b]} |
| 481 | pyridin-4-yl | Me | 2,2-dimethylpropane-1,3-diyl | Me | 6-methylpyridin-2-yl | 2.41^{[b]} |
| 482 | 2-methyl-2,3-dihydro-1- | Me | 2,2-dimethylpropane-1,3-diyl | Me | 6-methylpyridin-2-yl | 5.65^{[b]} |
| | benzofuran-5-yl | | | | | |
| 483 | 3-Cl-phenyl | Me | 2,2-dimethylpropane-1,3-diyl | Me | 6-methylpyridin-2-yl | 6.22^{[b]} |
| 484 | 3-OMe-phenyl | Me | 2,2-dimethylpropane-1,3-diyl | Me | 6-methylpyridin-2-yl | 5.31^{[b]} |
| 485 | 4-Cl-phenyl | Me | 2,2-dimethylpropane-1,3-diyl | Me | 6-methylpyridin-2-yl | 6.22^{[b]} |
| 486 | 6-methylpyridin-2-yl | Me | 2,2-dimethylpropane-1,3-diyl | Me | 5-chloro-2-thienyl | 5.84^{[b]} |
| 487 | 6-methylpyridin-2-yl | Me | 2,2-dimethylpropane-1,3-diyl | Me | 4,6-dimethylpyridin-2-yl | 3.33^{[b]} |
| 488 | 2-methylpyridin-3-yl | Me | 2,2-dimethylpropane-1,3-diyl | Me | 6-methylpyridin-2-yl | 2.20^{[b]} |
| 489 | 6-propylpyridin-2-yl | Me | 2,2-dimethylpropane-1,3-diyl | Me | 6-methylpyridin-2-yl | 6.36^{[b]} |
| 490 | 6-methylpyridin-2-yl | Me | 2,2-dimethylpropane-1,3-diyl | Me | 3-methylpyridin-2-yl | 3.89^{[b]} |
| 491 | 6-isopropylpyriidn-2-yl | Me | 2,2-dimethylpropane-1,3-diyl | Me | 6-methylpyridin-2-yl | 6.87^{[b]} |
| 492 | 6-methylpyridin-2-yl | Me | 2,2-dimethylpropane-1,3-diyl | Me | 6-methylpyrazin-2-yl | 4.41^{[b]} |
| 493 | pyridin-4-yl | Me | 2-methylpropane-1,3-diyl | Me | 6-methylpyridin-2-yl | 2.04^{[b]} |
| 494 | 2-methyl-2,3-dihydro-1- | Me | 2-methylpropane-1,3-diyl | Me | 6-methylpyridin-2-yl | 5.27^{[a]} |
| | benzofuran-5-yl | | | | | |
| 495 | 3-OMe-phenyl | Me | 2-methylpropane-1,3-diyl | Me | 6-methylpyridin-2-yl | 4.80^{[b]} |
| 496 | 4-Cl-phenyl | Me | 2-methylpropane-1,3-diyl | Me | 6-methylpyridin-2-yl | 5.78^{[b]} |
| 497 | 2-naphthyl | Me | 2-methylpropane-1,3-diyl | Me | 6-methylpyridin-2-yl | 5.84^{[b]} |
| 498 | 6-methylpyridin-2-yl | Me | 2-methylpropane-1,3-diyl | Me | 5-chloro-2-thienyl | 5.36^{[b]} |
| 499 | 6-methylpyridin-2-yl | Me | 2-methylpropane-1,3-diyl | Me | 4,6-dimethylpyridin-2-yl | 2.76^{[b]} |
| 500 | 2-methylpyridin-3-yl | Me | 2-methylpropane-1,3-diyl | Me | 6-methylpyridin2--yl | 1.85^{[b]} |
| 501 | 6-methylpyridin-2-yl | Me | 2-methylpropane-1,3-diyl | Me | 4-methoxy-6-methylpyridin-2-yl | 3.37^{[b]} |
| 502 | 6-propylpyridin-2-yl | Me | 2-methylpropane-1,3-diyl | Me | 6-methylpyridin-2-yl | 5.84^{[b]} |
| 503 | 6-methylpyridin-2-yl | Me | 2-methylpropane-1,3-diyl | Me | 3-methylpyridin-2-yl | 3.37^{[b]} |
| 504 | 6-isopropylpyridin-2-yl | Me | 2-methylpropane-1,3-diyl | Me | 6-methylpyridin-2-yl | 6.29^{b]} |
| 505 | 6-methylpyridin-2-yl | Me | 2-methylpropane-1,3-diyl | Me | 6-methylpyrazin-2-yl | 3.85^{[b]} |
| 506 | 6-methylpyridin-2-yl | Me | 2-methylpropane-1,3-diyl | Me | 4-methylpyrimidin-2-yl | 2.59^{[b]} |
| 507 | 6-methylpyridin2--yl | Me | 2-methylpropane-1,3-diyl | Me | pyrazin-2-yl | 3.28^{[b]} |
| 508 | 6-methylpyridin-2-yl | Me | —(CH₂)₄— | Me | 4-methylpyrimidin-2-yl | 2.37^{[b]} |
| 509 | 6-methylpyridin-2-yl | Me | bexane-1,6-diyl | Me | 4-methypyrimidin-2-yl | 3.39^{[b]} |
| 510 | 6-methylpyridin-2-yl | Me | 2-methylenepropane-1,3-diyl | Me | 4-methylpyrimidin-2-yl | 2.37^{[b]} |
| 511 | 6-methylpyridin-2-yl | Me | 2,2-dimethylpropane-1,3-diyl | Me | 4-methylpyrimidin-2-yl | 3.02^{[b]} |
| 512 | 6-methylpyridin-2-yl | Me | —(CH₂)₄— | Me | pyrazin-2-yl | 3.02^{[b]} |
| 513 | 6-methylpyridin-2-yl | Me | hexane-1,6-diyl | Me | pyraizn-2-yl | 4.25^{[b]} |
| 514 | 6-methylpyridin-2-yl | Me | 2-methylenepropane-1,3-diyl | Me | pyrazin-2-yl | 2.98^{[b]} |
| 515 | 6-methylpyridin-2-yl | Me | 2,2-dimethylpropane-1,3-diyl | Me | pyrazin-2-yl | 3.70^{[b]} |
| 516 | 4,5,6-trimethylpyrimdiin-2-yl | Et | 2-methylenepropane-1,3-diyl | Et | 4,5,6-trimethylpyrimidin-2-yl | 4.07^{[b]} |
| 517 | 2-methyl-1,3-thiazol-4-yl | Me | 2-methylpropane-1,3-diyl | Me | 2-methyl-1,3-thiazol-4-yl | 3.52^{[a]} |
| | | | | | | 3.52^{[b]} |
| 518 | 2-methyl-1,3-thiazol-4-yl | Me | 2,2-dimethylpropane-1,3-diyl | Me | 2-methyl-1,3-thiazol-4-yl | 3.99^{[a]} |
| | | | | | | 3.99^{[b]} |
| 519 | 5,6-dimethylpyridin-2-yl | Me | butane-1,3-diyl | Me | 5,6-dimethylpyridin-2-yl | 6.27^{[c]} |
| | | | | | | 2.75^{[b]} |
| 520 | 6-methylpyridin-2-yl | Me | —(CH₂)₄— | nPr | 3-methylpyridin-2-yl | (isomer B) |
| 521 | 6-(difluoromethyl)pyridin-2-yl | Me | —CH₂—CH₂—CH₂— | Me | 6-(difluoromethyl)pyridin-2-yl | 5.21^{[a]} |
| 522 | 2-methyl-1,3-thiazol-4-yl | Me | 2-methylenepropane-1,3-diyl | Me | 2-methyl-1,3-thiazol-4-yl | 3.22^{[a]} |
| | | | | | | 3.22^{[b]} |
| 523 | 6-methylpyridin-2-yl | Me | hexane-1,6-diyl | But-3-en-1-yl | 3-methylpyridin-2-yl | 4.91^{[b]} (isomer A) |
| 524 | 6-methylpyridin-2-yl | Me | hexane-1,6-diyl | But-3-en-1-yl | 3-methylpyridin-2-yl | 5.65^{[b]} (isomer B) |
| 525 | 6-methylpyridin-2-yl | Me | —(CH₂)₄— | iPr | 6-methylpyridin-2-yl | 3.65^{[b]} (isomer A) |
| 526 | 6-methylpyridin-2-yl | Me | hexane-1,6-diyl | iPr | 6-methylpyridin-2-yl | 4.91^{b]}(isomer A) |
| 527 | 6-methylpyridin-2-yl | Me | hexane-1,6-diyl | iPr | 6-methylpyridin-2-yl | 6.03^{[b]}(isomer B) |
| 528 | 6-methylpyridin-2-yl | Me | —(CH₂)₄— | Bzl | 3-methylpyridin-2-yl | 3.24^{[b]} |
| 529 | 6-methylpyridin-2-yl | Me | —(CH₂)₄— | Et | 3-methylpyridin-2-yl | 2.98^{[b]} (isomer )A |
| 530 | 6-methylpyridin-2-yl | Me | —(CH₂)₄— | iBu | 6-methylpyridin-2-yl | 5.48^{[b]} (isomer B) |
| 531 | 6-methylpyridin-2-yl | Me | hexane-1,6-diyl | Bzl | 3-methylpyridin-2-yl | 4.97^{b]} (isomer )A |
| 532 | 6-methylpyridin-2-yl | Me | hexane-1,6-diyl | Et | 3-methylpyridin-2-yl | 3.99^{b]} (isomer )A |
| 533 | 2-methylpyridin-4-yl | Me | 2-methylenepropane-1,3-diyl | Me | 6-methylpyridin-2-yl | 1.74^{[b]} (isomer A) |
| 534 | 6-methylpyridin-2-yl | Me | 2-methylenepropane-1,3-diyl | nPr | 3-methylpyridin-2-yl | 3.65^{[b]} (isomer A) |
| 535 | 6-methylpyridin-2-yl | Me | 2-methylenepropane-1,3-diyl | nPr | 3-methylpyridin-2-yl | 4.46^{[b]} (isomer B) |
| 536 | 6-methylpyridin-2-yl | Me | 2-methylenepropane-1,3-diyl | cPr | 6-methylpyridin-2-yl | 3.19^{[b]} (isomer A) |
| 537 | 6-methylpyridin-2-yl | Me | 2-methylenepropane-1,3-diyl | cPr | 6-methylpyridin-2-yl | 3.46^{[b]} (isomer B) |
| 538 | 6-methylpyridin-2-yl | Me | 2,2-dimethylpropane-1,3-diyl | iBu | 6-methylpyridin-2-yl | 6.47^{[b]} |
| 539 | 6-methylpyridin-2-yl | Me | hexane-1,6-diyl | Me | 4,5-dimethyl-1,3-thiazol-2-yl | 4.86^{[b]} v(isomer B) |
| 540 | 6-methylpyridin-2-yl | Me | —(CH₂)₄— | Me | 2,4-dimethyl-1,3-thiazol-5-yl | 3.56^{[b]} (isomer A) |
| 541 | pyridin-2-yl | Me | (2Z)-but-2-ene-1,4-diyl | Me | pyridin-2-yl | 3.04^{[b]} |
| 542 | 6-methylpyridin-2-yl | Me | hexane-1,6-diyl | Me | 2,4-dimethyl-1,3-thiazol-5-yl | 3.99^{[b]} (isomer A) |
| 543 | 6-methylpyridin-2-yl | Me | hexane-1,6-diyl | Me | 2,4-dimethyl-1,3-thiazol-5-yl | 4.74^{[b]} (isomer B) |
| 544 | 6-methylpyridin-2-yl | Me | 2-methylenepropane-1,3-diyl | Me | 2,4-dimethyl-1,3-thiazol-5-yl | 3.00^{b]} (Isomer A) |
| 545 | 6-methylpyridin-2-yl | Me | 2-methylenepropane-1,3-diyl | Me | 2,4-dimethyl-1,3-thiazol-5-yl | 3.58^{[b]} (isomer B) |
| 546 | 6-methylpyridin-2-yl | Me | 2,2-dimethylpropane-1,3-diyl | Me | 2,4-dimethyl-1,3-thiazol-5-yl | 4.37^{[b]} |
| 547 | 6-methylpyridin-2-yl | Me | 2,2-dimethylpropane-1,3-diyl | iPr | 6-methylpyridin-2-yl | 4.57^{[b]} |
| 548 | 6-methylpyridin-2-yl | Me | 2-methylpropane-1,3-diyl | nPr | 3-methylpyridin-2-yl | 3.85^{[b]} (isomer A) |
| 549 | 6-methylpyridin-2-yl | Me | 2-methylpropane-1,3-diyl | nPr | 3-methylpyridin-2-yl | 4.68^{[b]} (isomer B) |
| 550 | 5-methyl-6-propylpyridin-2-yl | Me | 2-methylpropane-1,3-diyl | Me | 5-methyl-6-propylpyridin-2-yl | 7.36^{[c]} |
| 551 | 6′-methyl-2,2′-bipyridin-6-yl | Me | 2-methylpropane-1,3-diyl | Me | 6′-methyl-2,2′-bipyridin-6-yl | 3.26^{[c]} |
| | | | | | | 6.24^{[b]} |
| 552 | 4,6-dimethylpyridin-2-yl | Me | 2-methoxypropane-1,3-diyl | Me | 4,6-dimethylpyridin-2-yl | 4.91^{[c]} |
| | | | | | | 1.56^{[b]} |
| 553 | 4,6-d9methylpyridin-2-yl | Me | cyclohexane-1,1-diylbis(methylene) | Me | 4,6-dimethylpyridin-2-yl | 7.36^{[c]} |
| | | | | | | 3.87^{[b]} |
| 554 | 4,6-dimethylpyridin-2-yl | Me | 2-phenylpropane-1,3-diyl | Me | 4,6-dimethylpyridin-2-yl | 6.33^{[c]} |
| | | | | | | 2.96^{[b]} |
| 555 | 4,6-dimethylpyridin-2-yl | Me | 2-n-propyl-propane-1,3-diyl | Me | 4,6-dimethylpyridin-2-yl | 7.15^{[c]} |
| | | | | | | 3.22^{[b]} |
| 556 | 4,6-dimethylpyridin-2-yl | Me | cyclopropane-1,1-diylbis(methylene) | Me | 4,6-dimethylpyridin-2-yl | 5.97^{[c]} |
| | | | | | | 2.11^{[b]} |
| 557 | 4,6-dimethylpyridin-2-yl | Me | pentane-2,4-diyl | Me | 4,6-dimethylpyridin-2-yl | 6.63^{[c]} |
| | | | | | | 2.39^{[b]} |
| 558 | 4,6-dimethylpyridin-2-yl | Me | butane-1,3-diyl | Me | 4,6-dimethylpyridin-2-yl | 6.11^{[c]} |
| | | | | | | 2.00^{[b]} |
| 559 | 4,6-dimethylpyridin-2-yl | Me | 2-tert.butyl-propane-1,3-diyl | Me | 4,6-dimethylpyridin-2-yl | 7.36^{[c]} |
| | | | | | | 3.60^{[b]} |
| 560 | 4,6-dimethylpyridin-2-yl | Me | 2-hydroxypropane-1,3-diyl | Me | 4,6-dimethylpyridin-2-yl | 3.68^{[c]} |
| | | | | | | 1.22[b] |
| 561 | 6-methylpyridin-2-yl | Me | cyclohexane-1,1-diylbis(methylene) | Me | 6-methylpyridin-2-yl | 7.15^{[c]} |
| | | | | | | 6.09^{[b]} |
| 562 | 4,6-dimethylpyridin-2-yl | Me | cyclobutane-1,1-diylbis(methylene) | Me | 4,6-dimethylpyridin-2-yl | 6.93^{[c]} |
| | | | | | | 2.90^{[b]} |
| 563 | 6-phenylpyridin-2-yl | Me | 2-methylpropane-1,3-diyl | Me | 6-phenylpyridin-2-yl | 7.36^{[c]} |
| | | | | | | 7.36^{[b]} |
| 564 | 1H-pyrrol-2-yl | Me | 2-methylenepropane-1,3-diyl | Me | 1H-pyrrol-2-yl | 3.20^{[c]} |
| | | | | | | 3.23^{[b]} |
| 565 | 4,6-dimethoxypyrimidin-2-yl | Me | 2-methylenepropane-1,3-diyl | Me | 4,6-dimethoxypyrimidin-2-yl | 4.10^{[c]} |
| | | | | | | 4.10^{[b]} |
| 566 | 1H-pyrrol-2-yl | Me | 2-methylpropane-1,3-diyl | Me | 1H-pyrrol-2-yl | 3.62^{[c]} |
| | | | | | | 3.47^{[b]} |
| | | | | | | (isomer mixture) |
| 567 | 6-phenylpyridin-2-yl | Me | 2-methylenepropane-1,3-diyl | Me | 6-phenylpyridin-2-yl | 7.27^{[c]}7.26^{[b]} |
| 568 | 6-chloropyridin-2-yl | Me | 2-methylpropane-1,3-diyl | Me | 6-chloropyridin-2-yl | 6.08^{[a]} |
| 569 | 6-chloropyridin-2-yl | Me | 2,2-dimethylpropane-1,3-diyl | Me | 6-chloropyridin-2-yl | 6.50^{[a]} |
| 570 | 4,6-dimethylpyridin-2-yl | Et | cyclopropane-1,1-diylbis(methylene) | Et | 4,6-dimethylpyridin-2-yl | 3.22^{[a]} |
| | | | | | | (isomer mixture) |
| 571 | 4,6-dimethylpyridin-2-yl | Et | 2,2-dimethylpropane-1,3-diyl | Et | 4,6-dimethylpyridin-2-yl | 3.92^{[a]} |
| | | | | | | (isomer mixture) |
| 572 | 4,6-dimethylpyridin-2-yl | Et | 2-methylpropane-1,3-diyl | Et | 4,6-dimethylpyridin-2-yl | 3.23^{[a]} |
| | | | | | | (isomer mixture) |
| 573 | 1H-benzimidazol-2-yl | Me | 2,2-dimethylpropane-1,3-diyl | Me | 1H-benzimidazol-2-yl | 2.10^{[a]} |
| 574 | 4,6-diethylpyridin-2-yl | Me | 2-methylpropane-1,3-diyl | Me | 4,6-diethylpyridin-2-yl | 5.95^{[a]} |
| 575 | 6-phenylpyridin-2-yl | Me | 2,2-dimethylpropane-1,3-diyl | Me | 6-phenylpyridin-2-yl | 7.36^{[a]} |
| 576 | 1-methyl-1H-imidazol-2-yl | Me | 2,2-dimethylpropane-1,3-diyl | Me | 1-methyl-1H-imidaozl-2-yl | 1.07^{[a]} |
| 577 | 6-[4-chloro-3-(trifluoromethyl)- | Me | 2,2-dimethylpropane-1,3-diyl | Me | 6-[4-chloro-3-(trifluoromethyl)- | 7.36^{[a]} |
| | phenyl]pyridin-2-yl | | | | phenyl]pyridin-2-yl | |
| 578 | 6-(3,5-dichlorophenyl)pyridin-2- | Me | 2,2-dimethylpropane-1,3-dyl | Me | 6-(3,5-dichlorophenyl)pyridin-2-yl | 7.36^{a]} |
| | yl | | | | | |
| 579 | 6-(4-methoxyphenyl)pyridin-2-yl | Me | 2,2-dimethylpropane-1,3-diyl | Me | 6-(4-methoxyphenyl)pyridin-2-yl | 7.36^{[a]} |
| 580 | 6-(4-tert-butylphenyl)pyridin-2-yl | Me | 2,2-dimethylpropane-1,3-diyl | Me | 6-(4-tert-butylphenyl)pyridin-2-yl | 7.36^{[a]} |
| 581 | 5,6-dimethylpyridin-2-yl | Me | 2-benzylpropane-1,3-diyl | Me | 5,6-dimethylpyridin-2-yl | 7.05^{[c]} |
| | | | | | | 5.01^{[b]} |
| 582 | 4,6-dimethylpyridin-2-yl | Me | 2-benzylpropane-1,3-diyl | Me | 4,6-dimethylpyridin-2-yl | 6.97^{[c]} |
| | | | | | | 3.62^{[b]} |
| 583 | 6-methylpyridin-2-yl | Me | 2-benzylpropane-1,3-diyl | Me | 6-methylpyridin-2-yl | 6.34^{[c]} |
| | | | | | | 5.37^{b]} |
| 584 | 1H-benzimidazol-2-yl | Me | 2-methylpropane-1,3-diyl | Me | 1H-benzimidazol-2-yl | 2.05^{[a]} |
| | | | | | | (isomer mixture) |
| 585 | 5-methyl-2-furyl | Me | 2-methylpropane-1,3-diyl | Me | 5-methyl-2-furyl | 4.70^{[a]} |
| | | | | | | (isomer mixture) |
| 586 | 6-methylpyridin-2-yl | Me | 2-ethoxypropane-1,3-diyl | Me | 6-methylpyridin-2-yl | 4.76^{[c]} |
| | | | | | | 2.87^{b]} |
| 587 | 6-methylpyridin-2-yl | Me | cyclopentane-1,1-diylbis(methylene) | Me | 6-methylpyridin-2-yl | 7.33^{[c]} |
| | | | | | | 5.49^{b]} |
| 588 | 5,6-diemthylpyridin-2-yl | Me | cyclopentane-1,1-diylbis(methylene) | Me | 5,6-dimethylpyridin-2-yl | 7.36^{[c]} |
| | | | | | | 4.92^{[b]} |
| 589 | 4,6-dimethylpyridin-2-yl | Me | cyclopentane-1,1-diylbis(methylene) | Me | 4,6-dimethylpyridin-2-yl | 7.36^{[c]} |
| | | | | | | 3.35^{[b]} |
| 590 | 3,6-dimethylpyridin-2-yl | Me | 2-benzylpropane-1,3-diyl | Me | 3,6-dimethylpyridin-2-yl | 6.01^{[c]} |
| | | | | | | 4.15^{[b]} |
| 591 | 6-methylpyridin-2-yl | Me | 2-benzylpropane-1,3-diyl | Me | 3,6-dimethylpyridin-2-yl | 5.07^{[c]} |
| | | | | | | 3.40^{[b]} |
| 592 | 6,7,8,9-tetrahydro-5H- | Me | 2,2-dimethylpropane-1,3-diyl | Me | 6,7,8,9-tetrahydro-5H- | 6.04^{[a]} |
| | cyclohepta[b]pyridin-2-yl | | | | cyclohepta[b]pyridin-2-yl | |
| 593 | 6-methylpyridin-2-yl | Me | (3-phenyl-4,5-dihydroisoxazole-5,5-diyl)bis(methylene) | Me | 6-methylpyridin-2-yl | 3.74^{[a]} |
| 594 | 6-methylpyridin-2-yl | Me | (3-ethyl-4,5-dihydroisoxazole-5,5-diyl)bis(methylene) | Me | 6-methylpyridin-2-yl | 2.68^{[a]} |
| 595 | 6-methylpyridin-2-yl | iPr | (3-ethyl-4,5-dihydroisoxazole-5,5-diyl)bis(methylene) | iPr | 6-methylpyridin-2-yl | 4.79^{[a]} |
| 596 | 6-methylpyridin-2-yl | Me | (3-methyl-4,5-dihydroisoxazole-5,5-diyl)bis(methylene) | Me | 6-methylpyridin-2-yl | 2.20^{[b]} |
| 597 | 6-methylpyridin-2-yl | Me | (3-i-propyl-4,5-dihydroisoxazole-5,5-diyl)bis(methylene) | Me | 6-methylpyridin-2-yl | 3.28^{[b]} |
| 598 | 6-methylpyridin-2-yl | Me | (3-n-propyl-4,5-dihydroisoxazole-5,5-diyl)bis(methylene) | Me | 6-methylpyridin-2-yl | 3.33^{[b]} |
| 599 | 6-methylpyridin-2-yl | Me | (3-i-butyl-4,5-dihydroisoxazole-5,5-diyl)bis(methylene) | Me | 6-methylpyridin-2-yl | 3.89^{[b]} |
| 600 | 6-methylpyridin-2-yl | Me | (3-t-butyl-4,5-dihydroisoxazole-5,5-diyl)bis(methylene) | Me | 6-methylpyridin-2-yl | 3.85^{[b]} |
| 601 | 6-methylpyridin-2-yl | Me | [3-(4-Cl-phenyl)-4,5-dihydroisoxazole-5,5-diyl]bis(methylene) | Me | 6-methylpyridin-2-yl | 4.67^{[b]} |
| 602 | 6-methylpyridin-2-yl | Me | [3-(2-Cl-phenyl)-4,5-dihydroisoxazole-5,5-diyl]bis(methylene) | Me | 6-methylpyridin-2-yl | 4.46^{[b]} |
| 603 | 6-methylpyridin-2-yl | Me | [3-(3-Cl-phenyl)-4,5-dihydroisoxazole-5,5-diyl]bis(methylene) | Me | 6-methylpyridin-2-yl | 4.67^{[b]} |
| 604 | 6-methylpyridin-2-yl | Me | [3-(2-methylprop-1-en-1-yl)-4,5-dihydroisoxazole-5,5-diyl]bis(methylene) | Me | 6-methylpyridin-2-yl | 3.70^{[b]} |
| 605 | 6-methylpyridin-2-yl | Et | (3-methyl-4,5-dihydroisoxazole-5,5-diyl)bis(methylene) | Et | 6-methylpyridin-2-yl | 3.42^{[b]} |
| 606 | 6-methylpyridin-2-yl | Et | (3-i-propyl-4,5-dihydroisoxazole-5,5-diyl)bis(methylene) | Et | 6-methylpyridin-2-yl | 4.67^{[b]} |
| 607 | 6-methylpyridin-2-yl | Et | (3-ethyl-4,5-dihydroisoxazole-5,5-diyl)bis(methylene) | Et | 5-methylpyridin-2-yl | 4.04^{[b]} |
| 608 | 6-methylpyridin-2-yl | Et | (3-n-propyl-4,5-dihydroisoxazole-5,5-diyl)bis(methylene) | Et | 6-methylpyridin-2-yl | 4.72^{[b]} |
| 609 | 6-methylpyridin-2-yl | Et | (3-i-butyl-4,5-dihydroisoxazole-5,5-diyl)bis(methylene) | Et | 6-methylpyridin-2-yl | 5.21^{[b]} |
| 610 | 6-methylpyridin-2-yl | Et | (3-t-butyl-4,5-dihydroisoxazole-5,5-diyl)bis(methylene) | Et | 6-methylpyridin-2-yl | 5.21^{[b]} |
| 611 | 6-methylpyridin-2-yl | Et | (3-phenyl-4,5-dihydroisoxazole-5,5-diyl)bis(methylene) | Et | 6-methylpyridin-2-yl | 5.08^{[b]} |
| 612 | 6-methylpyridin-2-yl | Et | [3-(4-Cl-phenyl)-4,5-dihydroisoxazole-5,5-diyl]bis(methylene) | Et | 6-methylpyridin-2-yl | 5.84^{[b]} |
| 613 | 6-methylpyridin-2-yl | Et | [3-(3-Cl-phenyl)-4,5-dihydroisoxazole-5,5-diyl]bis(methylene) | Et | 6-methylpyridin-2-yl | 5.84^{[b]} |
| 614 | 6-methylpyridin-2-yl | Et | [3-(4-methylprop-1-en-1-yl)-4,5-dihydroisoxazole-5,5-diyl]bis(methylene) | Et | 6-methylpyridin-2-yl | 4.98^{b]} |
| 615 | 6-methylpyridin-2-yl | nPr | (3-methyl-4,5-dihydroisoxazole-5,5-diyl)bis(methylene) | nPr | 6-methylpyridin-2-yl | 4.56^{[b]} |
| 616 | 6-methylpyridin-2-yl | nPr | (3-i-propyl-4,5-dihydroisoxazole-5,5-diyl)bis(methylene) | nPr | 6-methylpyridin-2-yl | 5.71^{[b]} |
| 617 | 6-methylpyridin-2-yl | nPr | (3-ethyl-4,5-dihydroisoxazole-5,5-diyl)bis(methylene) | nPr | 6-methypyridin-2-yl | 5.08^{[b]} |
| 618 | 6-methylpyridin-2-yl | nPr | (3-n-propyl-4,5-dihydroisoxazole-5,5-idyl)bis(methylene) | nPr | 6-methylpyridin-2-yl | 5.71^{[b]} |
| 619 | 6-methylpyridin-2-yl | nPr | (3-i-butyl-4,5-dihydroisoxazole-5,5-diyl)bis(methylene) | nPr | 6-methylpyridin-2-yl | 6.22^{[b]} |
| 620 | 6-methylpyridin-2-yl | nPr | (3-t-butyl-4,5-dihydroisoxazole-5,5-diyl)bis(methylene) | nPr | 6-methylpyridin-2-yl | 6.22^{[b]} |
| 621 | 6-methylpyridin-2-yl | nPr | (3-phenyl-4,5-dihydroisoxazole-5,5-diyl)bis(methylene) | nPr | 6-methylpyridin-2-yl | 5.9^{[b]} |
| 622 | 6-methylpyridin-2-yl | nPr | [3-(4-Cl-phenyl)-4,5-dihydroisoxazole-5,5-diyl]bis(methylene) | nPr | 6-methylpyridin-2-yl | 6.58^{[b]} |
| 623 | 6-methylpyridin-2-yl | nPr | [3-(2-Cl-phenyl)-4,5-dihydroisoxazole-5,5-diyl]bis(methylene) | nPr | 6-methylpyridin-2-yl | 6.51^{[b]} |
| 624 | 6-methylpyridin-2-yl | nPr | [3-(3-Cl-phenyl)-4,5-dihydroisoxazole-5,5-diyl]bis(methylene) | nPr | 6-methylpyridin-2-yl | 6.58^{[b]} |
| 625 | 6-methylpyridin-2-yl | nPr | [3-(2-methylprop-1-en-1-yl)-4,5-dihydroisoxazole-5,5-diyl]bis(methylene) | nPr | 6-methylpyridin-2-yl | 5.97^{[b]} |
| 626 | 5,6-dimethylpyridin-2-yl | Me | (3-methyl-4,5-dihydroisoxazole-5,5-diyl)bis(methylene) | Me | 5,6-dimethylpyridin-2-yl | 2.07^{[b]} |
| 627 | 5,6-dimethylpyridin-2-yl | Me | (3-i-propyl-4,5-dihydroisoxazole-5,5-diyl)bis(methylene) | Me | 5,6-dimethylpyridin-2-yl | 3.06^{[b]} |
| 628 | 5,6-dimethylpyridin-2-yl | Me | (3-ethyl-4,5-dihydroisoxazole-5,5-diyl)bis(methylene) | Me | 5,6-dimethylpyridin-2-yl | 2.51^{[b]} |
| 629 | 5,6-dimethylpyridin-2-yl | Me | (3-n-propyl-4,5-dihydroisoxazole-5,5-diyl)bis(methylene) | Me | 5,6-dimethylpyridin-2-yl | 3.11^{[b]} |
| 630 | 5,6-dimethylpyridin-2-yl | Me | (3-i-butyl-4,5-dihydroisoxazole-5,5-diyl)bis(methylene) | Me | 5,6-dimethylpyridin-2-yl | 3.70^{[b]} |
| 631 | 5,6-dimethylpyridin-2-yl | Me | (3-t-butyl-4,5-dihydroisoxazole-5,5-diyl)bis(methylene) | Me | 5,6-dimethylyridin-2-yl | 3.65^{[b]} |
| 632 | 5,6-dimethylpyridin-2-yl | Me | (3-phenyl-4,5-dihydroisoxazole-5,5-diyl)bis(methylene) | Me | 5,6-dimethylpyridin-2-yl | 3.61^{[b]} |
| 633 | 5,6-dimethylpyridin-2-yl | Me | [3-(4-Cl-phenyl)-4,5-dihydroisoxazole-5,5-diyl]bis(methylene) | Me | 5,6-dimethylpyridin-2-yl | 4.51^{[b]} |
| 634 | 5,6-dimethylpyridin-2-yl | Me | [3-(2-Cl-phenyl)-4,5-dihydroisoxazole-5,5-diyl]bis(methylene) | Me | 5,6-dimethylpyridin-2-yl | 4.3^{[b]} |
| 635 | 5,6-dimethylpyridin-2-yl | Me | [3-(3-Cl-phenyl)-4,5-dihydroisoxazole-5,5-diyl]bis(methylene) | Me | 5,6-dimethylpyridin-2-yl | 4.51^{[b]} |
| 636 | 5,6-dimethylpyridin-2-yl | Me | [3-(2-methylprop-1-en-1-yl)-4,5-dihydroisoxazole-5,5-diyl]bis(methylene) | Me | 5,6-dimethylpyridin-2-yl | 3.46^{[b]} |
| 637 | 4,6-dimethylpyridin-2-yl | Me | (3-methyl-4,5-dihydroisoxazole-5,5-diyl)bis(methylene) | Me | 4,6-dimethylpyridin-2-yl | 1.60^{[b]} |
| 638 | 4,6-dimethylpyridin-2-yl | Me | (3-i-propyl-4,5-dihydroisoxazole-5,5-diyl)bis(methylene) | Me | 4,6-dimethylpyridin-2-yl | 2.18^{[b]} |
| 639 | 4,6-dimethylpyridin-2-yl | Me | (3-ethyl-4,5-dihydroisoxazole-5,5-diyl)bis(methylene) | Me | 4,6-dimethylpyridin-2-yl | 1.88^{[b]} |
| 640 | 4,6-dimethylpyridin-2-yl | Me | (3-n-propyl-4,5-dihydroisoxazole-5,5-diyl)bis(methylene) | Me | 4,6-dimethylpyridin-2-yl | 2.20^{[b]} |
| 641 | 4,6-dimethylpyridin-2-yl | Me | (3-i-butyl-4,5-dihydroisoxazole-5,5-diyl)bis(methylene) | Me | 4,6-dimethylpyridin-2-yl | 2.66^{[b]} |
| 642 | 4,6-dimethylpyridin-2-yl | Me | (3-t-butyl-4,5-dihydroisoxazole-5,5-diyl)bis(methylene) | Me | 4,6-dimethylpyridin-2-yl | 2.62^{[b]} |
| 643 | 4,6-dimethylpyridin-2-yl | Me | (3-phenyl-4,5-dihydroisoxazole-5,5-diyl)bis(methylene) | Me | 4,6-dimethylpyridin-2-yl | 2.55^{[b]} |
| 644 | 4,6-dimethylpyridin-2-yl | Me | [3-(4-Cl-phenyl)-4,5-dihydroisoxazole-5,5-diyl]bis(methylene) | Me | 4,6-dimethylpyridin-2-yl | 3.28^{[b]} |
| 645 | 4,6-dimethylpyridin-2-yl | Me | [3-(2-Cl-phenyl)-4,5-dihydroisoxazole-5,5-diyl]bis(methylene) | Me | 4,6-dimethylpyridin-2-yl | 3.19^{[b]} |
| 646 | 4,6-dimethylpyridin-2-yl | Me | [3-(3-Cl-phenyl)-4,5-dihydroisoxazole-5,5-diyl]bis(methylene) | Me | 4,6-dimethylpyridin-2-yl | 3.28^{[b]} |
| 647 | 4,6-dimethylpyridin-2-yl | Me | [3-(2-methylprop-1-en-1-yl)-4,5-dihydroisoxazole-5,5-diyl]bis(methylene) | Me | 4,6-dimethylpyridin-2-yl | 2.48^{[b]} |
| 648 | 6-methylpyridin-2-yl | cPr | [3-(2-Cl-phenyl)-4,5-dihydroisoxazole-5,5-diyl]bis(methylene) | cPr | 6-methylpyridin-2-yl | 4.46^{[a]} |
| 649 | 6-methylpyridin-2-yl | cPr | [3-(4-Cl-phenyl)-4,5-dihydroisoxazole-5,5-diyl]bis(methylene) | cPr | 6-methylpyridin-2-yl | 4.53^{[a]} |
| 650 | 6-methylpyridin-2-yl | cPr | (3-phenyl-4,5-dihydroisoxazole-5,5-diyl)bis(methylene) | cPr | 6-methylpyridin-2-yl | 3.80^{[a]} |
| 651 | 6-methylpyridin-2-yl | cPr | (3-t-butyl-4,5-dihydroisoxazole-5,5-diyl)bis(methylene) | cPr | 6-methylpyridin-2-yl | 3.98^{[a]} |
| | | | | | | (isomer mixture) |
| 652 | 6-methylpyridin-2-yl | cPr | (3-ethyl-4,5-dihydroisoxazole-5,5-diyl)bis(methylene) | cPr | 6-methylpyridin-2-yl | 2.95^{[a]} |
| 653 | 6-methylpyridin-2-yl | cPr | (3-i-butyl-4,5-dihydroisoxazole-5,5-diyl)bis(methylene) | cPr | 6-methylpyridin-2-yl | 3.99^{[a]} |
| 654 | 6-methylpyridin-2-yl | Et | [3-(2-Cl-phenyl)-4,5-dihydroisoxazole-5,5-diyl]bis(methylene) | Et | 6-methylpyridin-2-yl | 5.69^{[a]} |
| 655 | 6-methylpyridin-2-yl | cPr | [3-(3-Cl-phenyl)-4,5-dihydroisoxazole-5,5-diyl]bis(methylene) | cPr | 6-methylpyridin-2-yl | 4.54^{[a]} |
| 656 | 6-methylpyridin-2-yl | Ph | —CH₂—CH₂—CH₂— | Ph | 6-methylpyridin-2-yl | 3.68^{[a]} |
| | | | | | | (isomer mixture) |
| 657 | 3-methylpyridin-2-yl | Ph | —CH₂—CH₂—CH₂— | Ph | 3-methylpyridin-2-yl | 4.17^{[a]} |
| 658 | 3-methylpyridin-2-yl | Ph | 2-methylpropane-1,3-diyl | Ph | 3-methylpyridin-2-yl | 4.54^{[a]} |
| 659 | 6-methylpyridin-2-yl | Ph | 2-methylpropane-1,3-diyl | Ph | 6-methylpyridin-2-yl | 4.09^{[a]} |
| | | | | | | (isomer mixture) |
| 660 | 6-methylpyridin-2-yl | Ph | 2,2-dimethylpropane-1,3-diyl | Ph | 6-methylpyridin-2-yl | 4.09^{[a]} |
| | | | | | | (isomer mixture) |
| 661 | 6-methylpyridin-2-yl | Ph | 2-methylpropane-1,3-diyl | Ph | 6-methylpyridin-2-yl | 4.10^{[a]} (isomer A) |
| 662 | 6-methylpyridin-2-yl | H | 2-methylenepropane-1,3-diyl | H | 6-methylpyridin-2-yl | 2.16^{[b]} (isomer B) |
| 663 | 6-methylpyridin-2-yl | Me | 2-methylenepropane-1,3-diyl | H | 6-methylpyridin-2-yl | 2.54^{[b]} |
| 664 | 6-methylpyridin-2-yl | H | 2-methylenepropane-1,3-diyl | H | 6-methylpyridin-2-yl | 2.13^{[b]} (isomer A) |
| 665 | pyridin-2-yl | H | ethane-1,2-diyl | H | pyridin-2-yl | 1.68^{[b]} |
| 666 | pyridin-2-yl | H | propane-1,3-diyl | H | pyridin-2-yl | 1.97^{[b]} |
| 667 | 6-methylpyridin-2-yl | H | 2-methylenepropane-1,3-diyl | H | 6-methylpyridin-2-yl | 2.16^{[b]} |
| 668 | 6-methylpyridin-2-yl | H | 2-methylpropane-1,3-diyl | H | 6-methylpyridin-2-yl | 2.28^{[b]} |
| 669 | 6-methylpyridin-2-yl | H | propane-1,3-diyl | H | 6-methylpyridin-2-yl | 2.54^{[b]} |
| 670 | 6-methylpyridin-2-yl | H | 2-methylenepropane-1,3-diyl | Me | 6-methylpyridin-2-yl | 2.54^{[b]} |
| 671 | 6-methyl-4-(trifluoromethyl)- | H | 2-methylenepropane-1,3-diyl | Me | 6-methylpyridin-2-yl | 4.61^{[b]} |
| | pyridin-2-yl | | | | | (isomer mixture) |
| 672 | 6-methylpyridin-2-yl | Me | 2-methylenepropane-1,3-diyl | iPr | 6-methylpyridin-2-yl | 3.87^{[b]} (isomer A) |
| 673 | 6-methylpyridin-2-yl | 2-thienyl | 2-methylpropane-1,3-diyl | 2-thienyl | 6-methylpyridin-2-yl | 4.49^{[b]} |
| 674 | 5,6-dimethylpyridin-2-yl | Me | (methylmimino)dicarbonyl | Me | 5,6-dimethylpyridin-2-yl | 2.79^{[b]} |
| 675 | 5,6-dimethylpyridin-2-yl | Me | 1,3-dioxo-2,2-diethylproane-1,3-diyl | Me | 5,6-dimethylpyridin-2-yl | 5.06^{[b]} |
| 676 | 4,6-dimethylpyridin-2-yl | Me | (methylimino)dicarbonyl | Me | 4,6-dimethylpyridin-2-yl | 2.21^{[b]} |
| 677 | 6-methylpyridin-2-yl | Me | 2-ethylpropane-1,3-diyl | Me | 6-methylpyridin-2-yl | 5.97^{[c]} |
| | | | | | | 4.38^{[b]} |
| 678 | 5,6-dimethylpyridin-2-yl | Me | 2-ethylpropane-1,3-diyl | Me | 5,6-dimethylpyridin-2-yl | 6.79^{[c]} |
| | | | | | | 3.89^{[b]} |
| 679 | 4,6-dimethylpyridin-2-yl | Me | 2-ethylpropane-1,3-diyl | Me | 4,6-dimethylpyridin-2-yl | 2.61^{[b]} |
| 680 | 6-methylpyridin-2-yl | Me | 2-isopropylpropane-1,3-diyl | Me | 6-methylpyridin-2-yl | 6.40^{[c]} |
| | | | | | | 5.03^{[b]} |
| 681 | 5,6-dimethylpyridin-2-yl | Me | 2-isopropylpropane-1,3-diyl | Me | 5,6-dimethylpyridin-2-yl | 7.18^{[c]} |
| | | | | | | 4.57^{[b]} |
| 682 | 4,6-dimethylpyridin-2-yl | Me | 2-isopropylpropane-1,3-diyl | Me | 4,6-dimethylpyridin-2-yl | 7.09^{[c]} |
| | | | | | | 3.12^{[b]} |
| 683 | 4-ethoxy-6-methylpyridin-2-yl | Me | 2-methylpropane-1,3-diyl | Me | 4-ethoxy-6-methylpyridin-2-yl | 1.71^{[a]} |
| 684 | 5,6-dimethylpyridin-2-yl | Me | (2E)-but-2-en-1,4-diyl | Me | 5,6-dimethylpyridin-2-yl | 2.67^{[a]} |
| 685 | 6-vinylpyridin-2-yl | Me | 2-methylpropane-1,3-diyl | Me | 6-vinylpyridin-2-yl | 6.6^{[b]} |
| 686 | 6-prop-1-yn-1-ylpyridin-2-yl | Me | 2-methylpropane-1,3-diyl | Me | 6-prop-1-yn-1-ylpyridin-2-yl | 5.65^{[b]} |
| 687 | 8-methylquinolin-2-yl | Me | 2-methylpropane-1,3-diyl | Me | 8-methylquinolin-2-yl | 7.36^{[c]} |
| | | | | | | 7.36^{[b]} |
| 688 | 6-methylpyridinium-2-yl chloride | Me | 2-methylpropane-1,3-diyl | Me | 6-methylpyridinium-2-ylchloride | |
| 689 | 6-methylpyridin-2-yl | NH₂ | 2-methylpropane-1,3-diyl | NH₂ | 6-methylpyridin-2-yl | 2.71^{[b]} |
| 690 | 6-benzoylpyridin-2-yl | Me | 2-methylpropane-1,3-diyl | Me | 6-methylpyridin-2-yl | 5.75^{[b]} |
| 691 | 4,6-dimethylpyridin-2-yl | Et | 2-methylpropane-1,3-diyl | Et | 4,6-dimethylpyridin-2-yl | 2.75^{[a]} (isomer A) |
| 692 | 6-ethyl-4-pehenylpyridin-2-yl | Me | 2-methylpropane-1,3-diyl | Me | 6-ethyl-4-phenylpyridin-2-yl) | 7.36^{[a]} |
| 693 | 6-methylpyridin-2-yl | Me | 2-methylpropane-1,3-diyl | Me | 6-[(methoxyimino)(phenyl)methyl]- | 6.22^{[b]} |
| | | | | | pyridin-2-yl | |
| 694 | 5,6-dimethylpyridin-2-yl | Me | 2-methylpropane-1,3-diyl | Me | 4,6-dimethylpyridin-2-yl | 6.17^{[c]} |
| | | | | | | 2.63^{[b]} |
| 695 | 5,6-dimethylpyridin-2-yl | Me | cyclopropane-1,1-diylbis(methylene) | Me | 4,6-dimethylpyridin-2-yl | 6.02^{[c]} |
| | | | | | | 2.61^{[b]} |
| 696 | 6-methylpyridin-2-yl | Cl | 2-methylenepropane-1,3-diyl | Cl | 6-methylpyridin-2-yl | 4.01^{[b]} |
| 697 | 5,6-dimethylpyridin-2-yl | Me | 2,2-dimethylpropane-1,3-diyl | Me | 4,6-dimethylpyridin-2-yl | 6.70^{[c]} |
| | | | | | | 3.15^{[b]} |
| 698 | 5-methyl-2-furyl | Me | 2,2-dimethylpropane-1,3-diyl | Me | 5-methyl-2-furyl | 5.00^{[b]} |
| 699 | 5,6-dimethylpyridin-2-yl | Me | propane-1,3-diyl | Me | 6-methylpyridin-2-yl | 5.33^{[c]} |
| | | | | | | 2.53^{[b]} |
| 700 | 4,6-dimethylpyrimidin-2-yl | NH₂ | 1,2-phenylenebis(methylene) | NH₂ | 4,6-dimethylpyrimidin-2-yl | 2.23^{[b]} |
| 701 | 4,6-dimethylpyrimidin-2-yl | NH₂ | 1,3-phenylenebis(methylene) | NH₂ | 4,6-dimethylpyrimidin-2-yl | 2.13^{[b]} |
| 702 | 4,6-dimethylpyrimidin-2-yl | NH₂ | 1,4-phenylenebis(methylene) | NH₂ | 4,6-dimethylpyrimidin-2-yl | 2.11^{[b]} |
| 703 | 6-methylpyridin-2-yl | Me | 1,2-phenylenebis(methylene) | Me | 6-methylpyridin-2-yl | 4.26^{[b]} |
| 704 | 6-methylpyridin-2-yl | Me | 1,3-phenylenebis(methylene) | Me | 6-methylpyridin-2-yl | 4.17^{[b]} |
| 705 | 6-methylpyridin-2-yl | Me | 1,4-phenylenebis(methylene) | Me | 6-methylpyridin-2-yl | 4.27^{[b]} |
| 706 | 6-methylpyridin-2-yl | Me | but-2-yn-1,4-diyl | Me | 6-methylpyridin-2-yl | 3.12^{[b]} |
| 707 | 4-methyl-5,6,7,8-tetra- | NH₂ | 1,2-phenylenebis(methylene) | NH₂ | 4-methyl-5,6,7,8-tetra- | 3.25^{[b]} |
| | hydroquinazolin-2-yl | | | | hydroquinazolin-2-yl | |
| 708 | 4,6-bis(methoxymethyl)-5- | NH₂ | 1,2-phenylenebis(methylene) | NH₂ | 4,6-bis(methoxymethyl)-5- | 2.16^{[b]} |
| | methylpyrimidin-2-yl | | | | methylpyrimidin-2-yl | |
| 709 | 5-chloro-4,6-dimethylpyrimidin- | NH₂ | 1,2-phenylenebis(methylene) | NH₂ | 5-chloro-4,6-dimethylpyrimidin-2-yl | 3.41^{[b]} |
| | 2-yl | | | | | |
| 710 | 3,5-dimethylphenyl | NH₂ | 1,2-phenylenebis(methylene) | NH₂ | 3,5-dimethylphenyl | 3.68^{[b]} |
| 711 | 4,6-dicyclopropylpyrimidin-2-yl | NH₂ | 1,2-phenylenebis(methylene) | NH₂ | 4,6-dicylcopropylpyrimidin-2-yl | 3.85^{[b]} |
| 712 | 4-methyl-5,6,7,8-tetra- | NH₂ | 1,3-phenylenebis(methylene) | NH₂ | 4-methyl-5,6,7,8-tetra- | 3.09^{[b]} |
| | hydroquinazolin-2-yl | | | | hydroquinazolin-2-yl | |
| 713 | 4,6-bis(methoxymethyl)-5- | NH₂ | 1,3-phenylenebis(methylene) | NH₂ | 4,6-bis(methoxymethyl)-5- | 2.06^{[b]} |
| | methylpyrimidin-2-yl | | | | methylpyrimidin-2-yl | |
| 714 | 3,5-difluoropyridin-2-yl | NH₂ | 1,3-phenylenebis(methylene) | NH₂ | 3,5-difluoropyridin-2-yl | 2.96^{[b]} |
| 715 | 5-chloro-4,6-dimethylpyrimidin- | NH₂ | 1,3-phenylenebis(methylene) | NH₂ | 5-chloro-4,6-dimethylpyrimidin-2-yl | 3.29^{[b]} |
| | 2-yl | | | | | |
| 716 | 4,6-dicyclopropylpyrimidin-2-yl | NH₂ | 1,3-phenylenebis(methylene) | NH₂ | 4,6-dicyclopropylpyrimidin-2-yl | 3.76^{[b]} |
| 717 | 4-methyl-5,6,7,8-tetra- | NH₂ | 1,4-phenylenebis(methylene) | NH₂ | 4-methyl-5,6,7,8-tetra- | 3.09^{[b]} |
| | hydroquinazolin-2-yl | | | | hydroquinazolin-2-yl | |
| 718 | 4,6-bis(methoxymethyl)-5- | NH₂ | 1,4-phenylenebis(methylene) | NH₂ | 4,6-bis(methoxymethyl)-5- | |
| | methylpyrimidin-2-yl | | | | methylpyrimidin-2-yl | |
| 719 | 3,5-difluoropyridin-2-yl | NH₂ | 1,4-phenylenebis(methylene) | NH₂ | 3,5-difluoropyridin-2-yl | 2.96^{[b]} |
| 720 | 4,6-dicyclopropylpyrimidin-2-yl | NH₂ | 1,4-phenylenebis(methylene) | NH₂ | 4,6-dicyclopropylpyrimidin-2-yl | 3.76^{[b]} |
| 721 | 4-methyl-5,6,7,8-tetra- | NH₂ | (2E)-but-2-ene-1,4-diyl | NH₂ | 4-methyl-5,6,7,8-tetra- | 2.51^{[b]} |
| | hydroquinazolin-2-yl | | | | hydroquinazolin-2-yl | |
| 722 | 4,6-dicyclopropylpyrimidin-2-yl | NH₂ | (2E)-but-2-ene-1,4-diyl | NH₂ | 4,6-dicylcopropylpyrimidin-2-yl | 3.13^{[b]} |
| 723 | 3,5-difluoropyridin-2-yl | NH₂ | (2E)-but-2-ene-1,4-diyl | NH₂ | 3,5-difluoropyridin-2-yl | 2.42^{[b]} |
| 724 | 4,6-dimethylpyridin-2-yl | NH₂ | 1,2-phenylenebis(methylene) | NH₂ | 4,6-dimethylpyridin-2-yl | 3.72^{[b]} |
| 725 | pyridin-2-yl | Me | 1,2-phenylenebis(methylene) | Me | pyridin-2-yl | 3.76^{[b]} |
| 726 | 4,6-dimethylpyridin-2-yl | NH₂ | 1,3-phenylenebis(methylene) | NH₂ | 4,6-dimethylpyridin-2-yl | 3.63^{[b]} |
| 727 | 6-methylpyridin-2-yl | Me | 5-fluoropyrimidine-4,6-diyl | Me | 6-methylpyridin-2-yl | 3.13^{[b]} |
| 728 | 6-methylpyridin-2-yl | Me | 5-methylpyrimidine-4,6-diyl | Me | 6-methylpyridin-2-yl | 3.45^{[c]} |
| | | | | | | 2.34^{[b]} |
| 729 | 6-methylpyridin-2-yl | Me | 5-chloropyrimidine-4,6-diyl | Me | 6-methylpyridin-2-yl | 3.31^{[b]} |
| 730 | 4,5-dimethyl-1,3-thiazol-2-yl | Me | 1,2-phenylenebis(methylene) | Me | 4,5-dimethyl-1,3-thiazol-2-yl | 5.71^{[b]} |
| 731 | pyridin-2-yl | Me | 1,3-phenylenebis(methylene) | Me | pyridin-2-yl | 3.72^{[b]} |
| 732 | 4,5-dimethyl-1,3-thiazol-2-yl | Me | 1,3-phenylenebis(methylene) | Me | 4,5-dimethyl-1,3-thiazol-2-yl | 5.71^{[b]} |
| 733 | 4,6-dimethylpyridin-2-yl | NH₂ | 1,4-phenylenebis(methylene) | NH₂ | 4,6-dimethylpyridin-2-yl | 3,59^{[b]} |
| 734 | pyridin-2-yl | Me | 1,4-phenylenebis(methylene) | Me | pyridin-2-yl | 3.76^{[b]} |
| 735 | 6-methylpyridin-2-yl | H | 1,4-phenylenebis(methylene) | H | 6-methylpyridin-2-yl | 2.80^{[b]} |
| 736 | 4,5-dimethyl-1,3-thiazol-2-yl | Me | 1,4-phenylenebis(methylene) | Me | 4,5-dimethyl-1,3-thiazol-2-yl | 5.84^{[b]} |
| 737 | 6-methylpyridin-2-yl | H | (2E)-but-2-ene-1,4-diyl | H | 6-methylpyridin-2-yl | 2.18^{[b]} |
| 738 | 6-methylpyridin-2-yl | Me | pyridin-2-yl-3-carbonyl | Me | 6-methylpyridin-2-yl | 2.44^{[b]} |
| 739 | 6-methylpyridin-2-yl | Me | 5-methylpyrimidine-4,6-diyl | Et | 3-methylpyridin-2-yl | 2.18^{[b]} |
| 740 | 6-methylpyridin-2-yl | Me | pyridine-2,6-diylbis(methylene) | Me | 6-methylpyridin-2-yl | 4.33^{[c]} |
| | | | | | | 2.49^{[b]} |
| 741 | 5,6-dimethylpyridin-2-yl | Me | pyridine-2,6-diylbis(methylene) | Me | 5,6-dimethylpyridin-2-yl | 5.08^{[c]} |
| | | | | | | 2.30^{[b]} |
| 742 | 4,6-dimethylpyridin-2-yl | Me | pyridine-2,6-diylbis(methylene) | Me | 4,6-dimethylpyridin-2-yl | 4.96^{[c]} |
| | | | | | | 1.l77^{[b]} |
| 743 | 6-methylpyridin-2-yl | Me | 3-methylpentane-2,4-diyl | Me | 6-methylpyridin-2-yl | 4.90^{[b]} |
| 744 | 6-methylpyridin-2-yl | Me | (methylimino)dicarbonyl | Me | 6-methylpyridin-2-yl | 2.57^{[b]} |
| 745 | 6-methylpyridin-2-yl | Me | (diethylmethylene)dicarbonyl | Me | 6-methylpyridin-2-yl | 4.49^{[b]} |
| 746 | 6-methylpyridin-2-yl | Br | 2-methylenepropane-1,3-diyl | Br | 6-methylpyridin-2-yl | 4.41^{[b]} |
| 747 | pyridin-2-yL | Me | 3-methylpentane-1,5-diyl | Me | pyridin-2-yl | 4.43^{[b]} |
| 748 | 7-methylquinolin-2-yl | Me | 2-methylpropane-1,3-diyl | Me | 7-methylqjinolin-2-yl | 7.36^{[c]} |
| | | | | | | 7.36^{[b]} |
| 749 | 7-methylquinolin-2-yl | Me | 2-methylenepropane-1,3-diyl | Me | 7-methylquinolin-2-yl | 7.36^{[c]} |
| | | | | | | 7.36^{[b]} |
| 750 | 3,6-dimethylpyridin-2-yl | Me | 2-ethylpropane-1,3-diyl | Me | 3,6-dimethylpyridin-2-yl | 4.46^{[c]} |
| | | | | | | 2.57^{[b]} |
| | | | | | | (isomer B) |
| 751 | 3,6-dimethylpyridin-2-yl | Me | 2-ethylpropane-1,3-diyl | Me | 3,6-dimethylpyridin-2-yl | 5.54^{[c]} |
| | | | | | | 3.19^{[b]} |
| | | | | | | (isomer )A |
| 752 | 3,6-dimethylpyridin-2-yl | Me | 2-isopropylpropane-1,3-diyl | Me | 3,6-dimethylpyridin-2-yl | 4.85^{[c]} |
| | | | | | | 2.89^{[b]} |
| | | | | | | (isomer B) |
| 753 | 3,6-dimethylpyridin-2-yl | Me | 2-isopropylpropane-1,3-diyl | Me | 3,6-dimethylpyridin-2-yl | 5.95^{[c]} |
| | | | | | | 4.51^{[b]} |
| | | | | | | (isomer A) |
| 754 | 3,6-dimethylpyridin-2-yl | Me | 2-phenylpropane-1,3-diyl | Me | 3,6-dimethylpyridin-2-yl | 4.59^{[c]} |
| | | | | | | 2.91^{[b]} |
| | | | | | | (isomer B) |
| 755 | 3,6-dimethylpyridin-2-yl | Me | 2-phenylpropane-1,3-diyl | Me | 3,6-dimethylpyridin-2-yl | 5.45^{[c]} |
| | | | | | | 3.62^{[b]} |
| | | | | | | (isomer A) |
| 756 | 3,6-dimethylpyridin-2-yl | Me | 2-tert-butylpropane-1,3-diyl | Me | 3,6-dimethylpyridin-2-yl | 5.20^{[c]} |
| | | | | | | 3.22^{[b]} |
| | | | | | | (isomer B) |
| 757 | 3,6-dimethylpyridin-2-yl | Me | 2-tert-butylpropane-1,3-diyl | Me | 3,6-dimethylpyridin-2-yl | 6.38^{[c]} |
| | | | | | | 5.09^{[b]} |
| | | | | | | (isomer A) |
| 758 | 3,6-dimethylpyridin-2-yl | Me | butane-1,3-diyl | Me | 3,6-dimethylpyridin-2-yl | 4.03^{[c]} |
| | | | | | | 2.13^{[b]} |
| | | | | | | (isomer B) |
| 759 | 3,6-dimethylpyridin-2-yl | Me | butane-1,3-diyl | Me | 3,6-dimethylpyridin-2-yl | 5.02^{[c]} |
| | | | | | | 3.12^{[b]} |
| | | | | | | (isomer A) |
| 760 | 3,6-dimethylpyridin-2-yl | Me | cyclobutane-1,1-diylbis(methylene) | Me | 3,6-dimethylpyridin-2-yl | 4.72^{[c]} |
| | | | | | | 2.72^{[b]} |
| | | | | | | (isomer B) |
| 761 | 3,6-dimethylpyridin-2-yl | Me | cyclobutane-1,1-diylbis(methylene) | Me | 3,6-dimethyolpyridin-2-yl | 5.84^{[c]} |
| | | | | | | 3.77^{[b]} |
| | | | | | | (isomer A) |
| 762 | 3,6-dimethylpyridin-2-yl | Me | cyclopentane-1,1-diylbis(methylene) | Me | 3,6-dimethylpyridin-2-yl | 5.14^{[c]} |
| | | | | | | 3.04^{[b]} |
| | | | | | | (isomer B) |
| 763 | 3,6-dimethylpyridin-2-yl | Me | cyclopentane-1,1-diylbis(methylene) | Me | 3,6-dimethylpyridin-2-yl | 6.32^{[c]} |
| | | | | | | 4.76^{[b]} |
| | | | | | | (isomer A) |
| 764 | 3,6-dimethylpyridin-2-yl | Me | cyclopropane-1,1-diylbis(methylene) | Me | 3,6-dimethylpyridin-2-yl | 4.00^{[c]} |
| | | | | | | 2.17^{[b]} |
| | | | | | | (isomer B) |
| 765 | 3,6-dimethylpyridin-2-yl | Me | cyclopropane-1,1-diylbis(methylene) | Me | 3,6-dimethylpyridin-2-yl | 4.95^{[c]} |
| | | | | | | 3.15^{[b]} |
| | | | | | | (isomer A) |
| 766 | 4,5-dimethyl-1,3-thiazol-2-yl | Me | (2E)-but-2-ene-1,4-diyl | Me | 4,5-dimethyl-1,3-thiazol-2-yl | 5.08^{[b]} (isomer A) |
| 767 | 4,5-dimethyl-1,3-thiazol-2-yl | Me | (2E)-but-2-ene-1,4-diyl | Me | 4,5-dimethyl-1,3-thiazol-2-yl | 4.92^{[b]} (isomer B) |
| 768 | 4,6-bis(methoxymethyl)-5- | NH₂ | (2E)-but-2-ene-1,4-diyl | NH₂ | 4,6-bis(methoxymethyl)-5- | 1.68^{[b]} (isomer A) |
| | methylpyrimidin-2-yl | | | | methylpyrimidin-2-yl | |
| 769 | 4,6-bis(methoxymethyl)-5- | NH₂ | (2E)-but-2-ene-1,4-diyl | NH₂ | 4,6-bis(methoxymethyl)-5- | 1.75^{[b]} (isomer B) |
| | methylpyrimidin-2-yl | | | | methylpyrimidin-2-yl | |
| 770 | 4,6-dimethylpyridin-2-yl | NH₂ | (2E)-but-2-ene-1,4-diyl | NH₂ | 4,6-dimethylpyridin-2-yl | 2.55^{[b]} (isomer B) |
| 771 | 4,6-dimethylpyridin-2-yl | NH₂ | (2E)-but-2-ene-1,4-diyl | NH₂ | 4,6-dimethylpyridin-2-yl | 2.70^{[b]} (isomer A) |
| 772 | 4,6-dimethylpyrimidin-2-yl | NH₂ | (2E)-but-2-ene-1,4-diyl | NH₂ | 4,6-dimethlpyrimidin-2-yl | 1.62^{[b]} |
| 773 | 4,6-dimethylpyrimidin-2-yl | NH₂ | (2Z)-but-2-ene-1,4-diyl | NH₂ | 4,6-dimethylpyrimidin-2-yl | 1.75^{[b]} |
| 774 | 5-chloro-4,6-dimethylpyrimidin- | NH₂ | (2E)-but-2-ene-1,4-diyl | NH₂ | 5-chloro-4,6-dimethylpyrimidin-2-yl | 2.70^{[b]} |
| | 2-yl | | | | | |
| 775 | 5-chloro-4,6-dimethylpyrimidin- | NH₂ | (2Z)-but-2-ene-1,4-diyl | NH₂ | 5-chloro-4,6-dimethylpyrimidin-2-yl | 2.77^{[b]} |
| | 2-yl | | | | | |
| 776 | 6-methylpyridin-2-yl | H | butane-1,4-diyl | H | 6-methylpyridin-2-yl | 2.16^{[b]} |
| 777 | 6-methylpyridin-2-yl | Me | (2D)-but-2-ene-1,4-diyl | Me | 6-methylpyridin-2-yl | 2.89^{[b]} |
| 778 | 6-methylpyridin-2-yl | Me | (2Z)-but-2-ene-1,4-diyl | Me | 6-methylpyridin-2-yl | 3.31^{[b]} |
| 779 | 6-methylpyridin-2-yl | Me | 2,5-dichloropyrimidine-4,6-diyl | Me | 6-methylpyridin-2-yl | 5.09^{[c]} |
| | | | | | | 4.62^{[b]} |
| | | | | | | (isomer A) |
| 780 | 6-methylpyridin-2-yl | Me | 2,5-dichloropyrimidine-4,6-diyl | Me | 6-methylpyridin-2-yl | 4.88^{[c]} |
| | | | | | | 4.22^{[b]} |
| | | | | | | (isomer B) |
| 781 | pyridin-2-yl | Me | (2E)-buy-2-ene-1,4-diyl | Me | pyridin-2-yl | 2.84^{[b]} |
| 782 | 6-methylpyridin-2-yl | Me | (diethylsilanediyl)dimethanediyl | Me | 6-methylpyridin-2-yl | 6.65^{[a]} |
| | | | | | | 7.01^{[c]} |
| | | | | | | 6.65^{[b]} |
| 783 | 6-pyrrolidin-1-ylpyridin-2-yl | Me | 2-methylpropane-1,3-diyl | Me | 6-pyrrolidin-1-ylpyridin-2-yl | 2.51^{[a]} |
| 784 | 6-methylpyridin-2-yl | Cyano | 2-methylenepropane-1,3-diyl | cyano | 6-methylpyridin-2-yl | 3.84^{[a]} |
| 785 | 6-methylpyridin-2-yl | Me | [ethyl(methyl)silanediyl)dimethanediyl | Me | 6-methylpyridin-2-yl | 5.98^{[a]} |
| | | | | | | 6.52^{[c]} |
| | | | | | | 5.98^{[b]} |
| 786 | 5,6-dimethylpyridin-2-yl | Me | cyclopropane-1,1-diylbis(methylene) | Me | 6-methylpyridin-2-yl | 3.99^{[a]} |
| | | | | | | 5.70^{[c]} |
| | | | | | | 3.99^{[b]} |
| 787 | 4,6-dimethylpyridin-2-yl | Me | (diethylsilanediyl)dimethanediyl | Me | 4,6-dimethylpyridin-2-yl | 5.05^{[a]} |
| | | | | | | 7.36^{[c]} |
| | | | | | | 5.05^{[b]} |
| 788 | 6-methylpyridin-2-yl | Me | 2-ethyl-2-methylpropane-1,3-diyl | Me | 6-methylpyridin-2-yl | 6.47^{[c]} |
| | | | | | | 5.73^{[b]} |
| 789 | 3,6-dimethylpyridin-2-yl | Me | 2-ethyl-2-methylpropane-1,3-diyl | Me | 3,6-dimethylpyridin-2-yl | 6.13^{[c]} |
| | | | | | | 4.45^{[c]} |
| 790 | 4,6-dimethylpyridin-2-yl | Me | 3-methylpentane-2,4-diyl | Me | 4,6-dimethylpyridin-2-yl | 3.60^{[a]} |
| | | | | | | 4.44^{[b]} |
| 791 | 6-[(cyclopropylmethyl)- | Me | 2-methylpropane-1,3-diyl | Me | 6-[(cyclopropylmethyl)- | 1.95^{[a]} |
| | amino]pyridin-2-yl | | | | amino]pyridin-2-yl | |
| 792 | 4-chloro-6-methylpyridin-2-yl | Me | 2-methylpropane-1,3-diyl | Me | 4-chloro-6-methylpyridin-2-yl | 7.36^{[a]} |
| | | | | | | 7.36^{[c]} |
| 793 | 5,6-dimethylpyridin-2-yl | Me | 2-ethyl-2-methylpropane-1,3-diyl | Me | 5,6-dimethylpyridin-2-yl | 7.27^{[c]} |
| | | | | | | 5.39^{[b]} |
| 794 | 4,6-dimethylpyridin-2-yl | Me | 2-ethyl-2-methylpropane-1,3-diyl | Me | 4,6-dimethylpyridin-2-yl | 7.18^{[c]} |
| | | | | | | 3.91^{[b]} |
| 795 | 4,6-dimethypyridin-2-yl | Cl | 2-methylenepropane-1,3-diyl | Cl | 4,6-dimethylpyriidn-2-yl | 4.62^{[a]} |
| 796 | 4,6-dimethylpyridin-2-yl | Me | cyclopentane-1,3-diyl | Me | 4,6-dimethylpyridin-2-yl | 2.55^{[a]} |
| 797 | 6-methylpyridin-2-yl | Me | cyclopentane-1,3-diyl | Me | 6-methylpyridin-2-yl | 4.60^{[a]} |
| 798 | 6-benzylpyridin-2-yl | Me | 2,2-dimethylpropane-1,3-diyl | Me | 6-benzylpyridin-2-yl | 7.36^{[b]} |
| | | | | | | 7.36^{[b][a]} |
| 799 | 5,6-dimethylpyridin-2-yl | Me | 2,2-dimethylpropane-1,3-diyl | Me | 6-methylpyridin-2-yl | 4.96^{[a]} |
| | | | | | | 6.34^{[c]} |
| | | | | | | 4.96^{[b]} |
| 800 | 6-cyanopyridin-2-yl | Me | 2-methylpropane-1,3-diyl | Me | 6-cyanopyridin-2-yl | 4.78^{[a]} |
| 801 | 6-methylpyridin-2-yl | Me | cyclohexane-1,3-diyl | Me | 6-methylpyridin-2-yl | 4.96^{[a]} |
| 802 | 6-methylpyridin-2-yl | vinyl | 2-methylenepropane-1,3-diyl | vinyl | 6-methlpyridin-2-yl | |
| 803 | 6-methylpyridin-2-yl | prop-1-yn- | 2-methylenepropane-1,3-diyl | prop-1-yn-1- | 6-methylpyridin-2-yl | 3.07^{[b]} |
| | | 1-yl | | yl | | |
| 804 | 4-chloro-6-methylpyridin-2-yl | Me | 2,2-dimethylpropane-1,3-diyl | Me | 4-chloro-6-methylpyridin-2-yl | 7.36^{[a]} |
| 805 | 4-chloro-6-methylpyridin-2-yl | Me | cyclopropane-1,1-diylbis(methylene) | Me | 4-chloro-6-methylpyridin-2-y | 7.02^{[a)} |
| 806 | 4-methoxy-6-methylpyridin-2-yl | Me | 2-methylpropane-1,3-diyl | Me | 4-methoxy-6-methylpyridin-2-yl | 1.31^{[a]} |
| | | | | | | 5.25^{[c]} |
| 807 | 6-methylpyridin-2-yl | Me | 2-methylenepropane-1,3-diyl | Cl | 5,6-dimethylpyridin-2-yl | 3.8^{[a]} |
| 808 | 6-methylpyridin-2-yl | Me | 2-methylenepropane-1,3-diyl | NH2 | 5,6-dimethylpyridin-2-yl | 2.38^{[a]} |
| 809 | 6-methylpyridin-2-yl | Me | 2,2-diethylpropane-1,3-diyl | Me | 6-methylpyridin-2-yl | 6.97^{[c]} |
| | | | | | | 6.68^{[b]} |
| 810 | 5,6-dimethylpyridin-2-yl | Me | 2,2-diethylpropane-1,3-diyl | Me | 5,6-dimethylpyridin-2-yl | 7.36^{[c]} |
| | | | | | | 6.50^{[b]} |
| 811 | 4,6-dimethylpyridin-2-yl | Me | 2,2-diethylpropane-1,3-diyl | Me | 4,6-dimethylpyridin-2-yl | 7.36^{[c]} |
| | | | | | | 4.79^{[b]} |
| 812 | 3,6-dimethylpyridin-2-yl | Me | 2,2-diethylpropane-1,3-diyl | Me | 3,6-dimethylpyridin-2-yl | 6.66^{[c]} |
| | | | | | | 5.24^{[b]} |
| 813 | 6-cyclopropylpyridin-2-yl | Me | cyclopentane-1,3-diyl | Me | 6-cyclopropylpyridin-2-yl | 7.36^{[a]} |
| 814 | 6-cyclopropylpyridin-2-yl | Me | 2,2-dimethylpropane-1,3-diyl | Me | 6-cyclopropylpyridin-2-yl | 7.36^{[a]} |
| 815 | 6-cyclopropylpyridin-2-yl | Me | 2-methylpropane-1,3-diyl | Me | 6-cyclopropylpyridin-2-yl | 7.36^{[a]} |
| 816 | 4-mthoxy-6-methylpyridin-2-yl | Me | cyclopropane-1,1-diylbis(methylene) | Me | 4-methoxy-6-methylpyridin-2-yl | 1.34^{[a]} |
| | | | | | | 4.94^{[c]} |
| 817 | 4-cyclopropyl-6-methylpyridin-2- | Me | 2,2-dimethylpropane-1,3-diyl | Me | 4-cyclopropyl-6-methylpyridin-2-yl | 3.27^{[a]} |
| | yl | | | | | 7.14^{[c]} |
| 818 | 6-methylpyridin-2-yl | Me | 2,2-dimethylpropane-1,3-diyl | Me | 4-cyclopropyl-6-methylpyridin-2-yl | 3.80^{[a]} |
| | | | | | | 6.57^{[c]} |
| 819 | 6-methylpyridin-2-yl | propylthio | 2-methylenepropane-1,3-diyl | propylthio | 6-methylpyridin-2-yl | 4.9^{[a]} |
| 820 | 6-methylpyridin-2-yl | ethylthio | 2-methylpropane-1,3-diyl | ethylthio | 6-methylpyridin-2-yl | 4.35^{[a]} |
| 821 | 6-methylpyridin-2-yl | Me | 2-methylenepropane-1,3-diyl | cyano | 5,6-dimethylpyridin-2-yl | 3.74^{[a]} |
| 822 | 5,6-dimethylpyridin-2-yl | Cl | 2-methylenepropane-1,3-diyl | Cl | 6-methylpyridin-2-yl | 4.12^{[a]} |
| 823 | 6-methylpyridin-2-yl | Me | 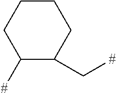 | Me | 6-methylpyridin-2-yl | 5.9^{[a]} |
| 824 | 6-methylpyridin-2-yl | Cl | 2-methylpropane-1,3-diyl | Cl | 6-methylpyridin-2-yl | 4.58^{[a]} |
| 825 | 5-methoxy-6-methylpyridin-2-yl | Me | 2-methylpropane-1,3-diyl | Me | 5-methoxy-6-methylpyridin-2-yl | 5.58^{[c]} |
| | | | | | | 5.21^{[b]} |
| 826 | 2-pyridyl | Me | 2-methylpropane-1,3-diyl | Me | 6-(trimethylsilyl)pyridin-2-yl | 7.29^{[c]} |
| | | | | | | 6.72^{[b]} |
| 827 | 6-methylpyridin-2-yl | cyano | 2-methylpropane-1,3-diyl | cyano | 6-methylpyridin-2-yl | 4.20^{[a]} |
| 828 | 5,6-dimethylpyridin-2-yl | cyano | 2-methylenepropane-1,3-diyl | cyano | 6-methylpyridin-2-yl | 4.20^{[a]} |
| 829 | 4,6-dimethylpyridin-2-yl | methoxy | 2-methylenepropane-1,3-diyl | methoxy | 4,6-dimethylpyridin-2-yl | |
| 830 | 4,6-dimethylpyridin-2-yl | cyano | 2-methylenepropane-1,3-diyl | cyano | 4,6-dimethylpyridin-2-yl | 4.50^{[a]} |
| 831 | 6-methylpyridin-2-yl | Cl | 2-methylenepropane-1,3-diyl | NH₂ | 5,6-dimethylpyridin-2-yl | 4.06^{[a]} |
| 832 | 5-(trifluoromethyl)pyridin-2-yl | Me | 3-methylpentane-1,5-diyl | Me | 5-(trilfuoromethyl)pyridin-2-yl | 7.36^{[a]} |
| 833 | 4,6-dimethylpyridin-2-yl | Cl | 2-methylpropane-1,3-diyl | Cl | 4,6-dimethylpyridin-2-yl | 4.17^{[a]} |
| 834 | 6-methylpyridin-2-yl | Me | [methyl(1-methylethyl)silanedyl]dimethanediyl | Me | 6-methylpyridin-2-yl | 6.72^{[a]} |
| | | | | | | 7.35^{[c]} |
| | | | | | | 6.72^{[b]} |
| 835 | 6-methylpyridin-2-yl | methoxy | 2-methylpropane-1,3-diyl | methoxy | 6-methylpyridin-2-yl | 1.95^{[a]} |
| 836 | 4,6-dimethylpyridin-2-yl | Me | [methyl(1-methylethyl)silanediyl)dimethanediyl | Me | 4,6-dimethylpyridin-2-yl | 5.04^{[a]} |
| | | | | | | 7.36^{[c]} |
| | | | | | | 5.04^{[b]} |
| 837 | 5,6-dimethylpyridin-2-yl | Me | [methyl(1-methylethyl)silanediyl9 dimethanediyl | Me | 5,6-dimethylpyridin-2-yl | 6.76^{[a]} |
| | | | | | | 7.36^{[c]} |
| | | | | | | 6.76^{[b]} |
| 838 | 4,6-dimethylpyridin-2-yl | cyano | 2-methylpropane-1,3-diyl | cyano | 4,6-dimethylpyridin-2-yl | 4.84^{[a]} |
| 839 | 4-chloro-5-methoxy-6- | Me | 2-methylpropane-1,3-diyl | Me | 4-chloro-5-methoxy-6- | 7.36^{[c]} |
| | methylpyridin-2-yl | | | | methylpyridin-2-yl | 7.36^{[b]} |
| 840 | 4-methoxy-6-methylpyridin-2-yl | Me | 2,2-dimethylpropane-1,3-diyl | Me | 4-methoxy-6-methylpyridin-2-yl | 1.5^{[a]} |
| | | | | | | 5.79^{[c]} |
| 841 | 4-ethoxy-6-methyl]pyridin-2-yl | Me | 2,2-dimethylpropane-1,3-diyl | Me | 4-ethoxy-6-methylpyridin-2-yl | 2.00^{[a]} |
| | | | | | | 6.90^{[c]} |
| 842 | 5,6-dimethylpyridin-2-yl | propylthio | 2-methylenepropane-1,3-diyl | propylthio | 6-methylpyridin-2-yl | |
| 843 | 5,6-dimethylpyridin-2-yl | ethoxy | 2-methylenepropane-1,3-diyl | ethoxy | 6-methylpyridin-2-yl | 2.25^{[a]} |
| 844 | 5,6-dimethylpyridin-2-yl | methoxy | 2-methylenepropane-1,3-diyl | methoxy | 6-methylpyridin-2-yl | |
| 845 | 6-methylpyridin-2-yl | Me | 2-methylpropane-1,3-diyl | Me | 4-cyclopropyl-6-methylpyridin-2-yl | 3.21^{[a]} |
| | | | | | | 6.40^{[c]} |
| 846 | 4-chloro-6-methylpyridin-2-yl | Me | 2-methylpropane-1,3-diyl | Me | 4-cyclopropyl-6-methylpyridin-2-yl | 5.23^{[a]} |
| | | | | | | 7.36^{[c]} |
| 847 | 6-methyl-4-pyrrolidin-1- | Me | 2-methylpropane-1,3-diyl | Me | 6-methyl-4-pyrrolidin-1-ylpyridin-2- | 1.40^{[a]} |
| | ylpyridin-2-yl | | | | yl | 5.93^{[c]} |
| 848 | 6-cyclopropylpyridin-2-yl | Me | cyclopropane-1,1-diylbis(methylene) | Me | 6-cyclopropylpyridin-2-yl | 7.36^{[a]} |
| 849 | 6- | Me | cyclopropane-1,1-diylbis(methylene) | Me | 6- | 1.93^{[b]} |
| | [(cyclopropylmethyl)amino]pyridin- | | | | [(cyclopropylmethyl)amino]pyridin- | |
| | 2-yl | | | | 2-yl | |
| 850 | 6-[(cyclopropylmethyl)- | Me | cyclobutane-1,1-diylbis(methylene) | Me | 6-[(cyclopropylmethyl)- | 2.28^{[b]} |
| | amino]pyridin-2-yl | | | | amino]pyridin-2-yl | |
| 851 | 5,6-dimethylpyridin-2-yl | Me | butane-1,4-diyldicarbamoyl | Me | 5,6-dimethylpyridin-2-yl | 2.04^{[a]} |
| 852 | 4,6-dimethylpyridin-2-yl | Me | butane-1,4-diyldicarbamoyl | Me | 4,6-dimethylpyridin-2-yl | 1.63^{[a]} |
| 853 | 6-methylpyridin-2-yl | Me | 2-(ethoxycarbonyl)-2-methylpropane-1,3-diyl | Me | 6-methylpyridin-2-yl | 5.00^{[c]} |
| | | | | | | 4.06^{[b]} |
| 854 | 5,6-dimethylpyridin-2-yl | Me | 2-(ethoxycarbonyl)-2-methylpropane-1,3-diyl | Me | 5,6-dimethylpyridin-2-yl | 5.82^{[c]} |
| | | | | | | 3.85^{[b]} |
| 855 | 4,6-dimethylpyridin-2-yl | Me | 2-(ethoxycarbonyl)-2-methylpropane-1,3-diyl | Me | 4,6-dimethylpyridin-2-yl | 5.72^{[c]} |
| | | | | | | 2.79^{[b]} |
| 856 | 6-methylpyridin-2-yl | Me | (2-oxo-1,3-oxazolidine-4,4-diyl)dimethanediyl | Me | 6-methylpyridin-2-yl | 2.97^{[c]} |
| | | | | | | 1.99^{[b]} |
| 857 | 5,6-dimethylpyridin-2-yl | Me | (2-oxo-1,3-oxazolidine-4,4-diyl)dimethanediyl | Me | 5,6-dimethylpyridin-2-yl | 3.70^{[c]} |
| | | | | | | 1.96^{[b]} |
| 858 | 4,6-dimethylpyridin-2-yl | Me | (2-oxo-1,3-oxazolidine-4,4-diyl)dimethanediyl | Me | 4,6-dimethylpyridin-2-yl | 3,55^{[c]} |
| | | | | | | 1.43^{[b]} |
| 859 | 3,6-dimethylpyridin-2-yl | Me | (2-oxo-1,3-oxazolidine-4,4-diyl)dimethanediyl | Me | 3,6-dimethylpyridin-2-yl | 2.81^{[c]} |
| | | | | | | 1.70^{[b]} |
| 860 | 3,6-dimethylpyridin-2-yl | Me | 2-(ethoxycarbonyl)-2-methylpropane-1,3-diyl | Me | 3,6-dimethylpyridin-2-yl | 4.72^{[c]} |
| | | | | | | 3.03[b] |
| 861 | 5,6-dimethylpyridin-2-yl | Me | 2-methylpentane-1,5-diyl)dicarbamoyl | Me | 5,6-dimethylpyridin-2-yl | 2.55^{[a]} |
| 862 | 4,6-dimethylpyridin-2-yl | Me | 2-methylpentane-1,5-diyl)dicarbamoyl | Me | 4,6-dimethylpyridin-2-yl | 2.06^{[a]} |
| 863 | 6-methylpyridin-2-yl | Me | 2-methylpentane-1,5-diyl)dicarbamoyl | Me | 6-methylpyridin-2-yl | 2.46^{[a]} |
| 864 | 6-methylpyridin-2-yl | Me | butane-1,4-diyldicarbamoyl | Me | 6-methylpyridin-2-yl | 1.97^{[a]} |
| 865 | 4-iodo-6-methylpyridin-2-yl | Me | 2-methylpropane-1,3-diyl | Me | 4-iodo-6-methylpyridin-2-yl | |
| 866 | 6-methyl-4-[(1Z)-prop-1- | Me | 2-methylpropane-1,3-diyl | Me | 6-methyl-4-[(1Z)-prop-1-en-1- | 4.57^{[a]} |
| | en-1-yl]pyridin-2-yl | | | | yl]pyridin-2-yl | 4.57^{[b]} |
| 867 | 6-methyl-5-(prop-2-yn-1- | Me | 2-methylpropane-1,3-diyl | Me | 6-methyl-5-(prop-2-yn-1- | 5.44^{[c]} |
| | yloxy)pyridin-2-yl | | | | yloxy)pyridin-2-yl | 5.33^{[b]} |
| 868 | 4-(hydroxymethyl)-6- | Me | 2-methylpropane-1,3-diyl | Me | 4-(hydroxymethyl)-6-methylpyridin- | 2.67^{[c]} |
| | methylpyridin-2-yl | | | | 2-yl | 1.31^{[b]} |
| 869 | 6-methyl-4- | Me | 2-methylpropane-1,3-diyl | Me | 6-methyl-4-(trifluoromethyl)pyridin- | 7.36^{[a]} |
| | (trifluoromethyl)pyridin-2-yl | | | | 2-yl | 5.53^{[c]} |
| | | | | | | 7.36^{[b]} |
| 870 | 5,6-dimethylpyridin-2-yl | Me | (dimethylsilanediyl)bis(methylene) | Me | 5,6-dimethylpyridin-2-yl | 7.05^{[c]} |
| 871 | 4-(methoxymethyl)-6- | Me | 2-methylpropane-1,3-diyl | Me | 4-(methoxymethyl)-6- | 5.57^{[c]} |
| | methylpyridin-2-yl | | | | methylpyridin-2-yl | 4.06^{[b]} |
| 872 | 6-methylpyridin-2-yl | Me | 2-(prop-2-en-1-yloxy)-2-methyl-1,3-butanediyl | Me | 6-methylpyridin-2-yl | 6.13^{[a]} |
| | | | | | | 6.96^{[c]} |
| | | | | | | 6.13^{[b]} |
| 873 | 4-ethynyl-6-methylpyridin-2-yl | Me | 2-methylpropane-1,3-diyl | Me | 4-ethynyl-6-methylpyridin-2-yl | 6.65^{[a]} |
| | | | | | | 6.61^{[c]} |
| | | | | | | 6.65^{[b]} |
| 874 | 4-(3-methoxyprop-1-yn-1-yl)-6- | Me | 2-methylpropane-1,3-diyl | Me | 4-(3-methoxyprop-1-yn-1-yl)-6- | 6.76^{[a]} |
| | methylpyridin-2-yl | | | | methylpyridin-2-yl | 6.87^{[c]} |
| | | | | | | 6.76^{[b]} |
| 875 | 4-[3-(acetyloxy)prop-1-yn-1-yl]- | Me | 2-methylpropane-1,3-diyl | Me | 4-[3-(acetyloxy)prop-1-yn-1-yl]-6- | 6.25^{[a]} |
| | 6-methylpyridin-2-yl | | | | methylpyridin-2-yl | 6.31^{[c]} |
| | | | | | | 6.25^{[b]} |
| 876 | 5,6-dimethylpyridin-2-yl | Me | 2-methylpropane-1,3-diyl | Me | 4-chloro-6-methylpyridin-2-yl | 6.52^{[a]} |
| | | | | | | 7.36^{[c]} |
| | | | | | | 6.52^{[b]} |
| 877 | 6-methylpyridin-2-yl | Me | 2-methylpropane-1,3-diyl | Me | 4-chloro-6-methylpyridin-2-yl | 6.58^{[a]} |
| | | | | | | 7.36^{[c]} |
| | | | | | | 6.58^{[b]} |
| 878 | 4-chloro-6-methylpyridin-2-yl | Me | 2-methylpropane-1,3-diyl | Me | 4,6-dimethylpyridin-2-yl | 5,69^{[a]} |
| | | | | | | 7.32^{[c]} |
| | | | | | | 5.69^{[b]} |
| 879 | 6-methylpyridin-2-yl | Me | 2,2-dimethylpropane-1,3-diyl | Ph | 6-methylpyridin-2-yl | 4.52^{[c]} |
| | | | | | | 4.61^{[b]} |
| 880 | 6-methylpyridin-2-yl | Me | 2,2-dimethylpropane-1,3-diyl | Ph | 6-methylpyridin-2-yl | 5.37^{[c]} |
| | | | | | | 4.72^{[b]} |
| 881 | 6-benzylpyridin-2-yl | Me | 2,2-dimethylpropane-1,3-diyl | Me | 6-benzylpyridin-2-yl | 7.36^{[b]} |
| 882 | 3,6-dimethylpyridin-2-yl | Me | 2,2-diethylpropane-1,3-diyl | Me | 3,6-dimethylpyridin-2-yl | 5.41^{[c]} |
| | | | | | | 3.90^{[b]} |
| 883 | 6-methylpyridin-2-yl | Et | 2,2-dimethylpropane-1,3-diyl | Et | 6-methylpyridin-2-yl | 7.36^{[c]} |
| | | | | | | 6.81^{[b]} |
| 884 | 3,6-dimethylpyridin-2-yl | Me | 2-(ethoxycarbonyl)-2-methylpropane-1,3-diyl | Me | 3,6-dimethylpyridin-2-yl | 3.84^{[c]} |
| | | | | | | 2.47^{[b]} |

In this table, the abbreviations have the following meanings:
Me = methyl,
Et = ethyl,
nPr = n-propyl,
iPr = isopropyl,
cPr = cyclopropyl,
iBu = isobutyl,
Bzl = benzyl,
Ph = phenyl

### Preparation of Starting Materials of the Formula (III)

### Example (III-1)

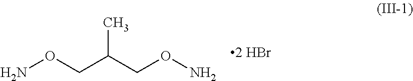

A mixture of 35.3 g (91.0 mmol) of 2,2′-[(2-methylpropane-1,3-diyl)bis(oxy)]bis(1H-isoindole-1,3(2H)-dione) (IX-1), 93 ml acetic acid and 137 ml of concentrated hydrobromic acid was heated under reflux until the starting material had dissolved completely. After cooling to room temperature, the resulting precipitate was filtered off and the filtrate was concentrated under reduced pressure. The residue obtained was taken up in 55 ml of water and filtered again after 1 h.

The solvent was removed from the filtrate, and the [(2-methylpropane-1,3-diyl)bis(oxy)]diammonium dibromide (27.2 g) obtained was directly reacted further, without further purification.

### Example (III-2)

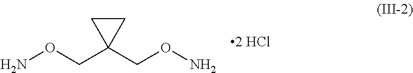

1,1-Bis[(aminooxy)methyl]cyclopropane dihydrochloride can be obtained analogously to Example (III-1).

### Preparation of Starting Materials of the Formula (VI)

### Example (VI-1)

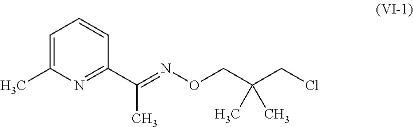

At room temperature, 43.9 ml (43.9 mmol) of a 1M solution of 1-tert-butyl-4,4,4-tris(dimethylamino)-2,2-bis[tris(dimethylamino)phosphoranylideneamino]-2⁵,4⁵-cateadi(phosphazene) (═P4-phosphazene) in hexane was added dropwise to a solution of 6 g (40 mmol) of (1E/Z)-1-(6-methylpyridin-2-yl)ethanone oxime in 600 ml of tetrahydrofuran. A solution with a deep orange colour was formed, and the solution was stirred for another 30 min and then cooled to 0° C. At this temperature, 16.3 g (87.9 mmol) of 1-chloro-3-bromo-2,2-dimethylpropane were rapidly added dropwise. After a further hour at 0° C., the mixture was warmed to room temperature for 16 h, whereupon the colour of the solution changed to light-brown. Water was added carefully, and the aqueous phase was extracted repeatedly with ethyl acetate. The organic phase was washed with saturated sodium chloride solution, dried over sodium sulphate and concentrated under reduced pressure, and the residue was then triturated with diethyl ether and the precipitated protonated P4 phosphazene base was filtered off. The residue that remained was purified by chromatography on silica gel (gradient cyclohexane/ethyl acetate).

This gave 8 g (76% of theory) of (1E)-1-(6-methylpyridin-2-yl)ethanone O-(3-chloro-2,2-dimethylpropyl)oxime of logP (HCO₂H)=3.76.

### Preparation of Starting Materials of the Formula (IX)

### Example (IX-1)

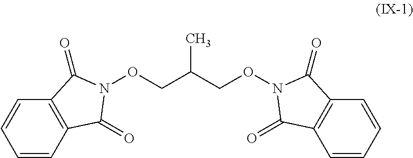

At 0° C., 76.5 g (355 mmol) of diisopropyl azodicarboxylate (dissolved in 50 ml of tetrahydrofuran) were added dropwise to a solution of 14.5 g (161 mmol) of 2-methyl-1,3-propanediol (VIII-1), 52.6 g (323 mmol) of N-hydroxyphthalimide (VII) and 93.2 g (355 mmol) of triphenylphosphane in 500 ml of tetrahydrofuran. The reaction mixture was stirred at room temperature for 16 h and then freed from the solvent under reduced pressure. The residue was taken up in 250 ml of MeOH/H₂O (5:1) and the precipitated solid was filtered off. The solid was again taken up in 240 ml of MeOH/H₂O (5:1), filtered again, washed with 60 ml of MeOH/H₂O (5:1) and air-dried.

This gave 2,2′-[(2-methylpropane-1,3-diyl)bis(oxy)]bis(1H-isoindole-1,3(2H)-dione) in a yield of 55.6 g (146 mmol, 90% of theory) of logP (HCO₂H)=2.87.

The stated logP values were determined in accordance with EEC Directive 79/831 Annex V.A8 by HPLC (high performance liquid chromatography) on a reversed-phase column (C 18). Temperature: 43° C.

### Preparation of Hydroxylamines of the Formula (XII)

### Example (XII-8)

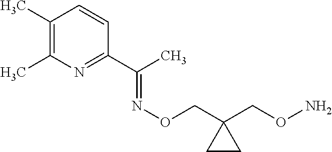

At room temperature, a suspension of 2.05 g (10 mmol) of 1,1-bis[(aminooxy)methyl]cyclopropane dihydrochloride (III-2) in 50 ml of ethanol and 130 mg of p-toluenesulphonic acid were added to a solution of 1.49 g (10 mmol) of 2-acetyl-5,6-dimethylpyridine (II-1) in 50 ml of ethanol, and the mixture was heated under reflux for 4 h. After cooling, the reaction mixture was added to ice-water and neutralized by addition of triethylamine. After extraction with dichloromethane, the combined organic phases were dried over Na₂SO₄ and freed from the solvent under reduced pressure.

Purification by column chromatography on silica gel using cyclohexane/ethyl acetate gave 1.37 g (5.2 mmol, 50% of theory) of (1E)-1-(5,6-dimethylpyridin-2-yl)ethanone O-({1-[(aminooxy)methyl]-cyclopropyl}methyl)oxime of logP (HCO₂H)=0.73.

The compounds of the formula (XII) listed in Table 2 below can be obtained analogously to the preceding example and in accordance with the general descriptions of the processes according to the invention.

TABLE 2

| | |
|---|---|
| | (XII) |
| 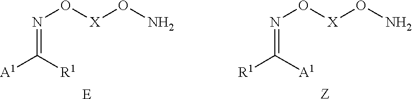 | |

| No. | A¹ | R¹ | X | logP | isomer |
|---|---|---|---|---|---|
| XII-1 | 6-methylpyridin-2-yl | iPr | —CH₂—CH₂—CH₂— | 1.12^{[a]} | E |
| XII-2 | 6-methylpyridin-2-yl | iPr | —CH₂—CH₂—CH₂— | 1.05^{[a]} | Z |
| XII-3 | 6-methylpyridin-2-yl | Me | 2-n-propyl-propane-1,3-diyl | 145^{[b]} | E |
| XII-4 | 6-methylpyridin-2-yl | Me | 2,2-dimethylpropane-1,3-diyl | 1.11^{[b]} | E |
| XII-5 | 6′-methyl-2,2′-bipyridin-6-yl | Me | 2-methylpropane-1,3-diyl | 3.36^{[c]} | E |
| | | | | 1.35^{[b]} | |
| XII-6 | 5,6-dimethylpyridin-2-yl | Me | 2,2-dimethylpropane-1,3-diyl | 0.98^{[a]} | E |
| | | | | 2.94^{[c]} | |
| | | | | 0.98^{[b]} | |
| XII-7 | 5,6-dimethylpyridin-2-yl | Me | 2-methylpropane-1,3-diyl | 0.37^{[a]} | E |
| | | | | 2.49^{[c]} | |
| | | | | 0.37^{[b]} | |
| XII-8 | 5,6-dimethylpyridin-2-yl | Me | cyclopropane-1,1-diylbis(methylene) | 0.53^{[a]} | E |
| | | | | 2.44^{[c]} | |
| | | | | 0.53^{[b]} | |
| XII-9 | 4,6-dimethylpyridin-2-yl | Me | 2-methylpropane-1,3-diyl | 0.49^{[a]} | E |
| | | | | 0.49^{[b]} | |
| XII-10 | 6-methylpyridin-2-yl | Me | —CH₂—CH₂—CH₂— | 0.27^{[a]} | E |
| | | | | 1.72^{[c]} | |
| | | | | 0.27^{[b]} | |
| XII-11 | 6-methylpyridin-2-yl | Me | 2-methylpropane-1,3-diyl | 0.7^{[a]} | E |
| | | | | 2.15^{[c]} | |
| | | | | 0.7^{[b]} | |
| XII-12 | 4,6-dimethylpyridin-2-yl | Me | cyclopropane-1,1-diylbis(methylene) | 0.61^{[a]} | E |
| | | | | 2.42^{[c]} | |
| | | | | 0.61^{[b]} | |
| XII-13 | 6-methylpyridin-2-yl | Me | cyclopropane-1,1-diylbis(methylene) | 0.78^{[a]} | E |
| | | | | 2.11^{[c]} | |
| | | | | 0.78^{[b]} | |
| XII-14 | 4,6-dimethylpyridin-2-yl | Me | 2,2-dimethylpropane-1,3-diyl | 0.8^{[a]} | E |
| | | | | 2.89^{[c]} | |
| | | | | 0.8^{[b]} | |
| XII-15 | 6-methyl-4-(trifluoromethyl)- | Me | 2-methylpropane-1,3-diyl | 2.33^{[a]} | E |
| | pyridin-2-yl | | | 3.38^{[c]} | |
| | | | | 2.33^{[b]} | |
| XII-16 | 6-methyl-4-(trifluoromethyl)- | Me | 2,2-dimethylpropane-1,3-diyl | 2.69^{[a]} | E |
| | pyridin-2-yl | | | 3.92^{[c]} | |
| | | | | 2.69^{[b]} | |
| XII-17 | 5,6-dimethylpyridin-2-yl | Me | —(CH₂)₄— | 0.51^{[a]} | E |

If isomer mixtures are described, the logP value of the main isomer is stated.
^{[a]}The determination was carried out in the acidic range at pH 2.3 using the mobile phases 0.1% aqueous phosphoric acid and acetonitrile; linear gradient from 10% acetonitrile to 95% acetonitrile.
^{[b]}The determination by LC-MS in the acidic range was carried out at pH 2.7 using the mobile phases 0.1% aqueous formic acid and acetonitrile (contains 0.1% formic acid); linear gradient from 10% acetonitrile to 95% acetonitrile.
^{[c]}The determination by LC-MS in the neutral range was carried out at pH 7.8 using the mobile phases 0.001 molar aqueous ammonium bicarbonate solution and acetonitrile; linear gradient from 10% acetonitrile to 95% acetonitrile.

Calibration was carried out using unbranched alkan-2-ones (of 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

### Use Examples

### Example A

Venturia test (apple)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day. The plants are then placed in a greenhouse at about 21° C. and a relative atmospheric humidity of about 90%. Evaluation is carried out 10 days after the inoculation. 0% means an efficacy which corresponds to that of the control, whereas an efficacy of 100% means that no infection is observed.

In this test, the compounds according to the invention 73, 141, 350, 420, 477, 499, 9, 182, 186, 188, 189, 1, 639, 14, 593, 88, 90, 91, 116, 112, 118, 134, 174, 175, 180, 239, 240, 311, 292, 225, 224, 235, 236, 262, 277, 276, 281, 280, 304, 15, 62, 270, 264, 67, 70, 177, 202, 204, 219, 248, 265, 295, 294, 296, 297, 298, 310, 313, 315, 316, 317, 321, 322, 519, 556, 555, 559, 562, 553, 557 and 558, and also 364, 370, 372, 460, 474, 487, 501, 502, 697, 694, 695, 570, 571, 572, 683, 592, 743, 659, 561, 677, 678, 587, 589, 679, 688, 680, 681 and 682, and furthermore XII-4, 690, 880, 359, 376, 420, 491, XII-8, 786, 782, 799, 834, 816, 788, 794, 809, 825, 823, 669, 312, 283, 310 and 319 show, at an active compound concentration of 100 ppm, an efficacy of 70% or more.

### Example B

Botrytis test (bean)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity. 2 days after the inoculation, the size of the infected areas on the leaves is evaluated. 0% means an efficacy which corresponds to that of the control, whereas an efficacy of 100% means that no infection is observed.

In this test, the compounds according to the invention 73, 141, 350, 420, 499, 9, 72, 81, 14, 212, 90, 91, 116, 112, 118, 134, 131, 136, 173, 174, 175, 239, 292, 301, 225, 224, 235, 236, 280, 302, 303, 1, 15, 23, 270, 289, 67, 70, 181, 219, 251, 254, 266, 295, 294, 296, 297, 298, 313, 315, 316, 317, 318, 319, 320, 322, 519, 550, 556, 555, 559, 554, 562, 553, 552, 557 and 558, and also 364, 370, 372, 460, 474, 487, 501, 502, 699, 697, 694, 695, 570, 571, 572, 683, 592, 743, 659, 561, 677, 678, 587, 588, 589, 763, 679, 688, 680, 681, 682 and 753, and furthermore 880, XII-8, 786, 785, 787, 806, 799, 834, 836, 837, 816, 788, 793, 794, 809, 810, 811, 825, 791 and 823 show, at an active compound concentration of 250 ppm, an efficacy of 70% or more.

### Example C

Puccinia test (wheat)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young wheat plants are sprayed with the active compound preparation at the stated application rate. 1 day after the treatment, the plants are inoculated with a spore suspension of Puccinia recondita and then remain at 100% relative humidity and 20° C. for 48 h. The plants then remain at 80% relative atmospheric humidity and a temperature of 20° C. Evaluation is carried out 8-10 days after the inoculation. 0% means an efficacy which corresponds to that of the control, whereas an efficacy of 100% means that no infection is observed.

In this test, the compounds according to the invention 250, 141, 538, 373, 375, 428, 460, 474, 487, 491, 499, 502, 9, 570, 571, 572, 14, 90, 91, 116, 112, 118, 239, 292, 225, 224, 235, 236, 277, 276, 280, 303, 304, 1, 15, 17, 18, 289, 248, 296, 298, 315, 316, 318, 319, 320, 556, 555, 559, 554, 562, 553, 552, 557 and 558, and also 772, 777, 699, XII-8, 697, 694, 695, 683, 743, 698, 14, 706, 269, 267, 561, 583, 582, 587, 589, 763, 679, 681 and 682, and furthermore 880, 798, 364, 835, 786, 782, 785, 787, 806, 799, XII-14, 834, 836, 837, 840, 841, 816, 817, 818, 847, 838, 843, 788, 793, 794, 809, 810, 811, 825, 870, 790, 783, 791, 800, 848 show, at an active compound concentration of 500 ppm, an efficacy of 70% or more.

### Example D

Alternaria test (tomato)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifer, and the concentrate is diluted with water to the desired concentration. To test for protective activity, young tomato plants are sprayed with the active compound preparation at the stated application rate. 1 day after the treatment, the plants are inoculated with a spore suspension of Alternaria solani and then remain at 100% relative humidity and 20° C. for 24 h. The plants then remain at 96% relative atmospheric humidity and a temperature of 20° C. Evaluation is carried out 7 days after the inoculation. 0% means an efficacy which corresponds to that of the control, whereas an efficacy of 100% means that no infection is observed.

In this test, the compounds according to the invention 250, 141, 327, 367, 370, 371, 372, 373, 375, 428, 429, 476, 483, 486, 487, 495, 499, 501, 502, 509, 9, 188, 1, 14, 593, 116, 112, 118, 119, 165, 180, 179, 239, 292, 301, 300, 225, 224, 235, 236, 278, 276, 281, 280, 15, 289, 67, 73, 70, 181, 204, 248, 272, 266, 295, 294, 296, 297, 298, 313, 322, 519 and 557, and also 346, 416, 10, 27, 725, 59, 541, 699, XII-8, 697, 695, 592, 743, 72, 13, 57, 203, 283, 561, 587, 588 and 589, and furthermore 880, 786, 787, 806, 799, 834, 836, 816, 825, 870, 871, 790, 791 show, at an active compound concentration of 500 ppm, an efficacy of 70% or more.

### Example E

Sphaerotheca test (cucumber)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young cucumber plants are sprayed with the active compound preparation at the stated application rate. 1 day after the treatment, the plants are inoculated with a spore suspension of Sphaerotheca fuliginea. The plants are then placed in a greenhouse at 70% relative atmospheric humidity and a temperature of 23° C. Evaluation is carried out 7 days after the inoculation. 0% means an efficacy which corresponds to that of the control, whereas an efficacy of 100% means that no infection is observed.

In this test, the compounds according to the invention 250, 73, 141, 538, 364, 367, 370, 372, 420, 423, 427, 428, 429, 460, 487, 491, 499, 502, 504, 571, 572, 573, 14, 32, 112, 97, 118, 134, 174, 175, 169, 180, 179, 239, 240, 292, 301, 225, 224, 229, 235, 236, 255, 262, 260, 277, 279, 278, 276, 280, 302, 303, 304, 1, 62, 264, 289, 206, 219, 248, 253, 265, 296, 310, 315, 316, 317, 318, 319, 320, 322, 519, 556, 555, 559, 562, 553, 557 and 558, and also 690, 693, 416, 777, 686, XII-8, 697, 694, 695, 683, 592, 575, 743, 115, 659, 269, 671, 283, 267, 561, 583, 581, 582, 677, 678, 587, 588, 589, 679, 688, 680, 681 and 682, and furthermore 824, 807, 786, 785, 806, 799, 834, 836, 840, 841, 816, 817, 818, 788, 793, 794, 809, 810, 811, 826, 870, 867, 790, 848 show, at an active compound concentration of 500 ppm, an efficacy of 70% or more.

### Example F

Pyrenophora teres test (barley)/protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are sprayed with a conidia suspension of Pyrenophora teres. The plants remain in an incubation cabin at 20° C. and 100% relative atmospheric humidity for 48 hours. The plants are then placed in a greenhouse at a temperature of about 20° C. and a relative atmospheric humidity of about 80%. Evaluation is carried out 8 days after the inoculation. 0% means an efficacy which corresponds to that of the control, whereas an efficacy of 100% means that no infection is observed.

In this test, the compounds according to the invention 73, 141, 502, 9, 188, 1, 12, 16, 72, 14, 115, 88, 90, 91, 116, 112, 118, 164, 167, 180, 179, 239, 240, 311, 301, 300, 225, 224, 235, 236, 276, 281, 303, 304, 312, 15, 11, 18, 62, 269, 270, 289, 67, 75, 70, 117, 181, 204, 217, 216, 219, 254, 295, 294, 296, 297, 298 and 213, and also XII-4, 672, 690, 359, 364, 370, 372, 420, 460, 474, 487, 491, 499, 501, 243, 244, 570, 571, 572, 683, 743, 637, 292, 277, 278, 264, 290, 248, 561, 315, 316, 321, 322, 519, 556, 555, 554, 562, 553, 552, 557, 558, 740, 583, 581, 582, 586, 677, 678, 587 and 679, and furthermore 784, 821, 829, 820, 699, XII-7, 694, 786, 782, 785, 787, 1, 788, 793, 790, 791, 823, 303, 304, 312, 62, 269, 270, 289, 67, 73, 75, 70, 177, 181, 177, 204, 217, 216, 219, 254, 295, 294 show, at an active compound concentration of 1000 ppm, an efficacy of 70% or more.

### Example G

Myzus test (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration. Discs of chinese cabbage (Brassica pekinensis) which are infested by all stages of the green peach aphid (Myzus persicae) are sprayed with an active compound preparation of the desired concentration. After the desired period of time, the effect in % is determined. 100% means that all aphids have been killed; 0% means that none of the aphids have been killed.

In this test, the compound according to the invention 600 shows (after 5 days), at an active compound concentration of 500 ppm, an efficacy of 100%.

### Example H

Tetranychus test, OP-resistant (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration. Discs of bean leaves (Phaseolus vulgaris) which are infested by all stages of the greenhouse red spidermite (Tetranychus urticae) are sprayed with an active compound preparation of the desired concentration. After the desired period of time, the effect in % is determined. 100% means that all spidermites have been killed; 0% means that none of the spidermites have been killed.

In this test, the compounds according to the invention 1 (after 4 days), 61, 66 (in each case after 5 days) show, at an active compound concentration of 500 ppm, an efficacy of 85% or more.

### Example I

Phaedon test (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration. Discs of chinese cabbage (Brassica pekinensis) are sprayed with an active compound preparation of the desired concentration and, after drying, populated with larvae of the mustard beetle (Phaedon cochleariae). After the desired period of time, the effect in % is determined. 100% means that all beetle larvae have been killed; 0% means that none of the beetle larvae have been killed.

In this test, the compound according to the invention 618 (after 7 days) shows, at an active compound concentration of 500 ppm, an efficacy of 100%.

### Example K

Leptosphaeria nodorum test (wheat)/protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are sprayed with a spore suspension of Leptosphaeria nodorum. The plants remain in an incubation cabin at 20° C. and 100% relative atmospheric humidity for 48 hours. The plants are placed in a greenhouse at a temperature of about 20° C. and a relative atmospheric humidity of 80%. Evaluation is carried out 10 days after the inoculation. 0% means an efficacy which corresponds to that of the control, whereas an efficacy of 100% means that no infection is observed.

In this test, the compounds according to the invention 141, XII-4, 690, 359, 364, 367, 370, 372, 420, 460, 474, 487, 491, 499, 501, 502, 9, 188, 695, 571, 572, 683, 743, 637, 639, 14, 90, 91, 116, 118, 167, 180, 239, 292, 301, 300, 225, 224, 235, 277, 278, 276, 281, 280, 303, 304, 1, 11, 62, 269, 270, 289, 67, 73, 75, 70, 177, 204, 219, 248, 253, 254, 561, 295, 296, 297, 298, 313, 315, 316, 321, 322, 519, 556, 555, 559, 554, 562, 553, 552, 557, 558, 740, 583, 582, 677, 678, 587, 589, 679 and 682, and furthermore 690, 699, XII-7, 694, 786, 782, 785, 787, 570, 788, 793, 790, 823, 311 show, at an active compound concentration of 1000 ppm, an efficacy of 70% or more.

### Example L

### Leptosphaeria Maculans Test (Cereal)/Seed Treatment

The active compounds are applied as liquid dressings, 1 part by weight of the active compound being mixed with 10 parts by weight of a solvent. For each concentration tested, the appropriate volume from the mixture is pipetted. For the dressing, the seed dressing is pipetted onto the seed in a closed glass bottle and the seed is shaken for 3 minutes. At 2×5 grains, the seed is sown into a soil/sand mixture in pots. Perlite colonized by Leptosphaeria maculans is scattered between the seed and then covered with the soil/sand mixture to a height of 3 cm. The pots are initially incubated at 10° C. for 14 days and then cultivated in a greenhouse at a temperature of about 18° C. and 15 hours of light per day for a further 7 days. Evaluation is carried out after about 3 weeks. 0% means an efficacy which corresponds to that of the control, whereas an efficacy of 100% means that no infection is observed.

In this test, the compounds according to the invention 118, 1 and 289 show, at an active compound concentration of 50 g/100 kg of seed, an efficacy of 70% or more.

### Example M

### Septoria tritici Test (Cereal)/Seed Treatment

The active compounds are applied as liquid dressings, 1 part by weight of the active compound being mixed with 10 parts by weight of a solvent. For each concentration tested, the appropriate volume from the mixture is pipetted. For the dressing, the preparation is pipetted onto the seed and the seed is shaken in a closed glass bottle for 3 minutes. At 3×12 grains, the seed is sown into a soil/sand mixture in pots. The pots are initially incubated at 5° C. in the dark for 14 days and then cultivated in a greenhouse at a temperature of about 10° C. and 15 hours of light per day for a further 14 days. The plants are then sprayed with a spore suspension of Septoria tritici and subsequently kept at a temperature of about 15° C. and a relative atmospheric humidity of 80% for 21 days. Evaluation is carried out 10 days after the inoculation. 0% means an efficacy which corresponds to that of the control, whereas an efficacy of 100% means that no infection is observed. In this test, the compounds according to the invention 90, 91, 116 and 562 show, at an active compound concentration of 50 g/100 kg of seed, an efficacy of 70% or more.

### Example N

### Puccinia Recondita Test (Cereal)/Seed Treatment

The active compounds are applied as liquid dressings, 1 part by weight of the active compound being mixed with 10 parts by weight of a solvent. For each concentration tested, the appropriate volume from the mixture is pipetted. For the dressing, the preparation is pipetted onto the seed and the seed is shaken in a closed glass bottle for 3 minutes. At 3×12 grains, the seed is sown into a soil/sand mixture in pots. The pots are initially incubated at 5° C. in the dark for 14 days and then cultivated in a greenhouse at a temperature of about 10° C. and 15 hours of light per day for a further 14 days. The plants are then sprayed with a spore suspension of Puccinia recondita and subsequently placed in an incubation cabin at 18° C. and 100% relative atmospheric humidity for 48 hours. The plants are then placed in a greenhouse at a temperature of about 18° C. and a relative atmospheric humidity of 80%. Evaluation is carried out about 7 days after the inoculation. 0% means an efficacy which corresponds to that of the control, whereas an efficacy of 100% means that no infection is observed. In this test, the compound according to the invention 141 shows, at an active compound concentration of 50 g/100 kg of seed, an efficacy of 70% or more.

1. A method for controlling unwanted microorganisms in crop protection and/or in the protection of materials comprising using a bisoxime of formula (I) 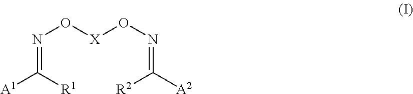
in which
R¹ and R² independently of one another represent hydrogen, halogen, C₁-C₈-alkyl, C₃-C₈-cycloalkyl, C₁-C₈-haloalkyl, OH, C₁-C₈-alkoxy, (C₁-C₆-alkoxy)carbonyl, SH, C₁-C₈-alkylthio, C₁-C₈-alkylsulphinyl, C₁-C₈-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₈-alkyl)amino, (C₁-C₈-alkyl)amino, C₂-C₈-alkenyl, C₂-C₈-alkynyl, in each case optionally substituted phenyl, benzyl, heterocyclyl or hetaryl,
A¹ and A² independently of one another represent one of the cycles below 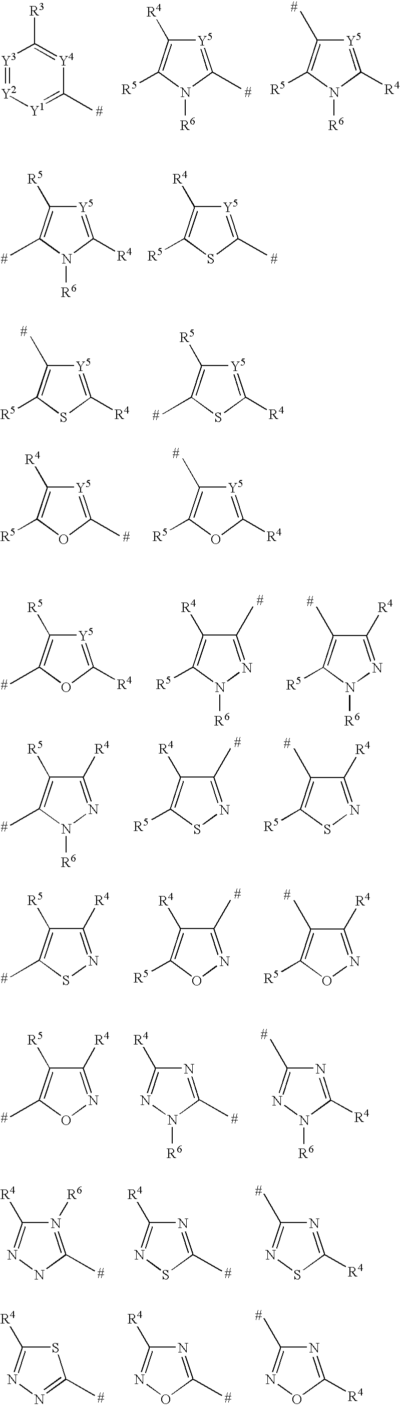 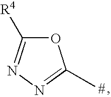
where A¹ and A² do not simultaneously represent optionally substituted phenyl,
Y¹ represents N or CR⁶,
Y² represents N or CR⁷,
Y³ represents N or CR⁸,
Y⁴ represents N or CR⁹,
Y⁵ represents N or CR¹⁰,
R³, R⁴, R⁵, R⁶, R⁷, R⁸, R⁹ and R¹⁰ independently of one another represent hydrogen, halogen, cyano, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, phenyl-(di-C₁-C₄-alkyl)-silyl, substituted or unsubstituted phenyl or hetaryl, substituted or unsubstituted benzyl or —CH₂-hetaryl,
moreover represent substituted C₂-C₈-alkenyl or C₂-C₈-alkynyl,
moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together with the carbon atoms to which they are attached form an unsubstituted or substituted unsaturated or fully or partially saturated heterocyclic or carbocyclic 5- to 7-membered ring,
R¹¹ represents hydrogen, unsubstituted or substituted C₁-C₈-alkyl, C₂-C₈-alkenyl, C₂-C₈-alkynyl, C₁-C₈-haloalkyl, C₃-C₈-cycloalkyl, tri-C₁-C₄-alkyl-silyl, in each case substituted or unsubstituted aryl, —CH₂-aryl, hetaryl or —CH₂-hetaryl,
moreover represents C₃-C₈-cycloalkyl-C₁-C₄-alkyl,
where, if two radicals R¹¹ are attached to a carbon or nitrogen atom, these radicals R¹¹ can be identical or different,
moreover, if two radicals R¹¹ are attached to a nitrogen atom, these two radicals R¹¹ together with the nitrogen atom to which they are attached form a 3- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
moreover the two radicals R¹¹ in the grouping NR¹¹COR¹¹ together with this grouping to which they are attached form a 4- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
R¹² represents hydrogen, unsubstituted or substituted C₁-C₈-alkyl, C₁-C₈-haloalkyl, —(CH₂)ₙOR¹¹, tri-C₁-C₄-alkyl-silyl,
m represents the number 1, 2, 3, 4, 5, 6, 7 or 8,
n represents the number 1, 2, 3 or 4,
X represents one of the groupings X-1 to X-10 below 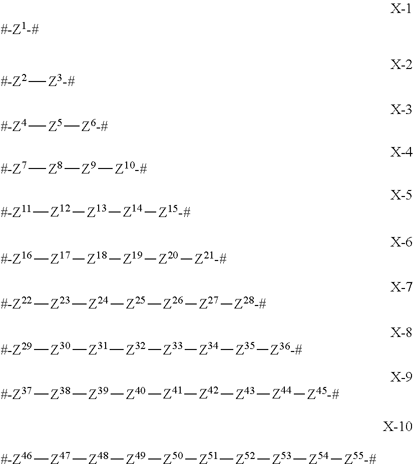
in which
Z¹ represents CR¹³R¹⁴ or SiR¹⁵R¹⁶, where in each case two radicals R¹³, R¹⁴ or R¹⁵, R¹⁶ together with the carbon or silicon atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z² and Z³ independently of one another represent CR¹³R¹⁴ or SiR¹⁵R¹⁶, but two silicon atoms are not adjacent, where in each case two radicals R¹³, R¹⁴ or R¹⁵, R¹⁶ or R¹³, R¹⁵ together with the carbon or silicon atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z⁴ and Z⁶ independently of one another represent CR¹³R¹⁴ or SiR¹⁵R¹⁶ and Z⁵ represents CR¹³R¹⁴, SiR¹⁵R¹⁶, O or NR¹⁷, but two silicon atoms are not adjacent, or in each case two adjacent radicals Z⁴, Z⁵ or Z⁵, Z⁶ represent a group selected from the group consisting of —CR¹³═CR¹⁴—, —CR¹³═N—, —N═CR¹³— or —C≡C—, where X-3 may contain at most one of those groups,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z⁷ and Z¹⁰ independently of one another represent CR¹³R¹⁴ or SiR¹⁵R¹⁶ and Z⁸ and Z⁹ independently of one another represent CR¹³R¹⁴, SiR¹⁵R¹⁶, O or NR¹⁷, but two silicon or two oxygen atoms are not adjacent, or in each case two adjacent radicals Z⁷, Z⁸ or Z⁸, Z⁹ or Z⁹, Z¹⁰ represent a group selected from the group consisting of —CR¹³═CR¹⁴—, —CR¹³═N—, —N═CR¹³— or —C≡C—, where X-4 may contain at most one of those groups, where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z¹¹ and Z¹⁵ independently of one another represent CR¹³R¹⁴ or SiR¹⁵R¹⁶ and Z¹², Z¹³ and Z¹⁴ independently of one another represent CR¹³R¹⁴, SiR¹⁵R¹⁶, O or NR¹⁷, but two silicon or two oxygen atoms are not adjacent, or in each case two adjacent radicals Z¹¹, Z¹² or Z¹², Z¹³ or Z¹³, Z¹⁴ or Z¹⁴, Z¹⁵ represent a group selected from the group consisting of —CR¹³═CR¹⁴—, —CR¹³═N—, —N═CR¹³— or —C≡C—, where X-5 may contain not more than 2 of those groups, which for their part must not be cumulated,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z¹⁶ and Z²¹ independently of one another represent CR¹³R¹⁴ or SiR¹⁵R¹⁶ and Z¹⁷, Z¹⁸, Z¹⁹ and Z²⁰ independently of one another represent CR¹³R¹⁴, SiR¹⁵R¹⁶, O or NR¹⁷, but two silicon or two oxygen atoms are not adjacent, or in each case two adjacent radicals Z¹⁶, Z¹⁷ or Z¹⁷, Z¹⁸ or Z¹⁸, Z¹⁹ or Z¹⁹, Z²⁰ or Z²⁰, Z²¹ represent a group selected from the group consisting of —CR¹³═CR¹⁴—, —CR¹³═N—, —N═CR¹³— or —C≡C—, where X-6 may not contain more than 2 of those groups, which for their part must not be cumulated,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z²² and Z²⁸ independently of one another represent CR¹³R¹⁴ or SiR¹⁵R¹⁶ and Z²³, Z²⁴, Z²⁵, Z²⁶ and Z²⁷ independently of one another represent CR¹³R¹⁴, SiR¹⁵R¹⁶, O or NR¹⁷, but two silicon or two oxygen atoms are not adjacent, or in each case two adjacent radicals Z²², Z²³ or Z²³, Z²⁴ or Z²⁴, Z²⁵ or Z²⁵, Z²⁶ or Z²⁶, Z²⁷ or Z²⁷, Z²⁸ represent a group selected from the group consisting of —CR¹³═CR¹⁴—, —CR¹³═N—, —N═CR¹³— or —C≡C—, where X-7 may not contain more than 2 of those groups, which for their part must not be cumulated,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z²⁹ and Z³⁶ independently of one another represent CR¹³R¹⁴ or SiR¹⁵R¹⁶ and Z³⁰, Z³¹, Z³², Z³³, Z³⁴ and Z³⁵ independently of one another represent CR¹³R¹⁴, SiR¹⁵R¹⁶, O or NR¹⁷, but two silicon or two oxygen atoms are not adjacent, or in each case two adjacent radicals Z²⁹, Z³⁰ or Z³⁰, Z³¹ or Z³¹, Z³² or Z³², Z³³ or Z³³, Z³⁴ or Z³⁴, Z³⁵ or Z³⁵, Z³⁶ represent a group selected from the group consisting of —CR¹³═CR¹⁴—, —CR¹³═N—, —N═CR¹³— or —C≡C—, where X-8 may not contain more than 2 of those groups, which for their part must not be cumulated,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z³⁷ and Z⁴⁵ independently of one another represent CR¹³R¹⁴ or SiR¹⁵R¹⁶ and Z³⁸, Z³⁹, Z⁴⁰, Z⁴¹, Z⁴², Z⁴³ and Z⁴⁴ independently of one another represent CR¹³R¹⁴, SiR¹⁵R¹⁶, O or NR¹⁷, but two silicon or two oxygen atoms are not adjacent, or in each case two adjacent radicals Z³⁷, Z³⁸ or Z³⁸, Z³⁹ or Z³⁹, Z⁴⁰ or Z⁴⁰, Z⁴¹ or Z⁴¹, Z⁴² or Z⁴², Z⁴³ or Z⁴³, Z⁴⁴ or Z⁴⁴, Z⁴⁵ represent a group selected from the group consisting of —CR¹³═CR¹⁴—, —CR¹³═N—, —N═CR¹³— or —C≡C—, where X-9 may not contain more than 2 of those groups, which for their part must not be cumulated,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
Z⁴⁶ and Z⁵⁵ independently of one another represent CR¹³R¹⁴ or SiR¹⁵R¹⁶ and Z⁴⁷, Z⁴⁸, Z⁴⁹, Z⁵⁰, Z⁵¹, Z⁵², Z⁵³ and Z⁵⁴ independently of one another represent CR¹³R¹⁴, SiR¹⁵R¹⁶, O or NR¹⁷, but two silicon or two oxygen atoms are not adjacent, or in each case two adjacent radicals Z⁴⁶, Z⁴⁷ or Z⁴⁷, Z⁴⁸ or Z⁴⁸, Z⁴⁹ or Z⁴⁹, Z⁵⁰ or Z⁵⁰, Z⁵¹ or Z⁵¹, Z⁵² or Z⁵², Z⁵³ or Z⁵³, Z⁵⁴ or Z⁵⁴, Z⁵⁵ represent a group selected from the group consisting of —CR¹³═CR¹⁴—, —CR¹³═N—, —N═CR¹³— or —C≡C—, where X-10 may not contain more than 2 of those groups, which for their part must not be cumulated,
where in each case two radicals R¹³, R¹⁴ or R¹⁵, R¹⁶ or R¹³, R¹⁵ or R¹³, R¹⁷ or R¹⁵, R¹⁷ together with the carbon, silicon or nitrogen atom to which they are attached may form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent,
and in which the groupings X-2 to X-10 contain at most one ring which, as ring members, contains either only one of the radicals Z² to Z⁵⁵ (spiro compounds) or two radicals Z² to Z⁵⁵ or three radicals Z² to Z⁵⁵ or four radicals Z² to Z⁵⁵,
R¹³, R¹⁴, R¹⁵, R¹⁶ and R¹⁷ independently of one another represent hydrogen, halogen, cyano, hydroxyl, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², unsubstituted or substituted C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, substituted or unsubstituted phenyl or hetaryl, substituted or unsubstituted benzyl or —CH₂-hetaryl,
R¹³ and R¹⁴ moreover together represent ═CR¹⁸R¹⁹, ═O, ═S or ═NOR²⁰,
R¹⁸, R¹⁹ and R²⁰ independently of one another represent hydrogen, unsubstituted or substituted C₁-C₈-alkyl, C₁-C₈-haloalkyl, C₂-C₈-alkenyl, C₂-C₈-alkynyl, C₃-C₈-cycloalkyl, substituted or unsubstituted phenyl or hetaryl,
R¹⁸ and R¹⁹ moreover together with the carbon atom to which they are attached form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent.

2. A method according to claim 1, where
R¹ and R² independently of one another represent hydrogen, chlorine, bromine, iodine, C₁-C₆-alkyl, C₃-C₆-cycloalkyl, C₁-C₆-haloalkyl having 1 to 13 fluorine, chlorine and/or bromine atoms, OH, C₁-C₆-alkoxy, (C₁-C₄-alkoxy)carbonyl, SH, C₁-C₆-alkylthio, C₁-C₆-alkylsulphinyl, C₁-C₆-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)-sulphonyl(C₁-C₄-alkyl), amino, di(C₁-C₆-alkyl)amine, (C₁-C₆-alkyl)amine, C₂-C₆-alkenyl, C₂-C₆-alkynyl, represent phenyl, benzyl, heterocyclyl or hetaryl, each of which is optionally substituted by halogen, cyano, C₁-C₄-haloalkyl having 1 to 9 fluorine, chlorine and/or bromine atoms, C₁-C₄-alkyl, C₂-C₄-alkenyl, C₂-C₄-alkynyl, C₁-C₄-alkoxy,
A¹ and A² independently of one another represent one of the cycles A-1 to A-43 below: 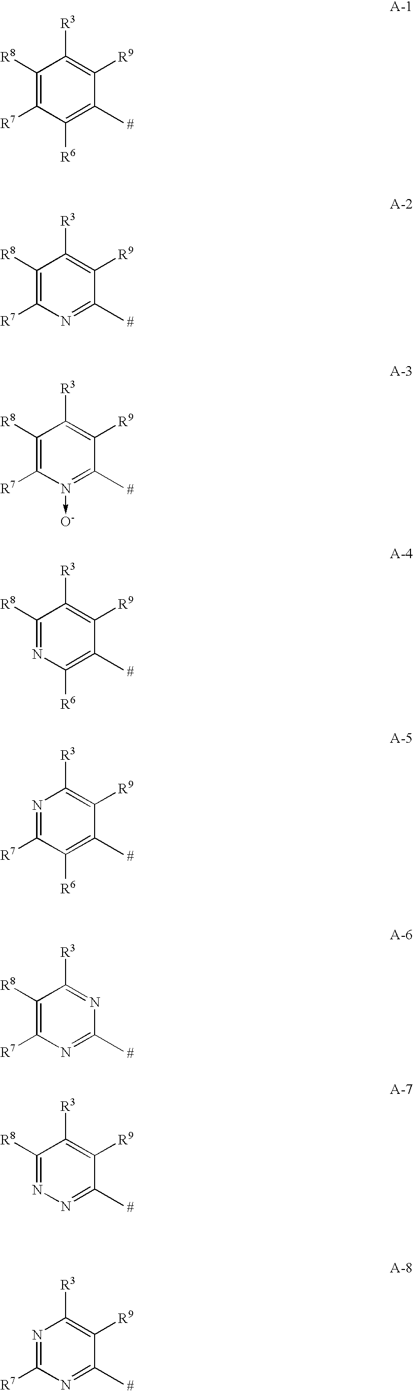 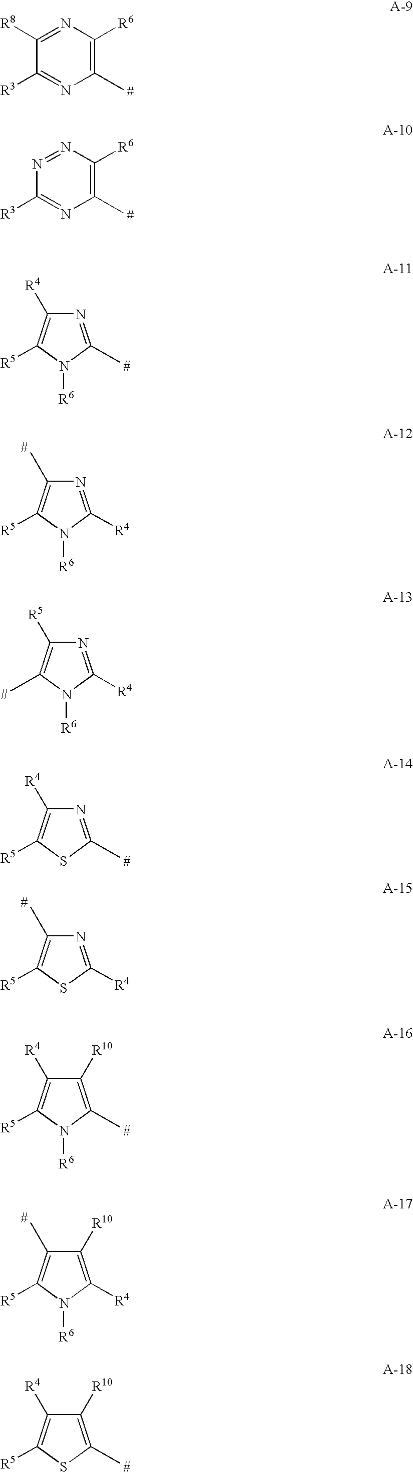 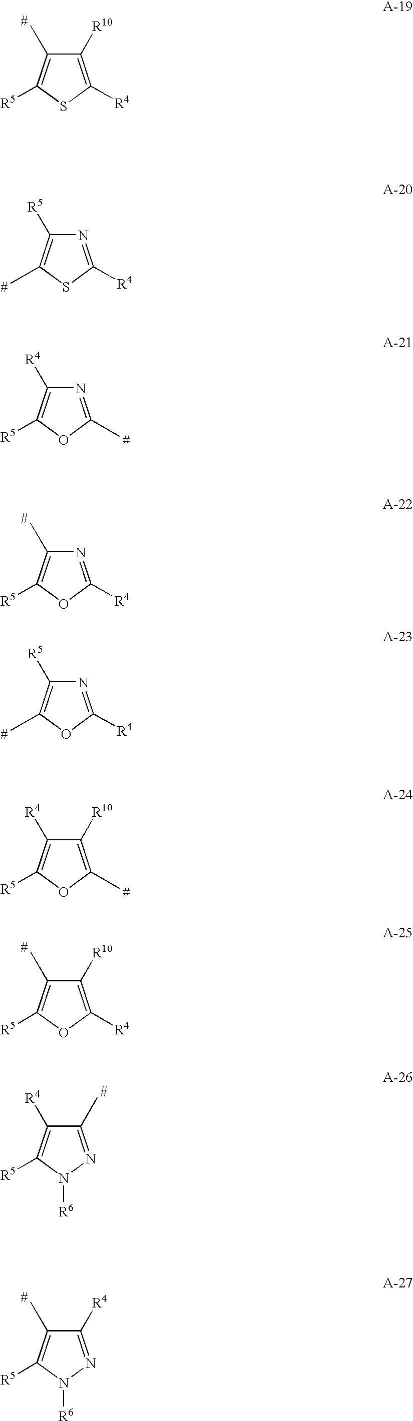 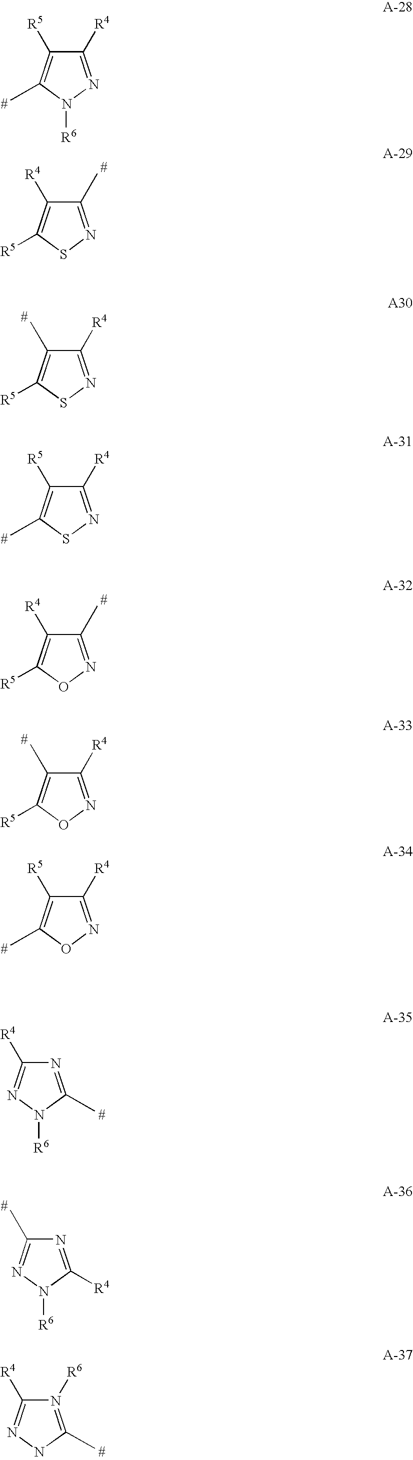 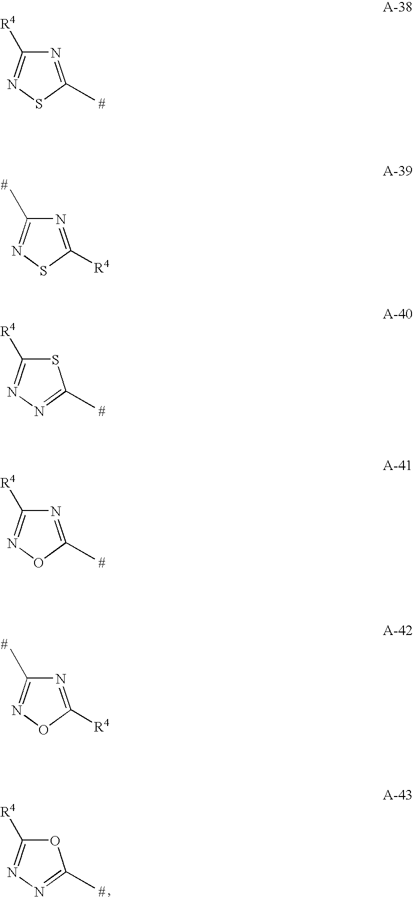
where A¹ and A² do not simultaneously represent the cycle A-1,
R³, R⁴, R⁵, R⁶, R⁷, R⁸, R⁹ and R¹⁰ independently of one another represent hydrogen, fluorine, chlorine, bromine, iodine, cyano, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², C₁-C₆-alkyl, C₂-C₆-alkenyl, C₂-C₆-alkynyl, C₁-C₆-haloalkyl having in each case 1 to 13 fluorine, chlorine and/or bromine atoms, C₃-C₆-cycloalkyl, C₃-C₆-cycloalkenyl, tri-C₁-C₄-alkyl-silyl, phenyl-(di-C₁-C₄-alkyl)-silyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl or pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms,
R³, R⁴, R⁵, R⁶, R⁷, R⁸, R⁹ and R¹⁰ moreover independently of one another represent C₁-C₄-alkoxy-C₂-C₆-alkenyl, C₁-C₄-alkoxy-C₂-C₆-alkynyl, (C₁-C₄-alkyl)carbonyloxy-C₂-C₆-alkenyl or (C₁-C₄-alkyl)carbonyloxy-C₂-C₆-alkynyl,
moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together form an unsubstituted or substituted unsaturated or fully or partially saturated heterocyclic or carbocyclic 5- to 7-membered ring,
R¹¹ represents hydrogen, C₁-C₆-alkyl, vinyl, allyl, ethynyl, propargyl, C₁-C₆-haloalkyl having in each case 1 to 13 fluorine, chlorine and/or bromine atoms, C₃-C₆-cycloalkyl, tri-C₁-C₄-alkyl-silyl, hydroxy-C₁-C₄-alkyl, C₁-C₄-alkoxy-C₁-C₄-alkyl, (C₁-C₄-alkyl)thio(C₁-C₄-alkyl), phenyl, benzyl, pyridinyl, pyridinylmethyl, represents phenyl or benzyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms, where, if two radicals R¹¹ are attached to a carbon or nitrogen atom, these radicals R¹¹ may be identical or different,
moreover, if two radicals R¹¹ are attached to a nitrogen atom, these two radicals R¹¹ together with the nitrogen atom to which they are attached form a 3- to 6-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
moreover the two radicals R¹¹ in the grouping NR¹¹COR¹¹ together with this grouping to which they are attached form a 4- to 6-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
R¹² represents hydrogen, C₁-C₆-alkyl, C₁-C₆-haloalkyl having in each case 1 to 13 fluorine, chlorine and/or bromine atoms, —(CH₂)ₙOR¹¹, tri-C₁-C₄-alkyl-silyl,
m represents the number 1, 2, 3, 4, 5 or 6,
n represents the number 1, 2, 3 or 4,
X represents one of the groupings X-1, X-2, X-3, X-4, X-5, X-6, X-7 or X-10,
Z¹ represents CR¹³R¹⁴ or SiR¹⁵R¹⁶, or represents in each case unsubstituted or substituted oxetane-3,3-diyl, cyclopropane-1,1-diyl, cyclobutane-1,1-diyl, cyclopentane-1,1-diyl, cyclohexane-1,1-diyl,
Z² and Z³ independently of one another represent CR¹³R¹⁴ or SiR¹⁵R¹⁶, but where two silicon atoms are not adjacent, or represent in each case unsubstituted or substituted oxetane-3,3-diyl, cyclopropane-1,1-diyl, cyclobutane-1,1-diyl, cyclopentane-1,1-diyl, cyclohexane-1,1-diyl, where X-2 contains at most one spiro group,
Z⁴, Z⁵ and Z⁶ independently of one another represent CR¹³R¹⁴ or SiR¹⁵R¹⁶, but where two silicon atoms are not adjacent, or Z⁴ and Z⁶ independently of one another represent in each case unsubstituted or substituted oxetane-3,3-diyl, cyclopropane-1,1-diyl, cyclo-butane-1,1-diyl, cyclopentane-1,1-diyl, cyclohexane-1,1-diyl, 4,5-dihydroisoxazole-5,5-diyl, and Z⁵ independently thereof represents in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydro-isoxazole-5,5-diyl or Z⁵ moreover represents 1,3-oxazolidine-4,4-diyl, where X-3 contains at most one spiro group, or two adjacent groups Z⁴, Z⁵ or Z⁵, Z⁶ represent —CR¹³═CR¹⁴—, —CR¹³═N—, —N═CR¹³— or —C≡C— or pyridinediyl, or two adjacent groups Z⁴, Z⁵ or Z⁵, Z⁶ moreover represent cyclopentane or cyclohexane, or X-3 represents optionally halogen- or C₁-C₃-alkyl-substituted pyrimidinediyl, or X-3 moreover represents cyclopentane-1,3-diyl or cyclohexane-1,3-diyl, where X-3 may contain at most one such group or such cycle, and, if Z⁴ and Z⁶ independently of one another represent C═O or C═S, Z⁵ represents CR¹³R¹⁴ or NR¹⁷,
Z⁷, Z⁸, Z⁹ and Z¹⁰ independently of one another represent CR¹³R¹⁴ or SiR¹⁵R¹⁶, but where two silicon or two oxygen atoms are not adjacent, or Z⁷ and Z¹⁰ independently of one another represent in each case unsubstituted or substituted oxetane-3,3-diyl, cyclo-propane-1,1-diyl, cyclobutane-1,1-diyl, cyclopentane-1,1-diyl, cyclohexane-1,1-diyl, 4,5-dihydroisoxazole-5,5-diyl, and Z⁸ and Z⁹ independently of one another represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-4 contains at most one spiro group, or two adjacent groups Z⁷, Z⁸ or Z⁸, Z⁹ or Z⁹, Z¹⁰ represent —CR¹³═CR¹⁴—, —CR¹³═N—, —N═CR¹³— or —C≡C— or represent in each case unsubstituted or substituted 1,3-dioxolane-4,5-diyl, 1,4-dioxane-2,3-diyl, oxirane-2,3-diyl, 1,2-phenylene, 2,3-pyridinediyl or 3,4-pyridinediyl, where X-4 may contain at most one such group or such cycle, and, if Z⁷ and Z¹⁰ independently of one another represent C═O or C═S, Z⁸ and Z⁹ represent CR¹³R¹⁴ or NR¹⁷,
Z¹¹, Z¹², Z¹⁴ and Z¹⁵ independently of one another represent CR¹³R¹⁴ or SiR¹⁵R¹⁶ and Z¹³ independently thereof represents CR¹³R¹⁴, SiR¹⁵R¹⁶, O or NR¹⁷, but where two silicon or two oxygen atoms are not adjacent, or Z¹¹ and Z¹⁵ independently of one another represent in each case unsubstituted or substituted oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z¹², Z¹³ and Z¹⁴ independently of one another represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-5 contains at most one spiro group, or in each case two adjacent groups Z¹¹, Z¹² or Z¹², Z¹³ or Z¹³, Z¹⁴ or Z¹⁴, Z¹⁵ represent —CR¹³═CR¹⁴—, —CR¹³═N—, —N═CR¹³— or —C≡C— or represent in each case unsubstituted or substituted 1,3-dioxolane-4,5-diyl, 1,4-dioxane-2,3-diyl, oxirane-2,3-diyl, or Z¹², Z¹³ and Z¹⁴ together represent in each case substituted or unsubstituted 1,3-phenylene, pyridine-2,6-diyl, pyridine-2,4-diyl or pyridine-3,5-diyl, where X-5 may contain at most one such group or one such cycle, and, if Z¹¹ and Z¹⁵ independently of one another represent C═O or C═S, Z¹² and Z¹⁴ represent CR¹³R¹⁴ or NR¹⁷,
Z¹⁶, Z¹⁷, Z²⁰ and Z²¹ independently of one another represent CR¹³R¹⁴ or SiR¹⁵R¹⁶ and Z¹⁸ and Z¹⁹ independently of one another represent CR¹³R¹⁴, SiR¹⁵R¹⁶, O or NR¹⁷, but where two silicon or two oxygen atoms are not adjacent, or Z¹⁶ and Z²¹ independently of one another represent in each case unsubstituted or substituted, oxetane-3,3-diyl, cyclopropane-1,1-diyl, cyclobutane-1,1-diyl, cyclopentane-1,1-diyl, cyclohexane-1,1-diyl, and Z¹⁸, Z¹⁷, Z¹⁹ and Z²⁰ independently of one another represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-6 contains at most one spiro group, or in each case two adjacent radicals Z¹⁶, Z¹⁷ or Z¹⁷, Z¹⁸ or Z¹⁸, Z¹⁹ or Z¹⁹, Z²⁰ or Z²⁰, Z²¹ represent —CR¹³═CR¹⁴—, —CR¹³═N—, —N═CR¹³— or —C≡C—, where X-6 may not contain more than 2 of such groups, which for their part must not be cumulated, or represent in each case unsubstituted or substituted 1,3-dioxolane-4,5-diyl, 1,4-dioxane-2,3-diyl or oxirane-2,3-diyl, or Z¹⁶, Z¹⁷ and Z¹⁸ or Z¹⁷, Z¹⁸ and Z¹⁹ together represent in each case substituted or unsubstituted 1,4-phenylene or pyridine-2,5-diyl, where X-6 may contain at most one such cycle, and, if Z¹⁶ and Z²¹ independently of one another represent C═O or C═S, Z¹⁷ and Z²⁰ represent CR¹³R¹⁴ or NR¹⁷,
Z²², Z²³, Z²⁷ and Z²⁸ independently of one another represent CR¹³R¹⁴ or SiR¹⁵R¹⁶ and Z²⁴, Z²⁵, and Z²⁶ independently of one another represent CR¹³R¹⁴, SiR¹⁵R¹⁶, O or NR¹⁷, but where two silicon or two oxygen atoms are not adjacent, or Z²² and Z²⁸ independently of one another represent in each case unsubstituted or substituted oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z²³, Z²⁴, Z²⁵, Z²⁶ and Z²⁷ independently of one another represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-7 contains at most one spiro group, or in each case two adjacent radicals Z²², Z²³ or Z²³, Z²⁴ or Z²⁴, Z²⁵ or Z²⁵, Z²⁶ or Z²⁶, Z²⁷ or Z²⁷, Z²⁸ represent —CR¹³═CR¹⁴—, —CR¹³═N—, —N═CR¹³— or —C≡C—, where X-7 may not contain more than 2 of such groups, which for their part must not be cumulated, or represent in each case unsubstituted or substituted 1,3-dioxolane-4,5-diyl, 1,4-dioxane-2,3-diyl or oxirane-2,3-diyl, where X-7 may contain at most one such cycle, and, if Z²² and Z²⁸ independently of one another represent C═O or C═S, Z²³ and Z²⁷ represent CR¹³R¹⁴ or NR¹⁷,
Z⁴⁶, Z⁴⁷, Z⁵⁴ and Z⁵⁵ independently of one another represent CR¹³R¹⁴ or SiR¹⁵R¹⁶ and Z⁴⁸, Z⁴⁹, Z⁵⁰, Z⁵¹, Z⁵² and Z⁵³ independently of one another represent CR¹³R¹⁴, SiR¹⁵R¹⁶, O or NR¹⁷, but where two silicon or two oxygen atoms are not adjacent, or Z⁴⁶ and Z⁵⁵ independently of one another represent in each case unsubstituted or substituted oxetane-3,3-diyl, cyclopropane-1,1-diyl, cyclobutane-1,1-diyl, cyclopentane-1,1-diyl, cyclohexane-1,1-diyl, or Z⁴⁷, Z⁴⁸, Z⁴⁹, Z⁵⁰, Z⁵¹, Z⁵², Z⁵³, and Z⁵⁴ independently of one another represent in each case unsubstituted or substituted oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, 4,5-dihydroisoxazole-5,5-diyl, where X-10 contains at most one spiro group, or in each case two adjacent radicals Z⁴⁶, Z⁴⁷ or Z⁴⁷, Z⁴⁸ or Z⁴⁸, Z⁴⁹ or Z⁴⁹, Z⁵⁰ or Z⁵⁰, Z⁵¹ or Z⁵¹, Z⁵² or Z⁵², Z⁵³ or Z⁵³, Z⁵⁴ or Z⁵⁴, Z⁵⁵ represent —CR¹³═CR¹⁴—, —CR¹³═N—, —N═CR¹³— or —C≡C—, where X-10 may not contain more than 2 of such groups, which for their part must not be cumulated, or represent in each case unsubstituted or substituted 1,3-dioxolane-4,5-diyl, 1,4-dioxane-2,3-diyl or oxirane-2,3-diyl, where X-10 may contain at most one such cycle, and, if Z⁴⁶ and Z⁵⁵ independently of one another represent C═O or C═S, Z⁴⁷ and Z⁵⁴ represent CR¹³R¹⁴ or NR¹⁷,
R¹³, R¹⁴, R¹⁵, R¹⁶ and R¹⁷ independently of one another represent hydrogen, fluorine, chlorine, bromine, iodine, cyano, hydroxyl, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², C₁-C₆-alkyl, C₂-C₆-alkenyl, C₂-C₆-alkynyl, C₁-C₆-haloalkyl having in each case 1 to 13 fluorine, chlorine and/or bromine atoms, C₃-C₆-cycloalkyl, C₃-C₆-cycloalkenyl, tri-C₁-C₄-alkyl-silyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms,
R¹³ and R¹⁴ moreover together represent ═CR¹⁸R¹⁹, ═O, ═S, ═NOR²⁰,
R¹⁸, R¹⁹ and R²⁰ independently of one another represent hydrogen, C₁-C₆-alkyl, C₁-C₆-haloalkyl having 1 to 13 fluorine, chlorine and/or bromine atoms, C₂-C₆-alkenyl, C₂-C₆-alkynyl, C₃-C₆-cycloalkyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms,
R¹⁸ and R¹⁹ moreover together with the carbon atom to which they are attached form an unsubstituted or substituted unsaturated or fully or partially saturated heterocyclic or carbocyclic 3- to 8-membered ring which, depending on the ring size, may contain up to 3 further nitrogen atoms or alternatively up to 2 further heteroatoms selected from the group consisting of N, O and S, where two oxygen atoms are not adjacent.

3. A method according to claim 2, where
R¹ and R² independently of one another represent hydrogen, chlorine, bromine, C₁-C₄-alkyl, cyclopropyl, cyclobutyl, cyclopentyl, cyclohexyl, C₁-C₄-haloalkyl having 1 to 9 fluorine, chlorine and/or bromine atoms, OH, C₁-C₄-alkoxy, (C₁-C₄-alkoxy)carbonyl, SH, C₁-C₄-alkylthio, C₁-C₄-alkylsulphinyl, C₁-C₄-alkylsulphonyl, C₁-C₂-alkoxy-C₁-C₂-alkyl, (C₁-C₂-alkyl)thio(C₁-C₂-alkyl), (C₁-C₂-alkyl)sulphinyl(C₁-C₂-alkyl), (C₁-C₂-alkyl)sulphonyl(C₁-C₂-alkyl), amino, di(C₁-C₄-alkyl)amine, (C₁-C₄-alkyl)amine, C₂-C₄-alkenyl, C₂-C₄-alkynyl, represent phenyl, benzyl, thienyl, piperazinyl or morpholinyl, each of which is optionally substituted by fluorine, chlorine, bromine, cyano, trifluoromethyl, difluoromethyl, trichloromethyl, dichloromethyl, C₁-C₄-alkyl, C₁-C₄-alkoxy,
A¹ and A² independently of one another represent one of the cycles A-1, A-2, A-4, A-5, A-6, A-7, A-8, A-9, A-10, A-11, A-12, A-13, A-14, A-15, A-16, A-17, A-18, A-19, A-20, A-21, A-22, A-23, A-24, A-25, A-26, A-27, A-28, A-29, A-30, A-31, A-32, A-33 or A-34 where A¹ and A² do not simultaneously represent the cycle A-1,
R³, R⁴, R⁵, R⁶, R⁷, R⁸, R⁹ and R¹⁰ independently of one another represent hydrogen, fluorine, chlorine, bromine, cyano, hydroxyl, methoxy, ethoxy, n-propoxy, i-propoxy, n-, i-, s-, t-butoxy, —O(CH₂)₂OH, —O(CH₂)₂OCH₃, —O(CH₂)₃OH, —O(CH₂)₃OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-Pr, SO-iPr, SO-Bu, SO-secBu, SO-isoBu, SO-tBu, SO₂-Me, SO₂-Et, SO₂—Pr, SO₂-iPr, SO₂-Bu, SO₂-secBu, SO₂-isoBu, SO₂-tBu, SONHMe, SONMe₂, SONHEt, SONEt₂, SONHPr, SONPr₂, SONHBu, SONBu₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, CH₂OH, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂CO₂Me, (CH₂)₂CO₂Me, (CH₂)₃CO₂Me, CH₂CO₂Et, (CH₂)₂CO₂Et, (CH₂)₃CO₂Et, CH₂CO₂Pr, (CH₂)₂CO₂Pr, (CH₂)₃CO₂Pr, CH₂CO₂iPr, (CH₂)₂CO₂iPr, (CH₂)₃CO₂iPr, CH₂CO₂tBu, (CH₂)₂CO₂tBu, (CH₂)₃CO₂tBu, CH₂CO₂(CH₂)₂OH CH₂CO₂(CH₂)₂OCH₃, CH₂CO₂(CH₂)₃OH, CH₂CO₂(CH₂)₃OCH₃, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, cyclohexyl, phenyl, benzyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one,
R³, R⁴, R⁵, R⁶, R⁷, R⁸, R⁹ and R¹⁰ moreover independently of one another represent iodine, vinyl, allyl, prop-1-en-1-yl, ethynyl, propargyl, vinyloxy, allyloxy, propargyloxy, methoxyprop-1-en-1-yl, methoxyprop-1-yn-1-yl, acetyloxyprop-1-en-1-yl, acetyloxyprop-1-yn-1-yl and pyrrolidin-1-yl,
moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together form an unsubstituted or substituted unsaturated or fully or partially saturated heterocyclic or carbocyclic 5- to 7-membered ring,
X represents one of the groupings X-1, X-3, X-5, X-6 or X-10,
Z¹ represents CR¹³R¹⁴ or SiR¹⁵R¹⁶, or represents oxetane-3,3-diyl, oxirane-2,2-diyl, cyclopropane-1,1-diyl, cyclobutane-1,1-diyl, cyclopentane-1,1-diyl, cyclohexane-1,1-diyl,
Z⁴, Z⁵ and Z⁶ independently of one another represent CR¹³R¹⁴ or SiR¹⁵R¹⁶, but where two silicon atoms are not adjacent, or Z⁴ and Z⁶ independently of one another represent oxetane-3,3-diyl, cyclopropane-1,1-diyl, cyclobutane-1,1-diyl, cyclopentane-1,1-diyl, cyclohexane-1,1-diyl, 4,5-dihydroisoxazole-5,5-diyl, and Z⁵ independently thereof represents oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represents 4,5-dihydroisoxazole-5,5-diyl, which is optionally mono- or polysubstituted by identical or different substituents from the group consisting of C₁-C₄-alkyl, C₂-C₆-alkenyl, phenyl and halophenyl, where X-3 contains at most one spiro group, or two adjacent groups Z⁴, Z⁵ or Z⁵, Z⁶ represent —CR¹³═CR¹⁴—, —CR¹³═N—, —N═CR¹³— or —C≡C— or pyridine-2,3-diyl, or X-3 represents pyrimidine-4,6-diyl which is optionally substituted in the 5-position by fluorine, chlorine or methyl, where X-3 may contain at most one such group or such cycle, and, if Z⁴ and Z⁶ independently of one another represent C═O or C═S, Z⁵ represents CR¹³R¹⁴ or NR¹⁷,
Z¹¹, Z¹², Z¹⁴ and Z¹⁵ independently of one another represent CR¹³R¹⁴ or SiR¹⁵R¹⁶ and Z¹³ independently thereof represents CR¹³R¹⁴, SiR¹⁵R¹⁶, O or NR¹⁷, but where two silicon or two oxygen atoms are not adjacent, or Z¹¹ and Z¹⁵ independently of one another represent oxetane-3,3-diyl, cyclopropane-1,1-diyl, cyclobutane-1,1-diyl, cyclopentane-1,1-diyl, cyclohexane-1,1-diyl, and Z¹², Z¹³ and Z¹⁴ independently of one another represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by C₁-C₄-alkyl, C₂-C₆-alkenyl, phenyl or halophenyl, where X-5 contains at most one spiro group, or in each case two adjacent groups Z¹¹, Z¹² or Z¹², Z¹³ or Z¹³, Z¹⁴ or Z¹⁴, Z¹⁵ represent —CR¹³═CR¹⁴—, —CR¹³═N—, —N═CR¹³ or —C≡C— or represent in each case unsubstituted or substituted 1,3-dioxolane-4,5-diyl, 1,4-dioxane-2,3-diyl or oxirane-2,3-diyl, or Z¹², Z¹³ and Z¹⁴ together represent 1,3-phenylene or pyridine-2,6-diyl, each of which is mono- or polysubstituted by identical or different substituents from the group consisting of fluorine, chlorine, methyl and trifluoromethyl, where X-5 may contain at most one such group or such cycle, and, if Z¹¹ and Z¹⁵ independently of one another represent C═O or C═S, Z¹² and Z¹⁴ represent CR¹³R¹⁴ or NR¹⁷,
Z¹⁶, Z¹⁷, Z²⁰ and Z²¹ independently of one another represent CR¹³R¹⁴ or SiR¹⁵R¹⁶ and Z¹⁸ and Z¹⁹ independently of one another represent CR¹³R¹⁴, SiR¹⁵R¹⁶, O or NR¹⁷, but where two silicon or two oxygen atoms are not adjacent, or Z¹⁶ and Z²¹ independently of one another represent oxetane-3,3-diyl, cyclopropane-1,1-diyl, cyclobutane-1,1-diyl, cyclopentane-1,1-diyl, cyclohexane-1,1-diyl, and Z¹⁷, Z¹⁸, Z¹⁹ and Z²⁰ independently of one another represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by C₁-C₄-alkyl, C₂-C₆-alkenyl, phenyl or halophenyl, where X-6 contains at most one spiro group, or in each case two adjacent radicals Z¹⁷, Z¹⁸ or Z¹⁸, Z¹⁹ or Z¹⁹, Z²⁰ represent —CR¹³═CR¹⁴—, —CR¹³═N—, —N═CR¹³— or —C≡C—, where X-6 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, or Z¹⁶, Z¹⁷ and Z¹⁸ or Z¹⁷, Z¹⁸ and Z¹⁹ together represent 1,4-phenylene which is mono- or polysubstituted by identical or different substituents from the group consisting of fluorine, chlorine, methyl and trifluoromethyl, where X-6 may contain at most one such cycle, and, if Z¹⁶ and Z²¹ independently of one another represent C═O or C═S, Z¹⁷ and Z²⁰ represent CR¹³R¹⁴ or NR¹⁷,
Z⁴⁶, Z⁴⁷, Z⁵⁴ and Z⁵⁵ independently of one another represent CR¹³R¹⁴ or SiR¹⁵R¹⁶ and Z⁴⁸, Z⁴⁹, Z⁵⁰, Z⁵¹, Z⁵² and Z⁵³ independently of one another represent CR¹³R¹⁴, SiR¹⁵R¹⁶, O or NR¹⁷, but where two silicon or two oxygen atoms are not adjacent, or Z⁴⁶ and Z⁵⁵ independently of one another represent oxetane-3,3-diyl, cyclopropane-1,1-diyl, cyclobutane-1,1-diyl, cyclopentane-1,1-diyl, cyclohexane-1,1-diyl, or Z⁴⁷, Z⁴⁸, Z⁴⁹, Z⁵⁰, Z⁵¹, Z⁵², Z⁵³ and Z⁵⁴ independently of one another represent oxetane-2,2-diyl, oxetane-3,3-diyl, oxirane-2,2-diyl, cyclopropane-1,1-diyl, cyclobutane-1,1-diyl, cyclopentane-1,1-diyl, cyclohexane-1,1-diyl, or represent 4,5-dihydroisoxazole-5,5-diyl which is optionally mono- or polysubstituted by identical or different substituents from the group consisting of C₁-C₄-alkyl, C₂-C₆-alkenyl, phenyl and halophenyl, where X-10 contains at most one spiro group, or in each case two adjacent radicals Z⁴⁷, Z⁴⁸ Z⁴⁸, Z⁴⁹ or Z⁴⁹, Z⁵⁰ or Z⁵⁰, Z⁵¹ or Z⁵¹, Z⁵² or Z⁵², Z⁵³ or Z⁵³, Z⁵⁴ represent —CR¹³═CR¹⁴—, —CR¹³═N—, —N═CR¹³— or —C≡C—, where X-10 may not contain more than 2 such groups, which for their part must not be cumulated, or represent in each case unsubstituted or substituted 1,3-dioxolane-4,5-diyl, 1,4-dioxane-2,3-diyl or oxirane-2,3-diyl, where X-10 may contain at most one such cycle, and, if Z⁴⁶ and Z⁵⁵ independently of one another represent C═O or C═S, Z⁴⁷ and Z⁵⁴ represent CR¹³R¹⁴ or NR¹⁷,
R¹³, R¹⁴, R¹⁵, R¹⁶ and R¹⁷ independently of one another represent hydrogen, fluorine, chlorine, bromine, hydroxyl, methoxy, ethoxy, n-propoxy, i-propoxy, n-, i-, s-, t-butoxy, —O(CH₂)₂OH, —O(CH₂)₂OCH₃, —O(CH₂)₃OH, —O(CH₂)₃OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-Pr, SO-iPr, SO-Bu, SO-secBu, SO-isoBu, SO-tBu, SO₂-Me, SO₂-Et, SO₂—Pr, SO₂-iPr, SO₂-Bu, SO₂-secBu, SO₂-isoBu, SO₂-tBu, SONHMe, SONMe₂, SONHEt, SONEt₂, SONHPr, SONPr₂, SONHBu, SONBu₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, CH₂OH, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂CO₂Me, (CH₂)₂CO₂Me, (CH₂)₃CO₂Me, CH₂CO₂Et, (CH₂)₂CO₂Et, (CH₂)₃CO₂Et, CH₂CO₂Pr, (CH₂)₂CO₂Pr, (CH₂)₃CO₂Pr, CH₂CO₂iPr, (CH₂)₂CO₂iPr, (CH₂)₃CO₂iPr, CH₂CO₂tBu, (CH₂)₂CO₂tBu, (CH₂)₃CO₂tBu, CH₂CO₂(CH₂)₂OH CH₂CO₂(CH₂)₂OCH₃, CH₂CO₂(CH₂)₃OH, CH₂CO₂(CH₂)₃OCH₃, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, cyclopropyl, cyclobutyl, cyclopentyl, cyclohexyl, 1-cyclohexenyl, 2-cyclohexenyl, 3-cyclohexenyl, phenyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one,
R¹³ and R¹⁴ moreover together represent ═CR¹⁸R¹⁹, ═O, ═S, ═NOR²⁰,
R¹⁸, R¹⁹ and R²⁰ independently of one another represent hydrogen, methyl, ethyl, n-propyl, i-propyl, n-, i-, s-, t-butyl, trifluoromethyl, cyclopropyl, cyclobutyl, cyclopentyl, cyclohexyl, phenyl, benzyl, pyridinyl, pyridinylmethyl, thienyl, thenyl, furyl, furfuryl, pyrrolyl, pyrrolylmethyl, represent phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of fluorine, chlorine, bromine, methyl, ethyl, n-propyl, i-propyl, methoxy, trifluoromethyl.

4. A method according to claim 2, where
R¹ and R² independently of one another represent hydrogen, chlorine, methyl, ethyl, n-propyl, i-propyl, n-, i-, s- or t-butyl, cyclopropyl, cyclopentyl, cyclohexyl, trifluoromethyl, di-fluoromethyl, trichloromethyl, 2,2,2-trifluoroethyl, pentafluoroethyl, heptafluoro-n-propyl, heptafluoro-i-propyl, OH, methoxy, ethoxy, n-propoxy, i-propoxy, n-, i-, s- or t-butoxy, methoxycarbonyl, ethoxycarbonyl, SH, methylthio, ethylthio, n-propylthio, i-propylthio, n-, i-, s- or t-butylthio, methylsulphinyl, ethylsulphinyl, n-propylsulphinyl, i-propylsulphinyl, n-, i-, s- or t-butylsulphinyl, methylsulphonyl, ethylsulphonyl, n-propylsulphonyl, i-propylsulphonyl, n-, i-, s- or t-butylsulphonyl, methoxymethyl, methoxyethyl, ethoxyethyl, ethoxymethyl, methylthiomethyl, methylthioethyl, ethylthioethyl, ethylthiomethyl, methylsulphonylmethyl, methyl-sulphonylethyl, ethylsulphonylethyl, ethylsulphonylmethyl, amino, dimethylamine, diethylamine, diisopropylamine, di-n-propylamine, methylamine, ethylamine, isopropylamine, n-propylamine, vinyl, allyl, butenyl, ethynyl, propargyl, represent phenyl, benzyl, thienyl, piperazinyl or morpholinyl, each of which is optionally substituted by fluorine, chlorine, bromine, cyano, trifluoromethyl, methyl, ethyl, n-propyl, i-propyl, n-, i-, s-, t-butyl, methoxy, ethoxy, n-propoxy, i-propoxy, n-, i-, s-, t-butoxy,
A¹ and A² independently of one another represent one of the cycles A-1, A-2, A-4, A-5, A-6, A-7, A-8, A-9, A-10, A-11, A-14, A-15, A-16, A-18, A-20, A-21, A-24, A-26, A-30 or A-33 where A¹ and A² do not simultaneously represent the cycle A-1,
R³, R⁴, R⁵, R⁶, R⁷, R⁸, R⁹ and R¹⁰ independently of one another represent hydrogen, fluorine, chlorine, bromine, cyano, hydroxyl, methoxy, ethoxy, n-propoxy, i-propoxy, n-, i-, s-, t-butoxy, —O(CH₂)₂OH, —O(CH₂)₂OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-tBu, SO₂-Me, SO₂-Et, SO₂—Pr, SO₂-iPr, SONHMe, SONMe₂, SONHEt, SONEt₂, SONHPr, SONPr₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCO(CH₂)₂OH, NHCO(CH₂)₂OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCOMe, OCOEt, OCOPr, OCOiPr, OCOBu, OCOsecBu, OCOisoBu, OCOtBu, CONHEt, CONEt₂, CONHMe, CONMe₂, CONHPr, CONPr₂, CONHBu, CONHsecBu, CONHisoBu, CONHtBu, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CH₂OH, (CH₂)₂OH, CH₂OMe, (CH₂)₂OMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, CH₂NMe₂, (CH₂)₂NHMe, (CH₂)₂NMe₂, CH₂CO₂Me, (CH₂)₂CO₂Me, CH₂CO₂Et, (CH₂)₂CO₂Et, CH₂CO₂Pr, (CH₂)₂CO₂Pr, CH₂CO₂iPr, (CH₂)₂CO₂iPr, CH₂CO₂tBu, (CH₂)₂CO₂tBu, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, cyclohexyl, phenyl, benzyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one,
R³, R⁴, R⁵, R⁶, R⁷, R⁸, R⁹ and R¹⁰ moreover independently of one another represent iodine, vinyl, allyl, prop-1-en-1-yl, ethynyl, propargyl, vinyloxy, allyloxy, propargyloxy, methoxyprop-1-yn-1-yl, acetyloxyprop-1-yn-1-yl and pyrrolidin-1-yl,
moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together form an unsubstituted or substituted unsaturated or fully or partially saturated heterocyclic or carbocyclic 5- or 6-membered ring,
X represents one of the groupings X-3, X-5, X-6 or X-10,
X-3 represents —CH₂—C(R¹³R¹⁴)—CH₂—, —C(R¹³R¹⁴)—CH₂—C(R¹³R¹⁴)—, —C(R¹³R¹⁴)—C(R¹³R¹⁴)—CH₂—, —C(R¹³R¹⁴)—C(R¹³R¹⁴)—C(R¹³R¹⁴), —C(R¹³R¹⁴)—C(═CH₂)—C(R¹³R¹⁴)—, —C(R¹³R¹⁴)—C(═O)—C(R¹³R¹⁴)—, —[C(R¹³R¹⁴)]₂—C(═O)—, —C(═O)—C(R¹³R¹⁴)—C(═O)—, —C(═O)—NR¹⁷—C(═O)—, —CH₂—Si(R¹⁵R¹⁶)—CH₂— or —CH₂—C(═NOR²⁰)—CH₂—,
X-5 represents —(CH₂)₂—C(R¹³R¹⁴)—(CH₂)₂—, —(CH₂)₂—O—(CH₂)₂—, —C(R¹³R¹⁴)—(CH₂)₃—C(R¹³R¹⁴)—, —(CH₂)₄—C(═O)—, —(CH₂)—C(═O)—(CH₂)₃, —(CH₂)₂—C(═O)—(CH₂)₂—, —C(═O)—(CH₂)₃—C(═O)— or —(CH₂)—₂—Si(R¹⁵R¹⁶)—(CH₂)₂— or one of the groupings below 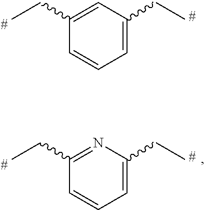
X-6 represents —(CH₂)₃—C(R¹³R¹⁴)—(CH₂)₂—, —C(R¹³R¹⁴)—(CH₂)₄—C(R¹³R¹⁴)—, —(CH₂)₅—C(═O)—, —C(═O)—(CH₂)₄—C(═O)—, —(CH₂)—C(═O)—(CH₂)₄—, —(CH₂)₂—C(═O)—(CH₂)₃— or —(CH₂)₃—Si(R¹⁵R¹⁶)—(CH₂)₂— or represents 1,4-phenylene,
X-10 represents —(CH₂)₅—C(R¹³R¹⁴)—(CH₂)₄—, —(CH₂)₅—O—(CH₂)₄—, —(CH₂)₃—O—(CH₂)₂—O—(CH₂)₃—, —C(R¹³R¹⁴)—(CH₂)₈—C(R¹³R¹⁴)—, —(CH₂)₉—C(═O)—, —C(═O)—(CH₂)₈—C(═O)— or —(CH₂)₅—Si(R¹⁵R¹⁶)—(CH₂)₄—,
R¹³, R¹⁴, R¹⁵, R¹⁶ and R¹⁷ independently of one another represent hydrogen, fluorine, chlorine, bromine, hydroxyl, methoxy, ethoxy, n-propoxy, i-propoxy, n-, i-, s-, t-butoxy, —O(CH₂)₂OH, —O(CH₂)₂OCH₃, trifluoromethoxy, trichloromethoxy, SH, S-Me, S-Et, S-Pr, S-iPr, S-Bu, S-secBu, S-isoBu, S-tBu, SCF₃, SO-Me, SO-Et, SO-tBu, SO₂-Me, SO₂-Et, SO₂—Pr, SO₂-iPr, SONHMe, SONMe₂, SONHEt, SONEt₂, SONHPr, SONPr₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, CO-isoBu, COtBu, COCF₃, NHCO₂Me, NHCO₂Et, NHCO₂Pr, NHCO₂iPr, NHCO₂Bu, NHCO₂secBu, NHCO₂ isoBu, NHCO₂tBu, NHCOMe, NHCOEt, NHCOPr, NHCOiPr, NHCOBu, NHCO(CH₂)₂OH, NHCO(CH₂)₂OCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHiPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCOMe, OCOEt, OCOPr, OCOiPr, OCOBu, OCOsecBu, OCOisoBu, OCOtBu, CONHEt, CONEt₂, CONHMe, CONMe₂, CONHPr, CONPr₂, CONHBu, CONHsecBu, CONHisoBu, CONHtBu, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CH₂OH, (CH₂)₂OH, CH₂OMe, (CH₂)₂OMe, (CH₂)ₘN(R¹²)₂, CH₂NH₂, CH₂NAc₂, CH₂N(COCF₃)₂, CH₂NHAc, CH₂NHCOCF₃, (CH₂)₂NH₂, (CH₂)₃NH₂, CH₂NMe₂, (CH₂)₂NHMe, (CH₂)₂NMe₂, CH₂CO₂Me, (CH₂)₂CO₂Me, CH₂CO₂Et, (CH₂)₂CO₂Et, CH₂CO₂Pr, (CH₂)₂CO₂Pr, CH₂CO₂iPr, (CH₂)₂CO₂iPr, CH₂CO₂tBu, (CH₂)₂CO₂tBu, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂ isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, cyclohexyl, 1-cyclohexenyl, 2-cyclohexenyl, 3-cyclohexenyl, phenyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one,
R¹³ and R¹⁴ moreover together represent ═CR¹⁸R¹⁹, ═O, ═S, ═NOR²⁰,
R¹⁸, R¹⁹ and R²⁰ independently of one another represent hydrogen, methyl, ethyl, n-propyl, i-propyl, n-, i-, s-, t-butyl, trifluoromethyl, phenyl, cyclopropyl.

5. A method according to claim 1, where R¹ and R² each represent methyl.

6. A method according to claim 2, where A¹ and A² each represent cycle A-2.

7. A method according to claim 2, where R¹ and R² each represent methyl and A¹ and A² each represent cycle A-2.

8. A Bisoxime of formula (I-1) 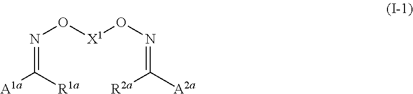
in which
R^{1a} and R^{2a} independently of one another represent halogen, C₁-C₈-alkyl, C₃-C₈-cycloalkyl, C₁-C₈-haloalkyl, OH, C₁-C₈-alkoxy, (C₁-C₆-alkoxy)carbonyl, SH, C₁-C₈-alkylthio, C₁-C₈-alkylsulphinyl, C₁-C₈-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₈-alkyl)amine, (C₁-C₈-alkyl)amine, C₂-C₈-alkenyl, C₂-C₈-alkynyl, in each case optionally substituted phenyl, benzyl, heterocyclyl or hetaryl,
A^{1a} and A^{2a} independently of one another represent one of the cycles below 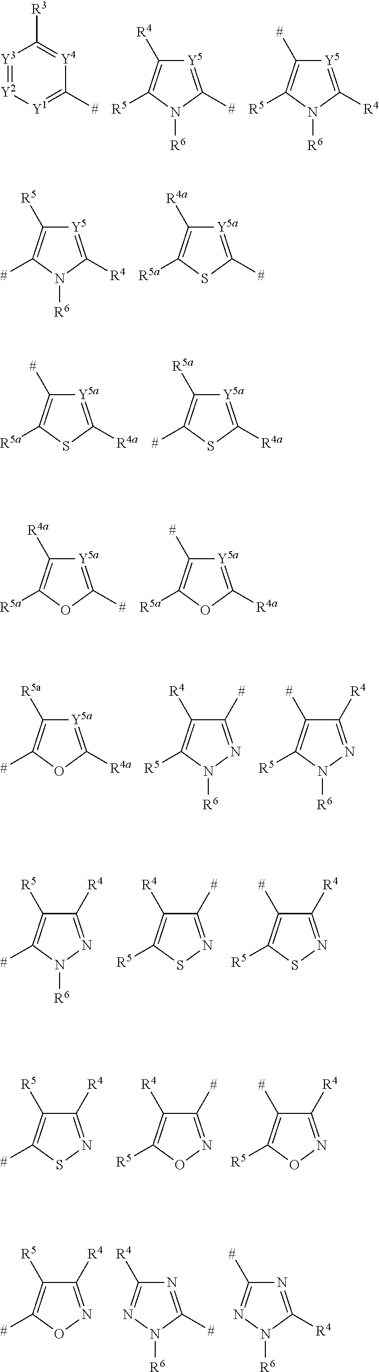 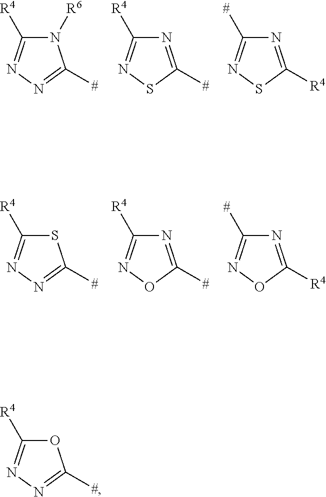
where A^{1a} and A^{2a} do not simultaneously represent optionally substituted phenyl,
Y¹ represents N or CR⁶,
Y² represents N or CR⁷,
Y³ represents N or CR⁸,
Y⁴ represents N or CR⁹,
Y⁵ represents N or CR¹⁰,
Y^{5a} represents N or CR^{10a}, where at least one substituent R^{4a} or R^{5a} or R^{10a} does not represent hydrogen if Y^{5a} represents CR^{10a},
R³, R⁴, R⁵, R⁶, R⁷, R⁸, R⁹ and R¹⁰ independently of one another represent hydrogen, halogen, cyano, nitro, OR¹¹, SR¹¹, SOR¹¹, SO₂R¹¹, SO₂N(R¹¹)₂, COR¹¹, NR¹¹CO₂R¹², N(R¹¹)₂, NR¹¹COR¹¹, NR¹¹SO₂R¹¹, OCON(R¹¹)₂, O(CO)R¹¹, CON(R¹¹)₂, CO₂R¹¹, (CH₂)ₘOR¹¹, (CH₂)ₘSR¹¹, (CH₂)ₘN(R¹¹)₂, (CH₂)ₘCO₂R¹², (CH₂)ₘNR¹¹CO₂R¹², C₁-C₈-alkyl, C₂-C₈-alkenyl, C₂-C₈-alkynl, C₁-C₈-haloalkyl, C₃-C₈-cycloalkyl, C₃-C₈-cycloalkenyl, tri-C₁-C₄-alkyl-silyl, phenyl-(di-C₁-C₄-alkyl)-silyl substituted or unsubstituted phenyl or hetaryl, substituted or unsubstituted benzyl or —CH₂-hetaryl,
moreover represent substituted C₂-C₈-alkenyl or C₂-C₈-alkynyl,
moreover in each case two adjacent radicals R⁶, R⁷ or R⁷, R⁸ or R⁸, R³ or R³, R⁹ or R⁴, R⁵ or R⁵, R¹⁰ or R⁴, R¹⁰ together with the carbon atoms to which they are attached form an unsubstituted or substituted unsaturated or fully or partially saturated heterocyclic or carbocyclic 5- to 7-membered ring,
R^{4a}, R^{5a} and R^{10a} have the meanings of R⁴, R⁵ and R¹⁰, respectively, where at least one substituent R^{4a} or R^{5a} or R^{10a} does not represent hydrogen,
X¹ represents one of the groupings X-1 to X-10 below 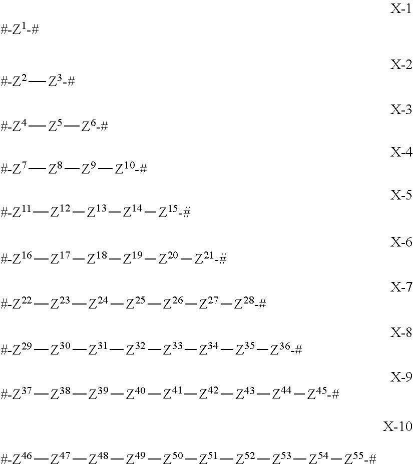
with the proviso that X¹ does not represent pyrimidine-2,4-diyl or pyrimidine-4,6-diyl.

9. A bisoxime of formula (I-1) according to claim 8, where R^{1a} and R^{2a} independently of one another represent chlorine, bromine, iodine, C₁-C₆-alkyl, C₃-C₆-cycloalkyl, C₁-C₆-haloalkyl having 1 to 13 fluorine, chlorine and/or bromine atoms, OH, C₁-C₆-alkoxy, (C₁-C₄-alkoxy)carbonyl, SH, C₁-C₆-alkylthio, C₁-C₆-alkylsulphinyl, C₁-C₆-alkylsulphonyl, C₁-C₄-alkoxy-C₁-C₄-alkyl, (C₁-C₄-alkyl)thio(C₁-C₄-alkyl), (C₁-C₄-alkyl)sulphinyl(C₁-C₄-alkyl), (C₁-C₄-alkyl)sulphonyl(C₁-C₄-alkyl), amino, di(C₁-C₆-alkyl)amine, (C₁-C₆-alkyl)amine, C₂-C₆-alkenyl, C₂-C₆-alkynyl, represent phenyl, benzyl, heterocyclyl or hetaryl, each of which is optionally substituted by halogen, cyano, C₁-C₄-haloalkyl having 1 to 9 fluorine, chlorine and/or bromine atoms, C₁-C₄-alkyl, C₂-C₄-alkenyl, C₂-C₄-alkynyl, C₁-C₄-alkoxy,
A^{1a} and A^{2a} independently of one another represent one of the cycles A-1 to A-43 below: 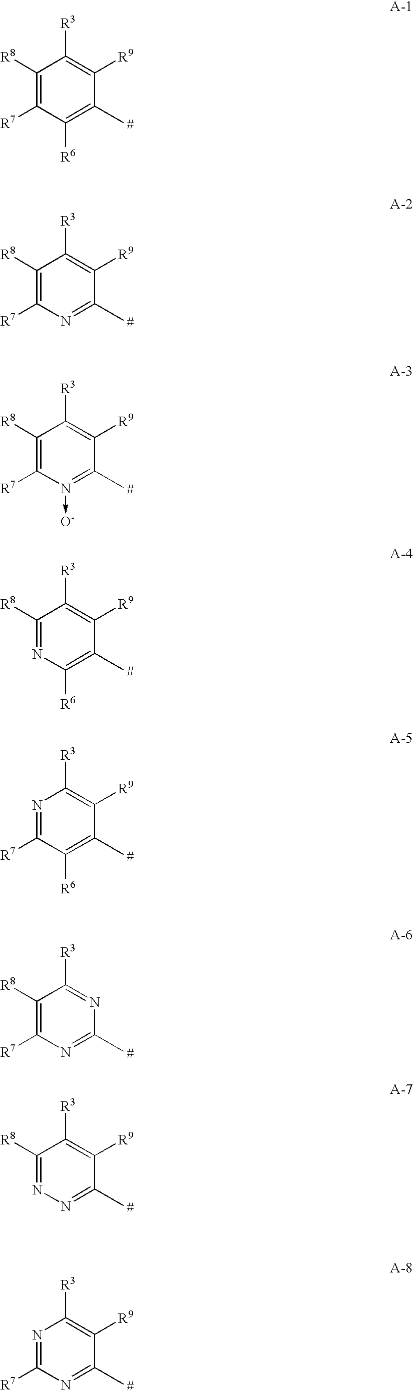 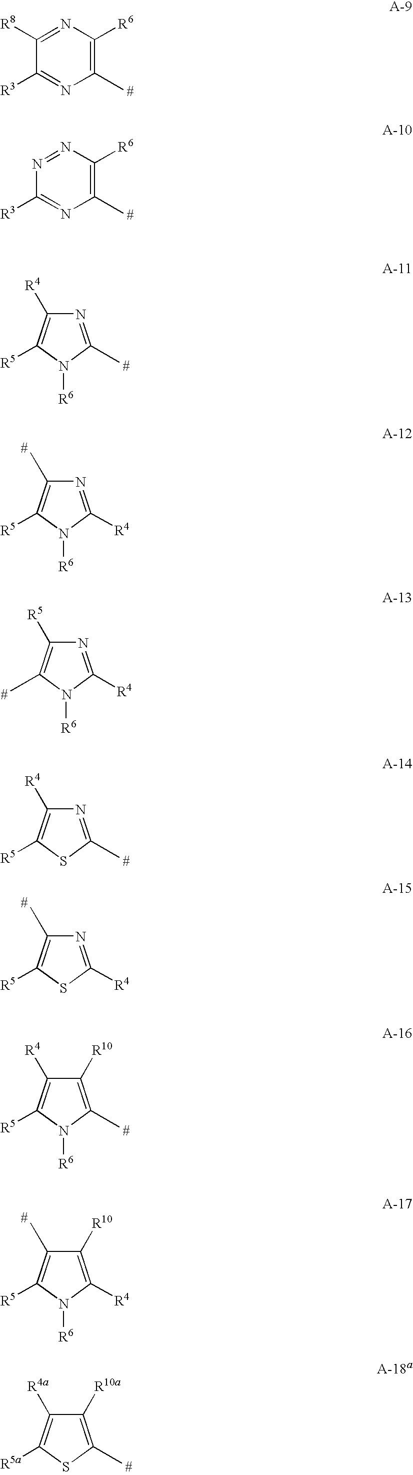 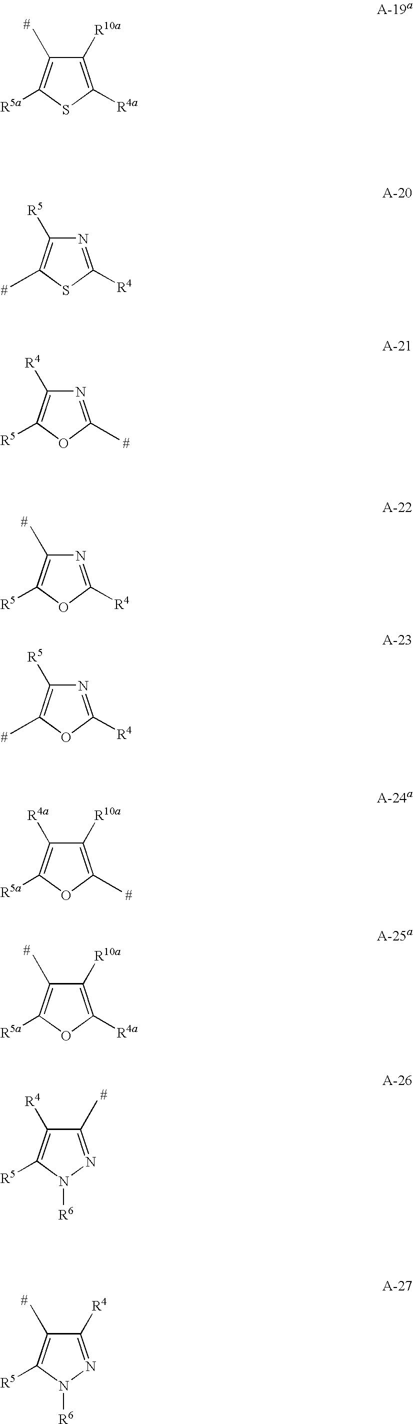 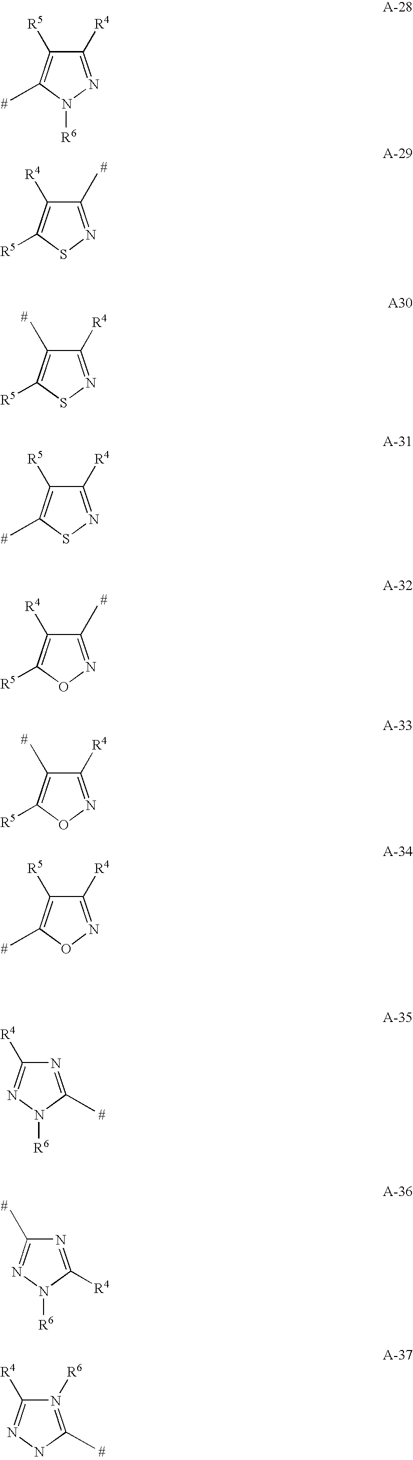 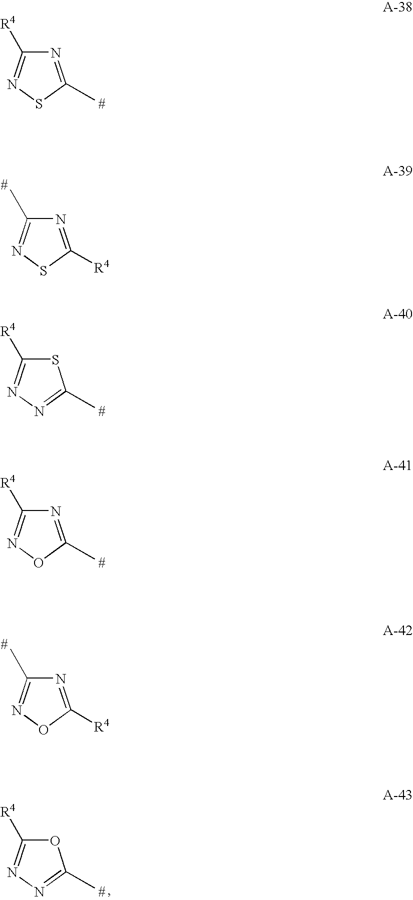
where A^{1a} and A^{2a} do not simultaneously represent the cycle A-1 and where in A-18^{a}, A-19^{a}, A-24^{a} and A-25^{a} at least one substituent R^{4a} or R^{5a} or R^{10a} does not represent hydrogen,
R^{4a}, R^{5a} and R^{10a} have the meanings of R⁴, R⁵ and R¹⁰, respectively, where at least one substituent R^{4a} or R^{5a} or R^{10a} does not represent hydrogen,
with the proviso that X¹ does not represent pyrimidine-2,4-diyl or pyrimidine-4,6-diyl.

10. A Bisoxime of formula (I-1) according to claim 9, where R^{1a} and R^{2a} independently of one another represent chlorine, bromine, C₁-C₄-alkyl, cyclopropyl, cyclobutyl, cyclopentyl, cyclohexyl, C₁-C₄-haloalkyl having 1 to 9 fluorine, chlorine and/or bromine atoms, OH, C₁-C₄-alkoxy, (C₁-C₄-alkoxy)carbonyl, SH, C₁-C₄-alkylthio, C₁-C₄-alkylsulphinyl, C₁-C₄-alkylsulphonyl, C₁-C₂-alkoxy-C₁-C₂-alkyl, (C₁-C₂-alkyl)thio(C₁-C₂-alkyl), (C₁-C₂-alkyl)sulphinyl(C₁-C₂-alkyl), (C₁-C₂-alkyl)sulphonyl(C₁-C₂-alkyl), amino, di(C₁-C₄-alkyl)amine, (C₁-C₄-alkyl)amine, C₂-C₄-alkenyl, C₂-C₄-alkynyl, represent phenyl, benzyl, thienyl, piperazinyl or morpholinyl, each of which is optionally substituted by fluorine, chlorine, bromine, cyano, trifluoromethyl, difluoromethyl, trichloromethyl, dichloromethyl, C₁-C₄-alkyl, C₁-C₄-alkoxy,
A^{1a} and A^{2a} independently of one another represent one of the cycles A-1, A-2, A-4, A-5, A-6, A-7, A-8, A-9, A-10, A-11, A-12, A-13, A-14, A-15, A-16, A-17, A-18^{a}, A-19^{a}, A-20, A-21, A-22, A-23, A-24^{a}, A-25^{a}, A-26, A-27, A-28, A-29, A-30, A-31, A-32, A-33 or A-34 where A^{1a} and A^{2a} do not simultaneously represent the cycle A-1 and where in A-18^{a}, A-19^{a}, A-24^{a} and A-25^{a} at least one substituent R^{4a} or R^{5a} or R^{10a} does not represent hydrogen,
R^{4a}, R^{5a} and R^{10a} have the meanings of R⁴, R⁵ and R¹⁰, respectively, where at least one substituent R^{4a} or R^{5a} or R^{10a} does not represent hydrogen,
with the proviso that X¹ does not represent pyrimidine-2,4-diyl or pyrimidine-4,6-diyl.

11. A Bisoxime of the formula (I-1) according to claim 9, where
R^{1a} and R^{2a} independently of one another represent chlorine, methyl, ethyl, n-propyl, i-propyl, n-, i-, s- or t-butyl, cyclopropyl, cyclopentyl, cyclohexyl, trifluoromethyl, difluoro-methyl, trichloromethyl, pentafluoroethyl, heptafluoro-n-propyl, heptafluoro-i-propyl, OH, methoxy, ethoxy, n-propoxy, i-propoxy, n-, i-, s- or t-butoxy, methoxycarbonyl, ethoxycarbonyl, SH, methylthio, ethylthio, n-propylthio, i-propylthio, n-, i-, s- or t-butylthio, methylsulphinyl, ethylsulphinyl, n-propylsulphinyl, i-propylsulphinyl, n-, i-, s- or t-butylsulphinyl, methylsulphonyl, ethylsulphonyl, n-propylsulphonyl, i-propylsulphonyl, n-, i-, s- or t-butylsulphonyl, methoxymethyl, methoxyethyl, ethoxyethyl, ethoxymethyl, methylthiomethyl, methylthioethyl, ethylthioethyl, ethylthiomethyl, methylsulphonylmethyl, methylsulphonylethyl, ethylsulphonylethyl, ethylsulphonylmethyl, amino, di(C₁-C₄-alkyl)amine, (C₁-C₄-alkyl)amine, C₂-C₄-alkenyl, C₂-C₄-alkynyl, represent phenyl, benzyl, thienyl, piperazinyl or morpholinyl each of which is optionally substituted by fluorine, chlorine, bromine, halogen, cyano, trifluoromethyl, methyl, ethyl, n-propyl, i-propyl, n-, i-, s-, t-butyl, methoxy, ethoxy, n-propoxy, i-propoxy, n-, i-, s-, t-butoxy,
A^{1a} and A^{2a} independently of one another represent one of the cycles A-1, A-2, A-4, A-5, A-6, A-7, A-8, A-9, A-10, A-11, A-14, A-15, A-16, A-18^{a}, A-20, A-21, A-24^{a}, A-26, A-30 or A-33 where A^{1a} and A^{2a} do not simultaneously represent the cycle A-1 and where in A-18^{a} and A-24^{a} at least one substituent R^{4a} or R^{5a} or R^{10a} does not represent hydrogen,
R^{4a}, R^{5a} and R^{10a} have the meanings of R⁴, R⁵ and R¹⁰, respectively, where at least one substituent R^{4a} or R^{5a} or R^{10a} does not represent hydrogen,
with the proviso that X¹ does not represent pyrimidine-2,4-diyl or pyrimidine-4,6-diyl.

12. A Bisoxime of formula (I-1) according to claim 8, where R^{1a} and R^{2a} each represent methyl.

13. A Bisoxime of formula (I-1) according to claim 9, where A^{1a} and A^{2a} each represent the cycle A-2.

14. A Bisoxime of formula (I-1) according to claim 9, where R^{1a} and R^{2a} each represent methyl and A^{1a} and A^{2a} each represent the cycle A-2.

15. A composition for controlling unwanted microorganisms and/or insects, comprising at least one bisoxime of the formula (I-1) according to claim 8, and an extender and/or a surfactant.

16. A method for controlling unwanted microorganisms and/or insects comprising using a bisoxime of claim 8.

17. Method for controlling unwanted microorganisms, comprising applying a bisoxime of the formula (I-1) according to claim 8 to the microorganisms and/or a habitat thereof.

18. A process for preparing a composition for controlling unwanted microorganisms, comprising mixing a bisoxime of formula (I-1) according to claim 8 with an extender and/or a surfactant.

19. A composition for treating transgenic plants comprising a bisoxime of claim 8.

20. A process for preparing a bisoxime of formula (I) according to claim 1, comprising reacting:
(A-1) a acyl(het)aryl of formula (II) 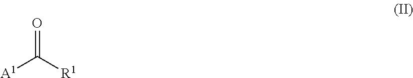
with a acyl(het)aryl of formula (IIa) 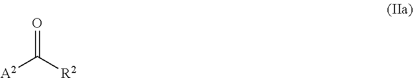
and with a bishydroxylamine (and/or a bishydrohalide thereof) of formula (III) 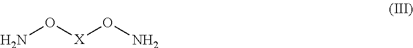
if appropriate in the presence of a catalyst and if appropriate in the presence of a diluent,
or
(A-2) an acyl(het)aryl of formula (II) 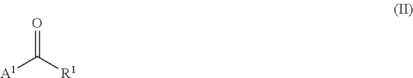
initially with a bishydroxylamines (and/or a bishydrohalide thereof) of formula (III) 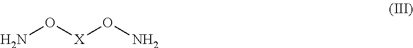
to give a hydroxylamine or a hydrohalide thereof of formula (XII) 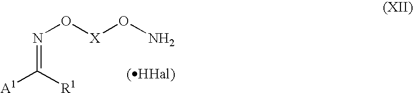
and then with an acyl(het)aryl of formula (IIa) 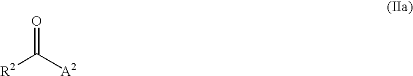
if appropriate in the presence of a catalyst and if appropriate in the presence of a diluent,
or
(B) a free oxime of formula (V) 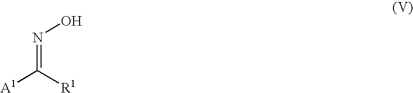
with a free oxime of formula (Va) 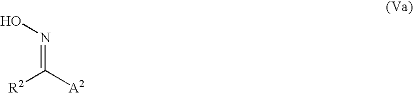
and with a bifunctional compound of formula (IV) 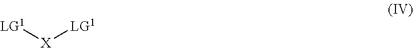
in which
LG¹ represents halogen or sulphonate, where the two LG¹ can be identical or different, and
if appropriate in the presence of a catalyst, if appropriate in the presence of an acid binder and if appropriate in the presence of a diluent,
or
(C) a free oxime of formula (V) 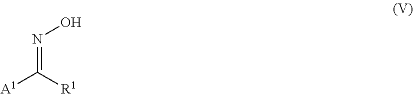
or a free oxime of formula (Va) 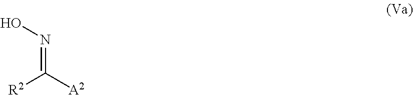
initially with a bifunctional compound of formula (IV) 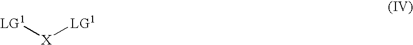
in which
LG¹ represents halogen or sulphonate, where the two LG¹ can be identical or different,
if appropriate in the presence of a catalyst, if appropriate in the presence of an acid binder and if appropriate in the presence of a diluent,
giving an intermediate of the formula (VI) 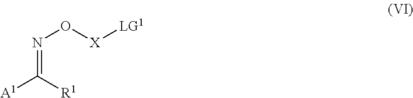
or an intermediate of formula (VIa) 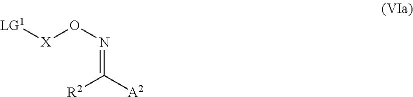
which are then reacted with a free oxime of the formula (Va) or (V),
if appropriate in the presence of a catalyst, if appropriate in the presence of an acid binder and if appropriate in the presence of a diluent,
or that a bisoxime of formula (Ia) 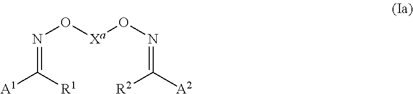
in which
X^{a} represents one of the groupings X-3 to X-10 with the proviso that exactly one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴ represents 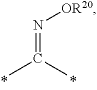
are obtained by reacting
(D) a bisoxime of formula (Ib) 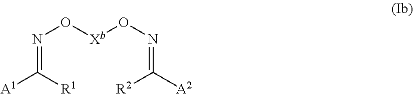
in which
X^{b} represents one of the groupings X-3 to X-10 with the proviso that exactly one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴ represents 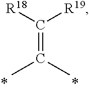
initially with ozone, if appropriate in the presence of a catalyst and if appropriate in the presence of a diluent, and then reacting the bisoximes, obtained as intermediates, of formula (Ic) 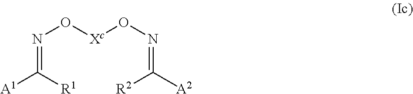
in which
X^{c} represents one of the groupings X-3 to X-10 with the proviso that exactly one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴ represents 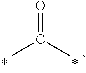
with a free oxime (or its hydrohalide) of formula (VII)
H₂N—OR²⁰ (VII)
if appropriate in the presence of a catalyst, if appropriate in the presence of an acid binder and if appropriate in the presence of a diluent,
or that a bisoxime of formula (Id) 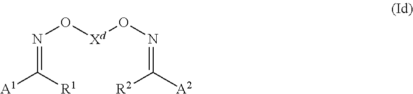
in which
X^{d} represents one of the groupings X-3 to X-10 with the proviso that at least one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴ represents 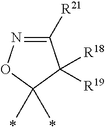
or represents 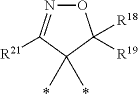
in which
R²¹ represents hydrogen, C₁-C₈-alkyl, C₂-C₈-alkenyl, C₂-C₈-alkynyl, in each case optionally substituted phenyl, benzyl, heterocyclyl or hetaryl,
are obtained by reacting
(E) a bisoximes of formula (Ib) 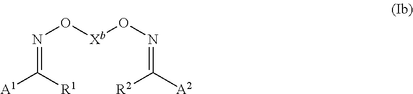
with an aldoxime of formula (VIII) 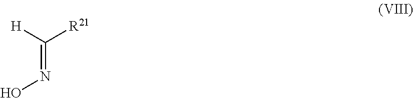
in the presence of N-chlorosuccinimide, if appropriate in the presence of a catalyst and if appropriate in the presence of a diluent,
or that a bisoxime of formula (Ie) 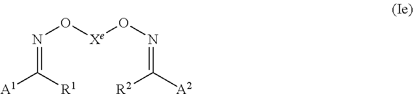
in which
X^{e} represents one of the groupings X-3 to X-10 with the proviso that at least one of the radicals Z⁵, Z⁸, Z⁹, Z¹² to Z¹⁴, Z¹⁷ to Z²⁰, Z²³ to Z²⁷, Z³⁰ to Z³⁵, Z³⁸ to Z⁴⁴, Z⁴⁷ to Z⁵⁴ represents 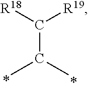
are obtained by reacting
(F) a bisoxime of formula (Ib) 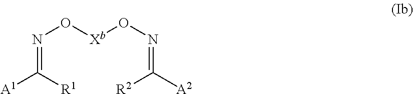
with hydrogen or a hydrogen source, if appropriate in the presence of a catalyst and if appropriate in the presence of a diluent.

21. An Acyl(het)aryl of formulae (II-1), (II-2) and (II-3)

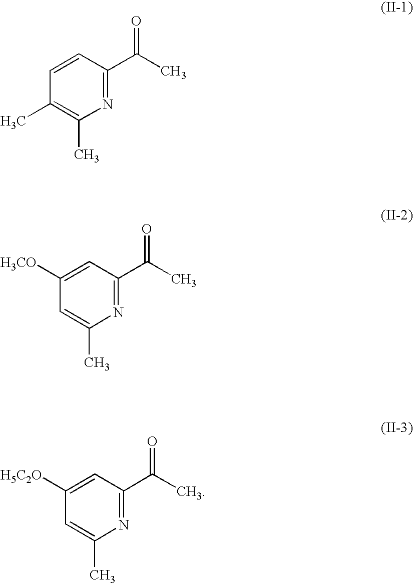

22. A Bishydroxylamine of formula (IIIa) 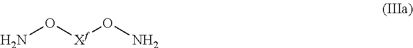
in which
X^{f} represents one of the groupings X-1 to X-10 below 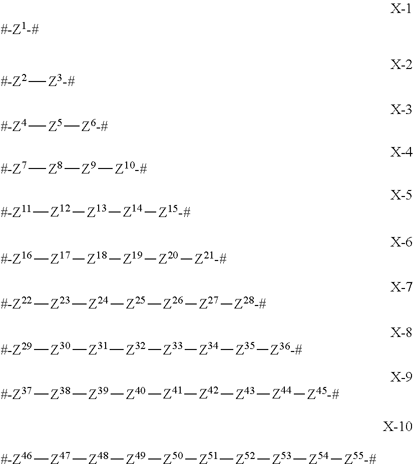
except for compounds of the formula (IIa), in which
X^{f} represents a saturated or unsaturated aliphatic, straight-chain or branched hydrocarbon radical having 2 to 12 carbon atoms which may be interrupted by 1 to 3 non-adjacent oxygen atoms or represents —SiMe₂-, —SitBu₂-, —Si(Me)tBu-, —SiH₂—, —C(═O)—C(═O)—, (1-phenyl)ethane-1,2-diyl, —CH(CO₂Et)-CH₂—, —CH₂—CH(OH)—CH₂—, —CH₂—CHCl—CH₂, —CH₂—N(NO₂)—CH₂, —CH(CO₂Et)-CH₂—CH(CO₂Et)-, —C(═O)—CH(OH)—CH₂—C(═O)—, —C(═O)—(CH₂)₂—C(═O)—, —CH(CO₂H)—(CH₂)₂—CH(CO₂H)—, —(CH₂)₂—CHBr—(CH₂)₂—, —CH₂—N(NO₂)—CH₂—N(NO₂)—CH₂—, —(CH₂)₂—N[(CH₂)₄NH₂]—(CH₂)₂—, —CH(CO₂H)—(CH₂)₃—CH(CO₂H)—, —CH(CO₂H)—(CH₂)₄—CH(CO₂H)—, —CH₂—C(═O)—NH—NH—C(═O)—CH₂—, —CH₂—CH(CO₂H)—NH—C(═O)—C(NO₂)═CH—CH₂—, —CH₂—CH(OH)—CH═N—CH₂—CH(OH)—CH₂, —CH(CO₂H)—(CH₂)₆—CH(CO₂H)—, —(CH₂)₂—NH—(CH₂)₄—NH—(CH₂)₂—, —C(═O)—NH—(CH₂)₆—NH—C(O)—, —CH₂—C(═O)—NH—(CH₂)₂—NH—C(═O)—CH₂—, —CH₂—C(═O)—NH—NH—C(═O)—CH₂—O—NH—C(═O)—CH₂—, or represents one of the groupings below

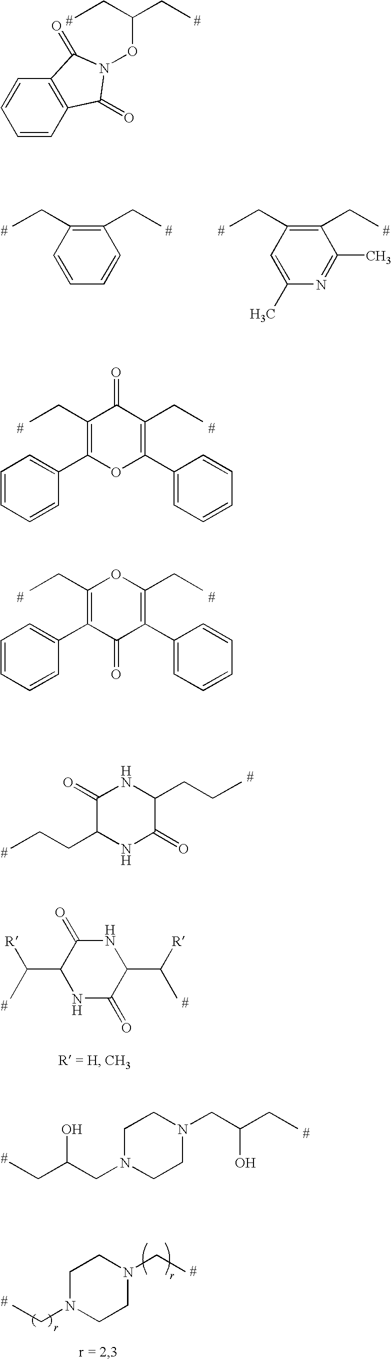

23. An intermediate of the formulae (VI) and (VIa) capable of being involved in a method of claim 1 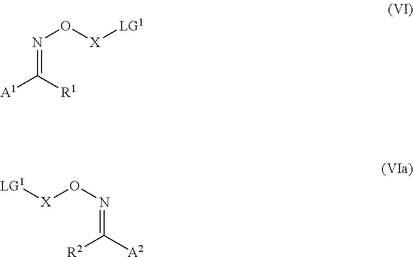
in which
LG¹ represents halogen or sulphonate, where the two LG¹ can be identical or different.

24. A bishydroxyphthalimide derivative of formula (XIa) capable of being involved in a method of claim 1 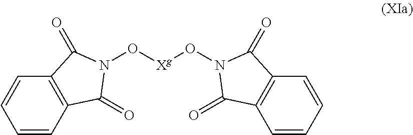
in which
X^{g} has the meanings of X
except for compounds of the formula (XIa), in which
X^{g} represents —C(═O)—, —C(═NH)—, —(CH₂)₂—, —(CH₂)₃—, —(CH₂)₄—, —(CH₂)₅—, —(CH₂)₆—, —C(═O)—C(═O)—, (1-phenyl)ethane-1,2-diyl, —CH₂—CCl₂—, —CH₂—CH(CH₂—ON═CH₂)— —CH₂—CH(CH₂OH)—, —CH₂—CH(OH)—CH₂—, —CH(CH₃)—(CH₂)₂—, —CH(CO₂Et)-CH₂—CH(CO₂Et)-, —CH(CO₂Et)-(CH₂)₂—CH(CO₂Et)-, —CH(CO₂Et)-(CH₂)₃—CH(CO₂Et)-, —CH(CO₂Et)-(CH₂)₄—CH(CO₂Et)-, —(CH₂)₂—O—(CH₂)₂—O—(CH₂)₂—, —(CH₂)₂—O—(CH₂)₂—, —CH(CO₂Et)-(CH₂)₆—CH(CO₂Et)-, —CH₂—C(═O)—NH—(CH₂)₂—NH—C(═O)—CH₂—,
or represents one of the groupings below

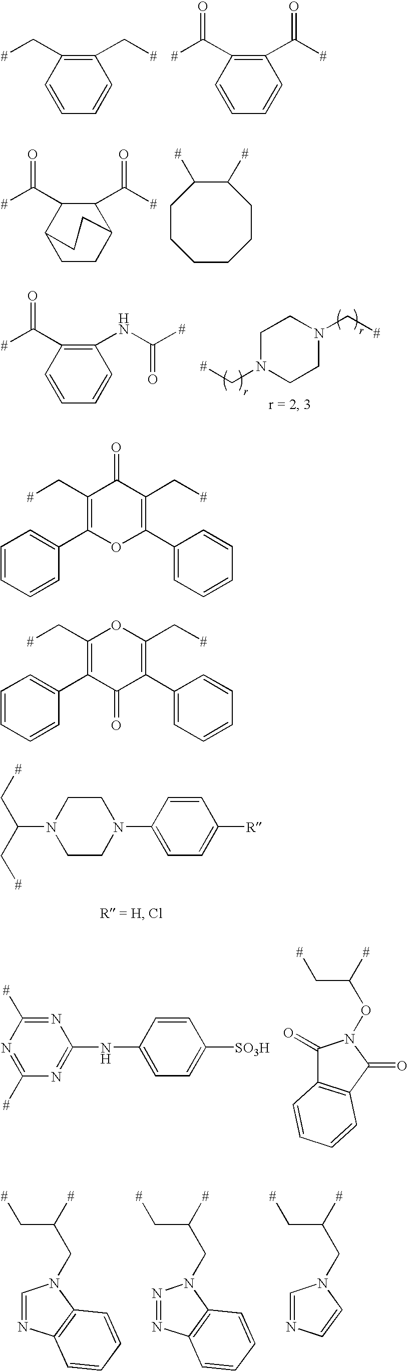

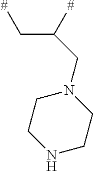

25. A method for using a hydroxylamine or a hydrohalide thereof of formula (XII) which could be relevant to a method of claim 1, comprising using said compound of formula (XII) 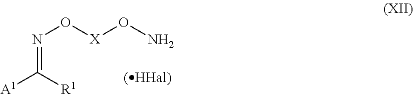
for controlling unwanted microorganisms in crop protection and/or in the protection of materials.

26. A hydroxylamine and/or a hydrohalide thereof of the formula (XIIa) capable of being relevant in a method of claim 1, 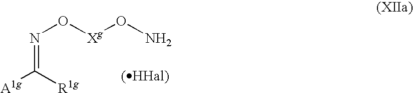
in which
R^{1g}, A^{1g} and X^{g} each have the meanings given for R¹, A¹ and X, respectively, where
a) A^{1g} does not represent phenyl, 4-fluorophenyl, 4-chlorophenyl or 2,4-dichlorophenyl if R^{1g} represents hydrogen and X^{g} represents —(CH₂)₄—, or
b) A^{1g} does not represent phenyl, 4-fluorophenyl, 4-chlorophenyl, 2,4-dichlorophenyl, 3,4-dichlorophenyl, 2-hydroxyphenyl, 2-hydroxy-5-bromophenyl, 3-{[(2S)-2-hydroxypropyl]oxy}phenyl, 2-hydroxy-3,5-bis-tert-butylphenyl, 2-hydroxy-3-methylthiophenyl, 2-hydroxy-3-methoxyphenyl or 4-nitrophenyl if R^{1g} represents hydrogen and X^{g} represents —(CH₂)₂—.

27. A composition for controlling unwanted microorganisms and/or insects, comprising at least one hydroxylamine and/or a hydrohalide of formula (XIIa) according to claim 26, and an extender and/or a surfactant.

28. A hydroxylamine and/or hydrohalide thereof of formula (XIIa) according to claim 26 which is useful for controlling unwanted microorganisms and insects.

29. A method for controlling unwanted microorganisms, comprising applying a hydroxylamine and/or a hydrohalide thereof of formula (XIIa) according to claim 26 to microorganisms and/or a habitat thereof.

30. A method for preparing a composition for controlling unwanted microorganisms, comprising mixing a hydroxylamine or a hydrohalide thereof of formula (XIIa) according to claim 26 with an extender and/or a surfactant.

31. A hydroxylamine and/or hydrohalides thereof of the formula (XIIa) according to claim 26, which is useful for treating transgenic plants.

32. A process for preparing a hydroxylamine and/or a hydrohalide thereof of formula (XIIa) according to claim 26, comprising reacting:
(A-2) a acyl(het)aryl of formula (II) 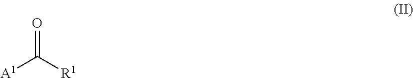
with a bishydroxylamine and/or a bishydrohalide thereof of formula (III) 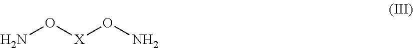
if appropriate in the presence of a catalyst and if appropriate in the presence of a diluent,
where
a) A¹ does not represent phenyl, 4-fluorophenyl, 4-chlorophenyl or 2,4-dichlorophenyl, if R¹ represents hydrogen and X represents —(CH₂)₄—, or
b) A¹ does not represent phenyl, 4-fluorophenyl, 4-chlorophenyl, 2,4-dichlorophenyl, 3,4-dichlorophenyl, 2-hydroxyphenyl, 2-hydroxy-5-bromophenyl, 3-{[(2S)-2-hydroxypropyl]oxy}phenyl, 2-hydroxy-3,5-bis-tert-butylphenyl, 2-hydroxy-3-methylthiophenyl, 2-hydroxy-3-methoxyphenyl or 4-nitrophenyl, if R¹ represents hydrogen and X represents —(CH₂)₂—.
